(19) United States
(12) Patent Application Publication
(10) Pub. No.: US 2009/0317799 A1
(43) Pub. Date: Dec. 24, 2009

(54) Variants at chr8q24.21 confer risk of cancer

(75) Inventors: Laufey Amundadottir, Gaithersburg, MD (US); Julius Gudmundsson, Reykjavik (IS); Patrick Sulem, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 11/920,590

(22) PCT Filed: May 18, 2006
(86) PCT No.: PCT/IS2006/000012

§ 371 Date: Mar. 31, 2008

Related U.S. Application Data
(60) Provisional application No. 60/682,147, filed on May 18, 2005.
(60) Provisional application No. 60/795,768, filed on Apr. 28, 2006.

(51) Int. Cl. C12Q 1/68

(52) U.S. Cl. 435/6

(57) ABSTRACT

A locus on chromosome 8q24.21 has been demonstrated to play a major role in particular forms of cancer. It has been discovered that certain markers and haplotypes are indicative of a susceptibility to particular cancers. Diagnostic applications for identifying susceptibilty to cancer are described.

### RELATED APPLICATIONS

This application relates to U.S. Provisional Application No. 60/682,147, filed on May 18, 2005, and U.S. Provisional Application No. 60/795,768, filed on Apr. 28, 2006. The entire teachings of the above applications are incorporated herein by reference.

### BACKGROUND OF THE INVENTION

Cancer, the uncontrolled growth of malignant cells, is a major health problem of the modern medical era and is one of the leading causes of death in developed countries. In the United States, one in four deaths is caused by cancer (Jemal, A. et al., CA Cancer J. Clin. 52:23-47 (2002)).

The incidence of prostate cancer has dramatically increased over the last decades and prostate cancer is now a leading cause of death in the United States and Western Europe (Peschel, R. E. and J. W. Colberg, Lancet 4:233-41 (2003); Nelson, W. G. et al., N. Engl. J. Med. 349(4):366-81 (2003)). Prostate cancer is the most frequently diagnosed noncutaneous malignancy among men in industrialized countries, and in the United States, 1 in 8 men will develop prostate cancer during his life (Simard, J. et al., Endocrinology 143(6):2029-40 (2002)). Although environmental factors, such as dietary factors and lifestyle-related factors, contribute to the risk of prostate cancer, genetic factors have also been shown to play an important role. Indeed, a positive family history is among the strongest epidemiological risk factors for prostate cancer, and twin studies comparing the concordant occurrence of prostate cancer in monozygotic twins have consistently revealed a stronger hereditary component in the risk of prostate cancer than in any other type of cancer (Nelson, W. G. et al., N. Engl. J. Med. 349(4):366-81 (2003); Lichtenstein P. et. al., N. Engl. J. Med. 343(2):78-85 (2000)). In addition, an increased risk of prostate cancer is seen in 1^{st} to 5^{th} degree relatives of prostate cancer cases in a nation wide study on the familiality of all cancer cases diagnosed in Iceland from 1955-2003 (Amundadottir et. al., PLoS Medicine 1(3):e65 (2004)). The genetic basis for this disease, emphasized by the increased risk among relatives, is further supported by studies of prostate cancer among particular populations: for example, African Americans have among the highest incidence of prostate cancer and mortality rate attributable to this disease: they are 1.6 times as likely to develop prostate cancer and 2.4 times as likely to die from this disease than European Americans (Ries, L. A. G. et al., NIH Pub. No. 99-4649 (1999)).

An average 40% reduction in life expectancy affects males with prostate cancer. If detected early, prior to metastasis and local spread beyond the capsule, prostate cancer can be cured (e.g., using surgery). However, if diagnosed after spread and metastasis from the prostate, prostate cancer is typically a fatal disease with low cure rates. While prostate-specific antigen (PSA)-based screening has aided early diagnosis of prostate cancer, it is neither highly sensitive nor specific (Punglia et.al., N Engl J Med. 349(4):335-42 (2003)). This means that a high percentage of false negative and false positive diagnoses are associated with the test. The consequences are both many instances of missed cancers and unnecessary follow-up biopsies for those without cancer. As many as 65 to 85% of individuals (depending on age) with prostate cancer have a PSA value less than or equal to 4.0 ng/mL, which has traditionally been used as the upper limit for a normal PSA level (Punglia et.al., N Engl J Med. 349(4):335-42 (2003); Cookston, M. S., Cancer Control 8(2):133-40 (2001); Thompson, I. M. et al., N Engl J Med. 350:2239-46 (2004)). A significant fraction of those cancers with low PSA levels are scored as Gleason grade 7 or higher, which is a measure of an aggressive prostate cancer. Id.

In addition to the sensitivity problem outlined above, PSA testing also has difficulty with specificity and predicting prognosis. PSA levels can be abnormal in those without prostate cancer. For example, benign prostatic hyperplasia (BPH) is one common cause of a false-positive PSA test. In addition, a variety of noncancer conditions may elevate serum PSA levels, including urinary retention, prostatitis, vigorous prostate massage and ejaculation. Id.

Subsequent confirmation of prostate cancer using needle biopsy in patients with positive PSA levels is difficult if the tumor is too small to see by ultrasound. Multiple random samples are typically taken but diagnosis of prostate cancer may be missed because of the sampling of only small amounts of tissue. Digital rectal examination (DRE) also misses many cancers because only the posterior lobe of the prostate is examined. As early cancers are nonpalpable, cancers detected by DRE may already have spread outside the prostate (Mistry K. J., Am. Board Fam. Pract. 16(2):95-101 (2003)).

Thus, there is clearly a great need for improved diagnostic procedures that would facilitate early-stage prostate cancer detection and prognosis, as well as aid in preventive and curative treatments of the disease. In addition, there is a need to develop tools to better identify those patients who are more likely to have aggressive forms of prostate cancer from those patients that are more likely to have more benign forms of prostate cancer that remain localized within the prostate and do not contribute significantly to morbidity or mortality. This would help to avoid invasive and costly procedures for patients not at significant risk.

Breast cancer is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No universally successful method for the treatment or prevention of breast cancer is currently available. Management of breast cancer currently relies on a combination of early diagnosis (e.g., through routine breast screening procedures) and aggressive treatment, which may include one or more of a variety of treatments, such as surgery, radiotherapy, chemotherapy and hormone therapy. The course of treatment for a particular breast cancer is often selected based on a variety of prognostic parameters including an analysis of specific tumor markers. See, e.g., Porter-Jordan and Lippman, Breast Cancer 8:73-100 (1994).

Although the discovery of BRCA1 and BRCA2 were important steps in identifying key genetic factors involved in breast cancer, it has become clear that mutations in BRCA1 and BRCA2 account for only a fraction of inherited susceptibility to breast cancer (Nathanson, K. L. et al., Human Mol. Gen. 10(7):715-720 (2001); Anglican Breast Cancer Study Group. Br. J. Cancer 83(10):1301-08 (2000); and Syrjakoski K. et.al., J. Natl. Cancer Inst. 92:1529-31 (2000)). In spite of considerable research into therapies for breast cancer, breast cancer remains difficult to diagnose and treat effectively, and the high mortality observed in breast cancer patients indicates that improvements are needed in the diagnosis, treatment and prevention of the disease.

deCODE has demonstrated an increased risk of breast cancer in 1^{st} to 5^{th} degree relatives of breast cancer cases in a nation wide study of the familiality of all cancers diagnosed in Iceland from 1955-2003 (Amundadottir et.al., PLoS Med. 1(3):e65 (2004); Lichtenstein P. et.al., N. Engl. J. Med. 343(2):78-85 (2000)), where the authors show that breast cancer has one of the highest heritability of all cancers tested in a cohort of close to 45,000 twins.

Lung cancer causes more deaths from cancer worldwide than any other form of cancer (Goodman, G. E., Thorax 57:994-999 (2002)). In the United States, lung cancer is the primary cause of cancer death among both men and women. In 2002, the death rate from lung cancer was an estimated 134,900 deaths, exceeding the combined total for breast, prostate and colon cancer. Id. Lung cancer is also the leading cause of cancer death in all European countries and is rapidly increasing in developing countries. While environmental factors, such as lifestyle factors (e.g., smoking) and dietary factors, play an important role in lung cancer, genetic factors also contribute to the disease. For example, a family of enzymes responsible for carcinogen activation, degradation and subsequent DNA repair have been implicated in susceptibility to lung cancer. Id. In addition an increased risk to familial members outside of the nuclear family has been shown by deCODE geneticists by analysing all lung cancer cases diagnosed in Iceland over 48 years. This increased risk could not be entirely accounted for by smoking indicating that genetic variants may predispose certain individuals to lung cancer (Jonson et.al., JAMA 292(24):2977-83 (2004); Amundadottir et. al., PLoS Med. 1(3):e65 (2004)).

The five-year survival rate among all lung cancer patients, regardless of the stage of disease at diagnosis, is only 13%. This contrasts with a five-year survival rate of 46% among cases detected while the disease is still localized. However, only 16% of lung cancers are discovered before the disease has spread. Early detection is difficult as clinical symptoms are often not observed until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for this and other cancers, lung cancer remains difficult to diagnose and treat effectively. Accordingly, there is a great need in the art for improved methods for detecting and treating such cancers.

The incidence of malignant melanoma is increasing more rapidly than any other type of human cancer in North America (Armstrong et al., Cancer Surv. 19-20:219-240 (1994)). Although melanoma is curable when identified at an early stage, it requires detection and removal of the primary tumor before it has spread to distant sites. Malignant melanomas have great propensity to metastasize and are notoriously resistant to conventional cancer treatments, such as chemotherapy and □-irradiation. Once metastases have occurred the prognosis is very poor. Thus, early detection of melanoma is of vital importance in melanoma treatment and control.

Studies have demonstrated that genetic factors play an important role in melanoma. Swedish and Icelandic population-based studies report a standardized incidence ratio of approximately 2 in first-degree relatives (Hemminki K., J. Invest. Dermatol. 120(2):217-23 (2003); Amundadottir et al., PLoS Med. 1(3):e65 (2004)). Familial cases tend to have earlier ages of onset and a higher risk of multiple primary tumors, further suggesting a genetic component (see, e.g., Tucker M., Oncogene 22(20):3042-52 (2003)). An interaction of genetic and environmental risk factors is likely to play a major role in melanoma. However, the molecular and biological mechanisms of how a normal melanocyte transforms into a melanoma cell remains unclear.

Clearly, identification of markers and genes that are responsible for susceptibility to particular forms of cancer (e.g., prostate cancer, breast cancer, lung cancer, melanoma) is one of the major challenges facing oncology today. There is a need to identify means for the early detection of individuals that have a genetic susceptibility to cancer so that more aggressive screening and intervention regimens may be instituted for the early detection and treatment of cancer. Cancer genes may also reveal key molecular pathways that may be manipulated (e.g., using small or large molecule weight drugs) and may lead to more effective treatments regardless of the cancer stage when a particular cancer is first diagnosed.

### SUMMARY OF THE INVENTION

As described herein, a locus on chromosome 8q24.21 has been demonstrated to play a role in cancer (e.g., prostate cancer (e.g., aggressive prostate cancer), breast cancer, lung cancer, melanoma). It has been discovered that particular markers and/or combinations of genetic markers (“haplotypes”) in a specific DNA segment within the locus are indicative of susceptibility to particular cancers.

In one embodiment, the invention is a method of diagnosing a susceptibility to a cancer in a subject, comprising detecting a marker or haplotype associated with LD Block A, wherein the presence of the marker or haplotype is indicative of a susceptibility to the cancer. In particular embodiments, the invention is a method of diagnosing a susceptibility to a cancer selected from the group consisting of prostate cancer, breast cancer, lung cancer and melanoma.

In certain embodiments, the marker or haplotype that is indicative of cancer or a susceptibility to cancer, comprises at least one marker selected from the group consisting of the markers listed in Table 13. In other embodiments, the method comprises detecting a haplotype consisting of at least two of the markers in Table 13.

In one embodiment, the presence of a marker or haplotype (e.g., a marker or haplotype associated with LD Block A) is indicative of a different response rate to a particular treatment modality (e.g., a particular therapeutic agent, antihormonal drug, a chemotherapeutic agent, radiation treatment). Thus, by determining whether a subject carries a marker or haplotype, one can determine whether that subject will respond better to, or worse to, a specific therapeutic, antihormonal drug and/or radiation therapy used to treat cancer.

In one embodiment, the presence of a marker or haplotype (e.g., a marker or haplotype associated with LD Block A) is indicative of a predisposition to a somatic rearrangement of Chr8q24.21 (e.g., one or more of an amplification, a translocation, an insertion and/or deletion) in a tumor or its precursor.

In one embodiment, the marker or haplotype comprises one or more markers associated with Chr8q24.21 in linkage disequilibrium (defined as the square of correlation coefficient, r², greater than 0.2) with one or more markers selected from the group consisting of the markers listed in Table 13.

In one embodiment, the invention is a method of diagnosing a susceptibility to a cancer (e.g., prostate cancer (e.g., aggressive prostate cancer), breast cancer, lung cancer, melanoma) comprising detecting a marker or haplotype associated with Chr8q24.21, wherein the presence of the marker or haplotype is indicative of a susceptibility to cancer.

In one embodiment, the invention is a method of predicting an increased risk for aggressive prostate cancer (e.g., having a Gleason score of 7(4+3) to 10, an increased stage, a worse outcome) in a subject comprising detecting a marker or haplotype associated with LD Block A, wherein the presence of the marker or haplotype is indicative of an increased risk for aggressive prostate cancer. In particular embodiments, the subject has been diagnosed with prostate cancer or has not yet been diagnosed with prostate cancer.

In one embodiment, the marker or haplotype has a relative risk of greater than one, i.e. the marker or haplotype confers increased risk of the cancer (the marker or haplotype is at-risk).

In another embodiment, the marker or haplotype has a relative risk of less than one, i.e. the marker or haplotype confers a decreased risk of the cancer (the marker or haplotype is protective).

In one embodiment, the invention is a kit for assaying a sample (e.g., tissue, blood) from a subject to detect an inherited susceptibility to cancer (e.g., prostate cancer (e.g., aggressive prostate cancer), breast cancer, lung cancer, melanoma). Such kits comprise one or more reagents for detecting a marker or haplotype associated with LD Block A. In a particular embodiment, such reagents comprise at least one contiguous nucleotide sequence that is completely complementary to a region comprising at least one of the markers selected from the group consisting of the markers listed in Table 13. In a particular embodiment, such reagents comprise at least one contiguous nucleotide sequence that is completely complementary to a region comprising the rs1447295 A allele or the DG8S737 −8 allele.

In one embodiment, the invention is a method for diagnosing an increased risk of cancer (e.g., prostate cancer (e.g., aggressive prostate cancer), breast cancer, lung cancer, melanoma) in a subject, comprising screening for a marker or haplotype associated with LD Block A, wherein the marker or haplotype is more frequently present in a subject having the cancer than in a subject not having the cancer, and wherein the presence of the marker or haplotype increases the risk of the subject having the cancer. In particular embodiments, the risk is increased by at least about 5%, or the increase in risk is identified as a relative risk of at least about 1.2.

In one embodiment, the invention is a method for diagnosing a susceptibility to cancer (e.g., prostate cancer (e.g., aggressive prostate cancer), breast cancer, lung cancer, melanoma) in a subject comprising obtaining a nucleic acid sample from a subject and analyzing the nucleic acid sample for the presence or absence of at least one marker or haplotype, wherein the marker or haplotype comprises one or more markers selected from the group consisting of the markers listed in Table 13. In this embodiment, the presence of the marker or haplotype is indicative of a susceptibility to the cancer.

In one embodiment, the invention is a method for diagnosing a susceptibility to cancer (e.g., prostate cancer (e.g., aggressive prostate cancer), breast cancer, lung cancer, melanoma) in a subject, comprising obtaining a nucleic acid sample from the subject and analyzing the nucleic acid sample for the presence or absence of at least one marker or haplotype associated with LD Block A, wherein the presence of the marker or haplotype is indicative of a susceptibility to the cancer. In a particular embodiment, the marker or haplotype comprises one or more markers selected from the group consisting of the markers listed in Table 13. In another embodiment, the marker or haplotype has a relative risk of greater than one and comprises the DG8S737 −8 allele or the rs1447295 A allele.

In one embodiment, the invention is a method for diagnosing a susceptibility to cancer in a subject, comprising analyzing a nucleic acid sample obtained from the subject for the presence of at least one marker or haplotype associated with LD Block A, wherein the presence of the marker or haplotype is indicative of susceptibility to the cancer. In a particular embodiment, the marker or haplotype comprises one or more markers selected from the group consisting of the markers in Table 13. In another embodiment, the marker or haplotype has a relative risk of greater than one and comprises the DG8S737 −8 allele or the rs1447295 A allele. In another embodiment, the subject is of black African ancestry.

In one embodiment of the invention, the cancer is selected from the group consisting of prostate cancer, breast cancer, lung cancer and melanoma. In one preferred embodiment, the cancer is prostate cancer, and the marker or haplotype has a relative risk of at least 1.5. In another embodiment, the prostate cancer is an aggressive prostate cancer as defined by a combined Gleason score of 7(4+3)−10. In another embodiment, the prostate cancer is a less aggressive prostate cancer as defined by a combined Gleason score of 2-7(3+4). In yet another embodiment, the presence of the marker or haplotype is indicative of a more aggressive prostate cancer and/or a worse prognosis. In another embodiment, the cancer is breast cancer, and the marker or haplotype has a relative risk of at least 1.3. In another embodiment, the cancer is lung cancer, and the marker or haplotype has a relative risk of at least 1.3. In yet another embodiment, the cancer is melanoma, and the marker or haplotype has a relative risk of at least 1.5. In another embodiment, the melanoma is malignant cutaneous melanoma.

In another embodiment of the invention, the presence of the marker or haplotype is indicative of a different response rate of the subject to a particular treatment modality.

In another embodiment, the presence of the marker or haplotype is indicative of a predisposition to a somatic rearrangement of Cbr8q24.21 in a tumor or its precursor. In a particular embodiment, the somatic rearrangement is selected from the group consisting of an amplification, a translocation, an insertion and a deletion.

In another embodiment of the invention the marker or haplotype used for diagnosing a susceptibility to cancer comprises one or more markers associated with Chr8q24.21 in strong linkage disequilibrium, as defined by (|D′|>0.8) and/or r²>0.2, with one or more markers selected from the group consisting of the markers in Table 13. In one embodiment, the one or more markers is selected from the group consisting of the markers in Table 13 comprises the rs1447295 A allele or the DG8S737 −8 allele.

In another embodiment, the at least one marker or haplotype for diagnosing a susceptibility to cancer has a relative risk of less than one and comprises rs12542685 allele T and rs7814251 allele C. In another embodiment, the at least one marker or haplotype comprises at least one of the markers shown in Table 13 having a relative risk of less than one. In a preferred embodiment, the cancer is prostate cancer. In another embodiment, the subject is of black African ancestry.

In one embodiment, the present invention pertains to a kit for assaying a sample from a subject to detect a susceptibility to a cancer, wherein the kit comprises one or more reagents for detecting a marker or haplotype associated with LD Block A. In one embodiment, the one or more reagents comprise at least one contiguous nucleotide sequence that is completely complementary to a region comprising at least one of the markers selected from the group consisting of the markers in Table 13. In one embodiment, the cancer is prostate cancer.

In a preferred embodiment, the one or more reagents comprise at least one contiguous nucleotide sequence that is completely complementary to a region comprising the rs1447295 A allele or the DG8S737 −8 allele. In a particular embodiment, the subject is of black African ancestry.

In one embodiment, the invention is a method of diagnosing Chr8q24.21-associated cancer in a subject, comprising detecting the presence of a marker or haplotype (e.g., the markers or haplotypes described herein) associated with Chr8q24.21, wherein the presence of the marker or haplotype is indicative of the Chr8q24.21-associated cancer. In particular embodiments, the Chr8q24.21-associated cancer is Chr8q24.21-associated prostate cancer, Chr8q24.21-associated breast cancer, Chr8q24.21-associated lung cancer or Chr8q24.21-associated melanoma.

In another embodiment, the invention is a method of diagnosing susceptibility to prostate cancer, or an increased risk for prostate cancer (e.g., aggressive prostate cancer), by detecting marker DG8S737 or marker rs1447295, wherein the presence of allele −8 at marker DG8S737 or allele A at marker rs1447295, is indicative of susceptibility to prostate cancer or increased risk for prostate cancer. In a further embodiment, the invention is a method of diagnosing susceptibility to prostate cancer in a human having ancestry that includes African ancestry, by detecting marker DG8S737, wherein the presence of allele −8 at marker DG8S737 is indicative of susceptibility to prostate cancer.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a linkage scan of chromosome 8 depicting a genome wide significant LOD score of 4.0 at chromosome 8q24.

FIG. 2 depicts an association analysis of haplotypes on Chr8q24.21 to prostate cancer using 352 microsatellite markers.

FIGS. 3A and 3B depict the LD structure (HAPMAP) in the area of the haplotype that associates with prostate cancer. Equivalent intervals means that each marker is shown in a sequential order with equal distances between two consecutive markers (FIG. 3A). Actual positions means that the correct interval NCBI Build 34) between any two markers is represented in the figure (FIG. 3B).

FIG. 4 depicts the Icelandic LD structure. Equivalent intervals means that each marker is shown in a sequential order with equal distances between two consecutive markers.

FIG. 5 depicts a schematic identifying known genes mapping to chromosome 8q24.21.

FIG. 6A1-6A31 depicts a genomic DNA sequence from 128.414-128.506 of NCBI Build 34 (SEQ ID NO: 1; Build 34, hg16_chr8:1284140007-128506000. Forward (+) strand). The numbering in FIG. 6, as well as the indicated bp in the tables contained herein, refer to the location within Chromosome 8 in NCBI Build 34.

FIGS. 7A-7D depict a schematic view of linkage and association results, marker density and LD structure in a region on chromosome 8q24.21 for prostate cancer, FIG. 7A shows linkage scan results for chromosome 8q performed with 871 Icelandic prostate cancer patients in 323 extended families. FIG. 7B depicts single marker association results for unrelated prostate cancer cases (case control group 1, n=869), using 358 microsatellites and indels (blue diamonds), distributed over a 10 Mb region. FIG. 7C shows single marker association results for all prostate cancer cases (n=1291), red boxes denote P values for the 63 SNPs and 12 microsatellites added to this region, blue diamonds denote the values for the other markers already typed in this region from 7B. FIG. 7D depicts pairwise LD from the CEU HapMap population (Phase II) for the 600 kb region from FIG. 7C, the gray triangles at the bottom indicate the location of the c-MYC gene and the AW183883 EST discussed in the main text. A scale for r² is provided on the right. Black vertical lines represent the density of microsatellites (FIG. 7B), and microsatellites and SNPs (FIG. 7C) used in the association analysis.

FIG. 8 depicts a phylogenetic network of 46 SNPs and the DG8S737 microsatellite for HapMap samples.

FIGS. 9A-9C depict linkage disequilibrium between 17 SNPs and the −8 allele of DG8S737 typed in the CEU and the African American populations. The linkage disequilibrium (LD) of the 17 SNPs and the −8 allele of DG8S737 is shown for CEU-in FIG. 9A and African American Michigan cohorts in 9B. Presented here is the D′ (upper left hand) and r2 (lower right hand) between pairs of alleles. Markers are plotted with an equal distance between them and physical locations given in FIG. 9C. Names of markers are shown on the vertical-axis and base pair positions on horizontal-axis.

FIG. 10 is a schematic representation of the AW splice variants identified. Exons are shown as boxes and introns as lines. The transcripts extend from 128,258-128,451 Mb on Chr8q24. The length of exons is as follows: exon 1:503 bp's; exon 2: 343 bp's; exon 3: 103 bp's; exon 4: 88 bp's; exon 5: 371 bp's; exon 6: 135 bp's; exon 6 long: 546 bp's; exon 7: 140 bp's and exon 8: 246 bp's. Note that the figure is not drawn to scale.

### DETAILED DESCRIPTION OF THE INVENTION

Extensive genealogical information for a population containing cancer patients has been combined with powerful gene sharing methods to map a locus on chromosome 8q24.21, which has been demonstrated to play a major role in cancer (e.g., breast cancer, prostate cancer, lung cancer, melanoma). Various cancer patients and their relatives were genotyped with a genome-wide marker set including 1100 microsatellite markers, with an average marker density of 3-4 cM. Presented herein are results from a genome wide search of causative genetic loci for cancer (e.g., breast cancer, prostate cancer, lung cancer, melanoma).

Loci Associated with Various Forms of Cancer Prostate Cancer

The incidence of prostate cancer has dramatically increased over the last decades. Prostate cancer is a multifactorial disease with genetic and environmental components involved in its etiology. It is characterized by heterogeneous growth patterns that range from slow growing tumors to very rapid highly metastatic lesions.

Although genetic factors are among the strongest epidemiological risk factors for prostate cancer, the search for genetic determinants involved in the disease has been challenging. Studies have revealed that linking candidiate genetic markers to prostate cancer has been more difficult than identifying susceptibility genes for other cancers, such as breast, ovary and colon cancer. Several reasons have been proposed for this increased difficulty including: the fact that prostate cancer is often diagnosed at a late age thereby often making it difficult to obtain DNA samples from living affected individuals for more than one generation; the presence within high-risk pedigrees of phenocopies that are associated with a lack of distinguishing features between hereditary and sporadic forms; and the genetic heterogeneity of prostate cancer and the accompanying difficulty of developing appropriate statistical transmission models for this complex disease (Simard, J. et al., Endocrinology 143(6):2029-40 (2002)).

Various genome scans for prostate cancer-susceptibilty genes have been conducted and several prostate cancer susceptibility loci have been reported. For example, HPC1 (1q24-q25), PCAP (1q42-q43), HCPX (Xq27-q28), CAPB (1p36), HPC20 (20q13), HPC2/ELAC2 (17p11) and 16q23 have been proposed as prostate cancer susceptibility loci (Simard, J. et al., Endocrinology 143(6):2029-40 (2002); Nwosu, V. et al., Hum. Mol. Genet. 10(20):2313-18 (2001)). In a genome scan conducted by Smith et al., the strongest evidence for linkage was at HPC1, although two-point analysis also revealed a LOD score of ≧1.5 at D4S430 and LOD scores ≧1.0 at several loci, including markers at Xq27-28 (Ostrander E. A. and J. L. Stanford, Am. J. Hum. Genet. 67:1367-75 (2000)). Another genome scan reported two-point LOD scores of ≧1.5 for chromosomes 10q, 12q and 14q using an autosomal dominant model of inheritance, and chromosomes 1q, 8q, 10q and 16p using a recessive model of inheritance. Id. Still another genome scan identified regions with nominal evidence for linkage on 2q, 12p, 15q, 16q and 16p. Id. A genome scan for prostate cancer predisposition loci using a small set of Utah high risk prostate cancer pedigrees and a set of 300 poymorphic markers provided evidence for linkage to a locus on chromosome 17p (Simard, J. et al., Endocrinology 143(6):2029-40 (2002)). Eight new linkage analyses were published in late 2003, which depicted remarkable heterogeneity. Eleven peaks with LOD scores higher than 2.0 were reported, none of which overlapped (see Actane consortium, Schleutker et.al., Wiklund et.al., Witte et.al., Janer et.al., Xu et.al., Lange et.al, Cunningham et.al; all of which appear in Prostate, vol. 57 (2003)).

As described above, identification of particular genes involved in prostate cancer has been challenging. One gene that has been implicated is RNASEL, which encodes a widely expressed latent endoribonuclease that participates in an interferon-inducible RNA-decay pathway believed to degrade viral and cellular RNA, and has been linked to the HPC locus (Carpten, J. et al., Nat. Genet. 30:181-84 (2002); Casey, G. et al., Nat. Genet. 32(4):581-83 (2002)). Mutations in RNASEL have been associated with increased susceptibility to prostate cancer. For example, in one family, four brothers with prostate cancer carried a disabling mutation in RNASEL, while in another family, four of six brothers with prostate cancer carried a base substitution affecting the initiator methionine codon of RNASEL. Id. Other studies have revealed mutant RNASEL alleles associated with an increased risk of prostate cancer in Finnish men with familial prostate cancer and an Ashkenazi Jewish population (Rokman, A. et al., Am J. Hum. Genet. 70:1299-1304 (2002); Rennert, H. et al., Am J. Hum. Genet. 71:981-84 (2002)). In addition, the Ser217Leu genotype has been proposed to account for approximately 9% of all sporadic cases in Caucasian Americans younger than 65 years (Stanford, J. L., Cancer Epidemiol. Biomarkers Prev. 12(9):876-81 (2003)). In contrast to these positive reports, however, some studies have failed to detect any association between RNASEL alleles with inactivating mutations and prostate cancer (Wang, L. et al., Am. J. Hum. Genet. 71:116-23 (2002); Wiklund, F. et al., Clin. Cancer Res. 10(21):7150-56 (2004); Maier, C. et.al., Br. J. Cancer 92(6): 1159-64(2005)).

The macrophage-scavenger receptor 1 (MSR1) gene, which is located at 8p22, has also been identified as a candidate prostate cancer-susceptibility gene (Xu, J. et al., Nat. Genet. 32:321-25 (2002)). A mutant MSR1 allele was detected in approximately 3% of men with nonhereditary prostate cancer but only 0.4% of unaffected men. Id. However, not all subsequent reports have confirmed these initial findings (see, e.g., Lindmark, F. et al., Prostate 59(2):132-40 (2004); Seppala, E. H. et al., Clin. Cancer Res. 9(14):5252-56 (2003); Wang, L. et al., Nat Genet. 35(2):128-29 (2003); Miller, D. C. et al., Cancer Res. 63(13):3486-89 (2003)). MSR1 encodes subunits of a macrophage-scavenger receptor that is capable of binding a variety of ligands, including bacterial lipopolysaccharide and lipoteicholic acid, and oxidized high-density lipoprotein and low-density lipoprotein in serum (Nelson, W. G. et al., N. Engl. J. Med. 349(4):366-81 (2003)).

The ELAC2 gene on Chr17 was the first prostate cancer susceptibility gene to be cloned in high risk prostate cancer families from Utah (Tavtigian, S. V., et al., Nat. Genet. 27(2):172-80 (2001)). A frameshift mutation (1641InsG) was found in one pedigree. Three additional missense changes: Ser217Leu; Ala541Thr, and Arg781His, were also found to associate with an increased risk of prostate cancer. The relative risk of prostate cancer in men carrying both Ser217Leu and Ala541Thr was found to be 2.37 in a cohort not selected on the basis of family history of prostate cancer (Rebbeck, T. R., et al., Am. J. Hum. Genet. 67(4):1014-19 (2000)). Another study described a new termination mutation (Glu216X) in one high incidence prostate cancer family (Wang, L., et al., Cancer Res. 61(17):6494-99 (2001)). Other reports have not demonstrated strong association with the three missense mutations, and a recent metaanalysis suggests that the familial risk associated with these mutations is more moderate than was indicated in initial reports (Vesprini, D., et al., Am. J. Hum. Genet. 68(4):912-17 (2001); Shea, P. R., et al., Hum. Genet. 111(4-5):398-400 (2002); Suarez, B. K, et al., Cancer Res. 61(13):4982-84 (2001); Severi, G., et al., J. Natl. Cancer Inst. 95(11):818-24 (2003); Fujiwara, H., et al., J. Hum. Genet. 47(12):641-48 (2002); Camp, N. J., et al., Am. J. Hum. Genet. 71(6): 1475-78 (2002)).

Polymorphic variants of genes involved in androgen action (e.g., the androgen receptor (AR) gene, the cytochrome P-450c17 (CYP17) gene, and the steroid-5-□-reductase type II (SRD5A2) gene), have also been implicated in increased risk of prostate cancer (Nelson, W. G. et al., N. Engl. J. Med, 349(4):366-81 (2003)). With respect to AR, which encodes the androgen receptor, several genetic epidemiological studies have shown a correlation between an increased risk of prostate cancer and the presence of short androgen-receptor polyglutamine repeats, while other studies have failed to detect such a correlation. Id. Linkage data has also implicated an allelic form of CYP17, an enzyme that catalyzes key reactions in sex-steroid biosynthesis, with prostate cancer (Chang, B. et al., Int. J. Cancer 95:354-59 (2001)). Allelic variants of SRD5A2, which encodes the predominant isozyme of 5-□-reductase in the prostate and functions to convert testosterone to the more potent dihydrotestosterone, have been associated with an increased risk of prostate cancer and with a poor prognosis for men with prostate cancer (Makridakis, N. M. et al., Lancet 354:975-78 (1999); Nam, R. K. et al., Urology 57:199-204 (2001)).

In short, despite the effort of many groups around the world, the genes that account for a substantial fraction of prostate cancer risk have not been identified. Although twin studies have implied that genetic factors are likely to be prominent in prostate cancer, only a handful of genes have been identified as being associated with an increased risk for prostate cancer, and these genes account for only a low percentage of cases. Thus, it is clear that the majority of genetic risk factors for prostate cancer remain to be found. It is likely that these genetic risk factors will include a relatively high number of low-to-medium risk genetic variants. These low-to-medium risk genetic variants may, however, be responsible for a substantial fraction of prostate cancer, and their identification, therefore, a great benefit for public health. Furthermore, none of the published prostate cancer genes have been reported to predict a greater risk for aggressive prostate cancer than for less aggressive prostate cancer.

As described herein, a locus on chromosome 8q24.21 has been demonstrated to play a role in prostate cancer and it has been discovered that particular markers and/or haplotypes in a specific DNA segment within the locus are present at a higher than expected frequency in prostate cancer subjects. Thus, in various embodiments of the invention, certain markers and/or SNPs, identified using the methods described herein, can be used for a diagnosis of a susceptibility to prostate cancer, and also for a diagnosis of a decreased susceptibility to prostate cancer or for identification of variants that are protective against prostate cancer. The diagnostic assays presented below can be used to identify the presence or absence of these particular variants.

Thus, in one embodiment, the invention is a method of diagnosing a susceptibility to prostate cancer (e.g., aggressive or high Gleason grade prostate cancer, less aggressive or low Gleason grade prostate cancer), comprising detecting a marker or haplotype associated with LD Block A (e.g., a marker as set forth in Table 13, having a value of RR greater than one, indicating the marker is associated with susceptibility to disease/increased risk of disease and thus is an “at-risk” variant; values of RR less than one indicate the marker is associated with decreased susceptibility to disease/decreased risk of disease and thus is a “protective” variant), wherein the presence of the marker or haplotype is indicative of a susceptibility to prostate cancer. In another embodiment, the invention is a method of diagnosing a susceptibility to, or an increased risk of, prostate cancer (e.g., aggressive or high Gleason grade prostate cancer, less aggressive or low Gleason grade prostate cancer), comprising detecting marker DG8S737 or marker rs1447295, wherein the presence of the −8 allele at marker DG8S737 or the presence of the A allele at marker rs1447295, is indicative of a susceptibility to prostate cancer or an increased risk of prostate cancer. In a further embodiment, the invention is a method of diagnosing a susceptibility to prostate cancer in an individual whose ancestry comprises African ancestry, comprising detecting marker DG8S737, wherein the presence of the −8 allele at marker DG8S737 is indicative of a susceptibility to prostate cancer or an increased risk of prostate cancer. In particular embodiments, the marker or haplotype that is associated with a susceptibility to prostate cancer has a relative risk of at least 1.5, or at least 2.0. In another embodiment, the prostate cancer is an aggressive prostate cancer, as defined by a combined Gleason score of 7(4+3) to 10 and/or an advanced stage of prostate cancer (e.g., Stages 2 to 4). In yet another embodiment, the prostate cancer is a less aggressive prostate cancer, as defined by a combined Gleason score of 2 to 7(3+4) and/or an early stage of prostate cancer (e.g., Stage 1). In another embodiment, the presence of a marker or haplotype associated with LD Block A, in conjunction with the subject having a PSA level greater than 4 ng/ml, is indicative of a more aggressive prostate cancer and/or a worse prognosis. In yet another embodiment, in patients who have a normal PSA level (e.g., less than 4 ng/ml), the presence of a marker or haplotype is indicative of a more aggressive prostate cancer and/or a worse prognosis.

In other embodiments, the invention is a method of diagnosing a decreased susceptibility to prostate cancer, comprising detecting a marker or haplotype associated with LD Block A, wherein the presence of that marker or haplotype is indicative of a decreased susceptibility to prostate cancer or of a protective marker or haplotype against prostate cancer. In certain embodiments, the marker is a marker as set forth in Table 13, or the haplotype comprises one or more markers as set forth in Table 13 (e.g., a marker as set forth in Table 13, or a haplotype comprising one or more markers set forth in Table 13 wherein the marker(s) has a value of RR less than one, indicating the marker is associated with decreased susceptibility to disease/decreased risk of disease and thus is a “protective” variant; values of RR greater than one indicate the marker is associated with increased susceptibility to disease/increased risk of disease and thus is an “at-risk” variant). In another embodiment, the invention is a method of diagnosing a decreased susceptibility to, or decreased risk of, prostate cancer, comprising detecting marker DG8S737 or marker rs1447295, wherein the presence of an allele other than the −8 allele at marker DG8S737 or the presence of the C allele at marker rs1447295, is indicative of a decreased susceptibility to prostate cancer or a decreased risk of prostate cancer (protective against prostate cancer). In a further embodiment, the invention is a method of diagnosing a decreased susceptibility to prostate cancer in an individual whose ancestry comprises African ancestry, comprising detecting marker DG8S737, wherein the presence of an allele other than the −8 allele at marker DG8S737 is indicative of a decreased susceptibility to prostate cancer or a decreased risk of prostate cancer (protective against prostate cancer).

### Breast Cancer

As described herein, although the discovery of BRCA1 and BRCA2 were important milestones in identifying two key genetic factors involved in breast cancer, it has become clear that mutations in BRCA1 and BRCA2 account for only a fraction of inherited susceptibility to breast cancer. It is estimated that only 5-10% of all breast cancers in women are associated with hereditary susceptibility due to mutations in autosomal dominant genes, such as BRCA1, BRCA2, p53, pTEN and STK11/LKB1 (Mincey, B. A. Oncologist 8:466-73 (2003)). One genetic locus, on Chromosome 8p, has been proposed as a locus for a breast cancer-susceptibility gene based on studies documenting allelic loss in this region in sporadic breast cancer (Seitz, S. et al., Br. J. Cancer 76:983-91 (1997); Kerangueven, F. et al., Oncogene 10:1023 (1995)). Studies have also suggested that a breast cancer-susceptibility gene may be located on 13q21 (Kainu, T. et al., Proc. Natl. Acad. Sci. USA 97:9603-08 (2000)). However, as with prostate cancer, identification of additional breast cancer-susceptibility genes has been difficult.

As described herein, a locus on chromosome 8q24.21 has been demonstrated to play a role in breast cancer and it has been discovered that particular markers and/or haplotypes in a specific DNA segment within the locus are present at a higher than expected frequency in breast cancer subjects. Thus, in one embodiment, the invention is a method of diagnosing a susceptibility to breast cancer comprising detecting a marker or haplotype associated with LD Block A, wherein the presence of the marker or haplotype is indicative of a susceptibility to breast cancer. In a particular embodiment, the marker or haplotype that is associated with a susceptibility to breast cancer has a relative risk of at least 1.3. In other embodiments, the invention is drawn to a method of diagnosing a decreased susceptibility to breast cancer comprising detecting a marker or haplotype associated with LD Block A, wherein the presence of that marker or haplotype is indicative of a decreased susceptibility to breast cancer or of a protective marker or haplotype against breast cancer (protective against breast cancer). In a particular embodiment, the marker or haplotype that is associated with a decreased susceptibility to breast cancer (protective against breast cancer) has a relative risk of less than 0.75.

### Lung Cancer

While environmental, lifestyle (e.g., smoking) and dietary factors play an important role in lung cancer, genetic factors are also important. Studies have revealed that defects in both the p53 and RB/p16 pathway are essential for the malignant transformation of lung epithelial cells (Yokota, J. and T. Kohno, Cancer Sci. 95(3):197-204 (2004)). Other genes, such as K-ras, PTEN and MYO18B, are genetically altered less frequently than p53 and RB/p16 in lung cancer cells, suggesting that alterations in these genes are associated with further malignant progression or unique phenotypes in a subset of lung cancer cells. Id. Molecular footprint studies that have been conducted at the sites of p53 mutations and RB/p16 deletions have further demonstrated that DNA repair activities and non-homologous end-joining of DNA double-strand breaks are important in the accumulation of genetic alterations in lung cancer cells. Id. In addition, studies have identified candidate lung adenocarcinoma susceptibility genes, for example, drug carcinogen metabolism genes, such as NQ01 (NAD(P)H:quinone oxidoreductase) and GSTT1 (glutathione S-transferase T1), and DNA repair genes, such as XRCC1 (X-ray cross-complementary group 1) (Yanagitani, N. et al., Cancer Epidemiol. Biomarkers Prev. 12:366-71 (2003); Lin, P. et al., J. Toxicol. Environ. Health A. 58:187-97 (1999); Divine, K. K. et al., Mutat. Res. 461:273-78 (2001); Sunaga, N. et al., Cancer Epidemiol. Biomarkers Prev. 11:730-38 (2002)). A region of chromosome 19q13.3, which encompasses locus D19S246, has also been suggested as containing a gene(s) associated with lung adenocarcinoma (Yanagitani, N. et al., Cancer Epidemiol. Biomarkers Prev. 12:366-71 (2003)).

As described herein, a locus on chromosome 8q24.21 has been demonstrated to play a role in lung cancer and it has been discovered that particular markers and/or haplotypes in a specific DNA segment within the locus are present at a higher than expected frequency in lung cancer subjects. In one embodiment, the invention is a method of diagnosing a susceptibility to lung cancer comprising detecting a marker or haplotype associated with LD Block A, wherein the presence of the marker or haplotype is indicative of a susceptibility to lung cancer. In a particular embodiment, the marker or haplotype that is associated with a susceptibility to lung cancer has a relative risk of at least 1.3. In other embodiments, the invention is drawn to a method of diagnosing a decreased susceptibility to lung cancer comprising detecting a marker or haplotype associated with LD Block A, wherein the presence of that marker or haplotype is indicative of a decreased susceptibility to lung cancer or of a protective marker or haplotype against lung cancer (protective against lung cancer). In a particular embodiment, the marker or haplotype that is associated with a decreased susceptibility to lung cancer (protective against lung cancer) has a relative risk of less than 0.75.

### Melanoma

Studies have demonstrated that genetic factors play an important role in the stepwise progression of normal pigment cells to atypical nevi to invasive primaiy melanoma and finally to cells with aggressive metastatic potential (Kim, C. J., et al., Cancer Control 9(1):49-53 (2002)). For example, genetic aberrations, such as rearrangements on chromosome 1, which harbors a tumor-suppressor gene, have been implicated in malignant melanomas. Id. However, the molecular and biological mechanisms of how a normal melanocyte of adult skin transforms into a melanoma cell remains unclear.

Various studies have implicated genetic factors in melanoma For example, elevated familial risk for early onset melanoma was noted by examination of a Utah population database (Cannon-Albright, L. A., et al., Cancer Res., 54(9):2378-85 (1994)). In addition, the Swedish Family-Cancer Database reported a familial standardized incidence ratios (SIR) of 2.54 and 2.98 for cutaneous malignant melanoma (CMM) in a individual with an affected parent or sib, respectively. For an offspring whose parent had multiple primary melanomas, the SIR rose to 61.78 (Hemminki, K., et al., J. Invest. Dermatol. 120(2):217-23 (2003)). Although figures vary, it has been reported that about 10% of CMM cases are familial (Hansen, C. B., et al., Lancet Oncol. 5(5):314-19 (2004)). Given the known environmental risk factors for melanoma, shared environment in addition to genetics is likely to factor into these estimates. However, familial cases tend to have earlier ages of onset and a higher risk of multiple primary tumors, suggesting a genetic component.

A series of linkage-based studies have implicated CDKN2a on Chr9p21 as a major CMM-susceptibility gene (Bataille, V., Eur. J. Cancer 39(10):1341-47 (2003)). CDK4 was identified as a pathway candidate shortly thereafter, however, mutations in CDK4 have only been observed in a few families worldwide (Zuo, L., et al., Nat. Genet. 12(1):97-99 (1996)). CDKN2a encodes the cyclin dependent kinase inhibitor p16, which inhibits CDK4 and CDK6, thereby preventing G1 to S cell cycle transit. An alternate transcript of CKDN2a produces p14ARF, which encodes a cell cycle inhibitor that acts through the MDM2-p53 pathway. It is likely that CDKN2a mutant melanocytes are deficient in cell cycle control or the establishment of senescence, either as a developmental state or in response to DNA damage (Ohtani, N., et al., J. Med. Invest. 51(3-4):146-53 (2004)). Overall penetrance of CDKN2a mutations in familial CMM cases is 67% by age 80. However, penetrance is increased in areas of high melanoma prevalence (Bishop, D. T., et al., J. Natl. Cancer Inst. 94(12):894-903 (2002)).

The Melanoma Genetics Consortium recently completed a genome-wide scan for CMM, using a set of predominantly Australian, high-risk families unlinked to 9p21 or CDK4 (Gillanders, E., et al., Am. J. Hum. Genet. 73(2):301-13 (2003)). The 10 cM resolution scan gave a non-parametric multipoint LOD score of 2.06 in the 1p22 region. Other locations on chromosomes 4, 7, 14, and 18 gave LODs in excess of 1.0. With additional markers to 1p22 and the application of an age-of-onset restriction, non-parametric LOD scores in excess of 5.0 were observed. Evidence suggests that a high-penetrance mutation of a tumor suppressor gene exists at this location, however the pattern of LOH is complex (Walker, G. J., et al., Genes Chromosomes Cancer, 41(1):56-64 (2004)).

Another genetic locus that has been implicated in CMM is that which encodes the Melanocortin 1 Receptor (MCIR). MC1R is a G-protein coupled receptor that is involved in promoting the switch from pheomelanin to eumelanin synthesis. Numerous well-characterized variants of the MC1R gene have been implicated in red-haired, pale-skinned and freckle-prone phenotypes. More than half of red-haired individuals carry at least one of these MC1R variants (Valverde, P., et al., Nat. Genet. 11(3):328-30 (1995); Palmer, J. S., et al., Am. J. Hum. Genet. 66(1):176-86 (2000)). Subsequently, it was shown that the same variants conferred risk for CMM with odds ratios of about 2.0 for a single variant and about 4.0 for compound heterozygotes. Recent studies have shown that the stronger variants of MC1R increase the penetrance of CDKN2a mutations and lower the age of onset (Box, N. F., et al., Am. J. Hum. Genet. 69(4):765-73 (2001); van der Velden, P. A., et al., Am. J. Hum. Genet., 69(4):774-79 (2001)).

A number of other candidate genes have been implicated in CMM. For example, a landmark study in cancer genomics identified somatic mutations in BRAF (the human B1 homolog of the v-raf murine sarcoma virus oncogene) in 60% of melanomas (Davies, H., et al., Nature 417(6892):949-54 (2002)). Mutations are also common in nevi, both typical and atypical, suggesting that mutation is an early event. Id. Germline mutations have not been reported, however, a germline SNP variant of BRAF has been implicated in CMM risk (Meyer, P., et al., J. Carcinog. 2(1):7 (2003)). Other candidate genes, which were identified through association studies and have been implicated in CMM risk include, e.g., XRCC3, XPD, EGF, VDR, NBS1, CYP2D6, and GSTMI (Hayward, N. K., Oncogene, 22(20):3053-62 (2003)). However, such association studies frequently suffer from small sample sizes, reliance on single SNPs and potential population stratification.

As described herein, a locus on chromosome 8q24.21 has been demonstrated to play a role in melanoma and it has been discovered that particular markers and/or haplotypes in a specific DNA segment within the locus are present at a higher than expected frequency in melanoma subjects. In one embodiment, the invention is a method of diagnosing a susceptibility to melanoma comprising detecting a marker or haplotype associated with LD Block A, wherein the presence of the marker or haplotype is indicative of a susceptibility to melanoma In a particular embodiment, the marker or haplotype that is associated with a susceptibility to melanoma has a relative risk of at least 1.5. In another embodiment, the melanoma is malignant cutaneous melanoma. In a further embodiment, the marker or haplotype that is associated with malignant cutaneous melanoma has a relative risk of at least 1.7.

In other embodiments, the invention is drawn to a method of diagnosing a decreased susceptibility to melanoma comprising detecting a marker or haplotype associated with LD Block A, wherein the presence of that marker or haplotype is indicative of a decreased susceptibility to melanoma or of a protective marker or haplotype against melanoma (protective against melanoma). In a particular embodiment, the marker or haplotype that is associated with a decreased susceptibility melanoma (protective against melanoma) has a relative risk of less than 0.7. In another embodiment, the melanoma is malignant cutaneous melanoma. In a further embodiment, the marker or haplotype that is associated with a decreased susceptibility to malignant cutaneous melanoma (protective against malignant cutaneous melanoma) has a relative risk of less than 0.6.

### Assessment for Marker and Haplotypes

Populations of individuals exhibiting genetic diversity do not have identical genomes. Rather, the genome exhibits sequence variability between individuals at many locations in the genome; in other words, there are many polymorphic sites in a population. In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a disease or abnormal phenotype). Alleles that differ from the reference are referred to as “variant” alleles.

A “marker”, as described herein, refers to a genomic sequence characteristic of a particular variant allele (i.e. polymorphic site). The marker can comprise any allele of any variant type found in the genome, including SNPs, microsatellites, insertions, deletions, duplications and translocations.

SNP nomenclature as reported herein refers to the official Reference SNP (rs) ID identification tag as assigned to each unique SNP by the National Center for Biotechnological Information (NCBI).

A “haplotype,” as described herein, refers to a segment of genomic DNA that is characterized by a specific combination of genetic markers (“alleles”) arranged along the segment. The combination of alleles, such as haplotype 1 and haplotype 1a, are described in Tables 2 and 4, respectively. In a certain embodiment, the haplotype can comprise one or more alleles, two or more alleles, three or more alleles, four or more alleles, or five or more alleles. The genetic markers are particular “alleles” at “polymorphic sites” associated with Chr8q24.21 and/or LD Block A. As used herein, “Chr8q24.21” and “8q24.21” refer to chromosomal band 8q24.21 or 127,200,001-131,400,000 bp in UCSC Build 34 (from the USCS Genome browser Build 34 at www.genome.ucsc.edu). As used herein, “LD Block A” refers to the LD block on Chr8q24.21 wherein association of variants to prostate, breast, lung cancer and melanoma is observed. NCBI Build 34 position of this LD block is from 128,414,000-128,506,000 bp. The term “African ancestry”, as described herein, refers to self-reported African ancestry of individuals.

The term “susceptibility”, as described herein, encompasses both increased susceptibility and decreased susceptibility. Thus, particular markers and/or haplotypes of the invention may be characteristic of increased susceptility of cancer, as characterized by a relative risk of greater than one. Alternatively, the markers and/or haplotypes of the invention are characteristic of decreased susceptibility of cancer, as characterized by a relative risk of less than one.

A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Alleles for SNP markers as referred to herein refer to the bases A, C, G or T as they occur at the polymorphic site in the SNP assay employed. The person skilled in the art will realise that by assaying or reading the opposite strand, the complementary allele can in each case be measured. Thus, for a polymorphic site containing an A/G polymorphism, the assay employed may either measure the percentage or ratio of the two bases possible, i.e. A and G. Alternatively, by designing an assay that determines the opposite strand on the DNA template, the percentage or ratio of the complementary bases T/C can be measured. Quantitatively (for example, in terms of relative risk), identical results would be obtained from measurement of either DNA strand (+ strand or − strand). Polymorphic sites can allow for differences in sequences based on substitutions, insertions or deletions. For example, a polymorphic microsatellite has multiple small repeats of bases (such as CA repeats) at a particular site in which the number of repeat lengths varies in the general population. Each version of the sequence with respect to the polymorphic site is referred to herein as an “allele” of the polymorphic site. Thus, in the previous example, the SNP allows for both an adenine allele and a thymine allele. SNPs and microsatellite markers associated with cancer (e.g., prostate cancer (e.g., aggressive prostate cancer), breast cancer, lung cancer, melanoma) are described in Tables 1 and 13.

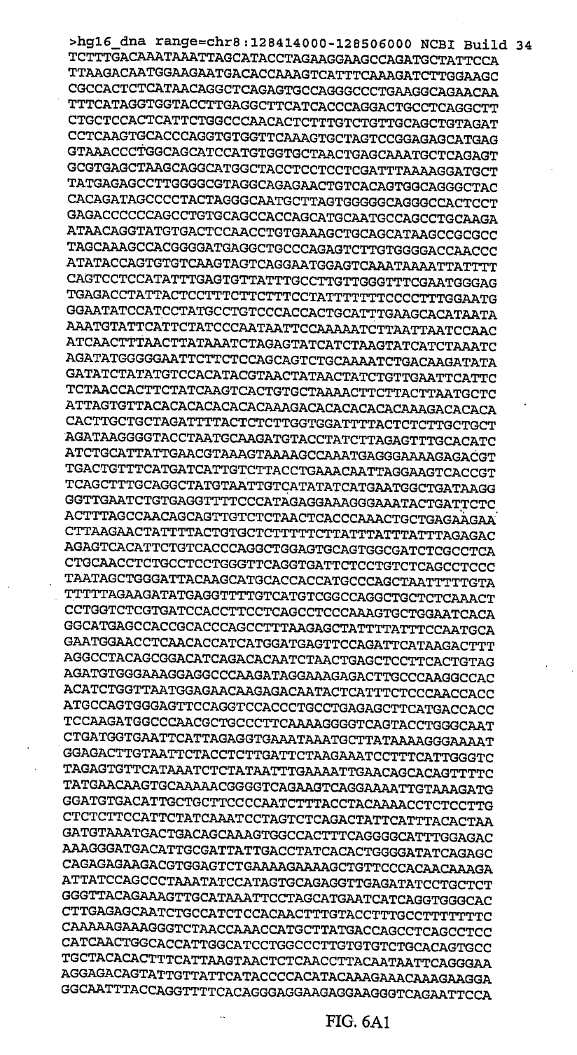

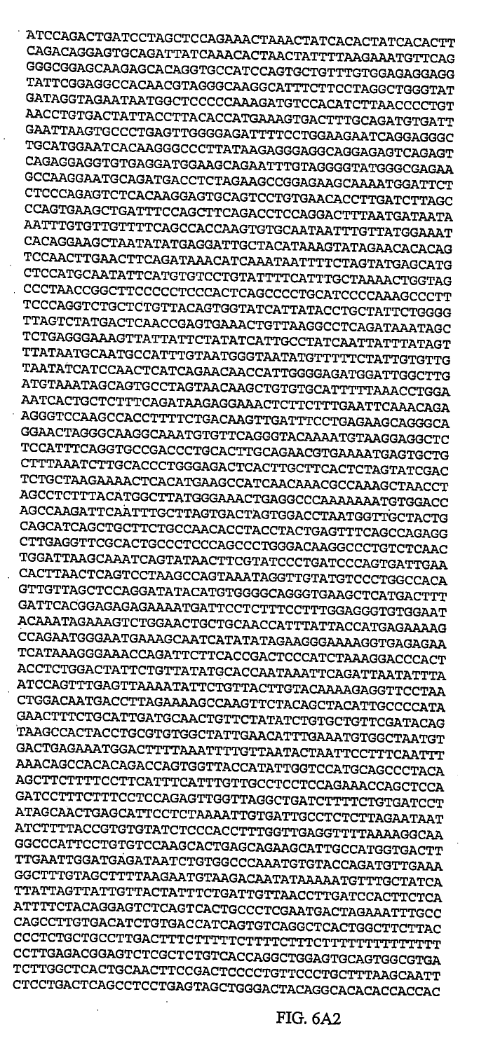

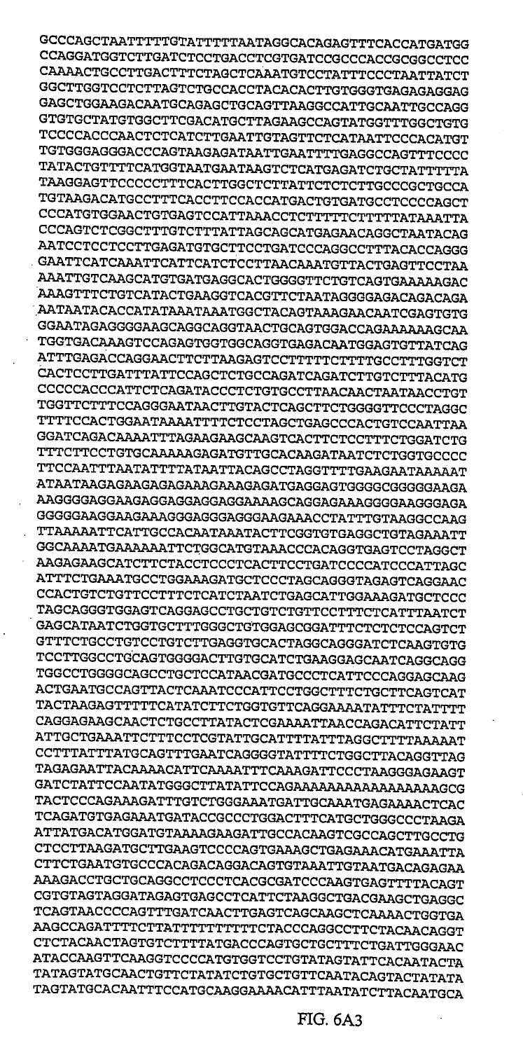

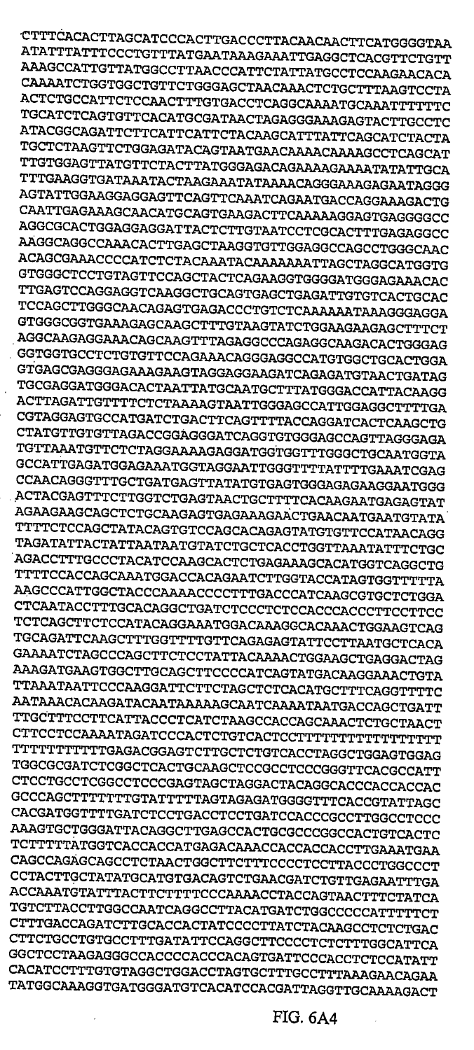

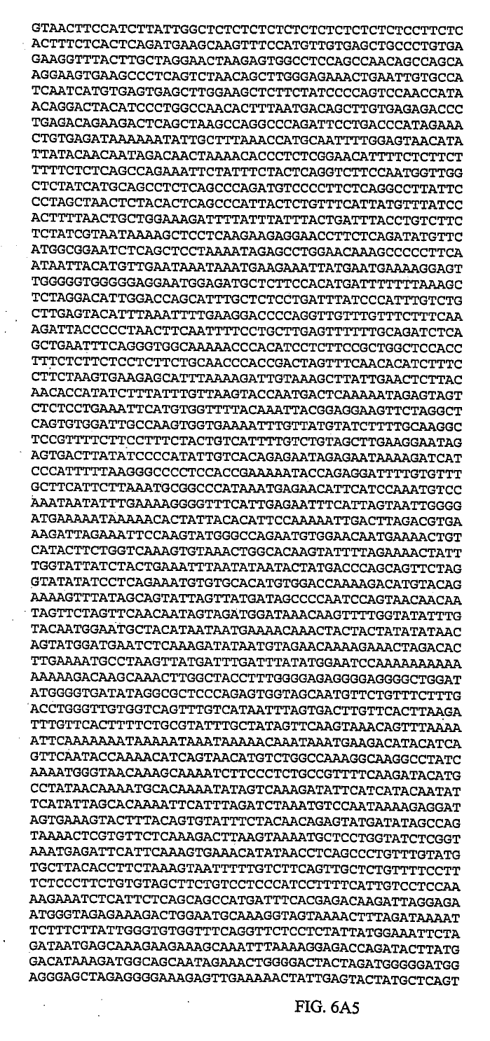

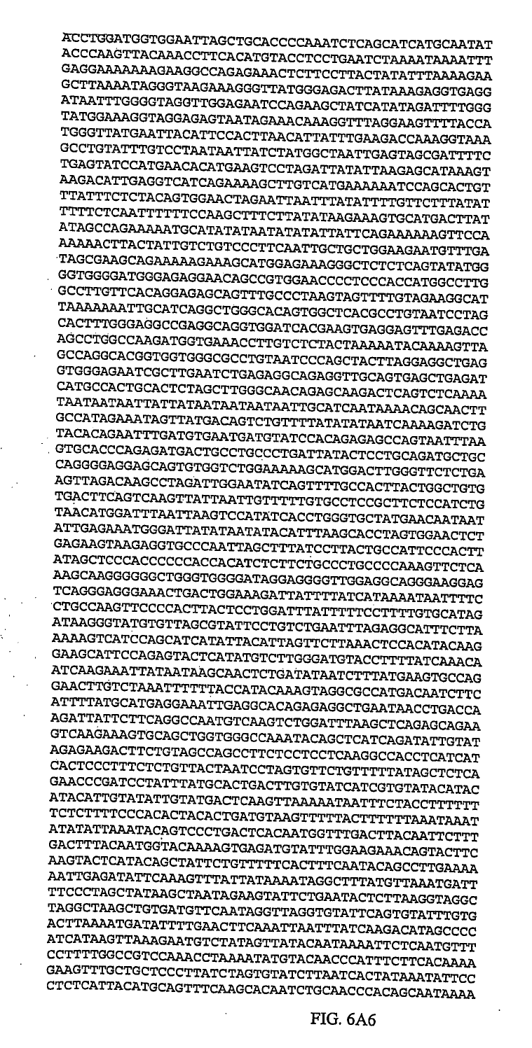

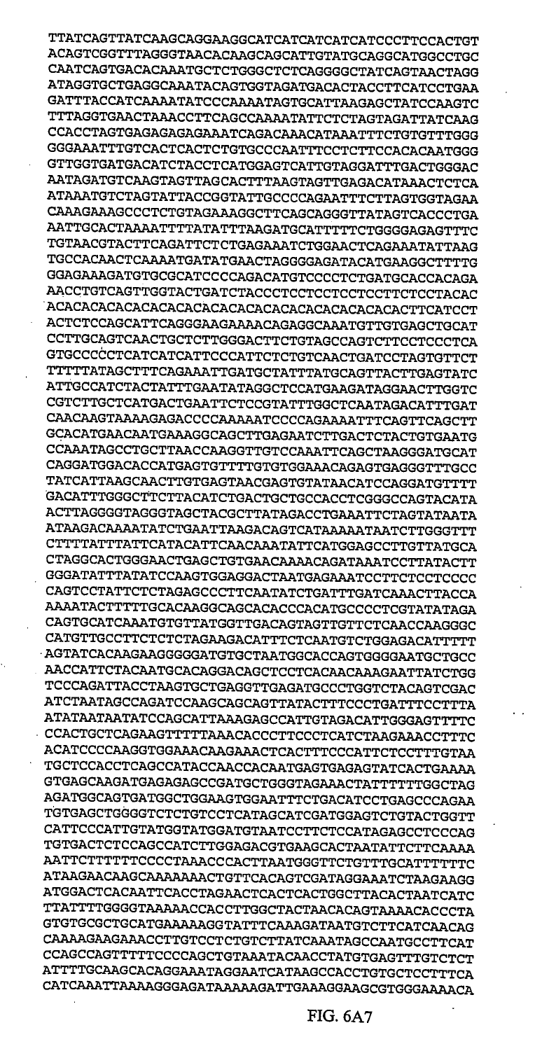

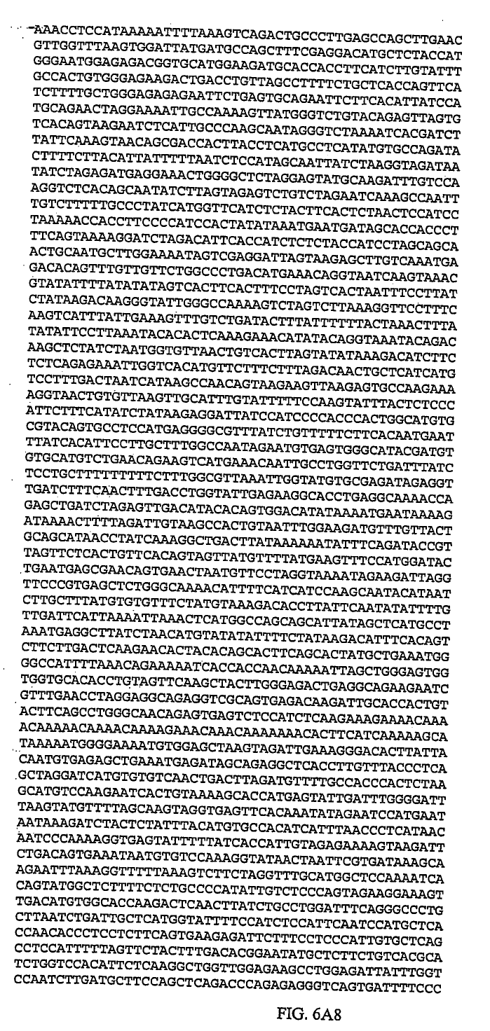

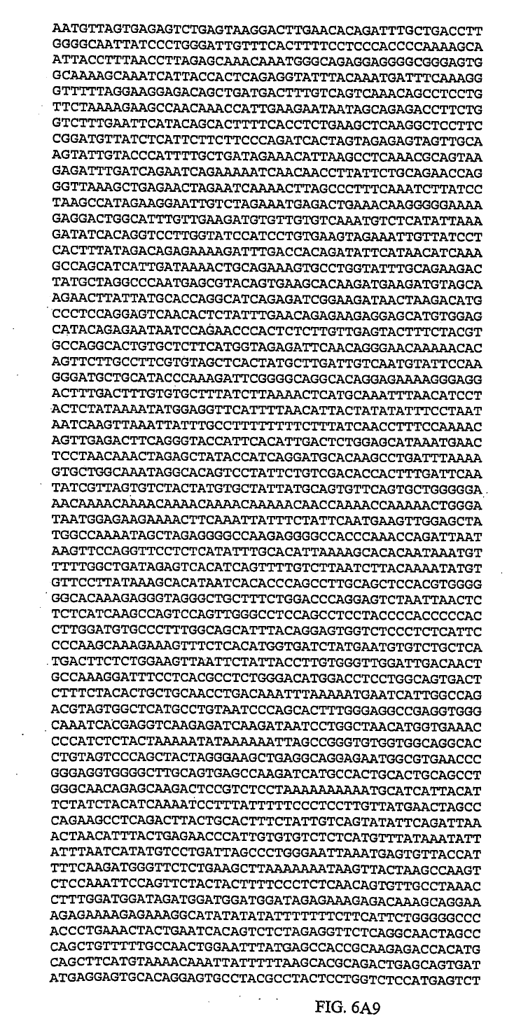

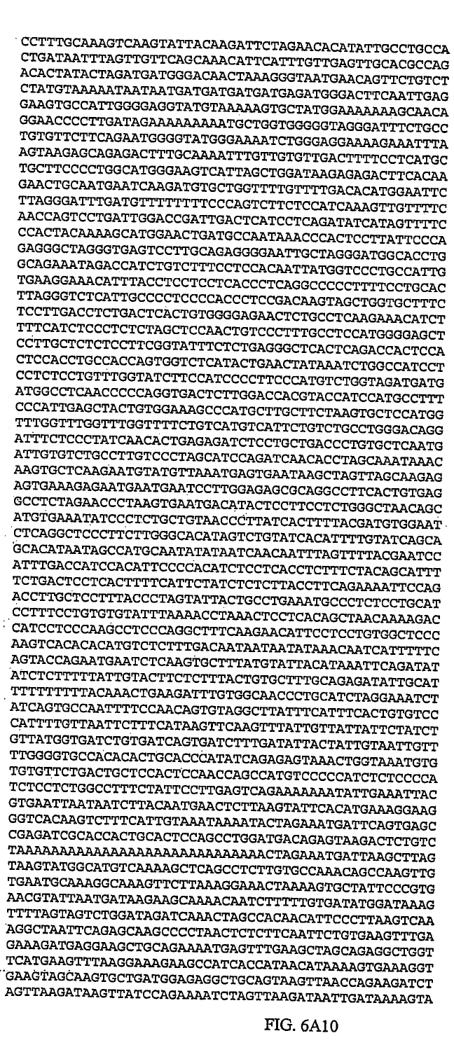

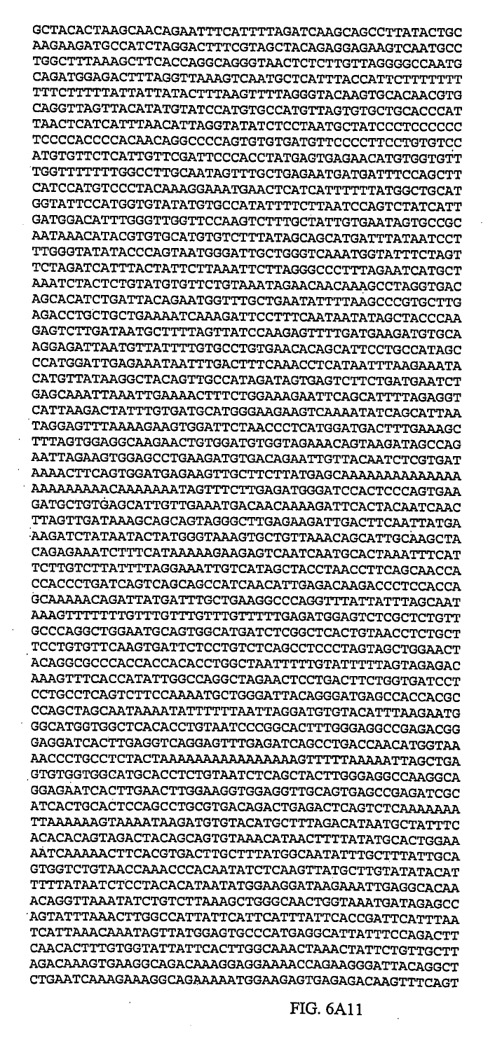

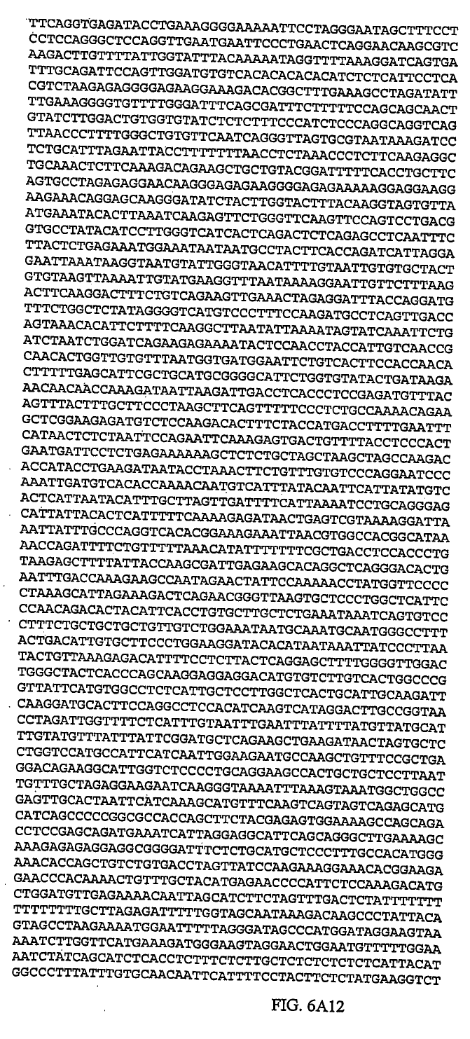

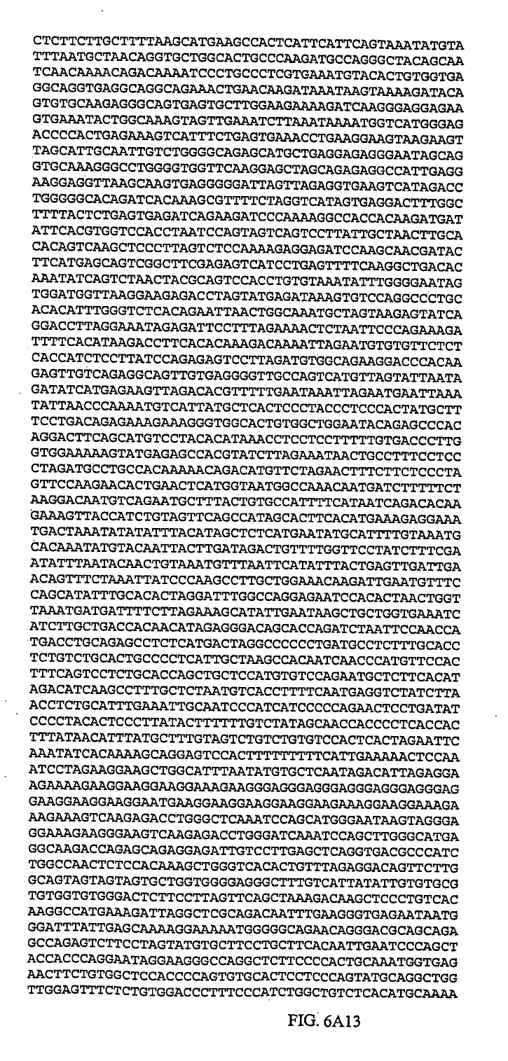

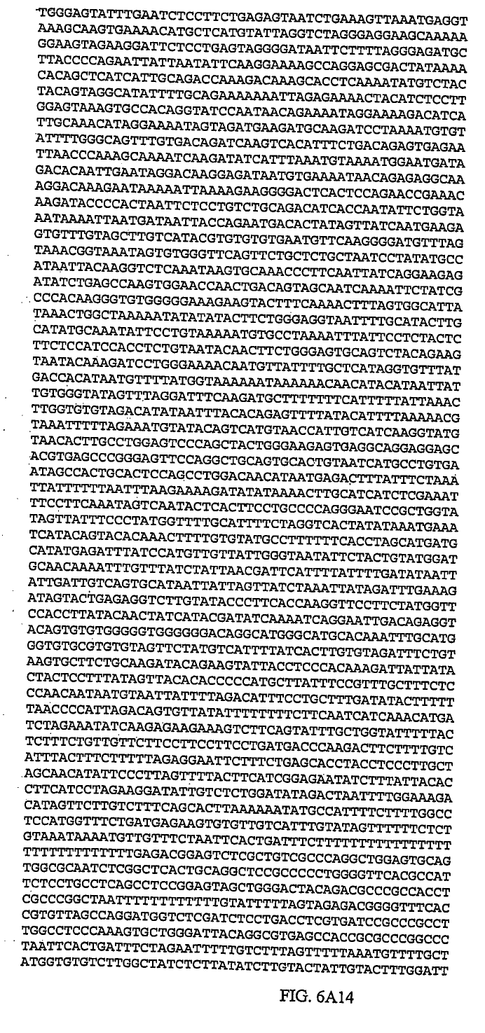

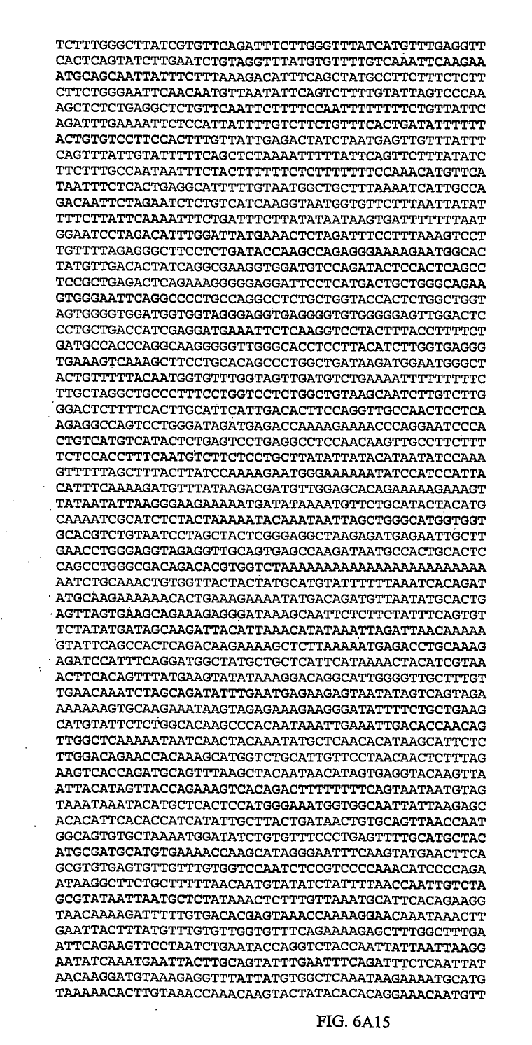

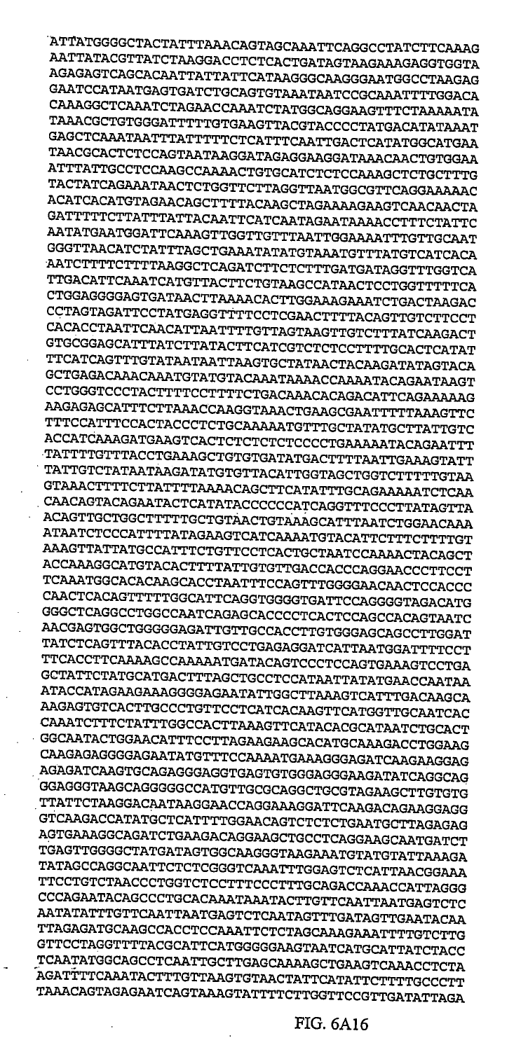

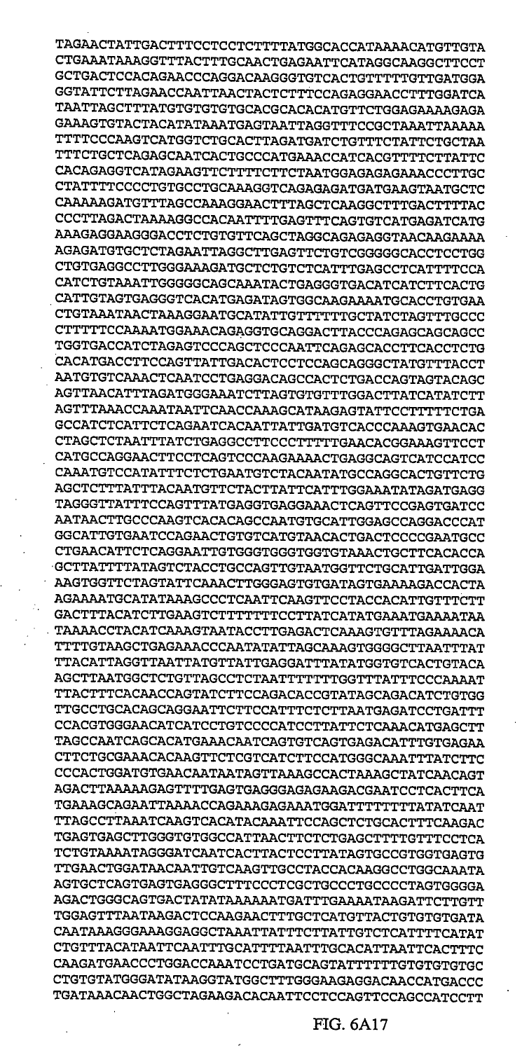

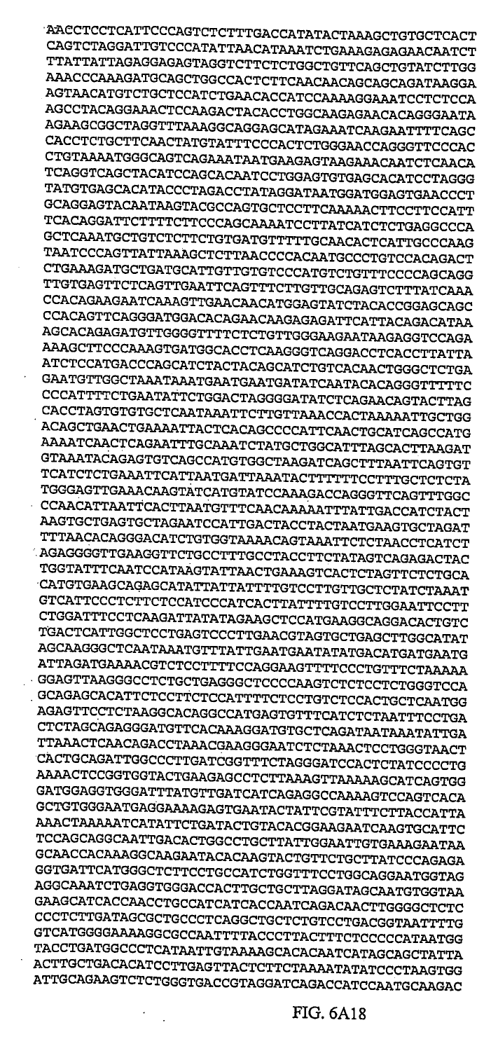

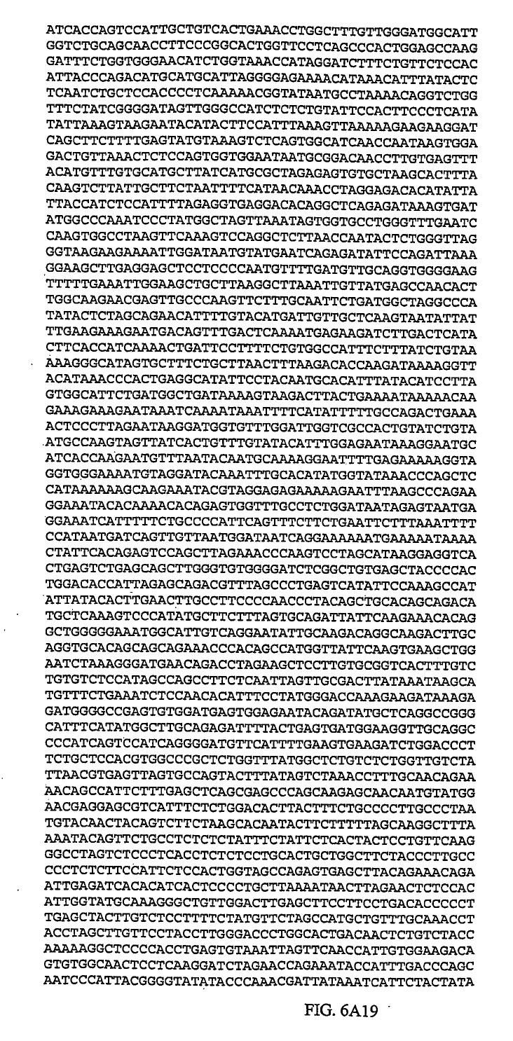

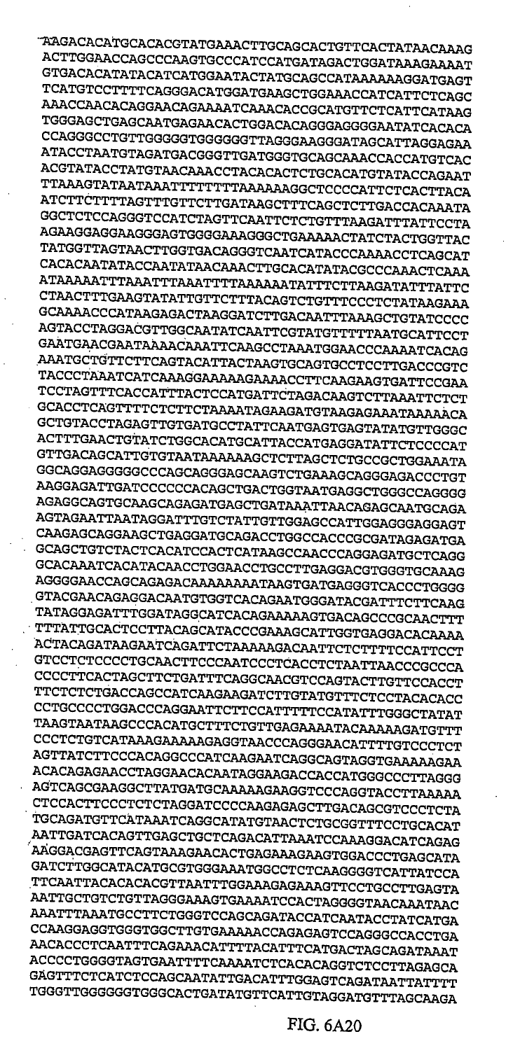

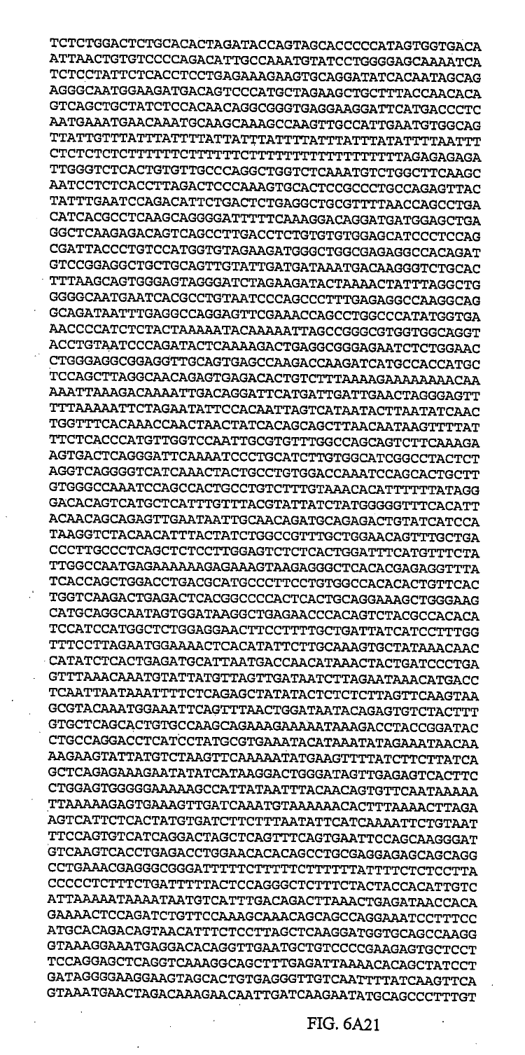

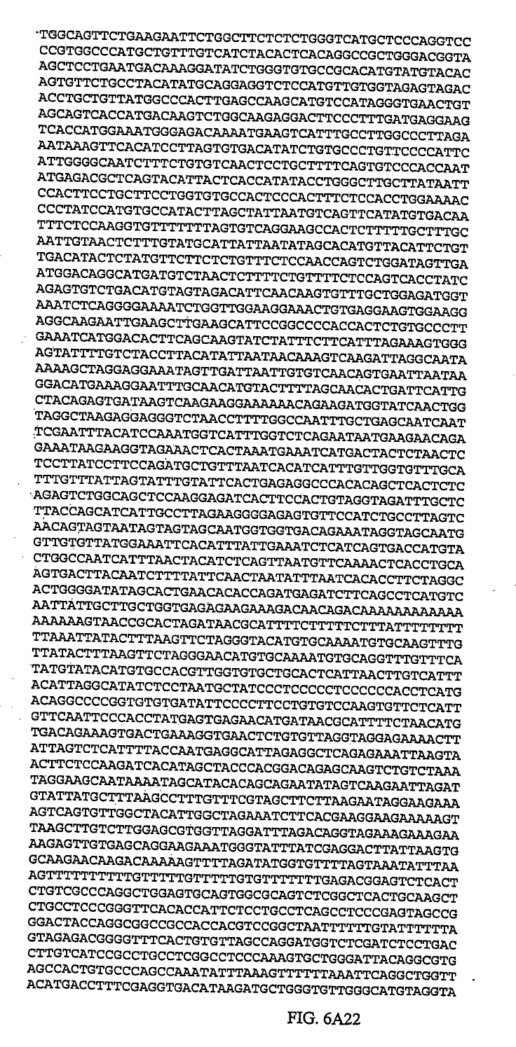

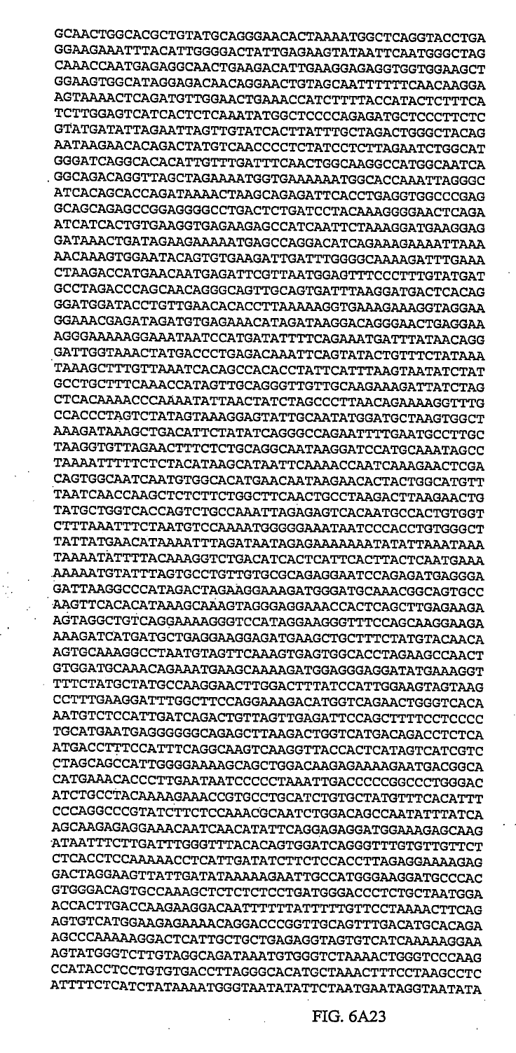

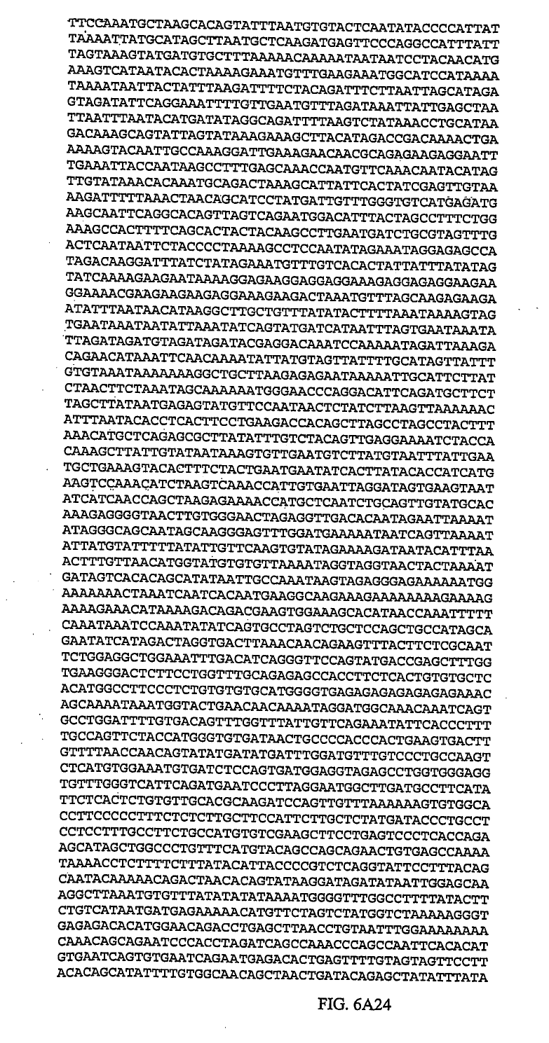

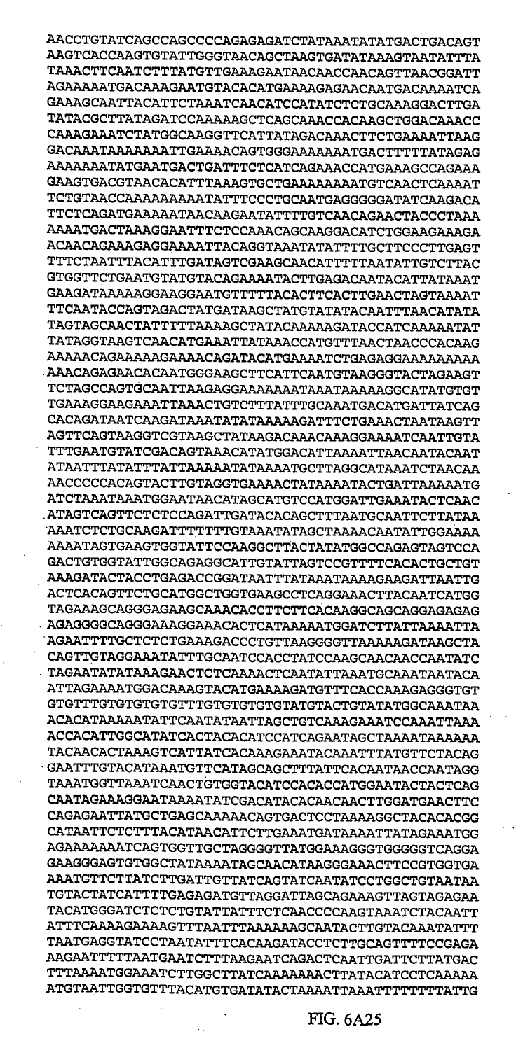

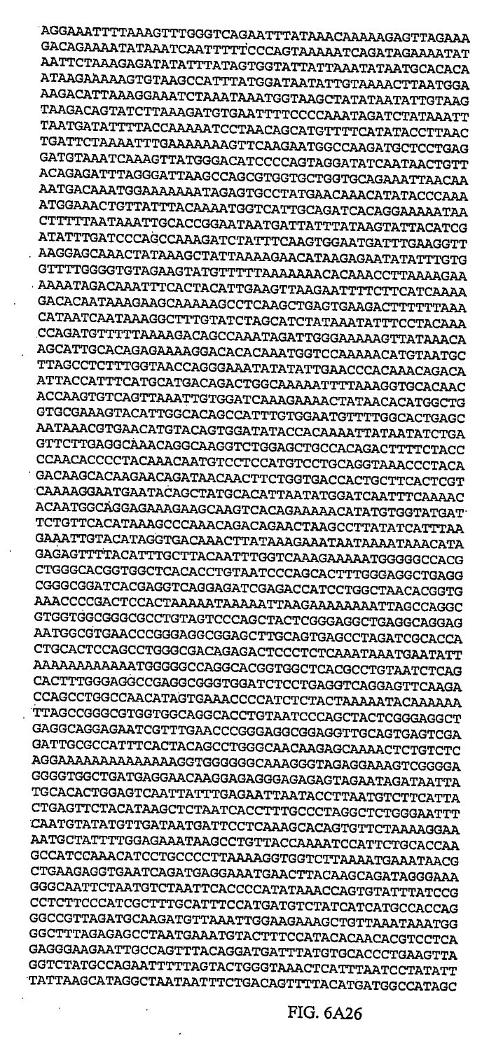

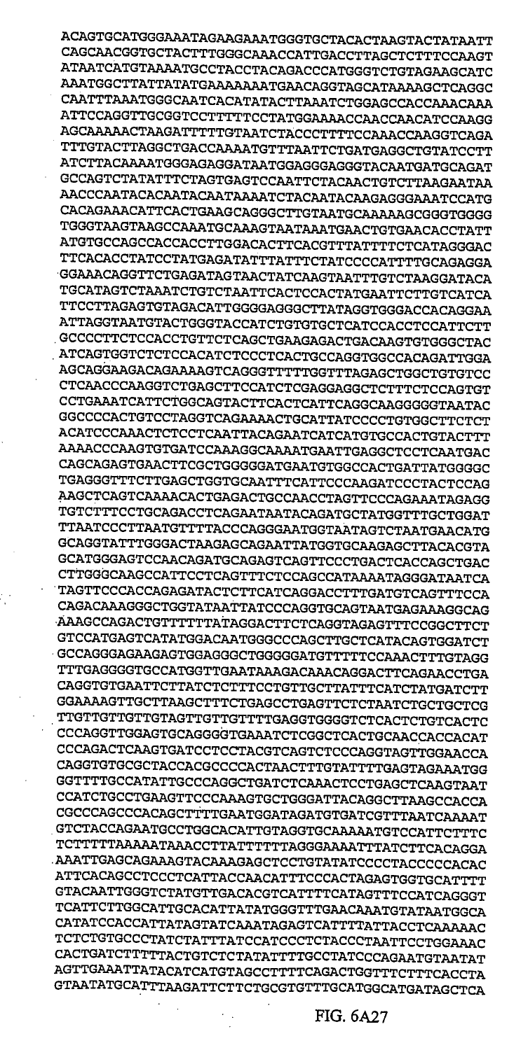

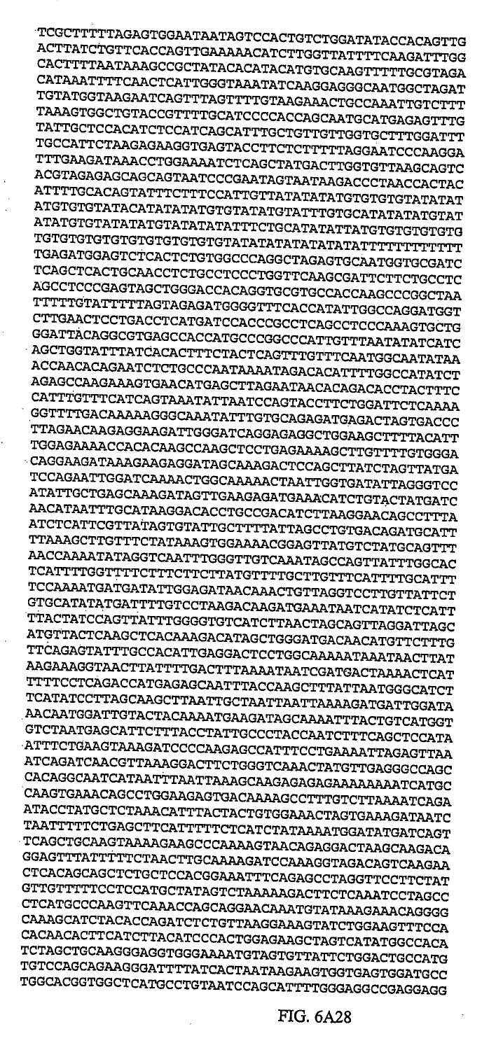

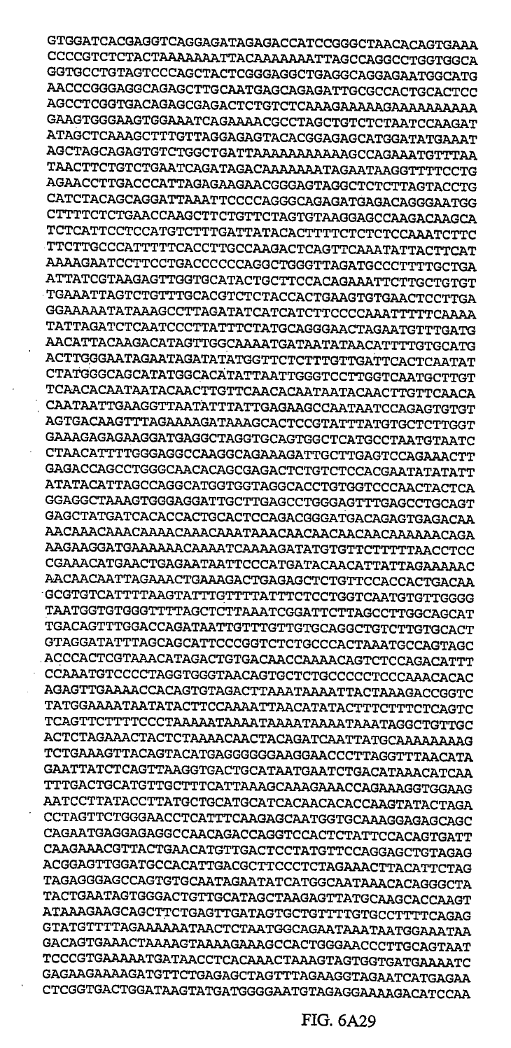

Typically, a reference sequence is referred to for a particular sequence. Alleles that differ from the reference are referred to as “variant” alleles. For example, the reference genomic DNA sequence from 128,414,000-128,506,000 bp of NCBI Build 34, which refers to the location within Chromosome 8, is described herein as SEQ ID NO:1 (FIG. 6A1-6A31). A variant sequence, as used herein, refers to a sequence that differs from SEQ ID NO:1 but is otherwise substantially similar. The genetic markers that make up the haplotypes described herein are variants. Additional variants can include changes that affect a polypeptide, e.g., a polypeptide encoded by SEQ ID NO:1. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail herein. Such sequence changes alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide, Alternatively, a polymorphism associated with cancer (e.g., prostate cancer (e.g., aggressive prostate cancer), breast cancer, lung cancer, melanoma) or a susceptibility to cancer can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level in tumors. The polypeptide encoded by the reference nucleotide sequence is the “reference” polypeptide with a particular reference amino acid sequence, and polypeptides encoded by variant alleles are referred to as “variant” polypeptides with variant amino acid sequences.

The haplotypes described herein are a combination of various genetic markets, e.g., SNPs and microsatellites, having particular alleles at polymorphic sites. The haplotypes can comprise a combination of various genetic markers, therefore, detecting haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), PCR, LCR, Nested PCR and other techniques for nucleic acid amplification. These markers and SNPs can be identified in at-risk haplotypes. Certain methods of identifying relevant markers and SNPs include the use of linkage disequilibrium (LD) and/or LOD scores.

### Linkage Disequilibrium

Linkage Disequilibrium (LD) refers to a non-random assortment of two genetic elements. For example, if a particular genetic element (e.g., “alleles” at a polymorphic site) occurs in a population at a frequency of 0.25 and another occurs at a frequency of 0.25, then the predicted occurrance of a person's having both elements is 0.125, assuming a random distribution of the elements. However, if it is discovered that the two elements occur together at a frequency higher than 0.125, then the elements are said to be in linkage disequilibrium since they tend to be inherited together at a higher rate than what their independent allele frequencies would predict. Roughly speaking, LD is generally correlated with the frequency of recombination events between the two elements. Allele frequencies can be determined in a population by genotyping individuals in a population and determining the occurence of each allele in the population. For populations of diploids, e.g. human populations, individuals will typically have two alleles for each genetic element (e.g., a marker or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. So, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.2, such as at least 0.2, 0.3, 0.4, 0.5, 0.6, 0.7, 0.8, 0.9 or 1.0.

Thus, LD represents a correlation between alleles of distinct markers. It is measured by correlation coefficient or |D′| (r² up to 1.0 and |D′| up to 1.0).

As described herein, a locus on chromosome 8q24.21 has been demonstrated to play a role in cancer (e.g., prostate cancer (e.g., aggressive prostate cancer), breast cancer, lung cancer, melanoma). It has been discovered that particular markers and/or haplotypes are present at a higher than expected frequency in particular cancer subjects. In one embodiment, the marker or haplotype comprises one or more markers associated with Chr8q24.21 in linkage disequilibrium (defined as the square of correlation coefficient, r², greater than 0.2) with one or more markers selected from the group consisting of the markers in Table 13.

### Haplotypes and LOD Score Definition of a Susceptibility Locus

In certain embodiments, a candidate susceptibility locus is defined using LOD scores. The defined regions are then ultra-fine mapped with SNP and microsatellite markers with an average spacing between markers of less than 100 kb. All usable microsatellite and SNP markers that are found in public databases and mapped within that region can be used. In addition, microsatellite markers identified within the deCODE genetics sequence assembly of the human genome can be used. The frequencies of haplotypes in the patient and the control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a linkage region, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of an significant marker and/or haplotype association.

A detailed discussion of haplotype analysis follows.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

### Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

### Statistical Analysis

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR2 times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Linkage Disequilibrium Using NEMO

LD between pairs of markers can be calculated using the standard definition of D′ and R² (Lewontin, R, Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower, right corner. In the LD plots the markers can be plotted equidistant rather than according to their physical location, if desired.

### Statistical Methods for Linkage Analysis

Multipoint, affected-only allele-sharing methods can be used in the analyses to assess evidence for linkage. Results, both the LOD-score and the non-parametric linkage (NPL) score, can be obtained using the program Allegro (Gudbjartsson et al., Nat. Genet. 25:12-3 (2000)). Our baseline linkage analysis uses the Sₚₐᵢᵣₛ scoring function (Whittemore, A. S., Halpern, J. Biometrics 50:118-27 (1994); Kruglyak L. et al., Am. J. Hum. Genet. 58:1347-63 (1996)), the exponential allele-sharing model (Kong, A. and Cox, N. J., Am. J. Hum. Genet. 61:1179-88 (1997)) and a family weighting scheme that is halfway, on the log-scale, between weighting each affected pair equally and weighting each family equally. The information measure that we use is part of the Allegro program output and the information value equals zero if the marker genotypes are completely uninformative and equals one if the genotypes determine the exact amount of allele sharing by decent among the affected relatives (Gretarsdottir et al., Am. J. Hum. Genet., 70:593-603 (2002)). The P-values were computed two different ways and the less significant result is reported here. The first P-value can be computed on the basis of large sample theory; the distribution of Zₗᵣ=□(2[logₑ(10)LOD]) approximates a standard normal variable under the null hypothesis of no linkage (Kong, A. and Cox, N. J., Am. J. Hum. Genet. 61:1179-88 (1997)). The second P-value can be calculated by comparing the observed LOD-score with its complete data sampling distribution under the null hypothesis (e.g., Gudbjartsson et al., Nat. Genet. 25:12-3 (2000)). When the data consist of more than a few families, these two P-values tend to be very similar.

### Haplotypes and “Haplotype Block” Definition of a Susceptibility Locus

In certain embodiments, marker and haplotype analysis involves defining a candidate susceptibility locus based on “haplotype blocks” (also called “LD blocks”). It has been reported that portions of the human genome can be broken into series of discrete haplotype blocks containing a few common haplotypes; for these blocks, linkage disequilibrium data provided little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the terms “haplotype block” Or “LD block” includes blocks defined by either characteristic.

Representative methods for identification of haplotype blocks are set forth, for example, in U.S. Published Patent Application Nos. 20030099964, 20030170665, 20040023237 and 20040146870. Haplotype blocks can be used readily to map associations between phenotype and haplotype status. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

### Haplotypes and Diagnostics

As described herein, certain markers and haplotypes are found to be useful for determination of susceptibility to cancer—i.e., they are found to be useful for diagnosing a susceptibility to cancer. Particular markers and haplotypes (e.g., haplotype 1, haplotype 1a, and other haplotypes containing one or more of the markers depicted in any of the Tables below) are found more frequently in individuals with cancer (e.g., prostate cancer (e.g., aggressive prostate cancer), breast cancer, lung cancer, melanoma) than in individuals without cancer. Therefore, these markers and haplotypes have predictive value for detecting cancer, or a susceptibility to cancer, in an individual. Haplotype blocks comprising certain tagging markers, can be found more frequently in individuals with cancer than in individuals without cancer. Therefore, these “at-risk” tagging markers within the haplotype blocks also have predictive value for detecting cancer, or a susceptibility to cancer, in an individual. “At-risk” tagging markers within the haplotype or LD blocks can also include other markers that distinguish among the haplotypes, as these similarly have predictive value for detecting cancer or a susceptibility to cancer. As a consequence of the haplotype block structure of the human genome, a large number of markers or other variants and/or haplotypes comprising such markers or variants in association with the haplotype block (LD block) may be found to be associated with a certain trait and/or phenotype. Thus, it is possible that markers and/or haplotypes residing within LD block A as defined herein or in strong LD (characterized by r² greater than 0.2) with LD block A are associated with cancer (e.g., prostate cancer (e.g., aggressive prostate cancer, breast cancer, lung cancer, melanoma). This includes markers that are described herein (Tables 13, 20 and 21), but may also include other markers that are in strong LD (characterized by r² greater than 0.2) with one or more of the markers listed in Tables 13, 20 and 21. The identification of such additional variants can be achieved by methods well known to those skilled in the art, for example by DNA sequencing of the LD block A genomic region, and the present invention also encompasses such additional variants.

As described herein (e.g., Table 13), certain markers within LD block A are found in decreased frequency in individuals with cancer, and haplotypes comprising two or more markers listed in Tables 13, 20 and 21 are also found to be present at decreased frequency in individuals with cancer. These markers and haplotypes are thus protective for cancer (e.g., prostate cancer (e.g., aggressive prostate cancer), breast cancer, lung cancer, melanoma), i.e. they confer a decreased risk of individuals carrying these markers and/or haplotypes developing cancer. One example of such protective haplotypes is comprised of the markers rs7814251 C allele and rs12542685 allele T allele (Table 22).

The haplotypes and markers described herein are, in some cases, a combination of various genetic markers, e.g., SNPs and microsatellites. Therefore, detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker or haplotype associated with LD Block A and/or Chr8q24.21 is one in which the marker or haplotype is more frequently present in an individual at risk for cancer (affected), compared to the frequency of its presence in a healthy individual (control), wherein the presence of the marker or haplotype is indicative of cancer or a susceptibility to cancer. In other embodiments, at-risk tagging markers in a haplotype block in linkage disequilibrium with one or more markers associated with LD Block A and/or Chr8q24.21, are tagging markers that are more frequently present in an individual at risk for cancer (affected), compared to the frequency of their presence in a healthy individual (control), wherein the presence of the tagging markers is indicative of susceptibility to cancer. In a further embodiment, at-risk markers in linkage disequilibrium with one or more markers associated with LD Block A and/or Chr8q24.21, are markers that are more frequently present in an individual at risk for cancer, compared to the frequency of their presence in a healthy individual (control), wherein the presence of the markers is indicative of susceptibility to cancer.

In particular embodiments of the invention, the marker(s) or ha plotypes are associated with LD Block A. As described and exemplified herein, genotype analysis revealed an association of markers and haplotypes on chromosome 8q24.21 with cancer. In particular, the studies described herein demonstrate an association of markers and haplotypes associated with LD Block A (i.e., the genomic DNA sequence from 128,414,000-128,506,000 bp of NCBI Build 34 (SEQ ID NO: 1; FIG. 6A1-6A31)) with cancer. It should be noted that markers and haplotypes within LD Block A, other than those described in particular herein, can associate with cancer (e.g., prostate cancer (e.g., aggressive prostate cancer), breast cancer, lung cancer, melanoma) and are encompassed by the invention. Based on the teachings described herein and the knowledge in the art, one could identify other markers and haplotypes without undue experimentation (e.g., by sequencing regions of LD Block A in subjects with, and without, cancer or by genotyping markers that are in strong LD with markers and/or haplotypes described herein).

In one embodiment, the marker(s) or haplotype comprises at least one of the markers in Table 13. In another embodiment, the marker(s) or haplotype comprises the rs1447295 A allele and/or the DG8S737 −8 allele.

In certain methods described herein, an individual who is at risk for cancer (e.g., prostate cancer (e.g., aggressive prostate cancer), breast cancer, lung cancer, melanoma) is an individual in whom an at-risk haplotype is identified, or an individual in whom at-risk markers are identified. In one embodiment, the strength of the association of a marker or haplotype is measured by relative risk (RR). RR is the ratio of the incidence of the condition among subjects who carry one copy of the marker or haplotype to the incidence of the condition among subjects who do not carry the marker or haplotype. This ratio is equivalent to the ratio of the incidence of the condition among subjects who carry two copies of the marker or haplotype to the incidence of the condition among subjects who carry one copy of the marker or haplotype. In one embodiment, the marker or haplotype has a relative risk of at least 1.2. In other embodiments, the marker or haplotype has a relative risk of at least 1.3, at least 1.4, at least 1.5, at least 2.0, at least 2.5, at least 3.0, at least 3.5, at least 4.0, or at least 5.0.

In one embodiment, the invention is a method of diagnosing susceptibility to prostate cancer comprising detecting a marker or haplotype associated with LD Block A and/or Chr8q24.21, wherein the presence of the marker or haplotype is indicative of a susceptibility to prostate cancer, and the marker or haplotype has a relative risk of at least 1.5. In another embodiment, the marker or haplotype has a relative risk of at least 2.0.

In one embodiment, the invention is a method of diagnosing susceptibility to breast cancer comprising detecting a marker or haplotype associated with LD Block A and/or Chr8q24.21, wherein the presence of the marker or haplotype is indicative of a susceptibility to breast cancer, and the marker or haplotype has a relative risk of at least 1.3.

In one embodiment, the invention is a method of diagnosing susceptibility to lung cancer comprising detecting a marker or haplotype associated with LD Block A and/or Chr8q24.21, wherein the presence of the marker or haplotype is indicative of a susceptibility to lung cancer, and the marker or haplotype has a relative risk of at least 1.3.

In one embodiment, the invention is a method of diagnosing susceptibility to melanoma comprising detecting a marker or haplotype associated with LD Block A and/or Chr8q24.21, wherein the presence of the marker or haplotype is indicative of a susceptibility to melanoma, and the marker or haplotype has a relative risk of at least 1.5. In another embodiment, the invention is a method of diagnosing susceptibility to malignant cutaneous melanoma comprising detecting a marker or haplotype associated with LD Block A and/or Chr8q24.21, wherein the presence of the marker or haplotype is indicative of a susceptibility to malignant cutaneous melanoma, and the marker or haplotype has a relative risk of at least 1.7.

In another embodiment, significance associated with a marker or haplotype is measured by a relative risk. In one embodiment, a significant increased risk is measured as a relative risk of at least about 1.2, including but not limited to: 1.2, 1.3, 1.4, 1.5, 1.6, 1.7, 1.8 and 1.9. In a further embodiment, a relative risk of at least 1.2 is significant. In a further embodiment, a relative risk of at least about 1.5 is significant. In a further embodiment, a significant increase in risk is at least about 1.7. In another embodiment, a significant decreased risk is measured as a relative risk of less than one, including but not limited to: less than 0.8, 0.7, 0.6, 0.5 and 0.4. In a further embodiment, a relative risk of less than 0.8 is significant. In a further embodiment, a relative risk of less than 0.6 is significant.

In still another embodiment, significance associated with a marker or haplotype is measured by a percentage. In one embodiment, a significant increase or decrease in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase or decrease in risk is at least about 50%. Thus, as used herein, the term “susceptibility to” a cancer indicates that there is an increased or decreased risk of the cancer, by an amount that is significant, when a certain marker (marker allele) or haplotype is present; significance is measured as indicated above. The terms “decreased susceptibility to” a cancer and “protection against” a cancer, as used herein, indicate that the relative risk is decreased accordingly when a certain other marker or haplotype is present. It is understood however, that identifying whether a risk is medically significant may also depend on a variety of factors, including the specific disease, the marker or haplotype, and often, environmental factors.

Particular embodiments of the invention encompass methods of diagnosing a susceptibility to cancer (e.g., prostate cancer (e.g., aggressive prostate cancer), breast cancer, lung cancer, melanoma) in an individual, comprising assessing in the individual the presence or frequency of SNPs and/or microsatellites in, comprising portions of, the nucleic acid region associated with LD Block A and/or Chr8q24.21, wherein an excess or higher frequency of the SNPs and/or microsatellites compared to a healthy control individual is indicative that the individual has cancer, or is susceptible to cancer (see, e.g., Tables 1 and 13 (below) for SNPs and microsatellite markers that that can be used as screening tools and/or are components of haplotypes). These microsatellite markers and SNPs can be identified in haplotypes. For example, a haplotype can include microsatellite markers and/or SNPs such as those set forth in the Tables below. The presence of the marker or haplotype is indicative of cancer, or a susceptibility to cancer, and therefore is indicative of an individual who is a good candidate for therapeutic and/or prophylactic methods. These markers and haplotypes can be used as screening tools. Other particular embodiments of the invention encompass methods of diagnosing a susceptibility to cancer in an individual, comprising detecting one or more markers at one or more polymorphic sites, wherein the one or more polymorphic sites are in linkage disequilibrium with LD Block A and/or Chr8q24.21.

### Utility of Genetic Testing

The knowledge about a genetic variant that confers a risk of developing cancer, offers the opportunity to apply a genetic-test to distinguish between individuals with increased risk of developing the disease (i.e. carriers of the risk variant) and those with decreased risk of developing the disease (i.e. carriers of the protective variant). The core values of genetic testing, for individuals belonging to both of the above mentioned groups, are the possibilities of being able to diagnose the disease at an early stage and provide information to the clinician about prognosis/aggressiveness of the disease in order to be able to apply the most appropriate treatment.

### 1. To Aid Early Detection

The application of a genetic test for prostate cancer can provide an opportunity for the detection of the disease at an earlier stage which leads to higher cure rates, if found locally, and increases survival rates by minimizing regional and distant spread of the tumor.

For prostate cancer, a genetic test will most likely increase the sensitivity and specificity of the already generally applied Prostate Specific Antigen (PSA) test and Digital Rectal Examination (DRE). This can lead to lower rates of false positives (thus minimize unnecessary procedures such as needle biopsies) and false negatives (thus increasing detection of occult disease and minimizing morbidity and mortality due to PCA).

### 2. To Determine Aggressiveness

Genetic testing can provide information about pre-diagnostic prognostic indicators and enable the identification of individuals at high or low risk for aggressive tumor types that can lead to modification in screening strategies. For example, an individual determined to be a carrier of a high risk allele for the development of aggressive prostate cancer will likely undergo more frequent PSA testing, examination and have a lower threshold for needle biopsy in the presence of an abnormal PSA value. Furthermore, identifying individuals that are carriers of high or low risk alleles for aggressive tumor types will lead to modification in treatment strategies. For example, if prostate cancer is diagnosed in an individual that is a carrier of an allele that confers increased risk of developing an aggressive form of prostate cancer, then the clinician would likely advise a more aggressive treatment strategy such as a prostatectomy instead of a less aggressive treatment strategy.

As is known in the art, Prostate Specific Antigen (PSA) is a protein that is secreted by the epithelial cells of the prostate gland, including cancer cells. An elevated level in the blood indicates an abnormal condition of the prostate, either benign or malignant. PSA is used to detect potential problems in the prostate gland and to follow the progress of prostate cancer therapy. PSA levels above 4 ng/ml are indicative of the presence of prostate cancer (although as known in the art and described herein, the test is neither very specific nor sensitive).

In one embodiment, the method of the invention is performed in combination with (either prior to, concurrently or after) a PSA assay. In a particular embodiment, the presence of a marker or haplotype, in conjunction with the subject having a PSA level greater than 4 ng/ml, is indicative of a more aggressive prostate cancer and/or a worse prognosis. As described herein, particular markers and haplotypes are associated with high Gleason (i.e., more aggressive) prostate cancer. In another embodiment, the presence of a marker or haplotype, in a patient who has a normal PSA level (e.g., less than 4 ng/ml), is indicative of a high Gleason (i.e., more aggressive) prostate cancer and/or a worse prognosis. A “worse prognosis” or “bad prognosis” occurs when it is more likely that the cancer will grow beyond the boundaries of the prostate gland, metastasize, escape therapy and/or kill the host.

In one embodiment, the presence of a marker or haplotype is indicative of a predisposition to a somatic rearrangement of Chr8q24.21 (e.g., one or more of an amplification, a translocation, an insertion and/or deletion) in a tumor or its precursor. The somatic rearrangement itself may subsequently lead to a more aggressive form of prostate cancer (e.g., a higher histologic grade, as reflected by a higher Gleason score or higher stage at diagnosis, an increased progression of prostate cancer (e.g., to a higher stage), a worse outcome (e.g., in terms of morbidity, complications or death)). As is known in the art, the Gleason grade is a widely used method for classifying prostate cancer tissue for the degree of loss of the normal glandular architecture (size, shape and differentiation of glands). A grade from 1-5 is assigned successively to each of the two most predominant tissue patterns present in the examined tissue sample and are added together to produce the total or combined Gleason grade (scale of 2-10). High numbers indicate poor differentiation and therefore more aggressive cancer.

Aggressive prostate cancer is cancer that grows beyond the prostate, metastasizes and eventually kills the patient. As described herein, one surrogate measure of aggressivity is a high combined Gleason grade. The higher the grade on a scale of 2-10 the more likely it is that a patient has aggressive disease.

As used herein and unless noted differently, the term “stage” is used to define the size and physical extent of a cancer (e.g., prostate cancer). One method of staging various cancers is the TNM method, wherein in the TNM acronym, T stands for tumor size and invasiveness (e.g., the primary tumor in the prostate); N relates to nodal involvement (e.g., prostate cancer that has spread to lymph nodes); and M indicates the presence or absense of metastates (spread to a distant site).

### Methods of the Invention

Methods for the diagnosis of susceptibility to cancer (e.g., prostate cancer (e.g., aggressive prostate cancer), breast cancer, lung cancer, melanoma) are described herein and are encompassed by the invention. Kits for assaying a sample from a subject to detect susceptibility to cancer (e.g., prostate cancer (e.g., aggressive prostate cancer), breast cancer, lung cancer, melanoma) are also encompassed by the invention. In other embodiments, the invention is a method for diagnosing Chr8q24.21-associated cancer (e.g., Chr8q24.21-associated prostate cancer, Chr8q24.21-associated breast cancer, Chr8q24.21-associated lung cancer, Chr8q24.21-associated melanoma) in a subject.

### Diagnostic and Screening Assays of the Invention

In certain embodiments, the present invention pertains to methods of diagnosing, or aiding in the diagnosis of, cancer or a susceptibility to cancer, by detecting particular genetic markers that appear more frequently in cancer subjects or subjects who are susceptible to cancer. In a particular embodiment, the invention is a method of diagnosing a susceptibility to prostate cancer (e.g., aggressive prostate cancer), breast cancer, lung cancer and/or melanoma by detecting one or more particular genetic markers (e.g., the markers or haplotypes described herein). The present invention describes methods whereby detection of particular markers or haplotypes is indicative of a susceptibility to cancer (e.g., prostate cancer (e.g., aggressive prostate cancer), breast cancer, lung cancer, melanoma). Such prognostic or predictive assays can also be used to determine prophylactic treatment of a subject prior to the onset of symptoms associated with such cancers.

In addition, in certain other embodiments, the present invention′ pertains to methods of diagnosing, or aiding in the diagnosis of, a decreased susceptibility to cancer, by detecting particular genetic markers or haplotypes that appear less frequently in cancer. In a particular embodiment, the invention is a method of diagnosing a decreased susceptibility to prostate cancer (e.g., aggressive prostate cancer), breast cancer, lung cancer and/or melanoma by detecting one or more particular genetic markers (e.g., the markers or haplotypes described herein). The present invention describes methods whereby detection of particular markers or haplotypes is indicative of a decreased susceptibility to cancer (e.g., prostate cancer (e.g., aggressive prostate cancer), breast cancer, lung cancer, melanoma), or of a protective marker or haplotype against the cancer.

As described and exemplified herein, particular markers or haplotypes associated with LD Block A and/or Chr8q24.21 (e.g., haplotypes) are linked to cancer (e.g., prostate cancer (e.g., aggressive prostate cancer), breast cancer, lung cancer, melanoma). In one embodiment, the marker or haplotype is one that confers a significant risk of susceptibility to prostate cancer, breast cancer, lung cancer and/or melanoma. In another embodiment, the invention pertains to methods of diagnosing a susceptibility to cancer (e.g., prostate cancer (e.g., aggressive prostate cancer), breast cancer, lung cancer, melanoma) in a subject, by screening for a marker or haplotype associated with LD Block A and/or Chr8q24.21 that is more frequently present in a subject having, or who is susceptible to, cancer (affected), as compared to the frequency of its presence in a healthy subject (control). In certain embodiments, the marker or haplotype has a p value <0.05.

In these embodiments, the presence of the marker or haplotype is indicative of a susceptibility to cancer (e.g., prostate cancer (e.g., aggressive prostate cancer), breast cancer, lung cancer, melanoma). These diagnostic methods involve detecting the presence or absence of a marker or haplotype that is associated with LD Block A and/or Chr8q24.21. The haplotypes described herein include combinations of various genetic markers (e.g., SNPs, microsatellites). The detection of the particular genetic markers that make up the particular haplotypes can be performed by a variety of methods described herein and/or known in the art. For example, genetic markers can be detected at the nucleic acid level (e.g., by direct nucleotide sequencing) or at the amino acid level if the genetic marker affects the coding sequence of a protein encoded by a Chr8q24.21-associated nucleic acid (e.g., by protein sequencing or by immunoassays using antibodies that recognize such a protein). As used herein, a “Chr8q24.21-associated nucleic acid” refers to a nucleic acid that is, or corresponds to, a fragment of a genomic DNA sequence of Chr8q24.21. A “LD Block A-associated nucleic acid” refers to a nucleic acid that is, or corresponds to, a fragment of a genomic DNA sequence of LD Block A.

In one embodiment, diagnosis of a susceptibility to cancer (e.g., prostate cancer (e.g., aggressive prostate cancer), breast cancer, lung cancer, melanoma) can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject suspected of having, being susceptible to, or predisposed for cancer (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of an allele of the haplotype can be indicated by sequence-specific hybridization of a nucleic acid probe specific for the particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to cancer (e.g., prostate cancer (e.g., aggressive prostate cancer), breast cancer, lung cancer, melanoma), a hybridization sample is formed by contacting the test sample containing a Chr8q24.21-associated and/or LD Block A-associated nucleic acid, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can be all or a portion of SEQ ID NO:1, optionally comprising at least one allele contained in the haplotypes described herein, or the probe can be the complementary sequence of such a sequence. In a particular embodiment, the nucleic acid probe is a portion of SEQ ID NO:1, optionally comprising at least one allele contained in the haplotypes described herein, or the probe can be the complementary sequence of such a sequence. Other suitable probes for use in the diagnostic assays of the invention are described herein.

The hybridization sample is maintained under conditions that are sufficient to allow specific hybridization of the nucleic acid probe to the Chr8q24.21-associated nucleic acid and/or LD Block A-associated nucleic acid. “Specific hybridization”, as used herein, indicates exact hybridization (e.g., with no mismatches). Specific hybridization can be performed under high stringency conditions or moderate stringency conditions as described herein. In one embodiment, the hybridization conditions for specific hybridization are high stringency (e.g., as described herein).

Specific hybridization, if present, is then detected using standard methods. If specific hybridization occurs between the nucleic acid probe and the Chr8q24.21-associated and/or LD Block A-associated nucleic acid in the test sample, then the sample contains the allele that is complementary to the nucleotide that is present in the nucleic acid probe. The process can be repeated for the other markers that make up the haplotype, or multiple probes can be used concurrently to detect more than one marker at a time. It is also possible to design a single probe containing more than one marker of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., an haplotype) and therefore is susceptible to cancer (e.g., prostate cancer (e.g., aggressive prostate cancer), breast cancer, lung cancer, melanoma).

In another hybridization method, Northern analysis (see Current Protocols in Molecular Biology, Ausubel, F. et al., eds., John Wiley & Sons, supra) is used to identify the presence of a polymorphism associated with cancer or a susceptibility to cancer (e.g., prostate cancer (e.g., aggressive prostate cancer), breast cancer, lung cancer, melanoma). For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the genetic markers of a haplotype that is associated with cancer (e.g., prostate cancer (e.g., aggressive prostate cancer), breast cancer, lung cancer, melanoma). Hybridization of the PNA probe is diagnostic for cancer or a susceptibility to cancer.

In one embodiment of the invention, diagnosis of cancer or a susceptibility to cancer (e.g., prostate cancer (e.g., aggressive prostate cancer), breast cancer, lung cancer, melanoma) is accomplished through enzymatic amplification of a nucleic acid from the subject. For example, a test sample containing genomic DNA can be obtained from the subject and the polymerase chain reaction (PCR) can be used to amplify a Chr8q24.21-associated nucleic acid and/or LD Block A-associated nucleic acid in the test sample. As described herein, identification of a particular marker or haplotype (e.g., an haplotype) associated with the amplified Chr8q24.21 region and/or LD Block A region can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.), to allow the identification of polymorphisms and haplotypes (e.g., haplotypes). The technique can assess the presence of an alteration in the expression or composition of a polypeptide or splicing variant(s) that is encoded by Chr8q24.21 and/or LD Block A. Further, the expression of the variant(s) can be quantified as physically or functionally different.

In another method of the invention, analysis by restriction digestion can be used to detect a particular allele if the allele results in the creation or elimination of a restriction site relative to a reference sequence. A test sample containing genomic DNA is obtained from the subject. PCR can be used to amplify particular regions of Chr8q24.21 and/or LD Block A in the test sample from the test subject. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with Chr8q24.21 and/or LD Block A. Therefore, in one embodiment, determination of the presence or absence of a particular marker or haplotype (e.g., an haplotype) comprises sequence analysis. For example, a test sample of DNA or RNA can be obtained from the test subject. PCR or other appropriate methods can be used to amplify a portion of Chr8q24.21 and/or LD Block A, and the presence of a specific allele can then be detected directly by sequencing the polymorphic site of the genomic DNA in the sample.

Allele-specific oligonucleotides can also be used to detect the presence of a particular allele at a polymorphic site associated with Chr8q24.21 and/or LD Block A, through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a region of Chr8q24.21 and/or LD Block A, and which contains a specific allele at a polymorphic site (e.g., a polymorphism described herein). An allele-specific oligonucleotide probe that is specific for one or more particular polymorphisms associated with Chr8q24.21 and/or LD Block A can be prepared using standard methods (see, e.g., Current Protocols in Molecular Biology, supra). PCR can be used to amplify the desired region of Chr8q24.21 and/or LD Block A. The DNA containing the amplified Chr8q24.21 region and/or LD Block A region can be dot-blotted using standard methods (see, e.g., Current Protocols in Molecular Biology, supra), and the blot can be contacted with the oligonucleotide probe. The presence of specific hybridization of the probe to the amplified Chr8q24.21 region and/or LD Block A region can then be detected. Specific hybridization of an allele-specific oligonucleotide probe to DNA from the subject is indicative of a specific allele at a polymorphic site associated with Chr8q24.21 and/or LD Block A (see, e.g., Gibbs, R. et al., Nucleic Acids Res., 17:2437-2448 (1989) and WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a Chr8q24.21-associated nucleic acid and/or LD Block A-associated nucleic acid. For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods or light directed synthesis methods that incorporate a combination of photolithographic methods and solid phase oligonucleotide synthesis methods (Fodor, S. et al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein and also in, e.g., published PCT Application Nos. WO 92/10092 and WO 95/11995, and U.S. Pat. No. 5,424,186, the entire teachings of each of which are incorporated by reference herein. In brief, a target nucleic acid sequence, which includes one or more previously identified polymorphic markers, is amplified by well-known amplification techniques (e.g., PCR). Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms (e.g., multiple polymorphisms of a particular haplotype (e.g., an haplotype)). In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of both of which are incorporated by reference herein.

Other methods of nucleic acid analysis can be used to detect a particular allele at a polymorphic site associated with Chr8q24.21 and/or LD Block A. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No.5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:2541 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:43974401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of cancer or a susceptibility to cancer (e.g., prostate cancer (e.g., aggressive prostate cancer), breast cancer, lung cancer, melanoma) can be made by examining expression and/or composition of a polypeptide encoded by a Chr8q24.21-associated nucleic acid and/or LD Block A-associated nucleic acid in those instances where the genetic marker(s) or haplotype described herein results in a change in the composition or expression of the polypeptide. As described herein, particular genes and predicted genes that map to Chr8q24.21 include, e.g., POU5FLC20 (Genbank Accession No. AF268618; known gene), Genbank Accession No. BE676854 (EST), Genbank Accession No. AL709378 (EST), Genbank Accession No. BX108223 (EST), Genbank Accession No. AA375336 (EST), Genbank Accession No. CB104826 (EST), Genbank Accession No. BG203635 (EST), Genbank Accession No. AW183883 (EST), Genbank Accession No. BM804611 (EST), C-MYC (Genbank Accession No. NM_{—}002467; known gene) and PVT1 (Genbank Accession No. XM_{—}372058; known gene). Thus, diagnosis of a susceptibility to cancer (e.g., prostate cancer (e.g., aggressive prostate cancer), breast cancer, lung cancer, melanoma) can be made by examining expression and/or composition of one of these polypeptides, or another polypeptide encoded by a Chr8q24.21-associated nucleic acid and/or LD Block A-associated nucleic acid, in those instances where the genetic marker or haplotype described herein results in a change in the composition or expression of the polypeptide. The haplotypes and markers described herein that show association to cancer may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

The c-myc gene on Chr8q24.21 encodes the c-MYC protein that was identified over 20 years ago as the cellular counterpart of the viral oncogene v-myc of the avian myelocytomatosis retrovirus (Vennstrom et al., J. Virology 42:773-79 (1982)). The c-MYC protein is a transcription factor that is rapidly induced upon treatment of cells with mitogenic stimuli. c-MYC regulates the expression of many genes by binding E-boxes (CACGTG) in a heterodimeric complex with a protein named MAX. Many of the genes regulated by c-MYC are involved in cell cycle control. c-MYC promotes cell-cycle progression, inhibits cellular differentiation and induces apoptosis. c-MYC also has a negative effect on double strand DNA repair (Karlsson, A, et al., Proc. Natl. Acad. Sci. USA 100(17):9974-79 (2003)). c-MYC also promotes angiogenesis (Ngo, C. V., et al., Cell Growth Differ. 11(4):201-10 (2000); Baudino T. A., et al., Genes Dev. 16(19):2530-43 (2002)).

The c-myc gene is highly tumorigenic in vitro and in vivo. c-MYC synergizes with proteins that inhibit apoptosis such as BCL, BCL-X_{L} or with the loss of p53 or ARF in lymphomagenesis in transgenic mice (Strasser et al., Nature 348:331-333 (1990); Blyth, K., et al., Oncogene 10:1717-23 (1990); Elson, A., et al., Oncogene 11:181-90 (1995); Eischen, C. M., et al., Genes Dev. 13:2658-69 (1999)).

Amplification and overexpression of the c-myc gene is seen in prostate cancer and is often associated with aggressive tumors, hormone independence and a poor prognosis (Jenkins, R. B., et al., Cancer Res. 57(3):524-31 (1997); El Gedaily, A., et al., Prostate 46(3):184-90 (2001); Saramaki, O., et al., Am. J. Pathol. 159(6):2089-94 (2001); Bubendorf, L., et al., Cancer Res. 59(4):803-06 (1999)). c-myc and the Chr8q24.21 region is furthermore gained in prostate, breast and lung tumors and in melanoma (Blancato J., et al., Br. J. Cancer 90(8):1612-9 (2004); Kubokura, H., et al., Ann. Thorac. Cardiovasc. Surg. 7(4):197-203 (2001); Treszl, A., et al., Cytometry 60B(1):37-46 (2004); Kraehn, G. M., et al., Br. J. Cancer 84(1):72-79 (2001)). In addition, many other tumor types show a gain of this region including colon, liver, ovary, stomach, intestinal and bladder cancer. Combining all tumor types shows that Chr8q24.21 is the most frequently gained chromosomal region with gain in approximately 17% of all tumor types (www.progenetix.com).

The oncogene is involved in Burkitt's lymphoma as a result of translocations that juxtapose c-myc to immunoglobulin enhancers, thereby activating expression of the gene (Dalla-Favera, R., et al., Proc. Natl. Acad. Sci. USA 79(24):7824-27 (1982); Taub, R., et al., Proc. Natl. Acad. Sci. USA 79(24):7837-41 (1982). It is also involved in cervical cancer by Human papillomavirus (HPV) integration close to the gene. In most cases, HPV integrations occur in a region spanning 500 kb centromeric and 200 kb telomeric of the c-myc gene (Ferber, J. M., et al., Cancer Genetics Cytogenetics 154:1-9 (2004); Ferber, M. J., et al., Oncogene 22:7233-7242 (2003)).

Two fragile sites, FRA8C and FRA8D, lie centromeric and telomeric to c-myc, respectively, on Chr8q24.21. Fragile sites are prone to breakage in the presence of agents that arrest DNA synthesis. Replication of fragile sites is thought to occur late in S-phase and upon induction even later. The involvement of fragile sites in chromosomal amplification, translocation and/or viral insertion may relate to the late replication of these sites and that a break is initiated at or close to stalled replication forks (Hellman, A., et al., Cancer Cell 1:89-97 (2002)).

It is possible that markers or haplotypes described here within LD Block A or in strong LD with LD block A (as measured by r² greater than 0.2) could affect the stability of the region leading to gene amplifications of the c-myc gene or other nearby genes. That is, a person could inherit the LD Block A or a region in strong LD with LD block A (as measured by r² greater than 0.2) from one or both parents and therefore be more likely to have a somatic mutational event later in one or more cells leading to progression of cancer to a more aggressive form. Thus, in one embodiment, identification of a marker or haplotype of the invention (e.g., a marker or haplotype associated with LD Block A) may be used to diagnose a susceptibility to a somatic mutational event, which can lead to progression of cancer to a more aggressive form

In one embodiment, the marker or haplotype does not comprise a marker that is located within the c-myc open reading frame (i.e., chr8:128,705,092-128,710,260 bp in NCBI Build 34). In another embodiment, the marker or haplotype does not comprise a marker that is located within the c-myc promoter or open reading frame. In yet another embodiment, the marker or haplotype does not comprise a marker that is located within the c-myc promoter, enhancer or open reading frame. In-still other embodiments, the marker or haplotype does not comprise a marker that is located within 1 kb, 2 kb, 5 kb, 10 kb, 15 kb, 20 kb or 25 kb of the c-myc open reading frame.

A variety of methods can be used to make such a detection, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a Chr8q24.21-associated nucleic acid and/or LD Block A-associated nucleic acid. An alteration in expression of a polypeptide encoded by a Chr8q24.21-associated nucleic acid and/or LD Block A-associated nucleic acid can be, for example, an alteration in the quantitative polypeptide expression (i.e., the amount of polypeptide produced). An alteration in the composition of a polypeptide encoded by a Chr8q24.21-associated nucleic acid and/or LD Block A-associated nucleic acid is an alteration in the qualitative polypeptide expression (e.g., expression of a mutant polypeptide or of a different splicing variant). In one embodiment, diagnosis of a susceptibility to cancer (e.g., prostate cancer (e.g., aggressive prostate cancer), breast cancer, lung cancer, melanoma) is made by detecting a particular splicing variant encoded by a Chr8q24.21-associated nucleic acid and/or LD Block A-associated nucleic acid, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of polypeptide encoded by a Chr8q24.21-associated nucleic acid and/or LD Block A-associated nucleic acid in a control sample. A control sample is a sample that corresponds to the test sample (e.g., is from the same type of cells), and is from a subject who is not affected by, and/or who does not have a susceptibility to, cancer (e.g., a subject that does not possess a marker or haplotype as described herein). Similarly, the presence of one or more different splicing variants in the test sample, or the presence of significantly different amounts of different splicing variants in the test sample, as compared with the control sample, can be indicative of a susceptibility to cancer (e.g., prostate cancer (e.g., aggressive prostate cancer), breast cancer, lung cancer, melanoma). An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a Chr8q24.21-associated nucleic acid and/or LD Block A-associated nucleic acid can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a Chr8q24.21-associated nucleic acid and/or LD Block A-associated nucleic acid can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a Chr8q24.21-associated nucleic acid and/or LD Block A-associated nucleic acid in a test sample is compared with the level or amount of the polypeptide encoded by a Chr8q24.21-associated nucleic acid and/or LD Block A-associated nucleic acid in a control sample. A level or amount of the polypeptide in the test sample that is higher or lower than the level or amount of the polypeptide in the control sample, such that the difference is statistically significant, is indicative of an alteration in the expression of the polypeptide encoded by the Chr8q24.21-associated nucleic acid and/or LD Block A-associated nucleic acid, and is diagnostic for a particular allele responsible for causing the difference in expression. Alternatively, the composition of the polypeptide encoded by a Chr8q24.21-associated nucleic acid and/or LD Block A-associated nucleic acid in a test sample is compared with the composition of the polypeptide encoded by a Chr8q24.21-associated nucleic acid and/or LD Block A-associated nucleic acid in a control sample. In another embodiment, both the level or amount and the composition of the polypeptide can be assessed in the test sample and in the control sample.

As described and exemplified herein, particular markers and haplotypes (e.g., haplotype 1, haplotype 1a, haplotypes containing two or more markers listed in the Tables below) associated with Chr8q24.21 and/or LD Block A are linked to cancer (e.g., prostate cancer (e.g., aggressive prostate cancer), breast cancer, lung cancer, melanoma). In one embodiment, the invention pertains to a method of diagnosing a susceptibility to cancer (e.g., prostate cancer (e.g., aggressive prostate cancer), breast cancer, lung cancer, melanoma) in a subject, comprising screening for a marker or haplotype associated with a Chr8q24.21-associated nucleic acid and/or LD Block A-associated nucleic acid that is more frequently present in a subject having, or who is susceptible to, cancer (affected), as compared to the frequency of its presence in a healthy subject (control). In this embodiment, the presence of the marker or haplotype is indicative of a susceptibility to cancer. Standard techniques for genotyping for the presence of SNPs and/or microsatellite markers associated with cancer can be used, such as fluorescence-based techniques (Chen, X., et al., Genome Res., 9:492-498 (1999)), PCR, LCR, Nested PCR and other techniques for nucleic acid amplification. In one embodiment, the method comprises assessing in a subject the presence or frequency of one or more specific SNP alleles and/or microsatellite alleles that are associated with Chr8q24.21 and/or LD Block A and are linked to cancer and/or susceptibility to cancer. In this embodiment, an excess or higher frequency of the allele(s), as compared to a healthy control subject, is indicative that the subject is susceptible to cancer.

In another embodiment, the diagnosis of a susceptibility to cancer (e.g., prostate cancer (e.g., aggressive prostate cancer), breast cancer, lung cancer, melanoma) is made by detecting at least one Chr8q24.21-associated allele and/or LD Block A-associated allele in combination with an additional protein-based, RNA-based or DNA-based assay (e.g., other cancer diagnostic assays including, but not limited to: PSA assays, carcinoembryonic antigen (CEA) assays, BRCA1 assays and BRCA2 assays). Such cancer diagnostic assays are known in the art. The methods of the invention can also be used in combination with an analysis of a subject's family history and risk factors (e.g., environmental risk factors, lifestyle risk factors).

As is known in the art, and as described herein, PSA testing has aided early diagnosis of prostate cancer, but it is neither highly sensitive nor specific (Punglia et al., N. Engl. J. Med. 349(4):335-42 (2003)). Accordingly, PSA testing alone leads to a high percentage of false negative and false positive diagnoses, which results in both many instances of missed cancers and unnecessary follow-up biopsies for those without cancer. In one embodiment, the diagnosis of prostate cancer or a susceptibility to prostate cancer is made by detecting at least one Chr8q24.21-associated allele and/or LD Block A-associated allele in combination with a PSA assay.

### Kits

Kits useful in the methods of diagnosis comprise components useful in any of the methods described herein, including for example, hybridization probes, restriction enzymes (e.g., for RFLP analysis), allele-specific oligonucleotides, antibodies that bind to an altered polypeptide encoded by a Chr8q24.21 nucleic acid and/or LD Block A-associated nucleic acid (e.g., antibodies that bind to a polypeptide comprising at least one genetic marker included in the haplotypes described herein) or to a non-altered (native) polypeptide encoded by a Chr8q24.21 nucleic acid and/or LD Block A-associated nucleic acid, means for amplification of a Chr8q24.21 nucleic acid and/or LD Block A-associated nucleic acid, means for analyzing the nucleic acid sequence of a Chr8q24.21 nucleic acid and/or LD Block A-associated nucleic acid, means for analyzing the amino acid sequence of a polypeptide encoded by a Chr8q24.21 nucleic acid and/or LD Block A-associated nucleic acid, etc. Additionally, kits can provide reagents for assays to be used in combination with the methods of the present invention, e.g., reagents for use with other cancer diagnostic assays (e.g., reagents for detecting PSA, CEA, BRCA1, BRCA2, etc.).

In one embodiment, the invention is a kit for assaying a sample from a subject to detect cancer or a susceptibility to cancer (e.g., prostate cancer (e.g., aggressive prostate cancer), breast cancer, lung cancer, melanoma) in a subject, wherein the kit comprises one or more reagents for detecting a marker or haplotype associated with Chr8q24.21 and/or LD Block A. In a particular embodiment, the kit comprises at least one contiguous nucleotide sequence that is completely complementary to a region comprising at least one of the markers associated with Chr8q24.21 and/or LD Block A. In another embodiment, the kit comprises one or more nucleic acids that are capable of detecting one or more specific markers or haplotypes. In still another embodiment, the kit comprises one or more reagents that comprise at least one contiguous nucleotide sequence that is completely complementary to a region comprising at least one of the markers from Table 1 or Table 13 (e.g., a region of SEQ ID NO:1 containing at least one of the markers from Table 1 or Table 13), or another Table below. Such contiguous nucleotide sequences or nucleic acids (e.g., oligonucleotide primers) can be designed using portions of the nucleic acids flanking SNPs or microsatellites that are indicative of cancer or a susceptibility to cancer. Such nucleic acids (e.g., oligonucleotide primers) are designed to amplify regions of Chr8q24.21 and/or LD Block A that are associated with a marker or haplotype for cancer. In another embodiment, the kit comprises one or more labeled nucleic acids capable of detecting one or more specific markers or haplotypes associated with Chr8q24.21 and/or LD Block A and reagents for detection of the label. Suitable labels include, e.g., a radioisotope, a fluorescent label, an enzyme label, an enzyme co-factor label, a magnetic label, a spin label, an epitope label.

In particular embodiments, the marker or haplotype to be detected by the reagents of the kit comprises one or more markers, two or more markers, three or more markers, four or more markers or five or more markers selected from the group consisting of the markers in Table 13. In another embodiment, the marker or haplotype to be detected comprises the rs1447295 A allele and/or the DG8S737 −8 allele. In such embodiments, the presence of the marker or haplotype is indicative of a susceptibility to cancer (e.g., prostate cancer (e.g., aggressive prostate cancer), breast cancer, lung cancer, melanoma).

### Diagnosis of Chr8q24.21-Associated Prostate Cancer

Although the methods of diagnosis have been generally described in the context of diagnosing susceptibility to cancer (e.g., prostate cancer (e.g., aggressive prostate cancer), breast cancer, lung cancer, melanoma), the methods can also be used to diagnose Chr8q24.21-associated cancer (e.g., Chr8q24.21-associated prostate cancer, Chr8q24.21-associated breast cancer, Chr8q24.21-associated lung cancer, Chr8q24.21-associated melanoma). For example, an individual having cancer can be assessed to determine whether the presence in the individual of a polymorphism in a Chr8q24.21-associated nucleic acid and/or LD Block A-associated nucleic acid, and/or the presence of a haplotype in the individual, could have been a contributing factor to the individual's cancer. As used herein, the terms, “Chr8q24.21-associated cancer”, “Chr8q24.21-associated prostate cancer”, “Chr8q24.21-associated breast cancer”, “Chr8q24.21-associated lung cancer” and “Chr8q24.21-associated melanoma” refer to the occurrence of cancer, or a particular form of cancer, in a subject who has a polymorphism in a Chr8q24.21-associated nucleic acid sequence or a haplotype associated with Chr8q24.21. Identification of Chr8q24.21-associated cancer (e.g., Chr8q24.21-associated prostate cancer, Chr8q24.21-associated breast cancer, Chr8q24.21-associated lung cancer, Chr8q24.21-associated melanoma) facilitates treatment planning, as treatment can be designed and therapeutics selected to target the appropriate Chr8q24.21-associated gene or protein.

In one embodiment of the invention, diagnosis of Chr8q24.21-associated cancer (e.g., Chr8q24.21-associated prostate cancer, Chr8q24.21-associated breast cancer, Chr8q24.21-associated lung cancer, Chr8q24.21-associated melanoma) is made by detecting a polymorphism in a Chr8q24.21-associated nucleic acid (e.g., using the methods described herein and/or other methods known in the art). Particular polymorphisms in Chr8q24.21-associated nucleic acid sequences are described herein (see, e.g., Table 1 and Table 13). A test sample of genomic DNA, RNA, or cDNA, is obtained from a subject having cancer to determine whether the cancer is associated with Chr8q24.21. The DNA, RNA or cDNA sample is then examined to determine whether a polymorphism in a Chr8q24.21-associated nucleic acid sequence is present. If the Chr8q24.21-associated nucleic acid sequence has the polymorphism then the presence of the polymorphism is indicative of the Chr8q24.21-associated cancer.

For example, in one embodiment, hybridization methods, such as Southern analysis, Northern analysis or in situ hybridization, can be used to detect the polymorphism. In other embodiments, mutation analysis by restriction digestion or sequence analysis can be used, as can allele-specific oligonucleotides, or quantitative PCR (kinetic thermal cycling). Diagnosis of Chr8q24.21-associated cancer can also be made by examining expression and/or composition of a polypeptide encoded by a Chr8q24.21-associated nucleic acid, using a variety of methods, including enzyme linked immunosorbent assays (ELISAs), Western blots, immunoprecipitations and immunofluorescence. A test sample from a subject is assessed for the presence of an alteration in the expression and/or an alteration in composition of the polypeptide encoded by a Chr8q24.21-associated nucleic acid, or for the presence of a particular variant encoded by a Chr8q24.21-associated nucleic acid. An alteration in expression of a polypeptide encoded by a Chr8q24.21-associated nucleic acid can be, for example, an alteration in the quantitative polypeptide expression (i.e., the amount of polypeptide produced); an alteration in the composition of a polypeptide encoded by a Chr8q24.21-associated nucleic acid is an alteration in the qualitative polypeptide expression (e.g., expression of an altered Chr8q24.21-associated polypeptide or of a different splicing variant).

In other embodiments, the invention pertains to a method for the diagnosis and identification of Chr8q24.21-associated cancer (e.g., Chr8q24.21-associated prostate cancer, Chr8q24.21-associated breast cancer, Chr8q24.21-associated lung cancer, Chr8q24.21-associated melanoma) in a subject, by identifying the presence of a marker or haplotype associated with Chr8q24.21, as described in detail herein. For example, the markers and/or haplotypes described herein in Tables 1, 2, 4, 5 and 13 are found more frequently in subjects with cancer (e.g., prostate cancer (e.g., aggressive prostate cancer), breast cancer, lung cancer, melanoma) than in subjects not affected by cancer. Therefore, these markers and/or haplotypes have predictive value for detecting Chr8q24.21-associated cancer. In one embodiment, the marker or haplotype having predictive value for detecting Chr8q24.21-associated cancer comprises one or more markers selected from the group consisting of the markers in Table 13. In another embodiment, the marker or haplotype having predictive value for detecting Chr8q24.21-associated cancer comprises one or more markers selected from the group consisting of the DG8S737 −8 allele and the rs1447295 A allele. In still other embodiments, the haplotype having predictive value for detecting Chr8q24.21-associated cancer comprises haplotype 1 or haplotype la. The methods! described herein can be used to assess a sample from a subject for the presence or absence of a marker or haplotype; the presence of a marker or haplotype is indicative of Chr8q24.21-associated cancer.

As is known in the art, individuals can have differential responses to a particular therapy (e.g., a thereapeutic agent). The basis of the differential response may be genetically determined in part. Accordingly, in one embodiment, the presence of a marker or haplotype is indicative of a different response rate to a particular treatment modality. This means that a cancer patient carrying a marker or haplotype on Chr8q24.21 would respond better to, or worse to, a specific therapeutic, antihormonal drug and/or radiation therapy used to treat cancer. Therefore, the presence or absence of the marker or haplotype could aid in deciding what treatment should be used for a cancer patient. For example, for a newly diagnosed prostate cancer patient, the presence of a marker or haplotype-on Chr8q24.21 may be assessed (e.g., through testing DNA derived from a blood sample, as described herein). If the patient is positive for a marker or haplotype at Chr8q24.21 (that is, the marker or haplotype is present), then the physician recommends one particular therapy, while if the patient is negative for a marker or baplotype, then a different course of therapy may be recommended (which may include recommending that no immediate therapy, other than serial monitoring for progression of prostate cancer, be performed). Thus, the patient's carrier status could be used to help determine whether a particular treatment modality (e.g., a chemotherapeutic agent, an antihormonal agent, radiation treatment) should be administered.

### Nucleic Acids and Polypeptides of the Invention

The nucleic acids and polypeptides described herein can be used in methods of diagnosis of a susceptibility to cancer (e.g., prostate cancer (e.g., aggressive prostate cancer), breast cancer, lung cancer, melanoma), as well as in kits useful for such diagnosis.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a haplotype described herein). In one embodiment, the invention includes variants that hybridize under high stringency hybridization and wash conditions (e.g., for selective hybridization) to a nucleotide sequence that comprises SEQ ID NO:1 or a fragment thereof (or a nucleotide sequence comprising the complement of SEQ ID NO:1 or a fragment thereof), wherein the nucleotide sequence comprises at least one polymorphic allele contained in the haplotypes (e.g., haplotypes) described herein.

Such nucleic acid molecules can be detected and/or isolated by allele- or sequence-specific hybridization (e.g., under high stringency conditions). Stringency conditions and methods for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998)), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Another non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The present invention also provides isolated nucleic acid molecules that contain a fragment or portion that hybridizes under highly stringent conditions to a nucleic acid that comprises, or consists of, SEQ ID NO:1 or a fragment thereof (or a nucleotide sequence comprising, or consisting of, the complement of SEQ ID NO:1 or a fragment thereof), wherein the nucleotide sequence comprises at least one polymorphic allele contained in the haplotypes (e.g., haplotypes) described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991).

A probe or primer comprises a region of nucleotide sequence that hybridizes to at least about 15, typically about 20-25, and in certain embodiments about 40, 50 or 75, consecutive nucleotides of a nucleic acid molecule comprising a contiguous nucleotide sequence from SEQ ID NO:1 and comprising at least one allele contained in one or more haplotypes described herein, and the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques and the sequence information provided in SEQ ID NO:1. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila, P. et al., Nucleic Acids Res., 19:4967-4973 (1991); Eckert, K. and Kunkel, T., PCR Methods and Applications, 1:17-24 (1991); PCR (eds. McPherson et al., IRL Press, Oxford); and U.S. Pat. No. 4,683,202, the entire teachings of each of which are incorporated herein by reference.

Other suitable amplification methods include the ligase chain reaction (LCR; see Wu, D. and Wallace, R., Genomics, 4:560469 (1989); Landegren, U. et al., Science, 241:1077-1080 (1988)), transcription amplification (Kwoh, D. et al., Proc. Nati. Acad. Sci. USA, 86:1173-1177 (1989)), self-sustained sequence replication (Guatelli, J. et al., Proc. Nat. Acad. Sci. USA, 87:1874-1878 (1990)) and nucleic acid based sequence amplification (NASBA). The latter two amplification methods involve isothermal reactions based on isothermal transcription, which produce both single-stranded RNA (ssRNA) and double-stranded DNA (dsDNA) as the amplification products in a ratio of about 30 and 100 to 1, respectively.

The amplified DNA can be labeled (e.g., radiolabeled) and used as a probe for screening a cDNA library derived from human cells. The cDNA can be derived from mRNA and contained in zap express (Stratagene, La Jolla, Calif.), ZIPLOX (Gibco BRL, Gaithesburg, Md.) or other suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen, X. et al., Genome Res., 9:492-498 (1999)) and polypeptides. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify cancer or a susceptibility to cancer (e.g., prostate cancer (e.g., aggressive prostate cancer), breast cancer, lung cancer, melanoma), and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

As used herein, two polypeptides (or a region of the polypeptides) are substantially homologous or identical when the amino acid sequences are at least about 45-55%. In other embodiments, two polypeptides (or a region of the polypeptides) are substantially homologous or identical when they are at least about 70-75%, at least about 80-85%, at least about 90%, at least about 95% homologous or identical, or are identical. A substantially homologous amino acid sequence, according to the present invention, will be encoded by a nucleic acid molecule comprising SEQ ID NO:1 or a portion thereof, and further comprising at least one polymorphism as shown in Table 1, wherein the encoding nucleic acid will hybridize to SEQ ID NO:1 under stringent conditions as more particularly described herein.

The present invention is now illustrated by the following Examples, which are not intended to be limiting in any way. The relevant teachings of all publications cited herein not previously incorporated by reference, are incorporated herein by reference in their entirety.

### Example 1

### Identification of Region Associated with Cancer

### Study

A region on chromosome 8q24.21 was identified that confers an increased risk for particular cancers (e.g., prostate cancer). This region was initially detected by linkage analysis of prostate cancer (PrCa) families with prostate cancer patients who are closely related to breast cancer cases.

### Patients Involved in the Genetics Study

The population of patients that were diagnosed with prostate cancer since 1955 included 3123 patients, about a third of whom were still alive at the time of study. The population of patients that were diagnosed with breast cancer included 4045 patients. About 950 prostate cancer patients were recruited at the time of the study. We were initially interested in finding genes that contributed to both prostate cancer and breast cancer. Therefore, we ran the list of our recruited patients against the genealogy database to cover all of Iceland. We only included families that had at least two prostate cancer patients related up to 6 meioses (6 meioses separate second cousins) and which also included at least one breast cancer patient who was closely related (up to 3 meioses) to a prostate cancer patient (we did not use the DNA or genotypes for the breast cancer patient—we only sought to fractionate our prostate cancer cohort by status of breast cancer in relatives). These criteria resulted in 75 large families that included 167 prostate cancer patients. The maximum distance between two prostate cancer patients was 6 meiosis, however, the average distance was 3.5 meiosis.

### Genome Wide Scan

The genealogy database was used to create families that included two or more prostate cancer patients and at least one breast cancer patient related to both of the prostate cancer patients within 3 meiotic events (generations). A genome wide scan was performed on 167 prostate cancer patients in 75 extended families. The procedure was similar to that described in Gretarsdóttir, et al., Am J Hum Genet., 70(3):593-603 (2002). In short, the DNA was genotyped with a framework marker set of 1200 microsatellite markers with an average resolution of 3 cM. Subjects in the study had 45 mL of blood drawn after they have signed an informed consent form approved by the Data Protection Authorities and the National Bioethics Committee in Iceland. DNA was isolated from whole blood using the Qiagen extraction method, which was adjusted for high-throughput. The microsatellite screening set used fluorescently labeled primers and all markers were extensively tested for multiplex PCR reactions to optimize the yield. The genotyping error rate was less than 0.2%, based on comparison of genotypes for over 5,000 individuals genotyped twice for this framework marker set. The PCR amplifications were set up and pooled using Cyberlab robots using a reaction volume of 5 μl containing 20 ng of genomic DNA. The alleles were called automatically with the DAC program or manually, and the program deCODE-GT was used to fractionate according to quality and edit the called genotypes (Palsson, B., et al., Genome Res., 9(10):1002-1012 (1999)). The population allele frequencies for the markers were constructed from a cohort of more than 30,000 Icelanders that have participated in genome-wide studies of various disease projects at deCODE genetics.

The microsatellite markers that were genotyped within the locus were either publicly available or designed at deCODE genetics; those markers are indicated with a DG designation. Repeats within the DNA sequence were identified that allowed us to choose or design primers that were evenly spaced across the locus. The identification of the repeats and location with respect to other markers was based on the work of the physical mapping team at deCODE genetics.

For the markers used in the genomewide scan, the genetic positions were taken from the recently published high-resolution genetic map (HRGM), constructed at deCODE genetics (Kong A., et al., Nat Genet., 31: 241-247 (2002)). The genetic position of the additional markers are either taken from the HRGM, when available, or by applying the same genetic mapping methods as were used in constructing the HRGM map to the family material genotyped for this particular linkage study.

### Statistical Methods for Linkage Analysis

The linkage analysis was done using the software Allegro (Gudbjartsson et al., Nat. Genet. 25:12-3, (2000)), which determines the statistical significance of excess sharing among related patients by applying non-parametric affected-only allele-sharing methods (without any particular disease inheritance model being specified). Allegro, a linkage program developed at deCODE genetics, calculates LOD scores based on multipoint calculations. Our baseline linkage analysis used the Sₚₐᵢᵣₛ scoring function (Whittemore, A. S. and Halpern, J., Biometrics 50:118-27 (1994); Kruglyak L, et al., Am J Hum Genet 58:1347-63, (1996)), the exponential allele-sharing model (Kong, A. and Cox, N. J., Am. J. Hum. Genet., 61:1179 (1997)), and a family weighting scheme, which was halfway on a log scale between weighting each affected pair equally and weighting each family equally. In the analysis, all genotyped individuals who were not affected were treated as “unknown”. Because of concern with small sample behavior, we computed corresponding P-values in two different ways for comparison. The first P-value was computed based on large sample theory; Zᵢᵣ=√(2 logₑ (10) LOD) and was approximately distributed as a standard normal distribution under the null hypothesis of no linkage. A second P-value was computed by comparing the observed LOD score to its complete data sampling distribution under the null hypothesis. When a data set consists of more than a handful of families, these two P-values tend to be very similar.

All suggestive loci with LOD scores greater than 2 were followed up with some extra markers to increase the information on the sharing within the families and to decrease the chance that a LOD score represents a false-positive linkage. The information measure that was used was defined by Nicolae (D. L. Nicolae, Thesis, University of Chicago (1999)) and was a part of the Allegro program output. This measure is closely related to a classical measure of information as previously described by Dempster et. al. (Dempster, A. P., et al., J. R. Stat. Soc. B, 39:1-38 (1977)); the information equals zero if the marker genotypes are completely uninformative and equals one if the genotypes determine the exact amount of allele sharing by descent among the affected relatives. Using the framework marker set with average marker spacing of 4 cM typically results in information content of about 0.7 in the families used in our linkage analysis. Increasing the marker density to one marker every centimorgan usually increases the information content above 0.85.

### Statistical Methods for Association and Haplotype Analysis

For single marker association to the disease, Fisher exact test was used to calculate a two-sided P-value for each individual allele. When presenting the results, we used allelic frequencies rather than carrier frequencies for microsatellites, SNPs and haplotypes. Haplotype analyses were performed using a computer program we developed at deCODE called NEMO (NEsted MOdels) (Gretarsdóttir, et al., Nat Genet. 2003 October;35(2):131-8). NEMO was used both to study marker-marker association and to calculate linkage disequilibrium (LD) between markers, and for case-control haplotype analysis. With NEMO, haplotype frequencies are estimated by maximum likelihood and the differences between patients and controls are tested using a generalized likelihood ratio test. The maximum likelihood estimates, likelihood ratios and P-values are computed with the aid of the EM-algorithm directly for the observed data, and hence the loss of information due to the uncertainty with phase and missing genotypes is automatically captured by the likelihood ratios, and under most situations, large sample theory can be used to reliably determine statistical significance. The relative risk (RR) of an allele or a haplotype, i.e., the risk of an allele compared to all other alleles of the same marker, is calculated assuming the multiplicative model (Terwilliger, J. D. & Ott, J. A haplotype-based ‘haplotype relative risk’ approach to detecting allelic associations. Hum. Hered. 42, 337-46 (1992) and Falk, C. T. & Rubinstein, P. Haplotype relative risks: an easy reliable way to construct a proper control sample for risk calculations. Ann. Hum. Genet. 51 (Pt 3), 227-33 (1987)), together with the population attributable risk (PAR).

In the haplotype analysis, it may be useful to group haplotypes together and test the group as a whole for association to the disease. This is possible to do with NEMO. A model is defined by a partition of the set of all possible haplotypes, where haplotypes in the same group are assumed to confer the same risk while haplotypes in different groups can confer different risks. A null hypothesis and an alternative hypothesis are said to be nested when the latter corresponds to a finer partition than the former. NEMO provides complete flexibility in the partition of the haplotype space. In this way, it is possible to test multiple haplotypes jointly for association and to test if different haplotypes confer different risk. As a measure of LD, we use two standard definitions of LD, D′ and R² (Lewontin, R., Genetics, 49:49-67 (1964) and Hill, W. G. and A. Robertson, Theor. Appl. Genet., 22:226-231 (1968)) as they provide complementary information on the amount of LD. For the purpose of estimating D′ and R², the frequencies of all two-marker allele combinations are estimated using maximum likelihood methods and the deviation from linkage disequilibrium is evaluated using a likelihood ratio test. The standard definitions of D′ and R² are extended to include microsatellites by averaging over the values for all possible allele combinations of the two markers weighted by the marginal allele probabilities.

The number of possible haplotypes that can be constructed out of the dense set of markers genotyped in the 1-LOD-drop is very large and even though the number of haplotypes that are actually observed in the patient and control cohort is much smaller, testing all of those haplotypes for association to the disease is a formidable task. It should be noted that we do not restrict our analysis to haplotypes constructed from a set of consecutive markers, as some markers may be very mutable and might split up an otherwise well conserved haplotype constructed out of surrounding markers.

In this study, only haplotypes that span less than 300 kb were considered.

### Results

As described herein, a region on chromosome 8q24.21 was identified that confers an increased risk for particular cancers (e.g., prostate cancer (e.g., aggressive prostate cancer), breast cancer, lung cancer, melanoma). Particular haplotypes and markers associated with an increased risk of cancer are depicted in Table 1. As indicated in Table 1, the haplotypes involve the following markers (e.g., SNP, microsatellite) and alleles: SG08S686 3 allele, SG08S710 2 allele, DG8S737 −8 allele, SG08S687 4 allele, SG08S717 1 allele, SG08S664 2 allele, SG08S722 2 allele, SG08S689 2 allele, SG08S690 4 allele, SG08S720 4 allele, DG8S1769 1 allele, SG08S691 2 allele and DG8S1407-1 allele. The hapolotypes are located in what we call LD Block A between 128,417,467 and 128,511,854 bp (NCBI Build 34) and positions of the individual markers are indicated in Table 1.

TABLE 1

| | | | Strand | | Decode | Base allele | | |
|---|---|---|---|---|---|---|---|---|
| | | | orientation | | allele name | name of | Control | |
| Decode | | rs | of SNP in | SNP alleles | in | SNPs in | freq. In | Build 34 |
| Name | SNP or Microsatellite | name | Build 34 | major/minor | Haplotype* | Haplotype | Iceland | start (bp) |
| SG08S686 | SNP | rs1447293 | − | A/G | 3 | G | 0.345 | 128428909 |
| DG8S737 | Microsatellite | | | | −8 | | 0.079 | 128433036 |
| SG08S687 | SNP | rs4871798 | + | C/T | 4 | T | 0.133 | 128436552 |
| SG08S717 | SNP | rs1447295 | + | A/C | 1 | A | 0.106 | 128441627 |
| SG08S664 | SNP | rs2290033 | + | C/G | 2 | C | 0.841 | 128449663 |
| DG8S1761 | Microsatellite | | | | 0 | | 0.556 | 128452660 |
| SG08S722 | SNP | rs7820229 | + | C/T | 2 | C | 0.851 | 128459172 |
| SG08S689 | SNP | rs4599773 | + | C/G | 2 | C | 0.441 | 128467013 |
| SG08S690 | SNP | rs4078240 | − | C/T | 4 | T | 0.842 | 128468152 |
| SG08S720 | SNP | rs7825823 | + | C/T | 4 | T | 0.986 | 128498506 |
| DG8S1769 | INDEL/MNR/Multiple | | | —/A and —/T | 1 | | 0.107 | 128501386 |
| SG08S691 | SNP | rs6991990 | + | C/T | 2 | C | 0.618 | 128501972 |
| DG8S1407 | INDEL/MNR | | | —/T | −1 | | 0.215 | 128503460 |

*Decode allele codes for SNPs in haplotypes are as follows: 1 = A, 2 = C, 3 = G, 4 = T; for microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.
INDEL refers to insertion (IN) or deletion (DEL),
MNR = Mono Nucleotide Repeat.

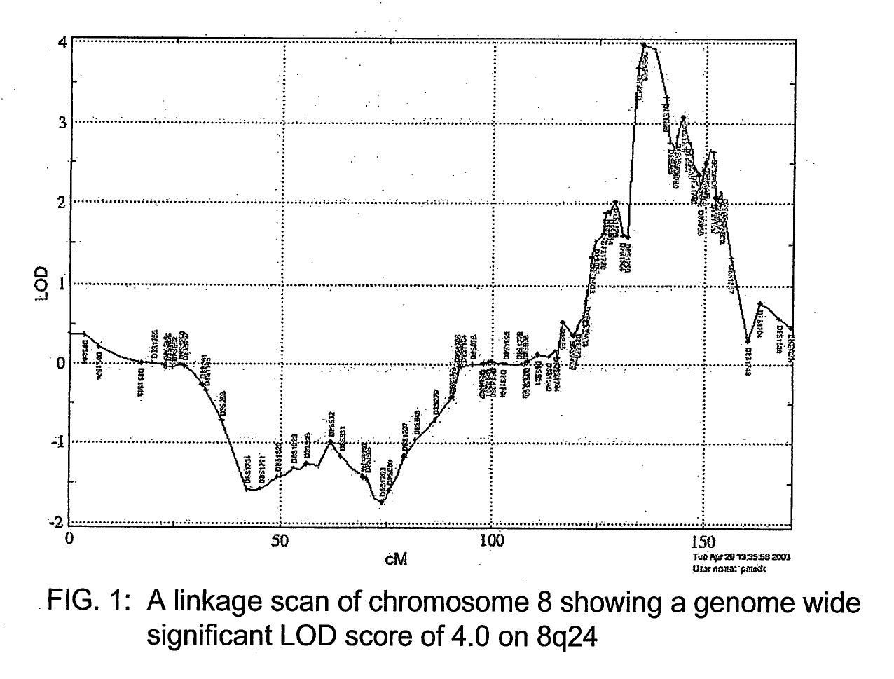

To find this cancer-associated haplotype, a genome wide linkage scan was first performed using families where both prostate and breast cancer segregate. Using those criteria, a total of 167 prostate cancer patients linked together into 75 families. FIG. 1 depicts the results of the linkage scan and details the peak seen at Chr8q24. Specifically, the linkage scan shows a genome wide significant LOD score of 4.0 at Cbr8q24.

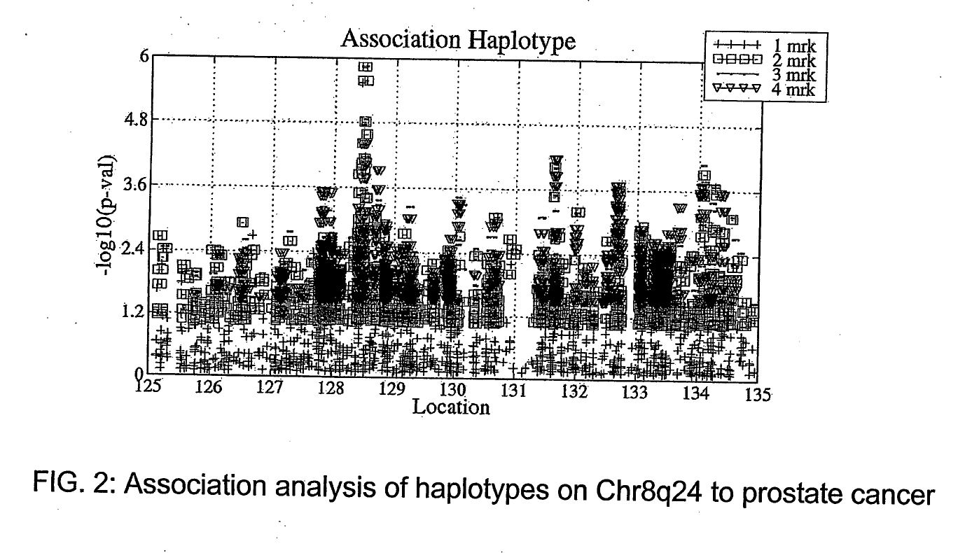

The peak marker on Chr8 is D8S1793 and the LOD score drops by one unit in the region extending from marker DG8S507 to marker D8S1746, or from 125,594,794-135,199,182 bp (NCBI Build 34). The region was genotyped with 352 microsatellite markers and 73 SNP markers for an average density of one marker every 22.8 kb. Association analysis with the resulting genotypes from both prostate cancer cases and controls yielded markers and haplotypes that signficantly associate with prostate cancer (FIG. 2, Tables 2-5). The results for prostate cancer, breast cancer, lung cancer, melanoma and benign prostatic hyperplasia are detailed in Tables 2 through 5.

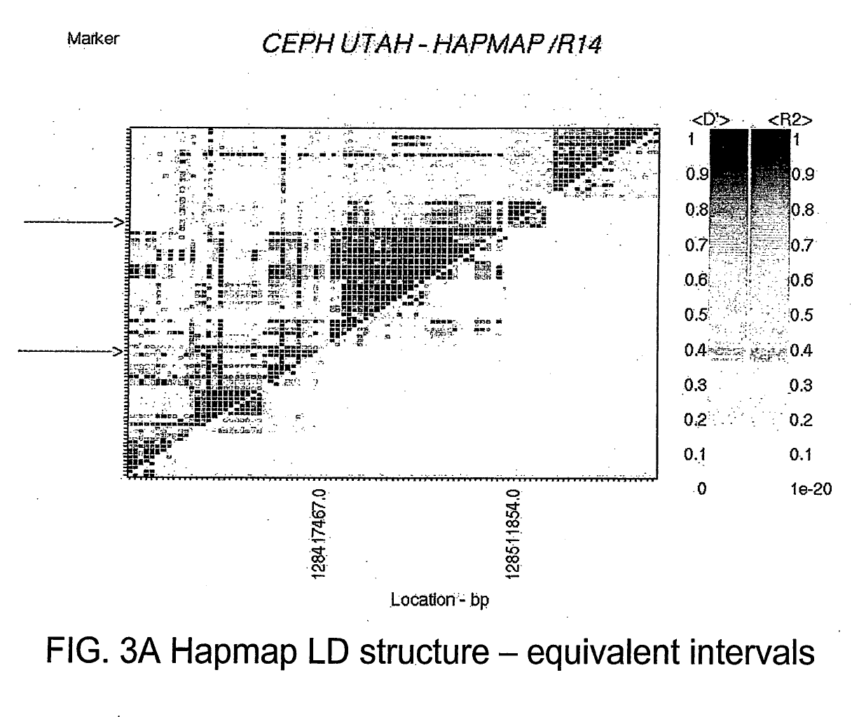

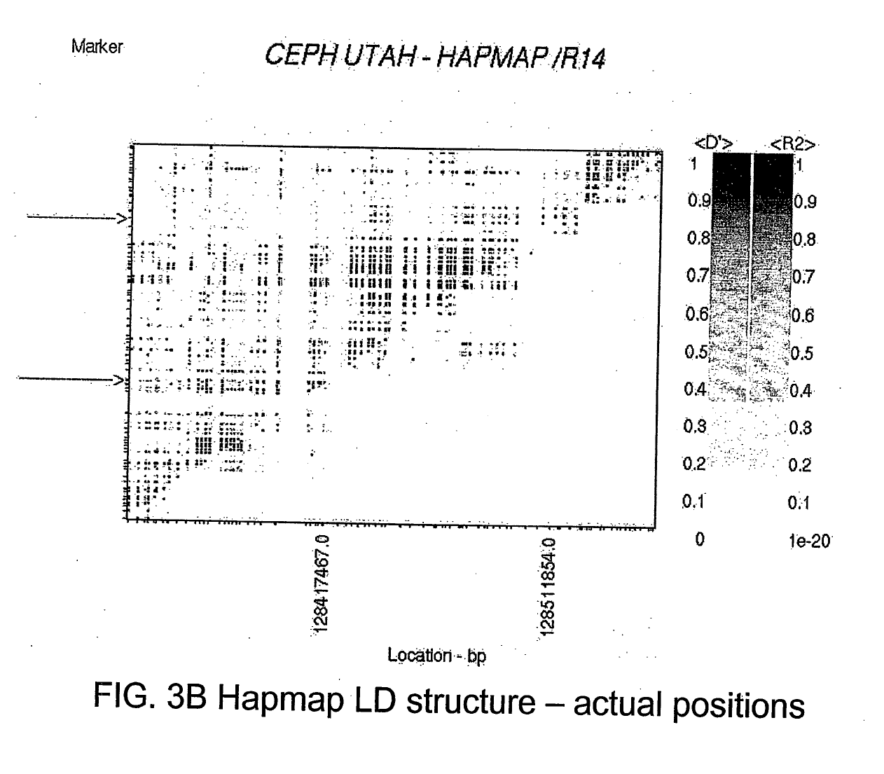

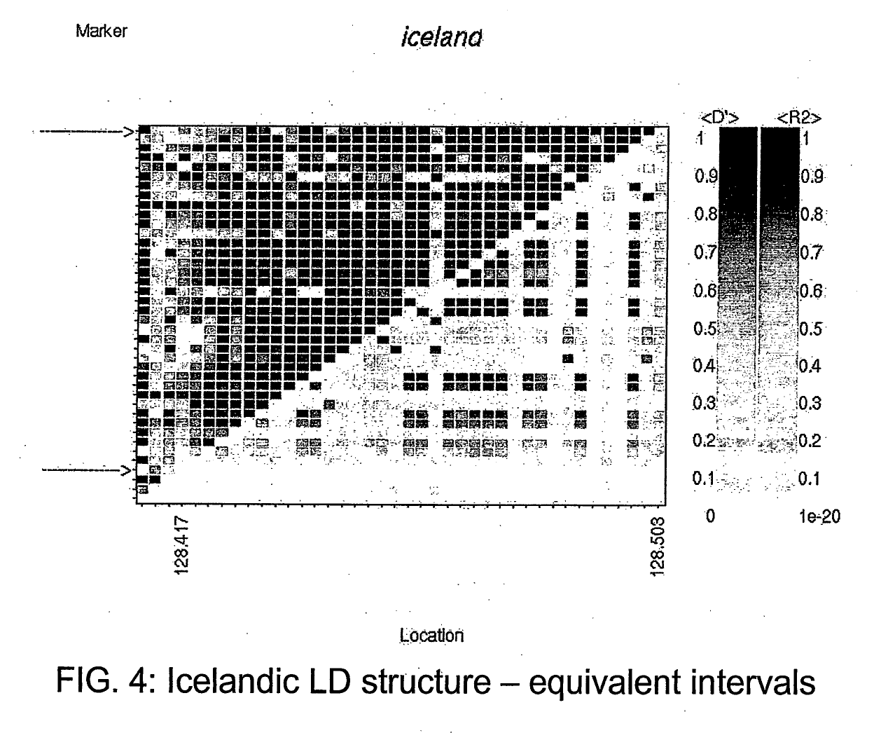

The LD structure in the area of the haplotype that associates with prostate cancer is shown in FIGS. 3A and 3B. The structure was derived from HAPMAP data release 14. In particular, the LD block that encompasses haplotype 1 is shown by the horizontal arrows on the left part of FIG. 3A. This LD block (LD Block A) was located at Chr8q24.21 between markers rs7841228, located at 128,417,467 bp, and rs7845403, located at 128,511,854 bp, and is almost 95 kb in length. LD Block A has now been refined to be located between 128,414,000 bp and 128,516,000 bp at Chr8q24.21. The LD structure is seen as a block of DNA that has a high r² and |D′| between markers as indicated by the red and blue colors in the figures. Markers are represented with the same distance between any two markers in FIG. 3A but with NCBI Build34 coordinates (actual distances between markers) in FIG. 3B. FIG. 4 shows the LD block in the Icelandic cohort of prostate cancer patients and controls in the area of the haplotypes that associate with prostate cancer, breast cancer, lung cancer and melanoma. It has a high |D′| for the majority of the pairs of markers |D′| >0.8) and r² going up to 1 for pairs of markers inside this block structure. This area includes recombination events that reveal themselves by a chessboard pattern best seen in FIG. 3. Markers in this block structure are also in moderate correlation (r² below 0.2) with more distant markers up to 200 kb away (including markers at 128515000 bps (rs7845403, rs6470531 and rs7829243) and markers around 128720000 bps (rs10956383 and rs6470572) in the area of the PVT1 gene).

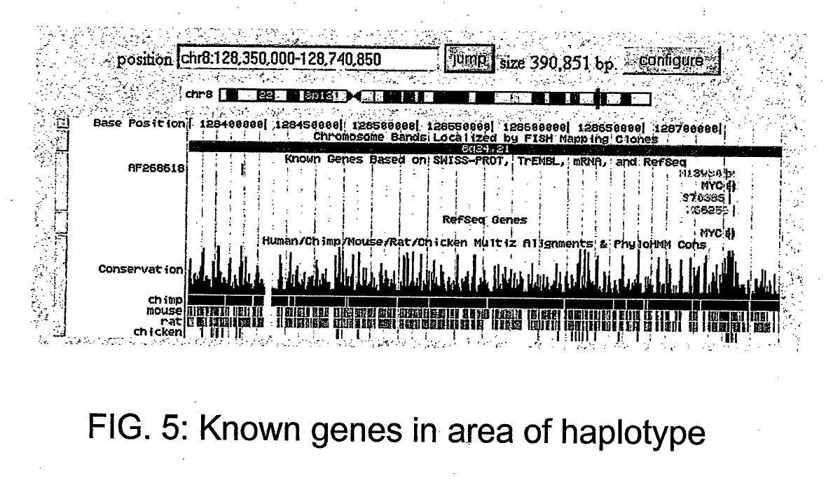

As described herein, genes and predicted genes that map to chromosome 8q24.21 of the human genome include the known genes POU5FLC20 (Genbank Accession No. AF268618), C-MYC (Genbank Accession No. NM_{—}002467) and PVT1 (Genbank Accession No. XM_{—}372058), as well as predicted genes (e.g., Genbank Accession Nos. BE676854, AL709378, BX108223, AA375336, CB104826, BG203635, AW183883 and BM804611. As depicted in FIG. 5, the markers and haplotypes of the invention are situated between two known genes, namely POU5FLC20/AF28618 and C-MYC (from the USCS Genome browser Build 34 at www.genome.ucsc.edu). The underlying variation in markers or haplotypes associated with this region and with cancer may affect expression of nearby genes, such as POU5FLC20, c-MYC, PVT1, and/or other known, unknown or predicted genes in the area. Furthermore, such variation may affect RNA or protein stability or may have structural consequences, such that the region is more prone to somatic rearrangement in haplotype carriers. This is in accordance with Chr8q24.21 being amplified in a large percentage of cancers, including, but not limited to, prostate cancer, breast cancer, lung cancer and melanoma (www.progenetix.com). In fact, Chr8q21-24 is the most frequently gained chromosomal region in all cancers combined (about 17%) and in prostate cancer (about 20%) (www.progenetix.com). Thus, the underlying variation could affect uncharacterized genes directly linked to the haplotypes described herein, or could influence neighbouring genes not directly linked to the haplotypes described herein. Table 2 describes one haplotype, haplotype 1 (SG08S686 3 allele, DG8S737 −8 allele, SG08S687 4 allele, SG08S717 1 allele, SG08S664 2 allele, DG8S1761 0 allele, SG08S722 2 allele, SG08S689 2 allele, SG08S690 4 allele, SG08S720 4 allele, DG8S1769 1 allele, SG08S691 2 allele, DG8S 1407-1 allele), and shows that this haplotype increases the risk for prostate cancer, with a greater risk for aggressive prostate cancer (as defined by a combined Gleason score of 7(4+3 only)-10). This haplotype was replicated in a second set of Icelandic prostate cancer cases and a new set of controls. As depicted in Table 2, haplotype 1 is carried by 21.4% of prostate cancer patients and 11.8% of controls. The relative risk of having prostate cancer for carriers of haplotype 1 is 1.92 (p-value=1.7×10⁻⁸). It should be noted that allelic frequencies are shown in all Tables, which are roughly one half of carrier frequencies.

The Gleason score is the most frequently used grading system for prostate cancer (DeMarzo, A. M. et al., Lancet 361:955-64 (2003)). The system is based on the discovery that prognosis of prostate cancer is intermediate between that of the most predominant pattern of cancer and that of the second most predominate pattern. Id. These predominant and second most prevalent patterns are identified in histological samples from prostate tumors and each is is graded from 1 (most differentiated) to 5 (least differentiated) and the two scores are added. The combined Gleason grade, also known as the Gleason sum or score, thus ranges from 2 (for tumors uniformly of pattern 1) to 10 (for undifferentiated tumors). Most cases with divergent patterns, especially on needle biopsy, do not differ by more than one pattern. Id.

The Gleason score is a prognostic indicator, with the major prognostic shift being between 6 and 7, as Gleason score 7 tumors behave much worse leading to more morbidity and higher mortality than tumors scoring 5 or 6. Score 7 tumors can further be subclassified into 3+4 or 4+3 (the first number is the predominant histologic subtype in the biopsied tumor sample and the second number is the next predominant histologic subtype), with the 4+3 score being associated with worse prognosis. A patient's Gleason score can also influence treatment options. For example, younger men with limited amounts of a Gleason score 5-6 on needle biopsy and low PSA concentrations may simply be monitored while men with Gleason scores of 7 or higher usually receive active management. In Table 2, the frequency of haplotype and the associated risk of aggressive prostate cancer (i.e., as indicated by a combined Gleason score of 7(4+3 only) to 10) and less aggressive prostate cancer (i.e., as indicated by a combined Gleason score of 2 to 7 (3+4 only)) are indicated. However, the Gleason score is not a perfect predictor of prognosis. Thus, patients with tumors with low Gleason scores may still have more aggressive prostate cancer (defined as tumors extending beyond the prostate locally or through distant metastasis).

TABLE 2
Frequencies and Risk of Haplotype 1 in Association with Prostate Cancer
(Haplotype 1: rs1447293 G allele, DG8S737 −8 allele, rs4871798 T allele,
rs1447295 A allele, rs2290033 C allele, DG8S1761 0 allele, rs7820229
C allele, rs4599773 C allele, rs4078240 T allele, rs7825823 T allele,
DG8S1769 1 allele, rs6991990 C allele, DG8S1407 −1 allele)

| | | | # | affected | # | control | |
|---|---|---|---|---|---|---|---|
| Phenotype | p-value | RR | affected | frequency | controls | frequency | info |
| PrCa | 1.85 × 10⁻⁸ | 2.02 | 821 | 0.114 | 896 | 0.060 | 0.982 |
| cohort#1 vs. | | | | | | | |
| Ctrls | | | | | | | |
| PrCa | 0.004 | 1.65 | 330 | 0.095 | 896 | 0.060 | 0.979 |
| cohort#2 | | | | | | | |
| vs. Ctrls | | | | | | | |
| PrCa vs. | 3.76 × 10⁻⁸ | 1.91 | 1151 | 0.108 | 896 | 0.060 | 0.984 |
| Ctrls | | | | | | | |
| High | 2.06 × 10⁻⁶ | 2.35 | 226 | 0.130 | 896 | 0.060 | 0.991 |
| Gleason* | | | | | | | |
| vs Ctrls | | | | | | | |
| Low | 6.54 × 10⁻⁶ | 1.79 | 810 | 0.102 | 896 | 0.060 | 0.983 |
| Gleason** | | | | | | | |
| vs Ctrls | | | | | | | |
| High | 0.049*** | 1.32 | 226 | 0.130 | 810 | 0.102 | 0.992 |
| Gleason* vs | | | | | | | |
| Low Gleason** | | | | | | | |

*High Gleason equals a total (combined) Gleason score of 7 (4 + 3 only) to 10;
**Low Gleason equals a Gleason score of 2 to 7 (3 + 4 only);
***p-value is one sided
RR = Relative Risk

The risk and significance associated with some of the individual markers of Haplotype 1 (listed in the header of Table 2) approaches that of Haplotype 1. Table 3 lists these markers and the risk associated with them.

TABLE 3
Frequencies and Risk of Individual Markers Associated with Prostate Cancer

| p-val | RR | #aff | aff freq | #con | con freq | H0 freq | X2 | info | Allele | Marker |
|---|---|---|---|---|---|---|---|---|---|---|
| 6.66E−09 | 1.69 | 1176 | 0.16752 | 956 | 0.10617 | 0.14001 | 33.6314 | 1 | A | rs1447295 |
| 1.31E−08 | 1.69 | 1190 | 0.15966 | 982 | 0.10132 | 0.13329 | 32.3201 | 1 | G | rs4314621 |
| 1.33E−08 | 1.68 | 1188 | 0.1633 | 974 | 0.10421 | 0.13668 | 32.2906 | 1 | A | rs4242382 |
| 1.34E−08 | 1.66 | 1254 | 0.16547 | 967 | 0.10652 | 0.1398 | 32.2708 | 1 | A | DG8S1769 |
| 2.42E−08 | 1.76 | 1231 | 0.13201 | 938 | 0.07942 | 0.10927 | 31.125 | 1 | −8 | DG8S737 |
| 3.56E−08 | 1.64 | 1190 | 0.16429 | 983 | 0.10682 | 0.13829 | 30.3745 | 1 | C | rs4242384 |
| 5.92E−08 | 1.65 | 1158 | 0.15976 | 970 | 0.10336 | 0.13409 | 29.3896 | 0.999 | A | rs7812894 |
| 6.86E−08 | 1.6 | 1196 | 0.176 | 984 | 0.11789 | 0.14977 | 29.1027 | 1 | G | rs4599771 |
| 3.16E−07 | 1.55 | 1168 | 0.18279 | 954 | 0.12579 | 0.15716 | 26.1498 | 1 | A | rs4498506 |
| 6.47E−07 | 1.52 | 1193 | 0.19084 | 948 | 0.13425 | 0.16577 | 24.7655 | 0.998 | T | rs4871798 |
| 9.80E−06 | 1.37 | 1283 | 0.27336 | 901 | 0.21488 | 0.24923 | 19.5504 | 0.999 | −A | DG8S1407 |
| 3.69E−05 | 1.52 | 1197 | 0.12239 | 981 | 0.08414 | 0.10517 | 17.0265 | 1 | A | rs2121630 |
| 0.00051 | 1.33 | 953 | 0.24082 | 857 | 0.19312 | 0.21823 | 12.0902 | 1 | C | rs921146 |
| 0.00079 | 1.24 | 1195 | 0.39414 | 973 | 0.34465 | 0.37194 | 11.2684 | 0.999 | G | rs1447293 |
| 0.00367 | 1.21 | 1093 | 0.60201 | 911 | 0.55653 | 0.58134 | 8.4416 | 1 | 0 | DG8S1761 |
| 0.0109 | 1.17 | 1203 | 0.45375 | 937 | 0.41486 | 0.43673 | 6.4818 | 1 | −C | DG8S1434 |
| 0.01354 | 1.16 | 1192 | 0.47861 | 950 | 0.44076 | 0.46183 | 6.0967 | 1 | C | rs4599773 |
| 0.01488 | 1.16 | 1186 | 0.47386 | 982 | 0.43686 | 0.4571 | 5.9303 | 1 | A | rs12155672 |
| 0.01982 | 1.17 | 1100 | 0.65407 | 903 | 0.61849 | 0.63802 | 5.4273 | 0.999 | C | rs6991990 |

A highly correlated haplotype to haplotype 1, which is detected using fewer microsatellite markers, is associated with an increased risk of other forms of cancer (e.g., breast cancer, lung cancer, melanoma). Table 4 shows that this haplotype (haplotype 1a, which contains the DG8S737 −8 allele, the DG8S1769 1 allele and the DG8S1407-1 allele) significantly (one-sided p-value<0.05) increases the risk of having prostate cancer, high Gleason (aggressive) prostate cancer, breast cancer, lung cancer, melanoma and malignant cutaneous melanoma, but does not increase the risk of having in situ melanoma. Haplotype la is carried by 22.2%, 16.0%, 15.4% and 18.0% of prostate, breast, lung cancer and melanoma patients, respectively. Again, it should be noted that allelic frequencies are shown in all Tables, which are roughly one half of carrier frequencies.

TABLE 4
Frequency and Risk of Haplotype 1a in association with Other
Forms of Cancer
(Haplotype 1a: DG8S737 −8 allele, DG8S1769 1 allele, DG8S1407 −1 allele)

| | | | # | Affected | # | control | |
|---|---|---|---|---|---|---|---|
| | p-value* | RR | affected | frequency | controls | frequency | info |
| Prostate cancer | 2.89 × 10−9 | 2.06 | 1062 | 0.111 | 791 | 0.057 | 0.989 |
| Prostate cancer | 2.98 × 10−7 | 2.56 | 206 | 0.135 | 791 | 0.057 | 0.990 |
| Gleason (4 + 3) − 10 | | | | | | | |
| Breast cancer | 0.0091 | 1.42 | 663 | 0.080 | 791 | 0.057 | 0.990 |
| Lung cancer | 0.0237 | 1.38 | 506 | 0.077 | 791 | 0.057 | 0.990 |
| Melanoma | 0.0009 | 1.62 | 504 | 0.090 | 791 | 0.057 | 0.993 |
| Malignant | 0.0002 | 1.86 | 322 | 0.102 | 791 | 0.057 | 0.992 |
| Cutaneous | | | | | | | |
| Melanoma | | | | | | | |
| In Situ | 0.2226 | 1.21 | 160 | 0.069 | 791 | 0.057 | 0.997 |
| Melanoma | | | | | | | |

*p-values are one sided

As depicted in Table 5, further studies revealed that haplotype 1a does not increase a subject's risk of having Benign Prostatic Hyperplasia (BPH), which is not considered prostate cancer. As shown in Table 5, haplotype 1 a is carried by 13.8% of BPH patients, as compared to 11.4% of controls, with a nonsignificant relative risk of 1.22.

TABLE 5
Frequency and Risk of Haplotype 1a in association with BPH
(Benign Prostatic Hyperplasia)
(Haplotype 1a: DG8S737 −8 allele, DG8S1769 1 allele, DG8S1407 −1 allele)

| | | | # | % | # | % | |
|---|---|---|---|---|---|---|---|
| Phenotype** | p-value | RR | affected | affected | controls | controls | info |
| BPH (not PrCa) vs | 0.1008 | 1.22 | 601 | 0.069 | 791 | 0.057 | 0.992 |
| Ctrls | | | | | | | |
| PrCa (not BPH) vs | 3.14 × 10−8 | 2.19 | 511 | 0.118 | 791 | 0.057 | 0.988 |
| Ctrls | | | | | | | |
| PrCa and BPH vs | 1.24 × 10−5 | 2.00 | 362 | 0.108 | 791 | 0.057 | 0.991 |
| Ctrls | | | | | | | |

*p-values are one sided
**First group (BPH (not PrCa)) includes men with BPH only Second group (PrCa (not BPH)) includes men with PrCa only Third group (PrCa and BPH) includes men diagnosed with both PrCa and BPH

Table 6 depicts the amplimers used to amplify sequences for detecting microsatellite markers. Table 7 depicts the amplimers used to amplify sequences for detecting SNP markers.

TABLE 6
Listing of Microsatellite amplimers and primers.

| Microsatellite amplimers | | |
|---|---|---|
| NAME | SEQUENCE | LENGTH |

| | | |
|---|---|---|
| DG8S1407 | F: CCAATAGCCTTCAATGTATCAAA | |
| Primer pair | (SEQ ID NO: 2) | |
| | R: TGAGGAAGAGCCACAACAGA | |
| | (SEQ ID NO: 3) | |
| Amplimer | CCAATAGCCTTCAATGTATCAAAagctggca | 236 |
| | cattactggttctgctcttG[N]tttttttttaaattatagtactttctttcagaaatat | |
| | actaacaaagaaaaaaagacaattgaaatttccaaatctggaacaactggatt | |
| | ggagaaaaatatacaaaataaaccccacgaggttttaattctaagtactttaga | |
| | ccttacaagcaccataaacatTCTGTTGTGGCTCTTCCTCA | |
| | (SEQ ID NO: 4) | |
| DG8S1769 | F: CCTCCCAAACACACAGAGTTG | |
| Primer pair | (SEQ ID NO: 5) | |
| | R: TGTTAAACCTAAGGGTTCCTTCC | |
| | (SEQ ID NO: 6) | |
| Amplimer: | CCTCCCAAACACACAGAGTTGaaaaccacagt | 262 |
| | gtagacttaaataaaattactaaagaccggtctatggaaaataatatact[/t]c | |
| | caaaattaacatatactttctttctcagtctcagttcttttccctaaaaataaaataa | |
| | aataaaataaataggctgttgcactctagaaactactctaaaacaactacagat | |
| | caattatgc[N]aaaaaaaagtctgaaagttacagtacatgaggggGGA | |
| | AGGAACCCTTAGGTTTAACA (SEQ ID NO: 7) | |
| | Note: IUPAC code: /t refers to either no | |
| | nucleotide or t | |
| DG8S1761 | F: TTGAAATTGCAATCCCATCA | |
| Primer pair | (SEQ ID NO: 8) | |
| | R: CCTCCCTACTTATTCCCATGC | |
| | (SEQ ID NO: 9) | |
| Amplimer | TTGAAATTGCAATCCCATCAtcccccagaactc | 392 |
| | ctgatatcccctacactcccttatacttttttgtctatagcaaccacccctcacca | |
| | ctttataacatttatgctttgtagtctgtctgtgtccactcactagaattcaaatatc | |
| | acaaaagcaggagtccactttttttttcattgaaaaactccaaatcctagaagg | |
| | aagctggcatttaatatgtgctcaatagacattagaggaagaaaagaaggaa | |
| | ggaaggaaagaagggagggagggagggagggagggaggaaggaagg | |
| | aaggaatgaaggaaggaaggaaggaagaaaggaaggaaagaaagaaag | |
| | tcaagagacctgggctcaaatccaGCATGGGAATAAGTAGG | |
| | GAGG (SEQ ID NO: 10) | |
| DG8S737 | F: TGATGCACCACAGAAACCTG | |
| Primer pair | (SEQ ID NO: 11) | |
| | R: CAAGGATGCAGCTCACAACA | |
| | (SEQ ID NO: 12) | |
| Amplimer | TGATGCACCACAGAAACCTGTCAGTTGG | 134 |
| | TACTGATCTACCCTCCTCCTCCTCCTTCTCCTaca | |
| | cacacacacacacacacacacacacacacacacacacacacacTTCAT | |
| | CCTACTCTCCAGCATTCAGGGAAGAAAACAGA | |
| | GGCAAATGTTGTGAGCTGCATCCTTG (SEQ ID | |
| | NO: 13) | |

TABLE 7

Listing of SNP amplimers and primers.
SNP amplimers

| | | |
|---|---|---|
| NAME | SEQUENCE | INFORMATION |

| | | |
|---|---|---|
| SNP: | gtttttaaacatatttttttcgctgacctccaccctgtaagagcttttatta | Genotype statistics: |
| SG08S664 | ccaagcgattgagaagcacaggctcagggacactgaatttgaccaaagaagc | old verified: None |
| | caatagaactattccaaaaacctatggttccccctaaagcattagaaagactca | snp human edit: |
| | gaacgggttaagtgctccctggctcattcccaacagacactacattcacctgtg | C/C: 1986 C/G: 749 |
| | cttgctctgaaataaatcagtgtccctttctgctgctgctgttgtctggaaataatg | G/G: 75 |
| | caaatgcaatgggcctttactgacattgtgcttccctggaaggatacacataata | Build34 position: |
| | aattatcccttaatactgttaaagagacattttcctcttactcaggagcttttggggt | chr8: 128.449663+ |
| | tggactgggctactcacccagcaaggaggaggacatgtgtcttgtcactggcc | Aliases: rs2290033 |
| | cggttattcatgtggcctctcattgctccttggctcactgcattgcaagattcaag | Equivalences: |
| | gatgcactt[C/G]caggcctccacatcaagtcataggacttgccggtaacct | Unique, no other |
| | agattggttttctcatttgtaatttgaatttattttatgttatgcatttgtatgtttattta | equivalent snps |
| | ttcggatgctcagaagctgaagataactagtgctcctggtccatgccattcatcaa | equivalence name: |
| | ttggaagaatgccaagctgtttccgctgaggacagaaggcattggtctcccctg | SG08S664 |
| | caggaagccactgctgctccttaattgtttgctagaggaagaatcaagggtaaa | |
| | atttaaagtaaatggctggccgagttgcactaattcatcaaagcatgtttcaagtc | |
| | agtagtcagagcatgcatcagcccccggcgccaccagcttctacgagagtgg | |
| | aaaagccagcagacctccgagcagatgaaatcattaggaggcattcagcagg | |
| | gcttgaaaagcaaagagagaggaggcggggatttctctgcatgctccctttgc | |
| | cacatgggaaacaccagctgtc (SEQ ID NO: 14) | |
| SNP: | cccaaattatcctcacctctttataagtctcccataaccctttcttaccct | Genotype statistics: |
| SG08S686 | attttaagcttcttttaaatatagtaaggaagagtttctctggccttctttttttcctca | old verified: None |
| | aattttattttagattcaggaggtacatgtgaaggtttgtaacttgggtatattgcat | snp human edit: |
| | gatgctgagatttggggtgcagctaattccaccatccaggtactgagcatagta | A/A: 1474 A/G: 1821 |
| | ctcaatagtttttcaactctttcccctctagctccctccatcccccatctagtagtcc | G/G: 629 |
| | ccagtttctattgctgccatctttatgtccataagtatctggtctccttttaaatttgct | Build34 position: |
| | ttcttctttgctcattatctagaatttccataatagaggagaacctgaaaccacacc | chr8: 128.428909- |
| | caataagaaagaattttatctaaagttttactacctttgcattccagtctttctctacc | Aliases: rs1447293 |
| | cattctcctaatcttgtctcgtgaaatcatggctgctgagaat[A/G]agatttctt | Equivalences: |
| | ttggaggacaatgaaaaggatgggaggacagaagctacacagaagggaga | Unique, no other |
| | aaggaaaacagagcaactgaagacaaaaattactttagaaggtgtaagcacat | equivalent snps |
| | acaaacagggctgaggttatatgtttcactttgaatgaatctcatttaccgagata | equivalence name: |
| | ccaggagcattttacttaagtctttgagaacacgagttttactggctatatcatact | SG08S686 |
| | ctgttgtagaaatacactgtaaagtactttcactatcctcttttattggacatttagat | |
| | ctaaatgaattttgtgctaatatgaatattgtatgatgaatatctttgactatattttgt | |
| | gcattttgttataggcatgtatcttgaaaacggcagagggaagattttgctttgtta | |
| | cccattttgataggccttgcctttggccagacatgttactgatgttttggtattgaa | |
| | ctgatgtatgtcttcatttatttgtttttatttatttttatt (SEQ ID NO: 15) | |
| SNP: | cagaactaggaaaattgccaaaagttatgggtctgtacagagttagt | Genotype statistics: |
| SG08S687 | gtcacagtaagaatctcattgcccaagcaatagggtctaaaatcacgatcttatt | old verified: None |
| | caaagtaacagcgaccacttacctcatgcctcatatgtgccagatacttttcttac | snp human edit: |
| | attatttttaatctccatagcaattatctaaggtagataatatctagagatgaggaa | C/C: 2738 C/T: 1010 |
| | actggggctctaggagtatgcaagatttgtccaaggtctcacagcaatatcttag | T/T: 111 |
| | tagagtctgtctagaatcaaagccaatttgtctttttgccctatcatggttcatctct | Build34 position: |
| | acttcactctaactccatcctaaaaaccaccttccccatccactatataaatgaat | chr8: 128.436552+ |
| | gatagcaccaccctttcagtaaaaggatctagacattcaccatctctctaccatc | Aliases: rs4871798 |
| | ctagcagcaactgcaatgcttggaaaatagtcgaggattagtaagagcttgtca | C08PoolseqSNPs_1287 |
| | aatgagacacagtttgttgttctggccctgacatgaaacaggtaatcaagtaaa | Equivalences: |
| | cgtatattttatatatagtcacttcactttcctagtcactaatttccttatctataagac | Unique, no other |
| | aagggtattgggccaaaagtctagtcttaaaggttcctttcaagtcatttattgaa | equivalent snps |
| | agtttgtctgatactttattttttactaaactttatatattccttaaatacacactcaaa | equivalence name: |
| | gaaacatatacaggtaaatacagacaagctctatctaatggtgttaactgtcactt | SG08S687 |
| | agtatataaagacatcttctctcagagaaattggtcacatgttctttctttagacaa | |
| | ctgctcatcatgtcctttgactaatcataagccaacagtaagaagttaagagtgc | |
| | caagaaaaggtaactgtgttaagttgcatttgtatttttccaagtatttactctccca | |
| | ttctttcatatctataagaggattatccatccccacccactggcatgtgcg[C/T] | |
| | acagtgcctccatgaggggcgtttatctgtttttcttcacaatgaatttatcacattc | |
| | cttgctttggccaatagaatgtgagtgggcatacgatgtgtgcatgtctgaacag | |
| | aagtcatgaaacaattgcctggttctgatttatctcctgctttttttttctttggcgtta | |
| | aattggtatgtgcgagatagaggttgatctttcaactttgacctggtattgagaag | |
| | gcacctgaggcaaaaccagagctgatctagagttgacatacacagtggacat | |
| | ataaaatgaataaaagataaaacttttagattgtaagccactgtaatttggaagat | |
| | gtttgttactgcagcataacctatcaaaggctgacttataaaaaatatttcagata | |
| | ccgttagttctcactgttcacagtagttatgttttatgaagtttccatggatactgaa | |
| | tgagcgaacagtgaactaatgttcctaggtaaaatagaagattaggttcccgtg | |
| | agctctgggcaaaacattttcatcatccaagcaatacataatcttgctttatgtgtg | |
| | tttctatgtaaagacaccttattcaatatattttgttgattcattaaaattaaactcatg | |
| | gccagcagcattatagctcatgcctaaatgaggcttatctaacatgtatatattttc | |
| | tataagacatttcacagtcttcttgactcaagaacactacacagcacttcagcact | |
| | atgctgaaatggggccattttaaacagaaaaatcaccaccaacaaaaattagct | |
| | gggagtggtggtgcacacctgtagttcaagctacttgggagactgaggcaga | |
| | agaatcgtttgaacctaggaggcagaggtcgcagtgagacaagattgcacca | |
| | ctgtacttcagcctgggcaacagagtgagtctccatctcaagaaagaaaacaa | |
| | aacaaaaacaaaacaaaagaaacaaacaaaaaaacacttcatcaaaaagcat | |
| | aa (SEQ ID NO: 16) | |
| SNP: | taaagctcttaaccccacaatgccctgtccacagactctgaaa | Genotype statistics: |
| SG08S689 | gatgctgatgcattgttgtgtcccatgtctgtttccccagcagcaggttgtgagtt | old verified: None |
| | ctcagttgaattcagtttcttgttgcagagtctttatcaaaccacagaagaat | snp human edit: |
| | caaagttgaacaacatggagtatctacaccggagcagcccacagttcag | C/C: 591 G/G: 1353 |
| | ggatggacacagaacaagagagattcattacagacataaagcacagag | G/G: 730 |
| | atgttggggttttctctgttgggaagaataagaggtccagaaaagcttccc | Build34 position: |
| | aaagtgatggcacctcaagggtcaggacctcaccttattaatctccatgac | cbr8: 128.467013+ |
| | ccagcatctactacagcatctgtcacaactgggctctgagaatgttggcta | Aliases: rs4599773 |
| | aataaatgaatgaatgatatcaatacacagggtttttccccattttctgaatat | Equivalences: |
| | tctggactaggggatatctcagaacagtacttagcacctagtgtgtg[C/ | Unique, no other |
| | G]tcaataaattcttgttaaaccactaaaaattgctggacagctgaactga | equivalent snps |
| | aaattactcacagccccattcaactgcatcagccatgaaaatcaactcag | equivalence name: |
| | aatttgcaaatctatgctggcatttagcacttaagatgtaaatacagagtgt | SG08S689 |
| | cagccatgtggctaagatcagctttaattcagtgttcatctctgaaattcatt | |
| | aatgattaaatacttttttcctttgctctctatgggagttgaaacaagtatcat | |
| | gtatccaaagaccagggttcagtttggcccaacattaattcacttaatgtttc | |
| | aacaaaaatttattgaccatctactaagtgctgagtgctagaatccattgac | |
| | tacctactaatgaagtgctagattttaacacagggacatctgtggtaaaac | |
| | agtaaattctctaacctcatctagaggggttgaaggttctgcctttgcctac | |
| | cttctatagtcagagactactggtatttcaatcc (SEQ ID NO: 17) | |
| SNP: | gaccaaaattaccgtcaggacagagcagcctgagggcagcgctat | Genotype statistics: |
| SG08S690 | caagaggggagagccccaagttgtctgattggtgatgatggcaggttggtgat | old verified: None |
| | gcttcttaccacattgctatcctaagcagcaagtggtcccacctcagatttgcctc | snp human edit: |
| | taccattcctgccaggaaaccagatggcaggaagagcccatgaatcacctctc | C/C: 65 C/T: 668 |
| | tgggataagcagaacagtacttgtgtattcttgcctttgtggttgcttattctttcac | T/T: 1961 |
| | aattccaataagcaggccagtgtcaattgcctgctggagaatgcacttgattctt | Build34 position: |
| | ccgtgtacagtatcagaatatgatttttagttttaatggtaagaaatacgaatagta | chr8: 128.468152− |
| | ttcactcttttcctcattcccacagctgtgactggacttttggcctctgatgatcaa | Aliases: rs4078240 |
| | cataaatcccacctccatcccactgatgctttttaactttaagaggctcttcagtac | Equivalences: |
| | caccggagt[C/T]ttcaggggatagagtggatccctagaaaccgatcaagg | Unique, no other |
| | gccaatctgcagtgagttacccaggagtttagagattcccttcgtttaggtctgtt | equivalent snps |
| | gagtttaatcaatatttattatctgagcacatcctttgtgaacatccctctgctaga | equivalence name: |
| | gtcaggaaattagagatgaaacactcatggcctgtgccttagaggaactctcca | SG08S690 |
| | ttgagcagtggagacaggagaaaatggagaaggagaatgtgctctgctggac | |
| | ccagaggagagacttggggagccctcagcagaggcccttaactcctttttaga | |
| | aacagggaaaacttcctggaaaaggagacgttttcatctaatcattcatcatgtc | |
| | atatattcattcaataaacatttattgagcccttgctatatgccaagctcagcacta | |
| | cgttcaagggactcaggagccaatgagtcagacagtgtcctgccttcatggag | |
| | cttctatataatcttgaggaaatcc (SEQ ID NO: 18) | |
| SNP: | aaattaacatatactttctttctcagtctcagttcttttccctaaaaataaa | Genotype statistics: o |
| SG08S691 | ataaaataaaataaataggctgttgcactctagaaactactctaaaacaactaca | verified: None |
| | gatcaattatgcaaaaaaaagtctgaaagttacagtacatgaggggggaagga | snp human edit: |
| | acccttaggtttaacatagaattatctcagttaaggtgactgcataatgaatctga | C/C: 1087 C/T: 1156 |
| | cataaacatcaatttgactgcatgttgctttcattaaagcaaagaaaccagaaag | T/T: 296 |
| | gtggaagaatccttataccttatgctgcatgcatcacaacacaccaagtatacta | Build34 position: |
| | gacctagttctgggaacctcatttcaagagcaatggtgcaaaggagagcagcc | chr8: 128.501972+ |
| | agaatgaggagaggccaacagaccaggtccactctattccacagtgattcaa | Aliases: rs6991990 |
| | gaaacgttactgaacatgttgactcctatgttccaggagctgtagagacggagt | Equivalences: |
| | tggatgccacattga[C/T]gcttccctctagaaacttacattctagtagaggga | Unique, no other |
| | gccagtgtgcaatagaatatcatggcaataaacacagggctatactgaatagtg | equivalent snps |
| | ggactgttgcatagctaagagttatgcaagcaccaagtataaagaagcagcttc | equivalence name: |
| | tgagttgatagtgctgttttgtgccttttcagaggtatgttttagaaaaaataactct | SG08S691 |
| | aatggcagaataaataatggaaataagacagtgaaactaaaagtaaaagaaa | |
| | gccactgggaacccttgcagtaattcccgtgaaaaatgataacctcacaaacta | |
| | aagtagtggtgatgaaaatcgagaagaaaagatgttctgagagctagtttagaa | |
| | ggtagaatcatgagaactcggtgactggataagtatgatggggaatgtagagg | |
| | aaaagacatccaagatgactctagcttcaaataagagaaaggattgaggaaca | |
| | agggaagtttggcattaaacaaacaaacaaaaa (SEQ ID NO: 19) | |
| SNP: | tagagaaagagacaaagcaggaaagagaaaagagaaaggcata | Genotype statistics: o |
| SG08S717 | tatatatttttttcttcattctgggggcccaccctgaaactactgaatcacagtctct | verified: None |
| | agaggttctcaggcaactagcccagctgtttttgccaactggaatttatgagcca | snp human edit: |
| | ccgcaagagaccacatgcagcttcatgtaaaacaaattatttttaagcacgcag | A/A: 93 A/C: 878 |
| | actgagcagtgatatgaggagtgcacaggagtgcctacgcctactcctggtct | C/C: 2923 |
| | ccatgagtctcctttgcaaagtcaagtattacaagattctagaacacatattgcct | Build34 position: |
| | gccactgataatttagttgttcagcaaacattcatttgttgagttgcacgccagac | chr8: 128.441627+ |
| | actatactagatgatgggacaactaaagggtaatgaacagttctgtctctatgta | Aliases: rs1447295 |
| | aaaataataatgatgatgatgatgagatgggacttcaattgaggaagtgccatt | Equivalences: |
| | ggggaggtatgaaaa[A/C]gtgctatggaaaaaaagcaacaggaacccc | Unique, no other |
| | ttgatagaaaaaaaaatgctggtgggggtagggatttctgcctgtgttcttcaga | equivalent snps |
| | atggggtatgggaaaatctgggaggaaaagaaatttaagtaagagcagagac | equivalence name: |
| | tttgcaaaatttgttgtgttgacttttcctcatgctgcttcccctggcatgggaagtc | SG08S717 |
| | attagctggataagagagacttcacaagaactgcaatgaatcaagatgtgctg | |
| | gttttgttttgacacatggaattcttagggatttgatgttttttttcccagtcttctccat | |
| | caaagttgttttcaaccagtcctgattggaccgattgactcatcctcagatatcat | |
| | agttttcccactacaaaagcatggaactgatgccaataaacccactccttattcc | |
| | cagagggctagggtgagtccttgcagaggggaattgctagggatggcacctg | |
| | gcagaaatagaccatctgtctttcctcc (SEQ ID NO: 20) | |
| SNP: | tggttttctttcttcttatgttttgcttgtttcattttgcattttccaaaatgat | Genotype statistics: o |
| SG08S720 | gatattggagataacaaactgttaggtccttgttattctgtgcatatatgattttgtc | verified: None |
| | ctaagacaagatgaaataatcatatctcattttactatccagttatttggggtgtca | snp human edit: C/T: |
| | tcttaactagcagttaggattagcatgttactcaagctcacaaagacatagctgg | T/T: 2668 |
| | gatgacaacatgttctttgttcagagtatttgccacattgaggactcctggcaaaa | Build34 position: |
| | ataaataacttataagaaaggtaacttattttgactttaaaataatcgatgactaaa | chr8: 128.498506+ |
| | actcatttttcctcagaccatgagagcaatttaccaagctttattaatgggcatctt | Aliases: rs7825823 |
| | catatccttagcaagcttaattgctaattaattaaaagatgattggataaacaatg | Equivalences: |
| | gattgtactacaaaatgaagatagcaaaatttactgtcatggtgtctaa[C/T]g | Unique, no other |
| | agcattctttacctattgccctaccaatctttcagctccataatttctgaagtaaag | equivalent snps |
| | atccccaagagccatttcctgaaaattagagttaaatcagatcaacgttaaagg | equivalence name: |
| | acttctgggtcaaactatgttgagggccagccacaggcaatcataatttaattaa | SG08S720 |
| | agcaagagagagaaaaaaaatcatgccaagtgaaacagcctggaagagtga | |
| | caaaagcctttgtcttaaaatcagaatacctatgctctaaacatttactactgtgga | |
| | aactagtgaaagataatctaatttttctgagcttcatttttctcatctataaaatggat | |
| | atgatcagttcagctgcaagtaaaagaagcccaaaagtaacagaggactaag | |
| | caagacaggagtttatttttctaacttgcaaaagatccaaaggtagacagtcaag | |
| | aactcacagcagctctgctccacggaaatttcagagcctaggttccttctatgtt | |
| | gttt (SEQ ID NO: 21) | |
| SNP: | taaaggacaggcattggggttgctttgttgaacaaatctagcagatat | Genotype statistics: o |
| SG08S722 | ttgaatgagaagagtaatatagtcagtagaaaaaaagtgcaagaaataagtag | verified: None |
| | agaaagaagggatattttctgctgaagcatgtattctctggcacaagcccacaa | snp human edit: |
| | taaattgaaattgacaccaacagttggctcaaaaataatcaactacaaatatgct | C/C: 1975 C/T: 700 |
| | caacacataagcattctcttggacagaaccacaaagcatggtctgcattgttcct | T/T: 62 |
| | aacaactctttagaagtcaccagatgcagtttaagctacaataacatagtgaggt | Build34 position: |
| | acaagttaattacatagttaccagaaagtcacagacttttttttcagtaataatgta | chr8: 128.459172+ |
| | gtaaataaatacatgctcactccatgggaaatggtggcaattattaagagcaca | Aliases: rs7820229 |
| | cattcacaccatcatattgcttactgataactgtgcagttaaccaatggcagtgtg | Equivalences: |
| | ctaaaatggatat[C/T]tgtgtttccctgagttttgcatgctacatgcgatgcat | Unique, no other |
| | gtgaaaaccaagcatagggaatttcaagtatgaacttcagcgtgtgagtgttgtt | equivalent snps |
| | tgtggtccaatctccgtccccaaacatccccagaataaggcttctgctttttaaca | equivalence name: |
| | atgtatatctattttaaccaattgtctagcgtataattaatgctctataaactctttgtt | SG08S722 |
| | aaatgcattcacagaaggtaacaaaagatttttgtgacacgagtaaaccaaaag | |
| | gaacaaataaacttgaattactttatgtttgtgttggtgtttcagaaaagagctttg | |
| | gctttgaattcagaagttcctaatctgaataccaggtctaccaattattaattaagg | |
| | aatatcaaatgaattacttgcagtatttgaatttcagatttctcaattataacaagga | |
| | tgaaagaggtttattatgtggctcaaataagaaaatgcatgtaaaaacacttgta | |
| | aaccaaaca (SEQ ID NO: 22) | |

### Discussion

As described herein, a locus on chromosome 8q24.21 has been demonstrated to play a role in cancer (e.g., prostate cancer (e.g., aggressive prostate cancer), breast cancer, lung cancer, melanoma). Particular markers and haplotypes (e.g., haplotype 1, haplotype 1a, haplotypes containing one or more of the markers depicted in Table 1) are present at a higher than expected frequency in subjects having cancer. Based on the haplotypes described herein, which are associated with a propensity for particular forms of cancer, genetic susceptibility assays (e.g., a diagnostic screening test) can be used to identify individuals at risk for cancer.

The markers and haplotypes described herein are not associated with benign prostatic disease and do have a higher relative risk in the high Gleason prostate cancer patients as compared to the low Gleason prostate cancer patients (Table 2), thereby indicating an increased risk for aggressive, fast growing prostate cancer. Given that a significant percentage of prostate cancer is a non-aggressive form that will not spread beyond the prostate and cause morbidity or mortality, and treatments of prostate cancer including prostatectomy, radiation, and chemotherapy all have side effects and significant cost, it would be valuable to have diagnostic markers, such as those described herein, that show greater risk for aggressive prostate cancer as compared to the less aggressive form(s).

The significantly increased relative risk of breast cancer, lung cancer and malignant melanoma in individuals with the markers and haplotypes described herein further support their use to identify increased risk of these forms of cancer. Given that the haplotypes result in an increased risk of prostate cancer (e.g., aggressive prostate cancer), breast cancer, lung cancer and malignant melanoma, it is possible that these markers and haplotypes also are associated with an increased risk of other forms of cancer.

### Example 2

### Verification of Association with Prostate Cancer in Several Cohorts

Additional analysis further supported the presence of the variant associated with prostate cancer on chromosome 8q24. Allele −8 of the microsatellite DG8S737 was associated with prostate cancer in three cohorts of European ancestry from Iceland, Sweden and the United States. The estimated relative risk of the allele is 1.62 (P=2.7×10⁻¹¹). About 19% of patients and 13% of the general population carry at least one copy (PAR=7.4%). The association was also replicated in an African American cohort with similar relative risk. A higher frequency of the allele, 41% of patients and 30% of the population are carriers, leads to a greater PAR (16.8%) and probably contributes to the higher incidence of prostate cancer in African Americans. The allele associates more with aggressive forms of prostate cancer.

### Materials and Methods

Icelandic study population. This cohort was based on a nation-wide list from the Icelandic Cancer Registry (ICR) that contains all 3815 Icelandic prostate cancer patients (International Classification of Disease Revision 10 code (ICD10) C61) diagnosed during the period Jan. 1, 1955 to Dec. 31, 2004 of which 1291 consented to the study. In addition, an average of three first-degree relatives and spouses also participated (88% participation rate for patients and relatives). Clinical information for patients from the ICR included age at diagnosis, SNOMED morphology codes and stage. Biopsy Gleason scores were obtained from medical records and reviewed by pathologists KRB and BAA. The mean age of diagnosis of genotyped patients was 71 years and the mean age of all prostate cancer patients in the ICR was 73 years.

The BPH population comprised 510 individuals diagnosed in Iceland with histopathologically confirmed diagnoses of BPH between the years 1982 to 2000 that were not diagnosed with prostate cancer.

A control group of 997 individuals was recruited from the general population. This group is unrelated at three meioses, has a sex ratio of one and an age range of 25-85 years (median age of 50 years). No sex differences were seen for allele −8 of DG8S737 and allele A of rs1447295 in control individuals.

The study was approved by the Data Protection Commission of Iceland and the National Bioethics Committee of Iceland. Written informed consent was obtained from all patients, relatives and controls. Personal identifiers associated with medical information and blood samples were encrypted with a third-party encryption system as previously described (Gulcher, J. R. et al., Eur. J. Hum. Genet. 8:739-42 (2000)).

Swedish and U.S. study populations. CAPS1 (CAncer Prostate in Sweden1) is a population based case-control study where prostate cancer patients (ICD10 C61) were recruited from four of the six regional cancer registries in Sweden from January 1st or Jul. 1, 2001 until September 2002. The study population consisted of 1435 cases and 779 controls matched for age, gender and place of residency. Clinical information including stage and Gleason scores, ˜80% from by biopsy and ˜20% from surgery, were obtained from cancer registries or the National Prostate Cancer Registry. The mean age at diagnosis was 66.6 years for patients and the mean age at inclusion 67.9 years for controls. The study was approved by the Ethics Committees at the Karolinska Institute and Umea University. Informed consent was obtained from all subjects (Zheng, S. L. et al., Cancer Res. 64:2918-22 (2004); Lindmark, F. et al., J. Natl. Cancer Inst. 96:1248-54 (2004)).

The Caucasian U.S. study population consisted of 458 prostate cancer patients (ICD10 C61), who underwent surgery at the Urology Department of Northwestern Memorial Hospital, Chicago, and 260 population based controls enrolled at the Department of Human Genetics, University of Chicago. Medical records were examined to retrieve clinical information including stage and biopsy Gleason score. The mean age at diagnosis was 59 years for patients. Both patients and controls were of self-reported European American ethnicity. This was confirmed by the estimation of genetic ancestry using 30 microsatellite markers distributed randomly throughout the genome (see below). The mean and median portion of European ancestry in this cohort were both greater than 0.99 (see methods described below for details). The study protocols were approved by the Institutional Review Boards of Northwestern University and the University of Chicago. All subjects gave written informed consent.

The African American study population consisted of 246 prostate cancer patients (ICD10 C61) and 352 controls recruited through the Flint Men's Health Study and the Prostate Cancer Genetics Project. The Flint Men's Health Study (FMIIS) is a community-based case-control study of prostate cancer in African-American men between the ages of 40-79 that was conducted in Genesee County, Michigan between 1996 and 2002 (Cooney, K. A. et al., Urology 57:91-6 (2001); Beebe-Dimmer, J. L. et al. Prostate Cancer Prostatic Dis. 9, 50-5 (2006)) and from that study 113 cases and 352 controls were analyzed. The Prostate Cancer Genetics Project (PCGP) conducted at the University of Michigan is a large family-based study with enrollment including men with two or more living family members with prostate cancer or men diagnosed with prostate cancer before age 56 years without a documented family history of disease (Douglas, J. A. et al., Cancer Epidemiol Biomarkers Prev. 14:2035-9 (2005)). From that cohort 153 patients coming from 109 families were analyzed, of which 78 patients were unrelated and 75 clustered in 31 families (majority first-degree relatives). Fifteen prostate cancer patients were present in both the FMHS and PCGP cohorts. Medical records were reviewed to extract information related to prostate cancer diagnosis including stage and biopsy Gleason score. Patients and controls were of self-reported African American ethnicity. The proportion of African and European ancestry in this cohort was assessed using the Structure software (Pritchard, J. K. et al., Am. J. Hum. Genet 67:170-81 (Epub 2000 May 26)) to analyse genotypes from 30 microsatellites distributed randomly throughout the genome (Helgadottir, A. et al., Am. J. Hum. Genet. 76:505-9 (Epub 2005 Jan. 7)). Each of these microsatellites has alleles that exhibit large differences in frequency (>0.4) between pairs of population samples used in the HapMap project (i.e. CEU, YRI or East Asian). Genotypes from the Michigan cohort were run in Structure with genotypes from the YRI (as an African reference sample), CEU HapMap samples, and a sample of 96 Icelanders (as a combined European reference sample). The USEPOPINFO option in Structure was employed with K=3, so that information about individuals with known ancestry (the African and European reference samples) could be used to help determine the ancestry of individuals with unknown ancestry (the African Americans from Michigan). The resulting mean proportion of European ancestry in the Michigan cohort was estimated as 0.224 (median=0.21) in patients and 0.215 (median=0.207) in controls. The difference in means was not statistically significant (P=0.11) according to a randomization test performed with 10,000 iterations. Association calculations for the Michigan cohort were adjusted for these genetic estimates of ancestry (see below for details). Informed consent was obtained from all study participants, and protocols were approved by the Institutional Review Board at the University of Michigan Medical School.

Statistical analysis. A genome-wide scan was performed with a framework scan of 1068 microsatellites, as previously described (Gretarsdottir, S. et al., Am. J. Hum. Genet. 70:593-603 (2002); Styrkarsdottir, U. et al., PLoS boil. 1:E69 (2003)). Genotypes from a total of 871 Icelandic patients diagnosed with prostate cancer and an average of three of their first-degree relatives were analyzed. Pedigrees were identified using our genealogical database of Icelanders (Gulcher, J. and Stefansson, K., Clin. Chem. Lab Med. 36:523-7 (1998); Gulcher, J. et al., Cancer J. 7:61-8 (2001); Gulcher, J. et al., Eur. J. Hum. Genet. 8:739-42 (2000)). Linkage analysis was performed by defining prostate cancer patients as affected and all others as unknown. For multipoint linkage analysis, an affected-only allele-sharing method (Kong, A. and Cox, N.J., Am. J. Hum. Genet. 61:1179-88 (1997)) was used, as implemented in the program Allegro (Gudbjartsson, D. F. et al, Nat. Genet. 25:12-3 (2000)), and the deCODE genetic map (Kong, A. et al., Nat. Genet. 31:241-7 (2002)) (see below). An additional 25 markers were typed in the region of suggestive linkage to increase the information content.

For single-marker association to prostate cancer, a likelihood ratio test was used to calculate a two-sided p-value for each allele. For the overall Icelandic cohort (1291 cases and 997 controls), formed by merging cohorts I and II, some of the individuals with prostate cancer were related to each other. To take account of this, a null distribution of the test statistic was obtained by simulating genotypes through the Icelandic genealogy (see below). A similar procedure was used to adjust for the relatedness of some individuals with prostate cancer in the Michigan African American cohort. Allelic frequencies rather than carrier frequencies are presented for the markers. Allele-specific RR was calculated assuming a multiplicative model (Falk, C. T. and Rubinstein, P., Ann. Hum. Genet. 51 (Pt 3):227-33 (1987)). When comparing risks of different haplotype groups, the program NEMO that employs a likelihood procedure was used (Gretarsdottir, S. et al., Nat. Genet. 35:131-8 (2003)). Results from multiple cohorts were combined using a Mantel-Haenszel model (Mantel, N. and Haenszel, W., J. Natl. Cancer Inst. 22:71948 (1959)) in which the cohorts were allowed to have different population frequencies for alleles or genotypes but were assumed to have common relative risks.

Linkage analysis. The Spairs scoring function (Whittemore, A. S. and Halpern, J., biometrics 50:118-27 (1994); Kruglyak, L. et al., Am. J. Hum. Genet. 58:1347-63 (1996)) and the exponential allele-sharing model (Kong, A. and Cox, N.J., Am. J. Hum. Genet. 61:1179-88 (1997)) were used to generate the relevant 1 df (degree of freedom) statistics. When combining the family scores to obtain an overall score, instead of weighting the families equally or weighting the affected pairs equally, a weighting scheme was used that is halfway between the two in the log scale; the family weights are the geometric means of the weights of the two schemes.

Correction for relatedness. The association of an allele to prostate cancer was tested using the signed (+ for excess in patients, − for deficit) square-root of a standard likelihood ratio statistic applied to the allele counts in the patients and controls, which, if the subjects were unrelated, would have asymptotically a standard normal distribution under the null hypothesis. Because some Icelandic patients were related and their genotypes not independent, the statistic as described has a standard deviation larger than 1 and ignoring that would lead to P-values that are anti-conservative. An adjustment was performed using a previously described procedure (Grant, S. F. et al., Nat. Genet. 38:320-3 (Epub 2006 Jan. 15); Stefansson, H. et al., Nat. Genet. 37:129-37 (Epub 2005 Jan. 16)). 10,000 sets of genotypes were simulated for the marker DG8S737 through the genealogy of 708,683 Icelanders. With each simulated set, the statistic was re-calculated by treating the simulated genotypes as real genotypes of the patients and controls in the study. From the simulations, the true standard deviation of the statistic under the null hypothesis is 1.018 for allele −8, and this value was used to calculate the P-values for the Icelandic total cohort of 1291 prostate cancer patients and 997 controls. Based on similar simulations, the adjustment factor for allele A of rs1447295 was found to be somewhat lower, as expected due to the higher frequency of allele A compared to allele −8. It was decided to use the higher adjustment factor of 1.018 throughout for simplicity. Hence the results reported for allele A are slightly conservative. Applying the same method to the Michigan African American cohort with the given relationships of some of the patients, the adjustment factor was found to be 1.032.

Evaluation of genetic ancestry. The program Structure (Pritchard, J. K. et al., Genetics 115:945-59 (2000)) was used to estimate the genetic ancestry of individuals. Structure infers the allele frequencies of K ancestral populations on the basis of multilocus genotypes from a set of individuals and a user-specified value of K, and assigns a proportion of ancestry from each of the inferred K populations to each individual. The analysis of the data set was run with K=3, with the aim of identifying the proportion of African and European ancestry in each individual. The statistical significance of the difference in mean European ancestry between African American patients and controls was evaluated by reference to a null distribution derived from 10000 randomized datasets.

To evaluate genetically estimated ancestry of the study cohorts from the US, 30 unlinked microsatellite markers were selected from about 2000 microsatellites genotyped in a previously described (Pritchard, J. K. et al., Genetics 115:945-59 (2000)) multi-ethnic cohort of 35 European Americans, 88 African Americans, 34 Chinese, and 29 Mexican Americans. Of the 2000 microsatellite markers the selected set showed the most significant differences between European Americans, African Americans, and Asians, and also had good quality and yield: D1S2630, D1S2847, D1S466, D1S493, D2S166, D3S1583, D3S4011, D3S4559, D4S2460, D4S3014, D5S1967, DG5S802, D6S1037, D8S1719, D8S1746, D9S1777, D9S1839, D9S2168, D10S1698, D11S1321, D11S4206, D12S1723, D13S152, D14S588, D17S1799, D17S745, D18S464, D19S113, D20S878 and D22S1172. The following primer pairs were used for DG5S802: DG5S802-F: CAAGTTTAGCTGTGATGTACAGGTTT (SEQ ID NO: 23) and DG5S802-R: TTCCAGAACCAAAGCCAAAT (SEQ ID NO: 24).

PCR screening of cDNA libraries. Commercially available cDNA libraries were screened for AW transcripts. The libraries screened were Prostate Marathon-Ready cDNA library (Clontech Cat. 7418-1), Testis Marathon-Ready cDNA library (Clontech Cat. 7414-1), Bone marrow-Ready cDNA library (Clontech Cat. 7431-1), In addition cDNA libraries were constructed for whole blood and EBV-transformed human lymphoblastoid cells. Total RNA was isolated from the lymphoblastoid cell lines and whole blood, using the RNeasy RNA isolation kit from Qiagen (Cat. 75144) and the RNeasy RNA isolation from whole blood kit (Cat 52304), respectively. RNA was subsequently analysed and quantitated using the Agilent 2001 Bioanalyser.

cDNA libraries were prepared using a random hexamer protocol from the RevertAid™ H Minus First Strand cDNA Synthesis Kit (Fermentas Cat. K1631). The PCR reactions were done in 10 ul volume at a final concentration of 3.5 μM of forward and reverse primers, 2 mM dNTP, 1× Advantage 2 PCR buffer and 0.5 ul of cDNA library. PCR screening was carried out using the Advantage® 2 PCR Enzyme RT_PCR System (Clontech) according to manufacturers instructions. PCR primer pairs (Operon Biotechnologies) used are shown in Table 8.

TABLE 8
Primers used for Genscan gene predictions

| Predicted gene | Forward primer | Predicted gene | Reverse primer |
|---|---|---|---|
| NT-008046.708 | AACTGCCTCTGACAACTCTTGTG (SEQ ID NO: 25) | NT-008046.708 | TTAAGATGCTTGAAGTCCCCAGT |
| | | | (SEQ ID NO: 26) |
| NT-008046.708 | AACTGCCTCTGACAACTCTTGTG (SEQ ID NO: 27) | NT-008046.708 | AAGCTGCTGTACGGATTTTTCAC |
| | | | (SEQ ID NO: 28) |
| NT-008046.709 | GGAGAGCCTATTTGTGGTCAAGA (SEQ ID NO: 29) | NT-008046.709 | AAGTGGATTGCAGAAGTCTCTGG |
| | | | (SEQ ID NO: 30) |
| NT-008046.709 | CTAATTGAGAAGGCTGGCTATGG (SEQ ID NO: 31) | NT-008046.709 | GTAGGATCAGACCATCCAATGC |
| | | | (SEQ ID NO: 32) |

B. Primers used for ESTs

| EST | | EST | |
|---|---|---|---|
| AW183883 | CAGGGATTTTGTCTGTTTTGTTG (SEQ ID NO: 33) | AW183883 | TTTATTCGGATGCTCAGAAGCTG |
| | | | (SEQ ID NO: 34) |
| AW183883 | GCAGGAAGCCACTGCTGCTCCTTA (SEQ ID NO: 35) | AW183883 | GCAGTGCCAGCACCTGTTAGCATTAAA |
| | | | (SEQ ID NO: 36) |
| CV364590 | TGCACAAGCCTGATTTAAAAGTG (SEQ ID NO: 37) | CV364590 | CCAGTTTTTGGTTTTGGTTGTTT |
| | | | (SEQ ID NO: 38) |
| AF119310* | CCAGACATGTTACTGATGTTTTGG (SEQ ID NO: 39) | AF119310* | CCAGAGTGGTAGCAATGTTCTGT |
| | | | (SEQ ID NO: 40) |
| BE144297 | GGAATGCTTCCTTGTATGTGGAG (SEQ ID NO: 41) | BE144297 | GAGGGAAACTGACTGGAAAGATT |
| | | | (SEQ ID NO: 42) |

C. Primers used to connect ESTs

| EST | | EST | |
|---|---|---|---|
| CV364590 | GCACAAGCCTGATTTAAAAGTGC (SEQ ID NO: 43) | AW183883 | CAGGGATTTTGTCTGTTTTGTTG |
| | | | (SEQ ID NO: 44) |
| CV364590 | GCACAAGCCTGATTTAAAAGTGC (SEQ ID NO: 45) | AW183883 | CTTCTGTCCTCAGCGGAAACAGCTT |
| | | | (SEQ ID NO: 46) |
| AF119310* | TCTGTTTCTTTGACCTGGGTTGT (SEQ ID NO: 47) | AW183883 | CAGGGATTTTGTCTGTTTTGTTG |
| | | | (SEQ ID NO: 48) |
| AF119310* | TCTGTTTCTTTGACCTGGGTTGT (SEQ ID NO: 49) | AW183883 | CTTCTGTCCTCAGCGGAAACAGCTT |
| | | | (SEQ ID NO: 50) |
| BE144297 | GGAGGGAAACTGACTGGAAAGAT (SEQ ID NO: 51) | AW183883 | CAGGGATTTTGTCTGTTTTGTTG |
| | | | (SEQ ID NO: 52) |
| BE144297 | GGAGGGAAACTGACTGGAAAGAT (SEQ ID NO: 53) | AW183883 | CTTCTGTCCTCAGCGGAAACAGCTT |
| | | | (SEQ ID NO: 54) |
| AF119310* | CCAGAGTGGTAGCAATGTTCTGT (SEQ ID NO: 55) | CV364590 | CCAGTTTTTGGTTTTGGTTGTTT |
| | | | (SEQ ID NO: 56) |
| AF119310* | CCAGAGTGGTAGCAATGTTCTGT (SEQ ID NO: 57) | BE144297 | GGAATGCTTCCTTGTATGTGGAG |
| | | | (SEQ ID NO: 58) |
| BE144297 | GAGGGAAACTGACTGGAAAGATT (SEQ ID NO: 59) | CV364590 | CCAGTTTTTGGTTTTGGTTGTTT |
| | | | (SEQ ID NO: 60) |

Gene prediction and EST names are from UCSC Build34 except AF119310* from BUILD 35.

RACE. 5′- and 3′-RACE of the AW transcript was carried out using the Marathon-Ready cDNA libraries (Clontech), according to the manufacturers instructions. The primers (Operon Biotechnologies) shown in Table 9 were used.

TABLE 9
Primers used for RACE

| AW-race 3.F | GCAGGAAGCCACTGCTGCTCCTTA (SEQ ID NO: |
|---|---|
| | 61) |
| AW-race 3.R | GCAGTGCCAGCACCTGTTAGCATTAAA (SEQ ID NO: |
| | 62) |
| AW-race1.F | AAGCTGTTTCCGCTGAGGACAGAAG (SEQ ID NO: |
| | 63) |
| AW-race1.R | CTTCTGTCCTCAGCGGAAACAGCTT (SEQ ID NO: |
| | 64) |
| AW-ex3.1R | TATACACCAGAATGCCCCGCATC (SEQ ID NO: 65) |
| AW-ex4.1R | GATAGGGCCGCTACCATTTGGAAAG (SEQ ID NO: |
| | 66) |
| AW-ex3.1F | TGTCAACCGCAACACTGGTTGTGT (SEQ ID NO: |
| | 67) |
| AW-ex4.1F | CTGGAGTGCCTCTCTTCCTTTTTGC (SEQ ID NO: |
| | 68) |

B. Primers used for nested PCR

| | |
|---|---|
| AW-race2.F | AAGATGCCAGGGCTACAGCAATCA (SEQ ID NO: |
| | 69) |
| AW-race2.R | TGATTGCTGTAGCCCTGGCATCTT (SEQ ID NO: |
| | 70) |
| AW-ex2.F1 | TTGCTTTTAAGCATGAAGCCACTCA (SEQ ID NO: |
| | 71) |
| AW-ex1.R1 | GGCATGGACCAGGAGCACTAGTTA (SEQ ID NO: |
| | 72) |
| AW-ex3.1Rne | AACACAACCAGTGTTGCGGTTGAC (SEQ ID NO: |
| | 73) |
| AW-ex4.1Rne | TGAAACAACAGTAAGCACTGGCTCTC (SEQ ID NO: |
| | 74) |
| AW-ex3.1Fne | GATGCGGGGCATTCTGGTGTA (SEQ ID NO: 75) |
| AW-ex4.1Fne | ACTCAATTGTTGCCATGGGCTTGAT (SEQ ID NO: |
| | 76) |

New splice variants of the AW transcript identified through RACE were verified using RT-PCR on the corresponding cDNA libraries. PCR products were all cloned and sequence verified to confirm the original RACE results.

Cell lines. The following prostate cancer cell lines were obtained from ATCC. DU 145, a prostate cancer cell line generated from brain metastasis; LNCaP, a prostate cancer cell line generated from lymph node metastasis; CA-HPV-10, a prostate cancer cell line generated from adenocarcinoma following HPV 18 transfection; PZ-HPV-7 and RWPE-1 both generated from normal prostate tissue following HPV18 transfection. In addition, lymphoblastoid cell lines were generated by EBV-transformation from the peripheral blood of certain Icelandic prostate cancer patients. These cell lines were used for Southern blot analysis.

Northern blot analysis. Commercial multiple tissue Northern blots were obtained from Clontech (Human MTN® Blot II Cat. 7759-1). In addition blots were made from the prostate cancer cell lines described above. Briefly, total RNA was isolated from cell lines using a combined Trizol (GIBCO BRL Catalog #15596-018) and RNAeasy (Qiagen Catalog #74106) purification protocol following the manufacturer's instructions. Poly (A) RNA was further purified using the Poly(A)Purist™ MAG Kit from Ambion (Cat. 1922) 1.5 μg poly (A) RNA was electrophoresed in an agarose-formaldehyde gel, blotted to Hybond N nylon membranes (Amersham), and fixed using UV-crosslinking.

Probes used included: i) The AW1838833 cDNA clone (IMAGp998M216650Q) obtained from RZPD Deutsches Ressourcenzentrum für Genomforschung GmbH, Germany (http://www.rzpd.de/products/genomecube.shtml); and ii) cDNA clone that corresponded to exon 6-8 of the AW transcripts obtained from RT-PCR experiments. The clone was sequence verified as follows:

(SEQ ID NO: 77)

TTGCTCCTCAGGAACCCTATTTTGGACTGACGTTTAATACAACATGGAA
GCCACCAAGGCTTACAGAATGTGCTTTCCAGAGCTGTGACCTGAACTGT
ACCTGGGGCCTTTTGAGTGAGGCTGGAACTGGAGTGGCCTGGATGCAG
AGAGCAGTGTCCTAAGGCTGTGCAGGTTGCAAGAAAGCTCAAGTAGCC
TATGGAGAGGATGCAAGGCTTCCAGCTGATGCCCTCAGCCAGGCTCAG
TAGCAGCCAGAACTAGCCTACCAACGAACCTGCTGATCATGTGCATAAG
CCACCTTGAACGTCGATCCTCCTGCCTGGTGGAGCCATCCCAGCTGATG
CCACATGAAGCAGACACAAGCTGTCCCTACTAAGCTCTGCTCAAGTTGGA
TATTCATGAGTGAAATAAATGACTGTTACTAAGTAATTAATTTTTGGGTG
GCTGTTATGTAGCAGTAGATAATTGGAACAAAGCTTATTGACATAATACA
TCTATATCMCATCCTCCAATCCATTTTTTTAAGTAATAAAGTTGATGTTT
GTTTTGAAAAAAAAAAAAAAAAAAAAAAAGACCTGCCCGGGCGGCCG
CTCGAGCCCTATAGTGAGTAAGGGCGAATCCAGCACACTGGCGCCGTA
CTAGTGATCCGAGCTCGTAGCA.

cDNA fragments were radiolabelled with [α⁻³²P]dCTP (specific activity 6000 Ci/mmol), using the Megaprime labelling kit (Amersham Cat. RPN 1607) and unincorporated nucleotides removed from the reaction using ProbeQuant G-50 microcolumns (Amersham Cat. 27-5335-01). Membranes were pre-hybridized in Rapid-hyb buffer (Amersham Cat. RPN 1635) for at least 30 minutes and subsequently hybridized with 100-300 ng of the labelled cDNA probe. Hybridizations were performed in Rapid-hyb buffer at 68° C. overnight and 0.1-0.15 μg/ml sheared, denatured salmon sperm DNA when using cDNA probes. The labelled probes were heated for 5 minutes at 95° C. before addition to the filters in the pre-hybridization solution. After hybridization, the membranes were washed at low stringency in 2×SSC, 0.05% SDS at room temperature for 30-40 minutes followed by two high stringency washes in 0.1×SSC, 0.1% SDS at 50° C. for 40 minutes. The blots were immediately sealed and exposed to Kodak BioMax MR X-ray film (Cat. 8715187).

Pulse-field Southern blot analysis High molecular weight DNA in agarose blocks was prepared by embedding lymphoblast cell lines, generated from peripheral blood of prostate cancer patients, within low-melting-point agarose (Incert, FMC bioproducts) with a Biorad 10 plug pleximould. (Biorad catalog no. 170-3591). Final cell concentration within the agarose was always adjusted to 2×10⁷ cells per ml. DNA was also isolated from fresh frozen normal and malignant prostate tissue. For each patient, DNA was isolated from four to five 20 micron slices of OCT embedded fresh frozen tissue samples (>70% tumor percentage) using the MasterPure™ DNA Purification Kit Epicentre Inc. Cat MC85200). DNA was subsequently amplified using the GenomiPhi DNA Amplification Kit (GE Healthcare, Cat. 25-6600-02) according to the manufacturer's protocol and diluted by an equal amount of TE-Buffer. Agarose blocks and WGA prostate tissue DNA samples corresponding to 10 ug of DNA were digested with the HindIII restriction endonuclease following standard protocols (New England Biolabs). Following digestion the agarose blocks or WGA DNA samples were loaded into a 0.8% agarose gel. After electrophoresis the gel was depurinated in 0.25M HCl for 30 min and denatured in 0.5M NaOH, 1.5M NaCl DNA then transferred to a nylon filter (Hybond N+). The membranes were then probed with a radiolabeled purified BAC insert RP11 367L7(Amersham megaprime) following standard protocols as described above for Northern blotting. After washing the membrane was exposed to film (Kodak MR) from 14 days at −80° C.

Confirmation in Icelandic Cohorts

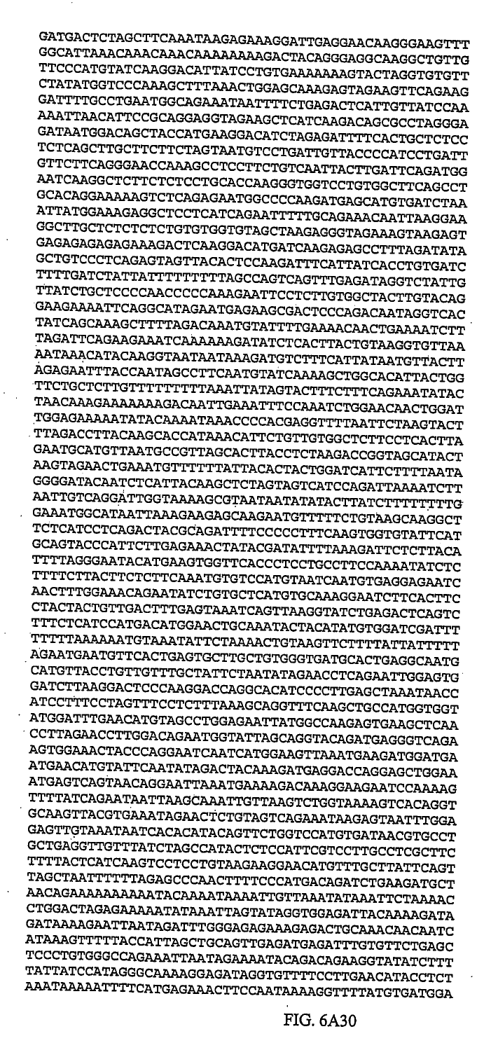

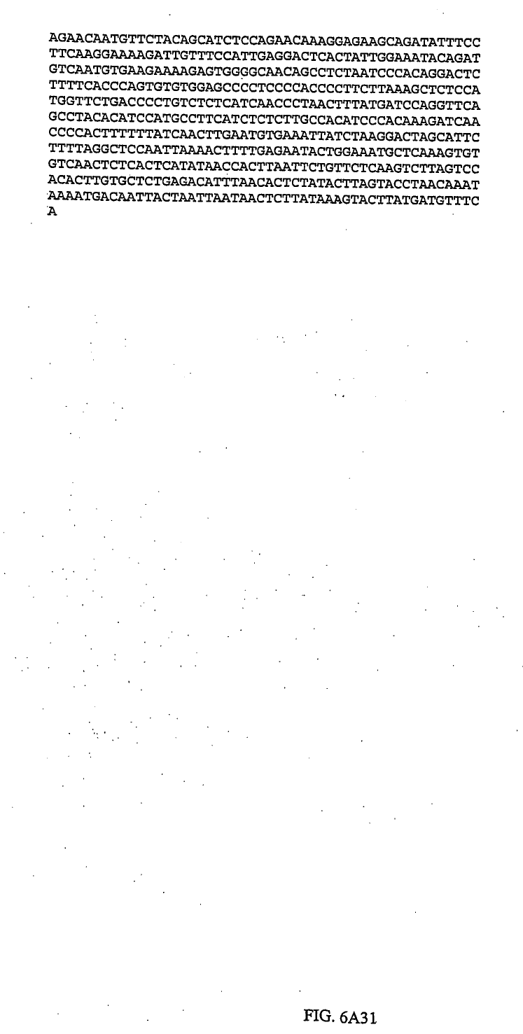

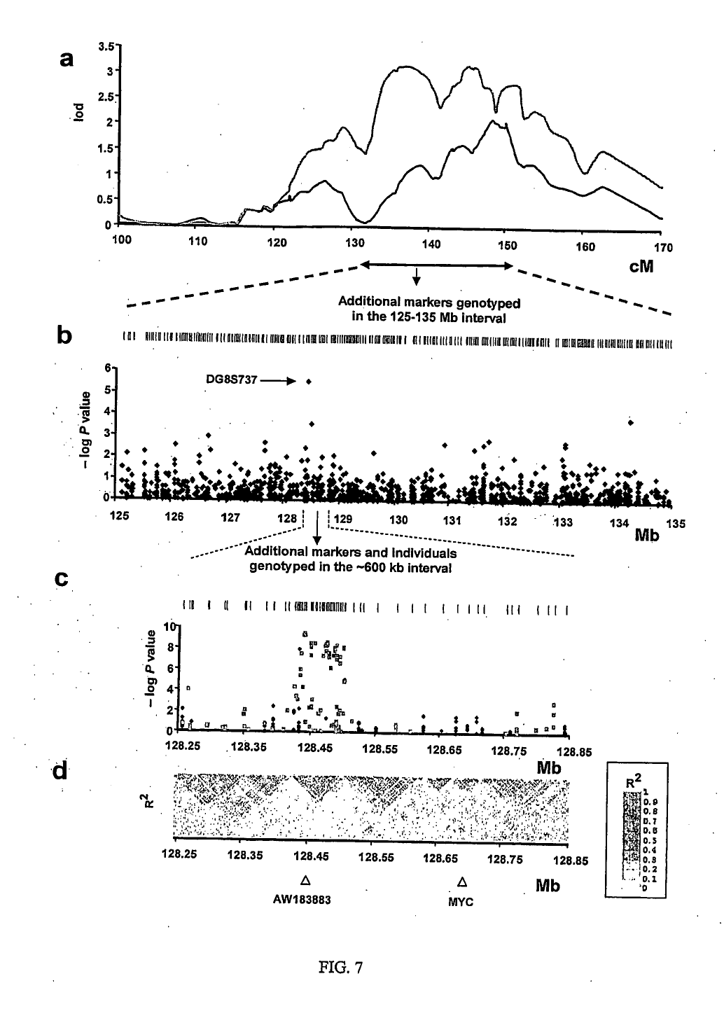

In an attempt to identify genetic variants underlying prostate cancer risk, a genome-wide linkage scan was conducted using 1068 microsatellite markers typed in a cohort of 871 Icelandic prostate cancer patients grouped into 323 extended families. This scan produced a suggestive linkage signal on chromosome 8q24 which after addition of markers to increase the information content gave a maximum load score of 2.11 (D8S529 at 148.25 cM) and 3.15 (D8S557 at 145.65 cM) (FIG. 7A). To refine the source of the linkage signal, 358 microsatellite and indel markers spanning 10 Mb (18.6 cM) on chromosome 8 from 125-135 Mb (NCBI Build 34) in 869 were genotyped in unrelated prostate cancer patients and 596 population controls (cohort I) (FIGS. 7A and 7B). Single marker association to prostate cancer was calculated based on a multiplicative model of risk (Falk, C. T. and Rubinstein, P., Ann. Hum. Genet. 51(pt 3), 227-33 (1987)). The strongest association was observed for allele −8 of the microsatellite DG8S737, with an estimated relative risk (RR) of 1.79 per copy carried (P=3.0×10⁻⁶) (FIG. 7B and Table 10). This association was replicated in a second Icelandic cohort of 422 prostate cancer patients and 401 population based controls (cohort II), where allele −8 carried an estimated RR of 1.72 (P=0.0018, all P-values are two-sided, including those obtained from replication studies). In the overall Icelandic cohort of 1291 prostate cancer patients and 997 controls (merging cohorts I and II), the DG8S737 −8 allele had a frequency of 13.1% in patients and 7.8% in controls. This results in an estimated RR of 1.77 (P=2.3×10⁻⁸), an estimated RR of 1.77 (P; 2.3×10-8) and a population attributable risk (PAR) of 11% (Table 10), after adjusting for relatedness between patients from cohorts I and II. The DG8S737 marker (128.433096 Mb) is located within a linkage disequilibrium (LD) block that spans 92 kb on chromosome 8q24.21 (from 128.414 to 128.506 Mb of NCBI Build 34) in HapMap CEU samples. The LD block is referred to herein as LD Block A.

TABLE 10
Association of alleles at chromosome 8q24 to prostate cancer
in Iceland

| Study | | | | | | |
|---|---|---|---|---|---|---|
| population | | | Allelic | | | |
| (N cases/N | | | Frequency | | | |
| controls) | Marker | Allele | Cases | Controls | RR | P value |
| Iceland | | | | | | |
| Group I^{a} | | | | | | |
| (869/596) | DG8S737 | −8 | 0.134 | 0.080 | 1.79 | 3.0 × 10⁻⁶ |
| Group II^{b} | | | | | | |
| (422/401) | DG8S737 | −8 | 0.124 | 0.076 | 1.72 | 1.8 × 10⁻³ |
| Combined | | | | | | |
| groups I | | | | | | |
| and II^{b} | | | | | | |
| (1291/997) | DG8S737 | −8 | 0.131 | 0.078 | 1.77 | 2.3 × 10⁻⁸ |
| ″ | rs1447295 | A | 0.169 | 0.106 | 1.72 | 1.7 × 10⁻⁹ |

Alleles for the markers DG8S737 and rs1447295 at 8q24.21 are shown and the corresponding numbers of cases and controls (N), allelic frequencies of variants in affected and control individuals, the odds-ratio (RR) and two-sided P values.
^{a}Individuals are unrelated at 3 meioses
^{b}The association analysis was adjusted for the relatedness of some of the individuals.

To investigate the extent of the association signal, 12 additional microsatellites and 63 SNPs in a 600 kb region surrounding DG8S737 were genotyped (FIG. 7C, Tables 11 and 12). After typing additional microsatellite and SNP markers in a 600 kb region surrounding DG8S737, it was found that allele A of the SNP SG08S717 (rs1447295) showed the strongest association (FIG. 7C). Other alleles/markers that were located in the same LD block as DG8S737 and SG08S717 (rs1447295) associated significantly with prostate cancer as shown in Table 13 and can be used to detect the risk variants that associate with prostate cancer. These markers and alleles are thus surrogates for the −8 DG8S737 and A SG08S717 (rs1447295) alleles, as are many of the possible haplotypes comprising at least two of the markers listed in Table 13.

TABLE 11
Microsatellite and indel markers genotyped in the 600 kb region on Chr8q24

| Marker | Location | | | |
|---|---|---|---|---|
| Name | (Mb)* | Size | Forward primer | Reverse primer |
| DG8S605 | 128.257 | 336 | CCACTTGGGTGGTATCAGGT (SEQ ID NO: 78) | ACTCAAGGAAAGGGCCAAA (SEQ ID NO: 79) |
| DG8S1339 | 128.272 | 189 | TCAGAAGGGCACATAAGAGGA (SEQ ID NO: 80) | GCTGCTTTCAGGATCAGGAG (SEQ ID NO: 81) |
| DG8S1766 | 128.296 | 195 | GGGATACCAACAACATCTATCACA (SEQ ID NO: 82) | GCTCTTTCTATTTGCACACCAA (SEQ ID NO: 83) |
| DG8S1767 | 128.319 | 116 | TGCAGACTGTGCAGCAGATA (SEQ ID NO: 84) | CTGCTAGAGATGTGTGCCCTA (SEQ ID NO: 85) |
| DG8S1778 | 128.323 | 323 | ATGGGTCTTGATGGACATGC (SEQ ID NO: 86) | GTGGATGGATCCAGAGAGGA (SEQ ID NO: 87) |
| DG8S1409 | 128.382 | 430 | CAGAGCATCACCTCAAACGA (SEQ ID NO: 88) | ATCCTGCCAACCTTAAGTCC (SEQ ID NO: 89) |
| DG8S540 | 128.395 | 236 | GGCAAGAAACACAAGGCAAT (SEQ ID NO: 90) | AGGTTGAATGAGCCAGATGC (SEQ ID NO: 91) |
| DG8S1434 | 128.426 | 403 | CCACAGTGATTCCCACCTCT (SEQ ID NO: 92) | AGTGTTGGCCAGGGATGTAG (SEQ ID NO: 93) |
| DG8S737 | 128.433 | 134 | TGATGCACCACAGAAACCTG (SEQ ID NO: 94) | CAAGGATGCAGCTCACAACA (SEQ ID NO: 95) |
| DG8S1761 | 128.453 | 392 | TTGAAATTGCAATCCCATCA (SEQ ID NO: 96) | CCTCCCTACTTATTCCCATGC (SEQ ID NO: 97) |
| DG8S422 | 128.475 | 378 | AAATGCAAGCAAAGCCAAGT (SEQ ID NO: 98) | GCTCCACACACAGAGGTCAA (SEQ ID NO: 99) |
| DG8S1769 | 128.501 | 262 | CCTCCCAAACACACAGAGTTG (SEQ ID NO: 100) | TGTTAAACCTAAGGGTTCCTTCC (SEQ ID NO: 101) |
| DG8S1407 | 128.503 | 236 | CCAATAGCCTTCAATGTATCAAA (SEQ ID NO: 102) | TGAGGAAGAGCCACAACAGA (SEQ ID NO: 103) |
| DG8S1351 | 128.526 | 200 | CAGAGAGACAGAAATGGTCTCA (SEQ ID NO: 104) | TTCTTAACACGCAGCACATT (SEQ ID NO: 105) |
| DG8S482 | 128.531 | 401 | GCCCTATTTCCTAACACATGC (SEQ ID NO: 106) | GCTAACATGCTAATGTGCTTCC (SEQ ID NO: 107) |
| D8S1128 | 128.552 | 241 | AAACAATCAAAGGCCCAGG (SEQ ID NO: 108) | CCCATTGGAAACAGAGTTGA (SEQ ID NO: 109) |
| DG8S1825 | 128.583 | 392 | CAAGGAGGGTGGATCACTTG (SEQ ID NO: 110) | AGAGGCTCCAAAGGGAGATT (SEQ ID NO: 111) |
| DG8S1817 | 128.606 | 223 | CCCTAAATGCAGATGGTTATGA (SEQ ID NO: 112) | GCTTGTGCTATCTGTCCCTTG (SEQ ID NO: 113) |
| DG8S432 | 128.626 | 198 | TGCACAAAGCTGTTCTACACA (SEQ ID NO: 114) | ACTGCTTCCAGCCAGACATT (SEQ ID NO: 115) |
| DG8S1324 | 128.654 | 243 | CTGCACTCCCAAGACAGACA (SEQ ID NO: 116) | GTTGAAGCAGGCTTTCTGGA (SEQ ID NO: 117) |
| DG8S471 | 128.677 | 128 | CAGCAACCGTTTCCTTTCAT (SEQ ID NO: 118) | TTTGAGGTTGGTGTCACTGG (SEQ ID NO: 119) |
| DG8S740 | 128.694 | 118 | ACATTTCCCGTATCGTCCAA (SEQ ID NO: 120) | AATGGGCTGGCACAGAAA (SEQ ID NO: 121) |
| DG8S1335 | 128.708 | 185 | GCTGGGATCTTCTCAGCCTA (SEQ ID NO: 122) | GCTGCAAATTGCTTGGTATG (SEQ ID NO: 123) |
| DG8S1143 | 128.717 | 251 | TCAGTCCTATGCTGCCTCCT (SEQ ID NO: 124) | ATGGGCTATTGTGTAAGCCTCT (SEQ ID NO: 125) |
| DG8S1816 | 128.754 | 359 | TCCCTACCACACCTACATCCA (SEQ ID NO: 126) | CTGCGTCGGCCAGATTAC (SEQ ID NO: 127) |
| DG8S1436 | 128.761 | 342 | ATTCAAGCCCGGTAACACAG (SEQ ID NO: 128) | CTGACAGTTGATGCCCAGTC (SEQ ID NO: 129) |
| DG8S1818 | 128.771 | 121 | AAACACACATTGGATTTCAGAGAC (SEQ ID NO: 130) | GCTGGGCAACAGGTGAGAC (SEQ ID NO: 131) |
| DG8S1824 | 128.800 | 334 | ATGCTTCCTGCCCTCAGAC (SEQ ID NO: 132) | TCCTGCCTCAGCCTCTGTAT (SEQ ID NO: 133) |
| DG8S1828 | 128.816 | 339 | GCCTCTGGAGTGGCTAGGAT (SEQ ID NO: 134) | ATGAGATGGCCAGGTCAAAG (SEQ ID NO: 135) |
| DG8S1820 | 128.827 | 278 | CGGTCCAACATGGTGAAATA (SEQ ID NO: 136) | CCAAACCGAAACCTCAAGAC (SEQ ID NO: 137) |
| DG8S455 | 128.844 | 123 | CTCGCTCTGCAGTCTTGGTT (SEQ ID NO: 138) | CATGGTGAAAGGGCAACTG (SEQ ID NO: 139) |
| DG8S548 | 128.844 | 238 | AGCAAGAAGGGAGAGGTGTG (SEQ ID NO: 140) | TGGCCACATCCCTTTAAATC (SEQ ID NO: 141) |

Shown are microsatellite markers typed in the 600 kb region around marker DG8S737.
*NCBI Build 34

TABLE 12
SNP markers genotyped in the 600 kb region on Chr8q24

| | | Location |
|---|---|---|
| SG-name | RS-name | (bp's)* |
| SG08S665 | rs283701 | 128258003 |
| SG08S667 | rs283720 | 128266554 |
| SG08S668 | rs283727 | 128269949 |
| SG08S669 | rs283728 | 128270089 |
| SG08S671 | rs424281 | 128296015 |
| SG08S661 | rs1949808 | 128351127 |
| SG08S660 | rs1562871 | 128358361 |
| SG08S675 | rs871135 | 128382982 |
| SG08S659 | rs1447294 | 128394275 |
| SG08S808 | rs6470517 | 128416993 |
| SG08S853 | rs10956372 | 128426845 |
| SG08S686 | rs1447293 | 128428909 |
| SG08S710 | rs921146 | 128431774 |
| SG08S663 | rs2121630 | 128434749 |
| SG08S829 | rs3999775 | 128436126 |
| SG08S687 | rs4871798 | 128436552 |
| SG08S848 | rs4871799 | 128439231 |
| SG08S982 | rs6470519 | 128440812 |
| SG08S983 | rs7818556 | 128440988 |
| SG08S717 | rs1447295 | 128441627 |
| SG08S984 | rs10109700 | 128442553 |
| SG08S849 | rs9297758 | 128443177 |
| SG08S850 | rs1992833 | 128448933 |
| SG08S664 | rs2290033 | 128449663 |
| SG08S908 | rs11989136 | 128450373 |
| SG08S827 | rs9643226 | 128451070 |
| SG08S826 | rs1447296 | 128451948 |
| SG08S688 | rs6985504 | 128453365 |
| SG08S985 | rs10808558 | 128457739 |
| SG08S722 | rs7820229 | 128459172 |
| SG08S805 | rs12155672 | 128463613 |
| SG08S689 | rs4599773 | 128467013 |
| SG08S690 | rs4078240 | 128468152 |
| SG08S851 | rs6981321 | 128469894 |
| SG08S986 | rs7832031 | 128473541 |
| SG08S802 | rs4242382 | 128474162 |
| SG08S811 | rs4314621 | 128474604 |
| SG08S812 | rs4242384 | 128475143 |
| SG08S987 | rs7812429 | 128476762 |
| SG08S813 | rs7812894 | 128477068 |
| SG08S988 | rs7814837 | 128478791 |
| -SG08S980 | rs10088308 | 128479503 |
| SG08S981 | rs9297760 | 128479761 |
| SG08S799 | rs7017300 | 128481857 |
| SG08S852 | rs6470527 | 128484420 |
| SG08S1045 | rs4498506 | 128485622 |
| SG08S990 | rs13255059 | 128487205 |
| SG08S991 | rs11986220 | 128488278 |
| SG08S911 | rs11988857 | 128488462 |
| SG08S836 | rs10090154 | 128488726 |
| SG08S807 | rs4599771 | 128490819 |
| SG08S1067 | rs9656967 | 128491176 |
| SG08S810 | rs9656816 | 128491243 |
| SG08S838 | rs12548153 | 128491281 |
| SG08S839 | rs12545648 | 128491344 |
| SG08S847 | rs12542685 | 128494172 |
| SG08S809 | rs7814251 | 128494806 |
| SG08S832 | rs7837688 | 128495949 |
| SG08S930 | rs13256658 | 128496050 |
| SG08S720 | rs7825823 | 128498506 |
| SG08S691 | rs6991990 | 128501972 |
| SG08S828 | rs4543510 | 128502208 |
| SG08S855 | rs6470531 | 128515746 |

Shown are SNP markers typed in the 600 kb region around marker DG8S737 to localize the boundaries of the association signal
*NCBI Build 34

TABLE 13
Significant single-marker association of markers in LD Block A at chromosome 8q24 to prostate cancer in
Iceland

| | | | | | N | Allelic Frequency | | | |
|---|---|---|---|---|---|---|---|---|---|
| Marker | rs-name | Allele* | Position | N Cases | Controls | Cases | Controls | RR | P value |
| SG08S808 | rs6470517 | A | 128.417 | 1121 | 927 | 0.910 | 0.885 | 1.33 | 0.0066 |
| SG08S808 | rs6470517 | G | 128.417 | 1121 | 927 | 0.090 | 0.115 | 0.75 | 0.0066 |
| SG08S853 | rs10956372 | A | 128.4268 | 1237 | 996 | 0.649 | 0.709 | 0.76 | 2.18E−05 |
| SG08S853 | rs10956372 | T | 128.4268 | 1237 | 996 | 0.351 | 0.291 | 1.32 | 2.18E−05 |
| SG08S686 | rs1447293 | A | 128.4289 | 1352 | 925 | 0.603 | 0.654 | 0.80 | 4.44E−04 |
| SG08S686 | rs1447293 | G | 128.4289 | 1352 | 925 | 0.397 | 0.346 | 1.25 | 4.44E−04 |
| SG08S710 | rs921146 | C | 128.4318 | 1060 | 827 | 0.246 | 0.196 | 1.33 | 3.00E−04 |
| SG08S710 | rs921146 | A | 128.4318 | 1060 | 827 | 0.754 | 0.784 | 0.84 | 0.0306 |
| SG08S1043 | rs3999773 | T | 128.4322 | 1348 | 1021 | 0.490 | 0.446 | 1.19 | 0.0025 |
| SG08S1043 | rs3999773 | A | 128.4322 | 1348 | 1021 | 0.510 | 0.554 | 0.84 | 0.0025 |
| DG8S737 | n.a. | −8 | 128.4331 | 1224 | 935 | 0.131 | 0.078 | 1.77 | 2.30E−08 |
| SG08S663 | rs2121630 | A | 128.4347 | 1173 | 931 | 0.122 | 0.083 | 1.54 | 3.39E−05 |
| SG08S663 | rs2121630 | C | 128.4347 | 1173 | 931 | 0.878 | 0.917 | 0.65 | 3.39E−05 |
| SG08S687 | rs4871798 | C | 128.4366 | 1332 | 979 | 0.813 | 0.874 | 0.63 | 2.40E−08 |
| SG08S687 | rs4871798 | T | 128.4366 | 1332 | 979 | 0.187 | 0.126 | 1.59 | 2.40E−08 |
| SG08S848 | rs4871799 | A | 128.4392 | 1222 | 989 | 0.724 | 0.783 | 0.73 | 7.58E−06 |
| SG08S848 | rs4871799 | G | 128.4392 | 1222 | 989 | 0.276 | 0.217 | 1.37 | 7.58E−06 |
| SG08S982 | rs6470519 | A | 128.4408 | 1329 | 686 | 0.167 | 0.109 | 1.64 | 4.66E−07 |
| SG08S982 | rs6470519 | C | 128.4408 | 1329 | 686 | 0.833 | 0.891 | 0.61 | 4.66E−07 |
| SG08S983 | rs7818556 | A | 128.441 | 1328 | 995 | 0.835 | 0.898 | 0.57 | 2.56E−10 |
| SG08S983 | rs7818556 | G | 128.441 | 1328 | 995 | 0.165 | 0.102 | 1.75 | 2.56E−10 |
| SG08S717 | rs1447295 | A | 128.4416 | 1363 | 1009 | 0.171 | 0.103 | 1.81 | 1.01E−11 |
| SG08S717 | rs1447295 | C | 128.4416 | 1363 | 1009 | 0.829 | 0.897 | 0.55 | 1.01E−11 |
| SG08S984 | rs10109700 | A | 128.4426 | 1344 | 1014 | 0.169 | 0.102 | 1.79 | 2.78E−11 |
| SG08S984 | rs10109700 | G | 128.4426 | 1344 | 1014 | 0.831 | 0.898 | 0.56 | 2.78E−11 |
| SG08S850 | rs1992833 | T | 128.4489 | 1242 | 996 | 0.442 | 0.399 | 1.19 | 0.0038 |
| SG08S850 | rs1992833 | G | 128.4489 | 1242 | 996 | 0.558 | 0.601 | 0.84 | 0.0038 |
| SG08S827 | rs9643226 | C | 128.4514 | 1353 | 993 | 0.168 | 0.101 | 1.81 | 2.29E−11 |
| SG08S827 | rs9643226 | G | 128.4514 | 1353 | 993 | 0.832 | 0.899 | 0.55 | 2.29E−11 |
| SG08S993 | rs1447296 | C | 128.4519 | 1350 | 1006 | 0.830 | 0.896 | 0.57 | 1.20E−10 |
| SG08S993 | rs1447296 | T | 128.4519 | 1350 | 1006 | 0.170 | 0.104 | 1.75 | 1.20E−10 |
| DG8S1761 | n.a | 0 | 128.45267 | 1067 | 895 | 0.598 | 0.565 | 1.15 | 0.0366 |
| DG8S1761 | n.a | −4 | 128.45267 | 1067 | 895 | 0.379 | 0.411 | 0.87 | 0.0411 |
| SG08S688 | rs6985504 | A | 128.4533 | 1240 | 956 | 0.282 | 0.239 | 1.25 | 0.0012 |
| SG08S688 | rs6985504 | G | 128.4533 | 1240 | 956 | 0.718 | 0.761 | 0.80 | 0.0012 |
| SG08S985 | rs10808558 | A | 128.4577 | 1338 | 999 | 0.169 | 0.102 | 1.80 | 2.87E−11 |
| SG08S985 | rs10808558 | G | 128.4577 | 1338 | 999 | 0.831 | 0.898 | 0.56 | 2.87E−11 |
| SG08S805 | rs12155672 | A | 128.4636 | 1161 | 945 | 0.472 | 0.440 | 1.14 | 0.0338 |
| SG08S805 | rs12155672 | G | 128.4636 | 1161 | 945 | 0.528 | 0.560 | 0.88 | 0.0338 |
| SG08S689 | rs4599773 | C | 128.467 | 1169 | 905 | 0.476 | 0.444 | 1.14 | 0.0386 |
| SG08S689 | rs4599773 | G | 128.467 | 1169 | 905 | 0.524 | 0.556 | 0.88 | 0.0386 |
| SG08S851 | rs6981321 | C | 128.4699 | 1211 | 953 | 0.341 | 0.266 | 1.43 | 9.93E−08 |
| SG08S851 | rs6981321 | G | 128.4699 | 1211 | 953 | 0.659 | 0.734 | 0.70 | 9.93E−08 |
| SG08S986 | rs7832031 | A | 128.4735 | 1351 | 1011 | 0.169 | 0.103 | 1.78 | 5.01E−11 |
| SG08S986 | rs7832031 | G | 128.4735 | 1351 | 1011 | 0.831 | 0.897 | 0.56 | 5.01E−11 |
| SG08S802 | rs4242382 | A | 128.4742 | 1161 | 940 | 0.163 | 0.105 | 1.67 | 3.20E−08 |
| SG08S802 | rs4242382 | G | 128.4742 | 1161 | 940 | 0.837 | 0.895 | 0.60 | 3.20E−08 |
| SG08S811 | rs4314621 | A | 128.4746 | 1344 | 1011 | 0.837 | 0.901 | 0.57 | 1.44E−10 |
| SG08S811 | rs4314621 | G | 128.4746 | 1344 | 1011 | 0.163 | 0.099 | 1.77 | 1.44E−10 |
| SG08S812 | rs4242384 | A | 128.4751 | 1166 | 947 | 0.836 | 0.893 | 0.61 | 7.17E−08 |
| SG08S812 | rs4242384 | C | 128.4751 | 1166 | 947 | 0.164 | 0.107 | 1.64 | 7.17E−08 |
| SG08S987 | rs7812429 | A | 128.4768 | 1285 | 996 | 0.167 | 0.106 | 1.70 | 1.97E−09 |
| SG08S987 | rs7812429 | G | 128.4768 | 1285 | 996 | 0.833 | 0.894 | 0.59 | 1.97E−09 |
| SG08S813 | rs7812894 | A | 128.4771 | 1169 | 1012 | 0.167 | 0.105 | 1.71 | 2.27E−09 |
| SG08S813 | rs7812894 | T | 128.4771 | 1169 | 1012 | 0.833 | 0.895 | 0.58 | 2.27E−09 |
| SG08S988 | rs7814837 | G | 128.4788 | 1273 | 958 | 0.834 | 0.897 | 0.58 | 1.51E−09 |
| SG08S988 | rs7814837 | T | 128.4788 | 1273 | 958 | 0.166 | 0.103 | 1.72 | 1.51E−09 |
| SG08S980 | rs10088308 | C | 128.4795 | 1337 | 1009 | 0.190 | 0.127 | 1.62 | 3.89E−09 |
| SG08S980 | rs10088308 | T | 128.4795 | 1337 | 1009 | 0.810 | 0.873 | 0.62 | 3.89E−09 |
| SG08S981 | rs9297760 | A | 128.4798 | 1326 | 983 | 0.192 | 0.126 | 1.64 | 1.90E−09 |
| SG08S981 | rs9297760 | G | 128.4798 | 1326 | 983 | 0.808 | 0.874 | 0.61 | 1.90E−09 |
| SG08S1006 | rs7824868 | C | 128.481 | 1122 | 613 | 0.824 | 0.885 | 0.61 | 1.47E−06 |
| SG08S1006 | rs7824868 | T | 128.481 | 1122 | 613 | 0.176 | 0.115 | 1.64 | 1.47E−06 |
| SG08S799 | rs7017300 | A | 128.4819 | 1319 | 920 | 0.832 | 0.876 | 0.71 | 6.08E−05 |
| SG08S799 | rs7017300 | C | 128.4819 | 1319 | 920 | 0.168 | 0.124 | 1.42 | 6.08E−05 |
| SG08S814 | rs4498506 | A | 128.4856 | 1357 | 1025 | 0.181 | 0.117 | 1.67 | 9.23E−10 |
| SG08S814 | rs4498506 | T | 128.4856 | 1357 | 1025 | 0.819 | 0.883 | 0.60 | 9.23E−10 |
| SG08S1044 | rs4297007 | A | 128.4857 | 1350 | 1017 | 0.819 | 0.884 | 0.60 | 5.80E−10 |
| SG08S1044 | rs4297007 | G | 128.4857 | 1350 | 1017 | 0.181 | 0.116 | 1.68 | 5.80E−10 |
| SG08S1030 | rs11992171 | A | 128.4865 | 1344 | 1018 | 0.804 | 0.875 | 0.59 | 5.40E−11 |
| SG08S1030 | rs11992171 | C | 128.4865 | 1344 | 1018 | 0.196 | 0.125 | 1.70 | 5.40E−11 |
| SG08S990 | rs13255059 | A | 128.4872 | 1350 | 1016 | 0.169 | 0.105 | 1.73 | 3.18E−10 |
| SG08S990 | rs13255059 | G | 128.4872 | 1350 | 1016 | 0.831 | 0.895 | 0.58 | 3.18E−10 |
| SG08S991 | rs11986220 | A | 128.4883 | 1348 | 602 | 0.166 | 0.096 | 1.87 | 3.35E−09 |
| SG08S991 | rs11986220 | T | 128.4883 | 1348 | 602 | 0.834 | 0.904 | 0.54 | 3.35E−09 |
| SG08S911 | rs11988857 | A | 128.4885 | 1340 | 1017 | 0.821 | 0.888 | 0.58 | 1.32E−10 |
| SG08S911 | rs11988857 | G | 128.4885 | 1340 | 1017 | 0.179 | 0.112 | 1.72 | 1.32E−10 |
| SG08S836 | rs10090154 | T | 128.4887 | 1288 | 998 | 0.169 | 0.109 | 1.66 | 6.58E−09 |
| SG08S836 | rs10090154 | C | 128.4887 | 1288 | 998 | 0.831 | 0.891 | 0.60 | 6.58E−09 |
| SG08S1071 | rs7824776 | C | 128.49 | 918 | 927 | 0.169 | 0.109 | 1.65 | 1.73E−07 |
| SG08S1071 | rs7824776 | T | 128.49 | 918 | 927 | 0.831 | 0.891 | 0.61 | 1.73E−07 |
| SG08S807 | rs4599771 | A | 128.4907 | 1172 | 949 | 0.824 | 0.882 | 0.63 | 1.05E−07 |
| SG08S807 | rs4599771 | G | 128.4907 | 1172 | 949 | 0.176 | 0.118 | 1.60 | 1.05E−07 |
| SG08S831 | rs4531012 | A | 128.4909 | 1347 | 1027 | 0.825 | 0.886 | 0.61 | 4.62E−09 |
| SG08S831 | rs4531012 | G | 128.4909 | 1347 | 1027 | 0.175 | 0.114 | 1.64 | 4.62E−09 |
| SG08S1067 | rs9656967 | A | 128.4915 | 1104 | 883 | 0.821 | 0.887 | 0.59 | 5.76E−09 |
| SG08S1067 | rs9656967 | T | 128.4915 | 1104 | 883 | 0.179 | 0.113 | 1.71 | 5.76E−09 |
| SG08S810 | rs9656816 | A | 128.4918 | 1131 | 897 | 0.844 | 0.904 | 0.58 | 1.68E−08 |
| SG08S810 | rs9656816 | G | 128.4918 | 1131 | 897 | 0.156 | 0.096 | 1.73 | 1.68E−08 |
| SG08S838 | rs12548153 | T | 128.4919 | 1120 | 896 | 0.626 | 0.589 | 1.17 | 0.0150 |
| SG08S838 | rs12548153 | C | 128.4919 | 1120 | 896 | 0.374 | 0.411 | 0.85 | 0.0150 |
| SG08S839 | rs12545648 | C | 128.492 | 1112 | 891 | 0.166 | 0.108 | 1.65 | 8.24E−08 |
| SG08S839 | rs12545648 | T | 128.492 | 1112 | 891 | 0.834 | 0.892 | 0.61 | 8.24E−08 |
| SG08S847 | rs12542685 | A | 128.4942 | 1226 | 992 | 0.594 | 0.559 | 1.15 | 0.0199 |
| SG08S847 | rs12542685 | T | 128.4942 | 1226 | 992 | 0.406 | 0.441 | 0.87 | 0.0199 |
| SG08S832 | rs7837688 | G | 128.4958 | 1348 | 1023 | 0.837 | 0.895 | 0.60 | 7.54E−09 |
| SG08S832 | rs7837688 | T | 128.4958 | 1348 | 1023 | 0.163 | 0.105 | 1.66 | 7.54E−09 |
| SG08S930 | rs13256658 | C | 128.4962 | 1221 | 952 | 0.616 | 0.578 | 1.17 | 0.0111 |
| SG08S930 | rs13256658 | T | 128.4962 | 1221 | 952 | 0.384 | 0.422 | 0.85 | 0.0111 |
| DG8S1769 | n.a | 0 | 128.50139 | 1275 | 953 | 0.833 | 0.890 | 0.61 | 4.13E−08 |
| DG8S1769 | n.a | A | 128.50139 | 1275 | 953 | 0.167 | 0.110 | 1.63 | 4.13E−08 |
| SG08S828 | rs4543510 | A | 128.5022 | 1217 | 940 | 0.274 | 0.220 | 1.34 | 4.89E−05 |
| SG08S828 | rs4543510 | G | 128.5022 | 1217 | 940 | 0.726 | 0.780 | 0.75 | 4.89E−05 |
| DG8S1407 | n.a | 0 | 128.50346 | 1368 | 905 | 0.726 | 0.780 | 0.75 | 3.85E−05 |
| DG8S1407 | n.a | −1 | 128.50346 | 1368 | 905 | 0.273 | 0.220 | 1.33 | 4.85E−05 |

Alleles for the markers at 8q24.21 are shown and the corresponding numbers of cases and controls (N), allelic frequencies of variants in affected and control individuals, the relative risk (RR) and two-sided P values. Values of RR greater than one indicate at-risk variants, while RR-values less than one indicate protective variants. All these markers can be used as surrogate markers to detect the association to prostate cancer in the region on Chr8q24.21.
*The CEPH sample (Centre d'Etudes du Polymorphisme Humain, genomics repository, CEPH sample 1347-02) is used as a reference for microsatellite alleles, the shorter allele of each microsatellite in this sample is set at 0 and all other alleles in other samples are numbered in relation to this reference.
n.a. Not applicable for microsatellite markers

Overall, 53 SNPs and 6 microsatellites from the LD block that also contains DG8S737 were genotyped. These loci captured most of the haplotype diversity in the LD block according to the Utah CEPH (CEU) HapMap data (Phase II, release 19). A total of 37 of the 53 SNPs were significantly associated with prostate cancer (P<0.001), with allele A of SNP rs1447295 showing the strongest association (RR=1.72, P=1.7×10⁻⁹) (Table 10). Sixteen of the SNPs belong to the same equivalence class (r²=1) as rs1447295 in the CEU HapMap sample, and therefore showed comparable association results.

In the Icelandic samples, allele −8 of DG8S737 and allele A of rs1447295 were substantially correlated (r²≈0.5). After typing the DG8S737 marker in the CEU HapMap sample, it was found that the correlation was lower there (r²≈0.3), but still no other SNP in HapMap (Phase II) had a higher correlation (Table 13). In other words, the SNPs that were most associated with allele −8 of DG8S737 are also those most associated with prostate cancer.

Replication in Two Cohorts of European Ancestry

Replication of this association using the markers DG8S737 and rs1447295 was performed in a Swedish cohort of 1435 unrelated prostate cancer patients and 779 population-based controls, and a cohort of 458 European American patients and 247 controls from Chicago. In both cohorts the frequency of the DG8S737 −8 allele was significantly higher in patients than controls, with a RR of 1.32 (P=0.013) and 2.10 (P=0.0029) for the Swedish and European American cohorts, respectively. A similar outcome was obtained for the rs1447295 A allele (Table 14), indicating that the variants initially identified in the Icelandic cohort are likely to be associated with increased risk of prostate cancer in most populations of European ancestry.

To investigate the risks of the DG8S737 −8 and rs1447295 A alleles jointly (Gretarsdottir, S. et al., Nat. Genet. 35:131-8 (2003)), chromosomes were partitioned into three groups: i) Chromosomes that carry the DG8S737 −8 allele and either rs1447295 allele (the vast majority carry the A allele) (−8 & AIG); ii) Chromosomes with the rs1447295 A allele and any allele of DG8S737 other than allele −8 (referred to as X) (X & A); and iii) Chromosomes that carry neither the −8 allele nor the A allele (X & G). Combining the data from the three cohorts using a Mantel-Haenszel model (Mantel, N. and Haenszel, W., J. Natl. Cancer Inst. 22:719-48 (1959)), the risk of (−8 & A/G) relative to (X& G) was estimated to be 1.61 (P=5.9×10⁻¹¹). The estimated risk of (X & A) relative to (X & G) was substantially lower at 1.27 but significant (P=0.0088). Since neither the DG8S737 −8 nor the rs1447295 A alleles by themselves can fully explain the risk profile, there may be multiple functional variants in the region, or these alleles are both in strong, but imperfect, LD.

Replication of the At-Risk Variant and Greater Population Attributable Risk in an African-American Cohort

A third replication study, in an African American cohort with 246 prostate cancer patients and 352 controls, was undertaken to determine whether the variants identified above are also associated with prostate cancer in a group with high incidence of the disease. Furthermore, if this were the case, it was postulated that the greater genetic diversity in African Americans, resulting from a large proportion of African ancestry, would provide more resolution to pinpoint the location of the unknown risk variant. This assumption was supported by an analysis of the region spanning the 92 kb LD block in the Nigerian Yoruba (YRI) HapMap sample, which revealed both greater genetic diversity and weaker LD in this group among the SNPs that were highly correlated in the populations of European ancestry. Specifically, while 19 SNPs, including rs1447295, are in the same equivalence class (r²=1) in the CEU HapMap data (Phase II), these SNPs belong to 13 different equivalence classes in the HapMap YRI sample (Table 14). Consequently, in addition to DG8S737, the African American cohort was genotyped with 17 of the 19 equivalent SNPs (including rs1447295). Of the two omitted, one was perfectly correlated with two other SNPs that were genotyped, and the other was non-polymorphic in the YRI samples. The differences in allele frequencies between the YRI HapMap sample and the controls from the European ancestry cohorts raised the possibility that false positive or negative association results could be caused by differences in the distribution of European ancestry among the African American patients and controls. Therefore, to control for ancestry, genotyping was performed for a set of 30 microsatellites that are randomly distributed in the genome and informative for distinguishing between African and European ancestry. An analysis of these data with Structure (Pritchard, J. K. et al., am. J. Hum. Genet. 67:170-81 (Epub 2000 May 26)) revealed no significant differences in European ancestry between patients and controls. Furthermore, association analyses performed with and without adjusting for ancestry gave practically identical results (Helgadottir, A. et al., Am. J. Hum. Genet. 76:505-9 (Epub 2005 Jan. 7); Pritchard, J. K. et al., am. J. Hum. Genet. 67:170-81 (Epub 2000 May 26)).

The frequency of allele −8 of DG8S737 was 23.4% in the African American prostate cancer patients and 16.1% in controls, with RR=1.60 (P=0.0022, with adjustment for relatedness between some of the patients). The SNP that gave the lowest P-value was rs1447295, where the frequency of the A allele was 34.4% in patients and 31.3% in controls (RR=1.15), but the association was not significant (P=0.29). These results indicate that DG8S737 −8 is either itself a functional variant or is very tightly associated with a presently unknown risk variant both in populations of European and African ancestry. In contrast, neither rs1447295 nor any of the other 16 SNPs were significantly associated with prostate cancer in the African American cohort (Table 14). Checking with the HapMap YRI data (Phase II), it was noticed that the three SNPs that have the strongest correlation with the −8 allele of DG8S737 there (r²=0.32 to 0.34), were included in the 17 SNPs genotyped in the African American samples (Table 14). Even though the RR is similar in populations of African and European ancestry, the PAR in African Americans is considerably greater (16.8% vs 5.8-11%) because of the higher frequency of DG8S737 −8 in the former group. This higher frequency can be explained by the frequency of this allele in African populations e.g. in the YRI HapMap sample the frequency is 22.5%. This raises the possibility that the PAR of DG8S737 −8 may even be greater in African populations.

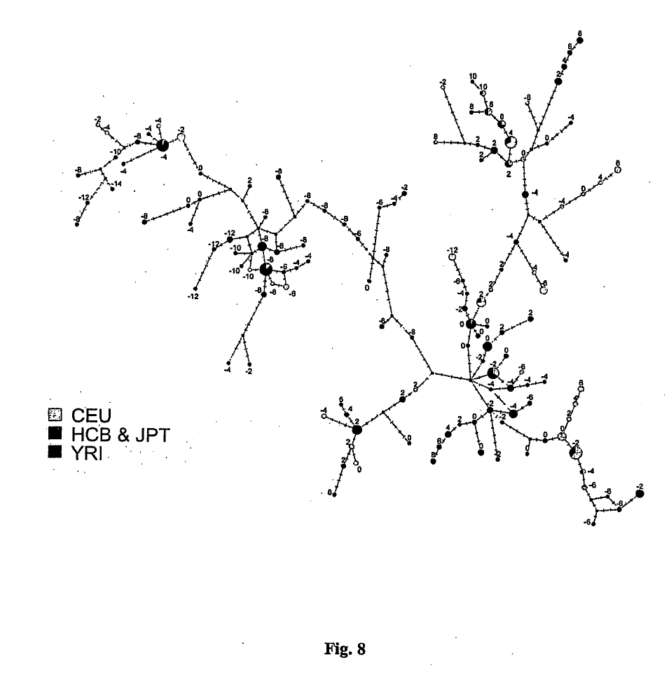

The DG8S737 marker is a dinucleotide AC repeat and the −8 allele derives from the fact that this allele is 8 bp smaller than the smallest allele of CEPH sample 1347-02, which Was used as a reference for microsatellite genotypes. Although DG8S737 exhibits a considerable range of allele sizes, a phylogenetic analysis indicates that it has a moderate mutation rate and that repeat sizes are strongly correlated with SNP background in the HapMap samples (FIG. 8). A median-joining network (Bandelt, H. J., Forster, P. & Rohl, Mol Biol Evol 16, 37-48 (1999)) describing the genealogical relationships between 136 distinct haplotypes inferred from the genotypes of 46 SNPs obtained from the HapMap project (Nature 437, 1299-320 (2005)) database (release 19) and one microsatellite, DG8S737. All these loci are contained within a ˜30 kb region (128,426,310-128,456,027, NCBI build 34) on chromosome 8. Haplotypes from the 60 Utah CEPH (CEU) parents with Northern and Western European ancestry, 60 Yoruban parents from Nigeria (YRI), 45 Chinese individuals from Beijing and 44 Japanese individuals from Tokyo (HCB & JPT), used in the HapMap project are shown. Phased haplotypes were generated using the EM algorithm, in combination with the family trio information for the Utah and Yoruba samples (where the genotypes from the 30 children in each of the population samples were used to help infer the allelic phase of the haplotypes). Each mutationally distinct haplotype is represented by a filled circle, whose area reflects the combined number of copies observed in the four population groups. In cases where haplotypes were inferred to be present in more than one population, pie slices indicate the number of haplotype copies from each population. The lines between the circles indicate differences between the allelic states of haplotypes, with length proportional to the number of differences and the loci at which alleles differ indicated by labels. The lines represent the most likely mutational pathways between the haplotypes according to the principle of evolutionary parsimony underlying the median-joining algorithm. Mutational differences between haplotypes are shown as short perpendicular lines that cross the evolutionary pathways connecting haplotypes. In this case, mutational events are considered to be both point mutations at individual SNPs, stepwise mutations of the DG8S737 microsatellite and recombination events. Parallelograms in the network are shown when the temporal order of two or more mutation events could not be resolved.

The evolutionary stability (mutation rate) of a microsatellite is reflected by the extent to which repeat sizes are correlated with SNP haplotypes. Thus, a relatively stable microsatellite would be expected to exhibit similar allele sizes on the background of identical and closely related SNP haplotypes, with greater differences between more distantly related SNP haplotypes. In contrast, such a correlation would not be expected for a rapidly mutating microsatellite, where substantial differences in repeat size may be found on closely related SNP haplotypes and identical repeat sizes may be found on distantly related SNP haplotypes due to recurrent mutation events at the microsatellite. FIG. 8 clearly shows that closely related SNP haplotypes tend to have similar repeat sizes for the DG8S737 microsatellite and distantly related SNP haplotypes tend to have more divergent repeat sizes. The correlation was estimated between the number of SNP alleles that differed between all pairs of haplotypes and the number of DG8S737 repeats that differed between all pairs of haplotypes. Spearman's non-parametric correlation coefficient ρ=0.334 with an empirical P-value<0.00001, based on the assessment of the correlation in 10,000 datasets where the microsatellite alleles were randomly assigned to the SNP haplotypes. This indicated a moderate mutation rate for the DG8S737 microsatellite, sufficient to generate a large number of different allele sizes, but insufficient to break down the correlation of repeat size with SNP haplotype background.

TABLE 14
Association of alleles at chromosome 8q24 to prostate cancer in Iceland, Sweden and the U.S.

| Study population (N | | | Allelic Frequency | | | | |
|---|---|---|---|---|---|---|---|
| cases/N controls) | Marker | Allele(s) | Cases | Controls | RR | P value | PAR |
| Iceland Cohort I^{a} | | | | | | | |
| (869/596) | DG8S737 | −8 | 0.134 | 0.080 | 1.79 | 3.0 × 10⁻⁶ | 0.115 |
| Iceland Cohort II | | | | | | | |
| (422/401) | DG8S737 | −8 | 0124 | 0.076 | 1.72 | 1.8 × 10⁻³ | 0.101 |
| Iceland all | | | | | | | |
| (1291/997) | DG8S737 | −8 | 0.131 | 0.078 | 1.77 | 2.3 × 10⁻⁸ | 0.110 |
| ″ | rs1447295 | A | 0.169 | 0.106 | 1.72 | 1.7 × 10⁻⁹ | 0.137 |
| Sweden | | | | | | | |
| (1435/779) | DG8S737 | −8 | 0.101 | 0.079 | 1.32 | 1.3 × 10⁻² | 0.058 |
| ″ | rs1447295 | A | 0.164 | 0.133 | 1.28 | 6.4 × 10⁻³ | 0.070 |
| European Americans | | | | | | | |
| Chicago | | | | | | | |
| (458/247) | DG8S737 | −8 | 0.082 | 0.041 | 2.10 | 2.9 × 10⁻³ | 0.084 |
| ″ | rs1447295 | A | 0.127 | 0.081 | 1.66 | 6.7 × 10⁻³ | 0.099 |
| African Americans | | | | | | | |
| Michigan | | | | | | | |
| (246/352) | DG8S737 | −8 | 0.234 | 0.161 | 1.60 | 2.2 × 10⁻³ | 0.168 |
| ″ | rs1447295 | A | 0.344 | 0.313 | 1.15 | 0.29 | 0.089 |

Alleles for the markers DG8S737 and rs1447295 at 8q24.21 are shown and the corresponding numbers of cases and controls (N), allelic frequencies of variants in affected and control individuals, the relative risk (RR), two-sided P values and population attributable risk (PAR).
^{a}Individuals are unrelated at 3 meioses

Analysis of the Multiple Cohorts

Table 15 shows the LD characteristics of DG8S737 −8 allele and 19 other SNPs that belong to the same equivalent class as SG08S717/rs1447295 in HapMap CEU, Iceland, HapMap Yorubans (YRI) and African Americans from the FMHS and PCGP studies at the University of Michigan. Markers in this block structure are also in moderate correlation (r² below 0.2) with more distant markers up to 200 kb away (including markers at 128515000 bps (rs7845403, rs6470531 and rs7829243) and markers around 128720000 bps (rs10956383 and rs6470572) in the area of the PVT1 gene).

TABLE 15A
LD characteristics, in the populations studied, of the −8 allele of DG8S737 and the 19 SNPs belonging to
the equivalent class of A allele of rs1447295 in HapMap Caucasians (CEU).

| | | | Populations | | | | | | | | | | |
|---|---|---|---|---|---|---|---|---|---|---|---|---|---|
| | | | CEU | | | | | Iceland | | | | | |
| | | | −8 | | A | | All | −8 | | A | | All | All |
| Marker | Allele | Location^{a} | D′ | r² | D′ | r² | freq | D′ | r² | D′ | r² | freq^{b} | freq^{c} |
| DG8S737 | −8 | 128433096 | 1.00 | 1.00 | 0.72 | 0.29 | 0.04 | 1.00 | 1.00 | 0.85 | 0.52 | 0.13 | 0.08 |
| rs6470519^{d} | A | 128440812 | 0.72 | 0.29 | 1.00 | 1.00 | 0.07 | 0.82 | 0.49 | 0.98 | 0.96 | 0.17 | 0.11 |
| rs7818556 | G | 128440988 | 0.72 | 0.29 | 1.00 | 1.00 | 0.07 | 0.84 | 0.52 | 0.99 | 0.99 | 0.17 | 0.11 |
| rs1447295 | A | 128441627 | 0.72 | 0.29 | 1.00 | 1.00 | 0.07 | 0.85 | 0.52 | 1.00 | 1.00 | 0.17 | 0.11 |
| rs10109700 | A | 128442553 | 0.72 | 0.29 | 1.00 | 1.00 | 0.07 | 0.85 | 0.52 | 1.00 | 0.99 | 0.17 | 0.11 |
| rs7826179 | T | 128445788 | 0.72 | 0.29 | 1.00 | 1.00 | 0.07 | Nd | | | | | |
| rs9643226^{d} | C | 128451070 | 0.72 | 0.29 | 1.00 | 1.00 | 0.07 | 0.83 | 0.50 | 0.99 | 0.97 | 0.17 | 0.11 |
| rs1447296 | T | 128451948 | 0.72 | 0.29 | 1.00 | 1.00 | 0.07 | 0.82 | 0.49 | 0.99 | 0.95 | 0.17 | 0.11 |
| rs10808558^{d} | A | 128457739 | 0.72 | 0.29 | 1.00 | 1.00 | 0.07 | 0.83 | 0.50 | 0.98 | 0.97 | 0.17 | 0.11 |
| rs7832031 | A | 128473541 | 0.72 | 0.29 | 1.00 | 1.00 | 0.07 | 0.83 | 0.50 | 0.98 | 0.96 | 0.17 | 0.11 |
| rs4242382 | A | 128474162 | 0.72 | 0.29 | 1.00 | 1.00 | 0.07 | 0.83 | 0.51 | 0.98 | 0.94 | 0.17 | 0.11 |
| rs4314621 | G | 128474604 | 0.72 | 0.29 | 1.00 | 1.00 | 0.07 | 0.83 | 0.51 | 0.98 | 0.96 | 0.17 | 0.11 |
| rs4242384 | C | 128475143 | 0.72 | 0.29 | 1.00 | 1.00 | 0.07 | 0.84 | 0.51 | 0.98 | 0.96 | 0.17 | 0.11 |
| rs7812429 | A | 128476762 | 0.72 | 0.29 | 1.00 | 1.00 | 0.07 | 0.83 | 0.51 | 0.98 | 0.96 | 0.17 | 0.11 |
| rs7812894 | A | 128477068 | 0.72 | 0.29 | 1.00 | 1.00 | 0.07 | 0.85 | 0.52 | 0.98 | 0.96 | 0.17 | 0.11 |
| rs7814837 | T | 128478791 | 0.72 | 0.29 | 1.00 | 1.00 | 0.07 | 0.84 | 0.50 | 0.98 | 0.95 | 0.17 | 0.11 |
| rs4582524 | G | 128485024 | 0.72 | 0.29 | 1.00 | 1.00 | 0.07 | Nd | | | | | |
| rs13255059 | A | 128487205 | 0.72 | 0.29 | 1.00 | 1.00 | 0.07 | 0.82 | 0.49 | 0.98 | 0.96 | 0.17 | 0.11 |
| rs11986220 | A | 128488278 | 0.72 | 0.29 | 1.00 | 1.00 | 0.07 | 0.78 | 0.50 | 0.90 | 0.72 | 0.17 | 0.10 |
| rs10090154 | T | 128488726 | 0.72 | 0.29 | 1.00 | 1.00 | 0.07 | 0.83 | 0.50 | 0.98 | 0.94 | 0.17 | 0.11 |
| | | | Populations | | | | | | | | | | |
| | | | YRI | | | | | Michigan | | | | | |
| | | | −8 | | A | | All | −8 | | A | | All | All |
| Marker | Allele | Location^{a} | D′ | r² | D′ | r² | freq | D′ | r² | D′ | r² | freq^{b} | freq^{c} |
| DG8S737 | −8 | 128433096 | 1.00 | 1.00 | 0.62 | 0.21 | 0.22 | 1.00 | 1.00 | 0.48 | 0.12 | 0.23 | 0.16 |
| rs6470519^{d} | A | 128440812 | 0.60 | 0.34 | 1.00 | 0.56 | 0.21 | 0.41 | 0.17 | 0.97 | 0.44 | 0.20 | 0.18 |
| rs7818556 | G | 128440988 | 0.74 | 0.31 | 1.00 | 0.93 | 0.34 | 0.62 | 0.17 | 0.99 | 0.89 | 0.37 | 0.33 |
| rs1447295 | A | 128441627 | 0.62 | 0.21 | 1.00 | 1.00 | 0.34 | 0.48 | 0.12 | 1.00 | 1.00 | 0.34 | 0.31 |
| rs10109700 | A | 128442553 | 0.56 | 0.20 | 1.00 | 1.00 | 0.29 | 0.48 | 0.12 | 1.00 | 1.00 | 0.34 | 0.31 |
| rs7826179 | T | 128445788 | Np | | | | 0.00 | Nd | | | | | |
| rs9643226^{d} | C | 128451070 | 0.76 | 0.33 | 1.00 | 0.32 | 0.14 | 0.68 | 0.22 | 1.00 | 0.23 | 0.10 | 0.10 |
| rs1447296 | T | 128451948 | 0.46 | 0.20 | 1.00 | 0.51 | 0.21 | 0.40 | 0.13 | 0.93 | 0.33 | 0.16 | 0.15 |
| rs10808558^{d} | A | 128457739 | 0.80 | 0.32 | 0.78 | 0.16 | 0.12 | 0.57 | 0.14 | 0.88 | 0.15 | 0.08 | 0.09 |
| rs7832031 | A | 128473541 | 1.00 | 0.01 | 1.00 | 0.02 | 0.04 | 0.09 | 0.00 | 0.13 | 0.00 | 0.05 | 0.04 |
| rs4242382 | A | 128474162 | 0.03 | 0.00 | 0.04 | 0.00 | 0.33 | 0.02 | 0.00 | 0.01 | 0.00 | 0.34 | 0.32 |
| rs4314621 | G | 128474604 | 0.25 | 0.05 | 0.28 | 0.03 | 0.18 | 0.21 | 0.03 | 0.41 | 0.06 | 0.13 | 0.15 |
| rs4242384 | C | 128475143 | 0.25 | 0.05 | 0.29 | 0.03 | 0.18 | 0.18 | 0.03 | 0.35 | 0.05 | 0.16 | 0.17 |
| rs7812429 | A | 128476762 | 0.36 | 0.05 | 0.22 | 0.01 | 0.11 | 0.21 | 0.02 | 0.26 | 0.01 | 0.08 | 0.08 |
| rs7812894 | A | 128477068 | 0.23 | 0.04 | 0.25 | 0.03 | 0.18 | 0.13 | 0.02 | 0.32 | 0.05 | 0.19 | 0.19 |
| rs7814837 | T | 128478791 | 0.30 | 0.04 | 0.18 | 0.01 | 0.10 | 0.19 | 0.01 | 0.24 | 0.01 | 0.09 | 0.08 |
| | | | | | | | | Nd | | | | | |
| rs4582524 | G | 128485024 | 1.00 | 0.02 | 1.00 | 0.04 | 0.07 | 0.00 | | | | | |
| rs13255059 | A | 128487205 | 1.00 | 0.02 | 1.00 | 0.04 | 0.07 | 0.03 | | 0.47 | 0.01 | 0.06 | 0.04 |
| rs11986220 | A | 128488278 | 1.00 | 0.02 | 1.00 | 0.04 | 0.08 | 0.05 | 0.00 | 0.41 | 0.00 | 0.05 | 0.04 |
| rs10090154 | T | 128488726 | 0.09 | 0.01 | 0.14 | 0.01 | 0.18 | 0.14 | 0.02 | 0.27 | 0.03 | 0.19 | 0.17 |

Shown are SNPs that have r² of 1.00 or greater to rs1447295 in HapMap CEU samples. LD characteristics are given for HapMap Caucasians (n = 60), Icelanders (n = 2288), HapMap Yorubans from Nigeria (YRI) (n = 60) and African American from Michigan (n = 598).
Nd: not done;
Np: not polymorphic.
All freq = allelic frequency.
^{a}Build34
^{b}cases
^{c}controls
^{d}These SNPs showed the strongest correlation with the −8 allele of DG8S737 in the HapMap YRI data (Phase II)

It was found that the multiplicative risk model used for testing fit the data adequately for both populations of European and African ancestry. Thus, we have replicated the association seen in Icelandic prostate cancer patients and controls using the markers DG8S737 and SG08S717 (rs1447295) in a Swedish case control sample

TABLE 16
Comparison of the relative risk of DG8S737 −8 and rs1447295
A under the multiplicative model with that of model-free estimates
of the genotype relative risks of the heterozygous-(0X),
homozygous-(XX) and non-carriers (00).

| | | | Allelic | Genotype RR | | | p- |
|---|---|---|---|---|---|---|---|
| N cases | Marker | Allele | RR | 0 | 0X | XX | value^{a} |
| Iceland | | | | | | | |
| 1291 | DG8S737 | −8 | 1.77 | 1 | 1.77 | 3.17 | 0.96 |
| ″ | rs1447295 | A | 1.72 | 1 | 1.71 | 3.03 | 0.84 |
| Sweden | | | | | | | |
| 1435 | DG8S737 | −8 | 1.32 | 1 | 1.33 | 1.64 | 0.78 |
| ″ | rs1447295 | A | 1.28 | 1 | 1.28 | 1.6 | 0.91 |
| European | | | | | | | |
| Americans- | | | | | | | |
| Chicago | | | | | | | |
| 458 | DG8S737 | −8 | 2.1 | 1 | 1.97 | 7.2 | 0.26 |
| ″ | rs1447295 | A | 1.66 | 1 | 1.61 | 3.38 | 0.52 |
| African | | | | | | | |
| Americans- | | | | | | | |
| Michigan | | | | | | | |
| 246 | DG8S737 | −8 | 1.6 | 1 | 1.42 | 3.2 | 0.18 |
| ″ | rs1447295 | A | 1.15 | 1 | 0.88 | 1.6 | 0.26 |

^{a}Test of the full model versus the multiplicative model

and in a case control sample including of 458 European American patients and 247 controls from Chicago, U.S. Individuals that are homozygote carriers of the DG8S737 −8 allele or the rs1447295 A allele have an even higher RR than heterozygous carriers o for all four populations studied as shown in Table 16 (XX genotype). Thus, individuals carrying two at risk alleles are at an even greater risk of developing prostate cancer than those carrying one at risk allele.

At Risk Variant Associates More Strongly with Aggressive Prostate Cancer

It was next determined whether the at-risk variants associate more strongly with aggressive forms of prostate cancer as reflected by high Gleason scores. In all four patient-control cohorts, the frequency of DG8S737 −8 was significantly greater in prostate cancer patients with combined Gleason scores of 7 to 10 than in controls (Table 17). The same is true for prostate cancer patients with Gleason scores of 2-6 compared to controls but the RR is higher in the Gleason 7-10 group compared to the Gleason 2-6 group. Moreover, the frequency of allele −8 was greater in patients with high (7-10) compared to low (2-6) Gleason scores in all four case-control groups combined (RR=1.21, P=0.02) and the three European ancestry case-control groups combined, (RR=1.18, P=0.07).

TABLE 17
Association of alleles at chromosome 8q24 to high and low Gleason scores in Iceland,
Sweden and the US.

| Study population | | | | | | | |
|---|---|---|---|---|---|---|---|
| (N cases/N controls) | Marker | Allele | Cases | Controls | RR | P value | PAR |
| Iceland | | | | | | | |
| Biopsy Gleason 7-10 | | | | | | | |
| (289/997) | DG8S737 | −8 | 0.146 | 0.078 | 2.00 | 4.0 × 10⁻⁶ | 0.141 |
| ″ | rs1447295 | A | 0.179 | 0.106 | 1.84 | 7.3 × 10⁻⁶ | 0.156 |
| Biopsy Gleason 2-6 | | | | | | | |
| (548/997) | DG8S737 | −8 | 0.131 | 0.078 | 1.78 | 3.4 × 10⁻⁶ | 0.112 |
| ″ | rs1447295 | A | 0.170 | 0.106 | 1.73 | 6.7 × 10⁻⁷ | 0.138 |
| Sweden | | | | | | | |
| Gleason 7-10 | | | | | | | |
| (625/779) | DG8S737 | −8 | 0.107 | 0.079 | 1.41 | 1.1 × 10⁻² | 0.061 |
| ″ | rs1447295 | A | 0.167 | 0.133 | 1.30 | 1.5 × 10⁻² | 0.075 |
| Gleason 2-6 | | | | | | | |
| (678/779) | DG8S737 | −8 | 0.094 | 0.079 | 1.22 | 0.15 | 0.033 |
| ″ | rs1447295 | A | 0.158 | 0.133 | 1.22 | 6.4 × 10⁻² | 0.055 |
| European Americans- | | | | | | | |
| Chicago | | | | | | | |
| Biopsy Gleason 7-10 | | | | | | | |
| (149/247) | DG88737 | −8 | 0.108 | 0.041 | 2.83 | 4.4 × 10⁻⁴ | 0.135 |
| ″ | rs1447295 | A | 0.151 | 0.081 | 2.03 | 2.7 × 10⁻³ | 0.148 |
| Biopsy Gleason 2-6 | | | | | | | |
| (306/247) | DG8S737 | −8 | 0.071 | 0.041 | 1.78 | 3.6 × 10⁻² | 0.061 |
| ″ | rs1447295 | A | 0.116 | 0.081 | 1.50 | 5.1 × 10⁻² | 0.076 |
| African Americans- | | | | | | | |
| Michigan | | | | | | | |
| Biopsy Gleason 7-10 | | | | | | | |
| (112/352) | DG8S737 | −8 | 0.273 | 0.161 | 1.96 | 3.3 × 10⁻⁴ | 0.25 |
| ″ | rs1447295 | A | 0.352 | 0.313 | 1.19 | 0.28 | 0.111 |
| Biopsy Gleason 2-6 | | | | | | | |
| (121/352) | DG8S737 | −8 | 0.211 | 0.161 | 1.40 | 8.2 × 10⁻² | 0.116 |
| ″ | rs1447295 | A | 0.341 | 0.313 | 1.14 | 0.43 | 0.079 |

Alleles for the markers DG8S737 and rs1447295 at 8q24.21 are shown and the corresponding numbers of cases and controls (N), frequencies of variants in affected and control individuals, the relative risk (RR), two-sided P values and population attributable risk (PAR). About 80% Swedish Gleason scores are from biopsy material and the rest from surgery.

Moreover, the frequency of allele −8 were greater in high Gleason patients (7-10) than in low Gleason patients (2-6) in all four cohorts (combined, odds-ratio=1.22, P=0.020). An analysis of 510 Icelandic men diagnosed with benign prostatic hyperplasia (BPH), but not prostate cancer, showed no significant excess of either allele −8 of DG8S737 or allele A of rs1447295 (Table 18) indicating that these variants only increase the risk of malignant prostate tumors, particularly the more aggressive forms.

TABLE 18
Association of alleles at chromosome 8q24 to benign prostatic
hyperplasia (BPH) in Iceland.

| Study | | | | | | | |
|---|---|---|---|---|---|---|---|
| population | | | Allelic | | | | |
| (N cases/N | | | Frequency | | | | |
| controls) | | | | Con- | | P | |
| BPH+ PrCa− | Marker | Allele(s) | Cases | trols | RR | value | PAR |
| (510/997) | DG8S737 | −8 | 0.085 | 0.078 | 1.09 | 0.527 | 0.015 |
| ″ | rs1447295 | A | 0.122 | 0.106 | 1.17 | 0.207 | 0.035 |

Alleles at 8q24.21 are shown and the corresponding numbers of cases and controls (N), allelic frequencies of variants in affected and control individuals, the relative risk (RR), P values and population attributable risk (PAR). Benign prostatic hyperplasia patients (BPH) were diagnosed on the basis of transurethral. excision of the prostate (TURP), fine needle biopsies or excision of the prostate gland. Individuals are unrelated at 3 meioses. Controls used in this analysis were the same individuals as used in the association analysis for the Icelandic prostate cancer cohorts.
BPH+ PrCa− indicates individuals diagnosed with BPH but not prostate cancer.

Functional Characterization of the LD Block Including the at Risk Variant

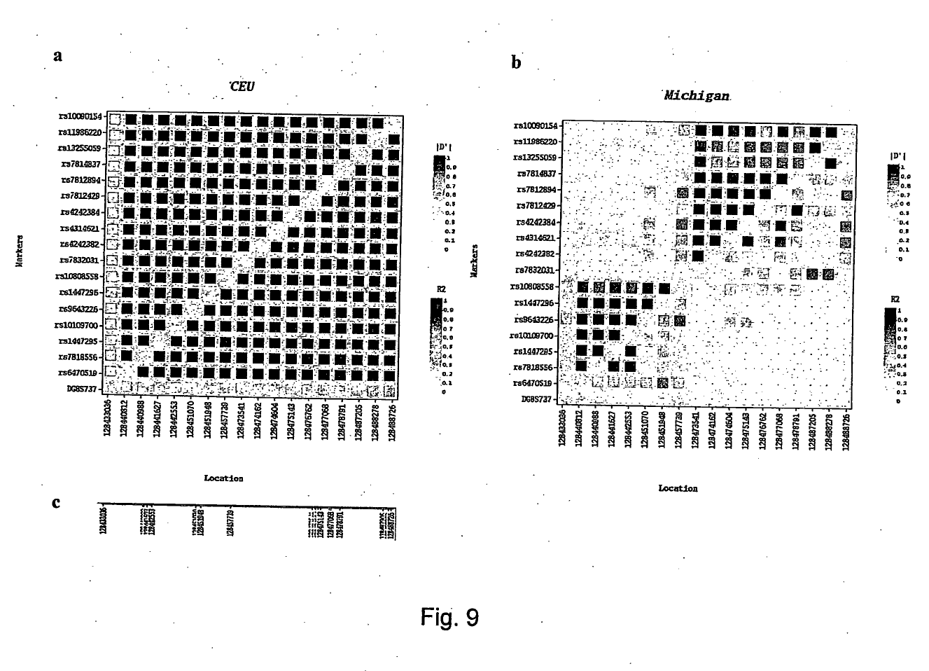

Since only the microsatellite allele showed significant association in the African American cohort and since the LD block containing this locus is smaller and is broken up into smaller units in African Americans (FIG. 9A-9C), it is possible that the region most likely to contain the functional variant can be narrowed down to positions 128.414-128.474 Mb NCBI build 34). This region contains one spliced EST (AW183883) and three single exon ESTs (BE144297, CV364590 and AF119310) in addition to a few predicted genes, but no known genes (Kent, W. J. et al., Genome Res. 12:996-1006 (2002)). No microRNAs were detected within the block (Griffiths-Jones, S., Nucleic Acids Res. 32:D109-11 (2004)).

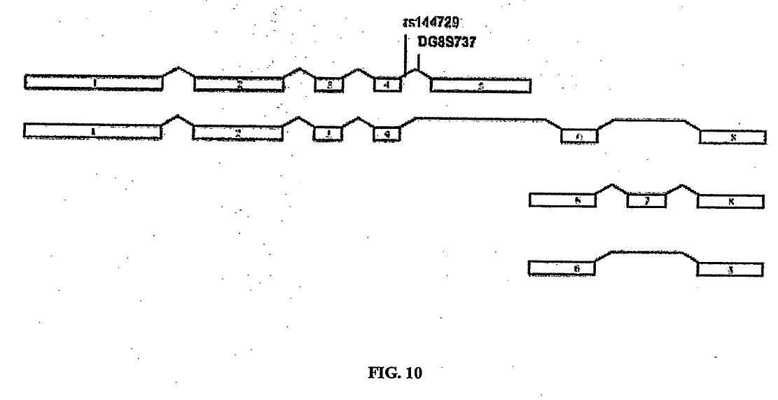

Expression analysis in various cDNA libraries confirmed the expression of the AW183883 EST but none of the other ESTs (see Materials and Methods above). Four different splice variants were identified from the AW183883 EST by 5′ and 3′ rapid amplification of cDNA ends (RACE) that were verified by RT-PCR and Northern blot analysis (FIG. 10). Two of these transcripts (1.5 kb), both harboring the AW183883 EST, were expressed in testis but not in spleen, thymus, prostate, ovary, small intestine, colon, peripheral blood leukocytes or prostate cell lines (data not shown). In contrast, the expression of the two other transcripts, harboring exons 6-8 were only detected in normal (0.6 kb transcript) and malignant prostate cell lines (0.6 and 0.9 kb transcripts) (data not shown). The predicted ORFs for these transcripts did not show significant homology to known proteins. The microsatellite DG8S737 and the SNP rs1447295 are located in the intron between exons 4 and 5 (or 6) in the testis transcripts and 5′ to the prostate specific transcripts (FIG. 10). It is conceivable that these markers or other markers in LD with these markers affect the splicing pattern of one or more transcripts in this region. It was noted that 8q24 is the most frequently gained chromosomal region in prostate tumors (Baudis, M. and Cleary, M. L., Bioinformatics 17:1228-9 (2001)). Gain in this region has been associated with aggressive tumors, hormone independence and poor prognosis (El Gedaily, A. et al., Prostate 46:184-90 (2001)). To assess whether chromosomes carrying the DG8S737-8 allele were associated with increased genomic instability, a Southern blot analysis was performed, covering the 92 kb LD region using germline and tumor DNA from prostate cancer patients that were carriers and non-carriers of the −8 allele. Only one tumor sample (non-carrier) out of 14 showed a polymorphic restriction pattern, but none was observed in germline DNA from either carriers or non-carriers (data not shown). Thus, it seems unlikely that the DG8S737 −8 germline variant is associated with rearrangement of the LD block A region.

Also of interest is the proximity of DG8S737 to the well-known oncogene c-MYC, at a distance of only ˜270 kb (telomeric). However, no significant correlation was observed between SNPs located in the c-MYC gene and either prostate cancer risk or the risk variants identified in this study (data not shown). Nevertheless, it is possible that the risk variant acts to modify c-MYC regulation by predisposing to genomic instability or by altering long-range regulation of expression.

Discussion

In summary, significant association of prostate cancer risk to the DG8S737 −8 and rs1447295 A alleles has been demonstrated in three cohorts of European ancestry (where the rs1447295 allele is perfectly correlated with alleles from at least 18 other nearby SNPs). Combining results from these cohorts gave an estimated RR of 1.59 (P=1.40×10⁻¹⁰) for DG8S737 −8 and an estimated relative risk of 1.50 (P=1.62×10⁻¹¹) for rs1447295 allele A. Assuming population frequencies of 6.6% and 10.7% (averages from the three cohorts), the corresponding PAR are 7.4% and 9.9%, respectively, for these two markers. The association was replicated between prostate cancer and the −8 allele in an African American cohort with nearly identical relative risk (RR=1.60, P=0.0022). At this time, association was not demonstrated with any of the HapMap SNPs in this region in the African Americans.

The variants described herein were identified through a positional cloning approach, starting with linkage analyses. Genome-wide association could also have been used, using common SNPs either through rs1447295 or one of its LD equivalents. The result would remain highly significant even if it were necessary to adjust for the testing of hundreds of thousands of common SNPs. In contrast, if based only on SNPs contained in release 19 of the HapMap project, the analyses suggest that a genome-wide association study would not have captured this association signal in African American or African cohorts. This is because none of the existing HapMap SNPs are sufficiently correlated with the DG8S737 −8 allele in populations of African ancestry. Consequently, it is postulated that either the −8 allele itself confers the risk or some variant that is more closely correlated with the −8 allele than any of the current HapMap SNPs. If the latter hypothesis is true, then the reduced LD in African Americans indicates that the unknown variant is located within a 60kb region containing DG8S737. Of equal importance is the relatively high population frequency of the −8 allele in African Americans, which confers an estimated PAR of 16.8%. Thus, the frequencies of the −8 allele alone could produce, a 14% greater incidence of prostate cancer in African Americans than in European Americans, and thereby partially account for the unusually high incidence of prostate cancer in African Americans.

It should also be noted that these at-risk variants described in relation to prostate cancer are also seen in higher frequencies in other forms of cancer (e.g., breast cancer, lung cancer, melanoma). Table 19 shows that the −8 allele of DG8S737 and allele A of SG08S717 (rs1447295) increases the risk of invasice breast cancer, lung cancer and malignant cutaneous melanoma. Again, it should be noted that allelic frequencies are shown in all Tables, which are roughly one half of carrier frequencies.

TABLE 19
Association of alleles and haplotypes at chromosome 8q24 to
melanoma, breast and lung cancer in Iceland.

| Study | | | | | | |
|---|---|---|---|---|---|---|
| population | | | Allelic | | | |
| (N cases/N | | | Frequency | | | P |
| controls) | Marker | Allele | Cases | Controls | RR | value |
| Cutaneous | | | | | | |
| malignant | | | | | | |
| melanoma | | | | | | |
| (410/997) | DG8S737 | −8 | 0.091 | 0.065 | 1.43 | 0.010 |
| ″ | rs1447295 | A | 0.096 | 0.078 | 1.26 | 0.060 |
| Invasive | | | | | | |
| breast | | | | | | |
| cancer (female) | | | | | | |
| (1504/997) | DG8S737 | −8 | 0.078 | 0.065 | 1.22 | 0.039 |
| ″ | rs1447295 | A | 0.090 | 0.078 | 1.17 | 0.063 |
| Lung cancer | | | | | | |
| (308/997) | DG8S737 | −8 | 0.081 | 0.065 | 1.27 | 0.090 |
| ″ | rs1447295 | A | 0.097 | 0.078 | 1.28 | 0.065 |

Alleles for the markers DG8S737 and rs1447295 at 8q24.21 are shown and the corresponding numbers of cases and controls (N), allelic frequencies of variants in affected and control individuals, the relative risk (RR) and one-sided P values.

Table 20 contains all known and described SNP markers, according to the NCBI database (db SNP 125), in the LD-block interval (128.414-128.506).

TABLE 20
All SNPs in the 92 Mb LD-block interval (128.414-128.506
Mb) from dbSNP 125 (A map of NCBI dbSNP Build 125)

| rs-name | chromosome | location* | Source |
|---|---|---|---|
| rs7012462 | 8 | 128414279 | dbSNP-125 |
| rs6992697 | 8 | 128414405 | dbSNP-125 |
| rs10109622 | 8 | 128414740 | dbSNP-125 |
| rs10109723 | 8 | 128414827 | dbSNP-125 |
| rs6996874 | 8 | 128414898 | dbSNP-125 |
| rs4871791 | 8 | 128415233 | dbSNP-125 |
| rs13282506 | 8 | 128415714 | dbSNP-125 |
| rs6470517 | 8 | 128416993 | dbSNP-125 |
| rs7008786 | 8 | 128417319 | dbSNP-125 |
| rs7841228 | 8 | 128417467 | dbSNP-125 |
| rs10094059 | 8 | 128418196 | dbSNP-125 |
| rs10719294 | 8 | 128418485 | dbSNP-125 |
| rs11778417 | 8 | 128418666 | dbSNP-125 |
| rs11786281 | 8 | 128420006 | dbSNP-125 |
| rs10095746 | 8 | 128420075 | dbSNP-125 |
| rs10109068 | 8 | 128420108 | dbSNP-125 |
| rs28626202 | 8 | 128420942 | dbSNP-125 |
| rs28451337 | 8 | 128421776 | dbSNP-125 |
| rs9642878 | 8 | 128421857 | dbSNP-125 |
| rs11781420 | 8 | 128421931 | dbSNP-125 |
| rs9643221 | 8 | 128422076 | dbSNP-125 |
| rs7836345 | 8 | 128422269 | dbSNP-125 |
| rs7836468 | 8 | 128422360 | dbSNP-125 |
| rs10537650 | 8 | 128422444 | dbSNP-125 |
| rs11308268 | 8 | 128422866 | dbSNP-125 |
| rs10107830 | 8 | 128423213 | dbSNP-125 |
| rs11271796 | 8 | 128423228 | dbSNP-125 |
| rs7841264 | 8 | 128423403 | dbSNP-125 |
| rs7828855 | 8 | 128423577 | dbSNP-125 |
| rs9643222 | 8 | 128423694 | dbSNP-125 |
| rs9643223 | 8 | 128423753 | dbSNP-125 |
| rs13273993 | 8 | 128423809 | dbSNP-125 |
| rs7017671 | 8 | 128424343 | dbSNP-125 |
| rs10099905 | 8 | 128424523 | dbSNP-125 |
| rs10100179 | 8 | 128424672 | dbSNP-125 |
| rs3999784 | 8 | 128425358 | dbSNP-125 |
| rs13250306 | 8 | 128425382 | dbSNP-125 |
| rs12544220 | 8 | 128425504 | dbSNP-125 |
| rs3999771 | 8 | 128426087 | dbSNP-125 |
| rs10555137 | 8 | 128426179 | dbSNP-125 |
| rs6990480 | 8 | 128426297 | dbSNP-125 |
| rs11785452 | 8 | 128426310 | dbSNP-125 |
| rs10956372 | 8 | 128426845 | dbSNP-125 |
| rs7825928 | 8 | 128427197 | dbSNP-125 |
| rs7830306 | 8 | 128427574 | dbSNP-125 |
| rs7830412 | 8 | 128427630 | dbSNP-125 |
| rs7830530 | 8 | 128428007 | dbSNP-125 |
| rs7830776 | 8 | 128428079 | dbSNP-125 |
| rs7387447 | 8 | 128428265 | dbSNP-125 |
| rs10112657 | 8 | 128428269 | dbSNP-125 |
| rs10094871 | 8 | 128428558 | dbSNP-125 |
| rs1447293 | 8 | 128428909 | dbSNP-125 |
| rs1447292 | 8 | 128429231 | dbSNP-125 |
| rs4871796 | 8 | 128430114 | dbSNP-125 |
| rs6651169 | 8 | 128431273 | dbSNP-125 |
| rs921146 | 8 | 128431774 | dbSNP-125 |
| rs3999772 | 8 | 128432143 | dbSNP-125 |
| rs3999773 | 8 | 128432171 | dbSNP-125 |
| rs3999774 | 8 | 128432275 | dbSNP-125 |
| rs7825118 | 8 | 128432406 | dbSNP-125 |
| rs13250904 | 8 | 128433758 | dbSNP-125 |
| rs13251194 | 8 | 128433845 | dbSNP-125 |
| rs2121630 | 8 | 128434749 | dbSNP-125 |
| rs2166689 | 8 | 128434904 | dbSNP-125 |
| rs4871797 | 8 | 128435349 | dbSNP-125 |
| rs10095293 | 8 | 128436099 | dbSNP-125 |
| rs3956790 | 8 | 128436116 | dbSNP-125 |
| rs3999775 | 8 | 128436126 | dbSNP-125 |
| rs4871798 | 8 | 128436552 | dbSNP-125 |
| rs12545929 | 8 | 128436814 | dbSNP-125 |
| rs10089310 | 8 | 128437573 | dbSNP-125 |
| rs7819102 | 8 | 128437938 | dbSNP-125 |
| rs4871799 | 8 | 128439231 | dbSNP-125 |
| rs4871800 | 8 | 128439304 | dbSNP-125 |
| rs6981424 | 8 | 128439685 | dbSNP-125 |
| rs7001513 | 8 | 128439754 | dbSNP-125 |
| rs4871801 | 8 | 128440503 | dbSNP-125 |
| rs6986285 | 8 | 128440524 | dbSNP-125 |
| rs6986469 | 8 | 128440699 | dbSNP-125 |
| rs6470518 | 8 | 128440770 | dbSNP-125 |
| rs6470519 | 8 | 128440812 | dbSNP-125 |
| rs6470520 | 8 | 128440922 | dbSNP-125 |
| rs7818556 | 8 | 128440988 | dbSNP-125 |
| rs1447295 | 8 | 128441627 | dbSNP-125 |
| rs4871802 | 8 | 128442229 | dbSNP-125 |
| rs6993074 | 8 | 128442270 | dbSNP-125 |
| rs10109700 | 8 | 128442553 | dbSNP-125 |
| rs9297758 | 8 | 128443177 | dbSNP-125 |
| rs6984861 | 8 | 128443731 | dbSNP-125 |
| rs10610521 | 8 | 128443970 | dbSNP-125 |
| rs13363309 | 8 | 128444111 | dbSNP-125 |
| rs9692964 | 8 | 128444780 | dbSNP-125 |
| rs7387935 | 8 | 128444971 | dbSNP-125 |
| rs7357547 | 8 | 128445291 | dbSNP-125 |
| rs13259396 | 8 | 128445300 | dbSNP-125 |
| rs13260378 | 8 | 128445339 | dbSNP-125 |
| rs1597019 | 8 | 128445342 | dbSNP-125 |
| rs7826042 | 8 | 128445690 | dbSNP-125 |
| rs7826179 | 8 | 128445788 | dbSNP-125 |
| rs13364857 | 8 | 128445897 | dbSNP-125 |
| rs13268049 | 8 | 128445908 | dbSNP-125 |
| rs11991386 | 8 | 128447040 | dbSNP-125 |
| rs10956373 | 8 | 128447165 | dbSNP-125 |
| rs7836840 | 8 | 128448381 | dbSNP-125 |
| rs16902165 | 8 | 128448411 | dbSNP-125 |
| rs7831028 | 8 | 128448618 | dbSNP-125 |
| rs1992833 | 8 | 128448933 | dbSNP-125 |
| rs2290033 | 8 | 128449663 | dbSNP-125 |
| rs28455156 | 8 | 128449949 | dbSNP-125 |
| rs11989136 | 8 | 128450373 | dbSNP-125 |
| rs9643224 | 8 | 128450700 | dbSNP-125 |
| rs9643225 | 8 | 128450980 | dbSNP-125 |
| rs9643226 | 8 | 128451070 | dbSNP-125 |
| rs11775749 | 8 | 128451255 | dbSNP-125 |
| rs11994384 | 8 | 128451916 | dbSNP-125 |
| rs1447296 | 8 | 128451948 | dbSNP-125 |
| rs16902168 | 8 | 128452197 | dbSNP-125 |
| rs9643227 | 8 | 128452685 | dbSNP-125 |
| rs11995378 | 8 | 128453001 | dbSNP-125 |
| rs16902169 | 8 | 128453095 | dbSNP-125 |
| rs13253127 | 8 | 128453180 | dbSNP-125 |
| rs11988454 | 8 | 128453351 | dbSNP-125 |
| rs11992194 | 8 | 128453353 | dbSNP-125 |
| rs6985504 | 8 | 128453365 | dbSNP-125 |
| rs13258548 | 8 | 128453436 | dbSNP-125 |
| rs13258812 | 8 | 128453456 | dbSNP-125 |
| rs4871804 | 8 | 128454118 | dbSNP-125 |
| rs16902171 | 8 | 128454315 | dbSNP-125 |
| rs12679900 | 8 | 128454604 | dbSNP-125 |
| rs16902172 | 8 | 128454631 | dbSNP-125 |
| rs7844561 | 8 | 128455093 | dbSNP-125 |
| rs1447297 | 8 | 128455211 | dbSNP-125 |
| rs12548204 | 8 | 128455431 | dbSNP-125 |
| rs7830797 | 8 | 128455565 | dbSNP-125 |
| rs7831150 | 8 | 128456027 | dbSNP-125 |
| rs13248046 | 8 | 128456232 | dbSNP-125 |
| rs10635608 | 8 | 128456241 | dbSNP-125 |
| rs13281765 | 8 | 128456338 | dbSNP-125 |
| rs7831722 | 8 | 128456407 | dbSNP-125 |
| rs7835553 | 8 | 128456440 | dbSNP-125 |
| rs4871024 | 8 | 128456500 | dbSNP-125 |
| rs7835701 | 8 | 128456514 | dbSNP-125 |
| rs4871025 | 8 | 128456569 | dbSNP-125 |
| rs723555 | 8 | 128456688 | dbSNP-125 |
| rs10808558 | 8 | 128457739 | dbSNP-125 |
| rs10685130 | 8 | 128458342 | dbSNP-125 |
| rs10685131 | 8 | 128458343 | dbSNP-125 |
| rs10686475 | 8 | 128458351 | dbSNP-125 |
| rs10103005 | 8 | 128458410 | dbSNP-125 |
| rs11393439 | 8 | 128459027 | dbSNP-125 |
| rs7820229 | 8 | 128459172 | dbSNP-125 |
| rs7820579 | 8 | 128459258 | dbSNP-125 |
| rs7013517 | 8 | 128459443 | dbSNP-125 |
| rs6993832 | 8 | 128459872 | dbSNP-125 |
| rs6994142 | 8 | 128460075 | dbSNP-125 |
| rs16902173 | 8 | 128460588 | dbSNP-125 |
| rs17766217 | 8 | 128461086 | dbSNP-125 |
| rs16902175 | 8 | 128461247 | dbSNP-125 |
| rs4871806 | 8 | 128461725 | dbSNP-125 |
| rs7818817 | 8 | 128462254 | dbSNP-125 |
| rs7010066 | 8 | 128462851 | dbSNP-125 |
| rs16902176 | 8 | 128462924 | dbSNP-125 |
| rs1562435 | 8 | 128463046 | dbSNP-125 |
| rs12155672 | 8 | 128463613 | dbSNP-125 |
| rs12156128 | 8 | 128463780 | dbSNP-125 |
| rs1562434 | 8 | 128463908 | dbSNP-125 |
| rs1562433 | 8 | 128464039 | dbSNP-125 |
| rs1562432 | 8 | 128464191 | dbSNP-125 |
| rs1562431 | 8 | 128464240 | dbSNP-125 |
| rs12056473 | 8 | 128464511 | dbSNP-125 |
| rs1374626 | 8 | 128464584 | dbSNP-125 |
| rs1374625 | 8 | 128464650 | dbSNP-125 |
| rs12056788 | 8 | 128464661 | dbSNP-125 |
| rs11365782 | 8 | 128464669 | dbSNP-125 |
| rs4599773 | 8 | 128467013 | dbSNP-125 |
| rs4078241 | 8 | 128467729 | dbSNP-125 |
| rs12545487 | 8 | 128467881 | dbSNP-125 |
| rs4461869 | 8 | 128467959 | dbSNP-125 |
| rs4078240 | 8 | 128468152 | dbSNP-125 |
| rs13269895 | 8 | 128468547 | dbSNP-125 |
| rs7013850 | 8 | 128468613 | dbSNP-125 |
| rs28609791 | 8 | 128469167 | dbSNP-125 |
| rs7813015 | 8 | 128469646 | dbSNP-125 |
| rs6981321 | 8 | 128469894 | dbSNP-125 |
| rs4871807 | 8 | 128469920 | dbSNP-125 |
| rs5894886 | 8 | 128470115 | dbSNP-125 |
| rs4871808 | 8 | 128470134 | dbSNP-125 |
| rs7817835 | 8 | 128470790 | dbSNP-125 |
| rs4412338 | 8 | 128471606 | dbSNP-125 |
| rs11408392 | 8 | 128472364 | dbSNP-125 |
| rs11393128 | 8 | 128472372 | dbSNP-125 |
| rs28475136 | 8 | 128472373 | dbSNP-125 |
| rs7827428 | 8 | 128472636 | dbSNP-125 |
| rs7832031 | 8 | 128473541 | dbSNP-125 |
| rs10113577 | 8 | 128473620 | dbSNP-125 |
| rs4242382 | 8 | 128474162 | dbSNP-125 |
| rs4242383 | 8 | 128474349 | dbSNP-125 |
| rs4314621 | 8 | 128474604 | dbSNP-125 |
| rs4242384 | 8 | 128475143 | dbSNP-125 |
| rs9297759 | 8 | 128475760 | dbSNP-125 |
| rs7018386 | 8 | 128476546 | dbSNP-125 |
| rs7812429 | 8 | 128476762 | dbSNP-125 |
| rs7812894 | 8 | 128477068 | dbSNP-125 |
| rs4871026 | 8 | 128477366 | dbSNP-125 |
| rs4871027 | 8 | 128478096 | dbSNP-125 |
| rs10099413 | 8 | 128478652 | dbSNP-125 |
| rs7814837 | 8 | 128478791 | dbSNP-125 |
| rs28429692 | 8 | 128479233 | dbSNP-125 |
| rs10088308 | 8 | 128479503 | dbSNP-125 |
| rs9297760 | 8 | 128479761 | dbSNP-125 |
| rs11457275 | 8 | 128479847 | dbSNP-125 |
| rs7007540 | 8 | 128480229 | dbSNP-125 |
| rs7841251 | 8 | 128480910 | dbSNP-125 |
| rs7824868 | 8 | 128481003 | dbSNP-125 |
| rs7017300 | 8 | 128481857 | dbSNP-125 |
| rs13275830 | 8 | 128481950 | dbSNP-125 |
| rs6470525 | 8 | 128482127 | dbSNP-125 |
| rs12547874 | 8 | 128482221 | dbSNP-125 |
| rs6470526 | 8 | 128482480 | dbSNP-125 |
| rs7004374 | 8 | 128482574 | dbSNP-125 |
| rs7005343 | 8 | 128483167 | dbSNP-125 |
| rs7010165 | 8 | 128483880 | dbSNP-125 |
| rs9693113 | 8 | 128484019 | dbSNP-125 |
| rs4871809 | 8 | 128484144 | dbSNP-125 |
| rs7461151 | 8 | 128484319 | dbSNP-125 |
| rs6470527 | 8 | 128484420 | dbSNP-125 |
| rs6470528 | 8 | 128484956 | dbSNP-125 |
| rs10108673 | 8 | 128485002 | dbSNP-125 |
| rs4582524 | 8 | 128485024 | dbSNP-125 |
| rs4641026 | 8 | 128485122 | dbSNP-125 |
| rs4498506 | 8 | 128485622 | dbSNP-125 |
| rs4297007 | 8 | 128485705 | dbSNP-125 |
| rs4242385 | 8 | 128485818 | dbSNP-125 |
| rs11992171 | 8 | 128486522 | dbSNP-125 |
| rs13255059 | 8 | 128487205 | dbSNP-125 |
| rs10091869 | 8 | 128487417 | dbSNP-125 |
| rs13265719 | 8 | 128487617 | dbSNP-125 |
| rs11986220 | 8 | 128488278 | dbSNP-125 |
| rs11988857 | 8 | 128488462 | dbSNP-125 |
| rs10090154 | 8 | 128488726 | dbSNP-125 |
| rs5894887 | 8 | 128488745 | dbSNP-125 |
| rs10103849 | 8 | 128488956 | dbSNP-125 |
| rs4515512 | 8 | 128488988 | dbSNP-125 |
| rs7388005 | 8 | 128489259 | dbSNP-125 |
| rs7824776 | 8 | 128490031 | dbSNP-125 |
| rs7843031 | 8 | 128490062 | dbSNP-125 |
| rs4645527 | 8 | 128490582 | dbSNP-125 |
| rs4599771 | 8 | 128490819 | dbSNP-125 |
| rs4531012 | 8 | 128490950 | dbSNP-125 |
| rs13277027 | 8 | 128491016 | dbSNP-125 |
| rs9656967 | 8 | 128491176 | dbSNP-125 |
| rs9656816 | 8 | 128491243 | dbSNP-125 |
| rs12548153 | 8 | 128491281 | dbSNP-125 |
| rs12545648 | 8 | 128491344 | dbSNP-125 |
| rs7005132 | 8 | 128492224 | dbSNP-125 |
| rs4871810 | 8 | 128492949 | dbSNP-125 |
| rs13264091 | 8 | 128493043 | dbSNP-125 |
| rs11985949 | 8 | 128493373 | dbSNP-125 |
| rs13272543 | 8 | 128493517 | dbSNP-125 |
| rs12547606 | 8 | 128493842 | dbSNP-125 |
| rs12542685 | 8 | 128494172 | dbSNP-125 |
| rs11987811 | 8 | 128494732 | dbSNP-125 |
| rs7814251 | 8 | 128494806 | dbSNP-125 |
| rs11268643 | 8 | 128494962 | dbSNP-125 |
| rs8180905 | 8 | 128495413 | dbSNP-125 |
| rs9694093 | 8 | 128495737 | dbSNP-125 |
| rs7837688 | 8 | 128495949 | dbSNP-125 |
| rs13256658 | 8 | 128496050 | dbSNP-125 |
| rs7824118 | 8 | 128496937 | dbSNP-125 |
| rs10551941 | 8 | 128496952 | dbSNP-125 |
| rs13265998 | 8 | 128496973 | dbSNP-125 |
| rs13266000 | 8 | 128496975 | dbSNP-125 |
| rs10107263 | 8 | 128496987 | dbSNP-125 |
| rs13268425 | 8 | 128496989 | dbSNP-125 |
| rs13268712 | 8 | 128497079 | dbSNP-125 |
| rs13266351 | 8 | 128497100 | dbSNP-125 |
| rs12549761 | 8 | 128497365 | dbSNP-125 |
| rs4871811 | 8 | 128497463 | dbSNP-125 |
| rs4242386 | 8 | 128497682 | dbSNP-125 |
| rs7825823 | 8 | 128498506 | dbSNP-125 |
| rs28489376 | 8 | 128499033 | dbSNP-125 |
| rs7465074 | 8 | 128499382 | dbSNP-125 |
| rs11308570 | 8 | 128499734 | dbSNP-125 |
| rs11988556 | 8 | 128500924 | dbSNP-125 |
| rs7007196 | 8 | 128501145 | dbSNP-125 |
| rs6470529 | 8 | 128501401 | dbSNP-125 |
| rs11323753 | 8 | 128501468 | dbSNP-125 |
| rs11300434 | 8 | 128501591 | dbSNP-125 |
| rs10106375 | 8 | 128501959 | dbSNP-125 |
| rs6991990 | 8 | 128501972 | dbSNP-125 |
| rs4543510 | 8 | 128502208 | dbSNP-125 |
| rs7846178 | 8 | 128503193 | dbSNP-125 |
| rs11786789 | 8 | 128503317 | dbSNP-125 |
| rs5894888 | 8 | 128503510 | dbSNP-125 |
| rs11368434 | 8 | 128503511 | dbSNP-125 |
| rs11988207 | 8 | 128503749 | dbSNP-125 |
| rs7003169 | 8 | 128504149 | dbSNP-125 |
| rs4871812 | 8 | 128504310 | dbSNP-125 |
| rs7837009 | 8 | 128504410 | dbSNP-125 |
| rs4871813 | 8 | 128504531 | dbSNP-125 |
| rs12386846 | 8 | 128505038 | dbSNP-125 |
| rs13258742 | 8 | 128505267 | dbSNP-125 |

*Location in bp and according to UCSC browser NCBI Build 34

Table 21 contains all microsatellite markers identified and tested by deCODE genetics in the LD-block interval on chromosome 8 (128.414-128.506).

TABLE 21

All Microsatellite Markers in the LD-block interval (128.414-128.506)
from Decode Inhouse Microsatellite Markers track in the UCSC browser

| | Amplimer | |
|---|---|---|
| Name | Start-End* | Primers |
| DG8S381 | 128415035-128415316 | F: TGTTGAATTCATTCTCTAACCACTTC (SEQ ID NO: 142) |
| | | R: TGATCATGAAACAGTCAACGTCT (SEQ ID NO: 143) |
| DG8S1000 | 128421282-128421645 | F: GCCCACTGTCCAATTAAGGA (SEQ ID NO: 144) |
| | | R: TCTACAGCCTCACACCGAAG (SEQ ID NO: 145) |
| DG8S1184 | 128421282-128421684 | F: GCCCACTGTCCAATTAAGGA (SEQ ID NO: 144) |
| | | R: TGTGGGTTTACATGCCAGAA (SEQ ID NO: 146) |
| DG8S1758 | 128425313-128425492 | F: GATCCCACTCTGTCACTCCTTT (SEQ ID NO: 147) |
| | | R: TGGGTGCCTGTAGTCCTAGC (SEQ ID NO: 148) |
| DG8S1434 | 128426022-128426425 | F: CCACAGTGATTCCCACCTCT (SEQ ID NO: 92) |
| | | R: AGTGTTGGCCAGGGATGTAG (SEQ ID NO: 93) |
| DG8S1775 | 128429995-128430409 | F: CTTGGCCTTGTTCACAGGAG (SEQ ID NO: 149) |
| | | R: TTTCTATGGCAAGTTGCTGTTT (SEQ ID NO: 150) |
| DG8S737 | 128433035-128433169 | F: TGATGCACCACAGAAACCTG (SEQ ID NO: 94) |
| | | R: CAAGGATGCAGCTCACAACA (SEQ ID NO: 95) |
| DG8S1759 | 128439725-128439956 | F: AGGATGCACAAGCCTGATTT (SEQ ID NO: 151) |
| | | R: TTGGCCATAGCTCCAACTTC (SEQ ID NO: 152) |
| DG8S1760 | 128441048-128441156 | F: TCTCCAAATTCCAGTTCTACTACTTT (SEQ ID NO: 153) |
| | | R: TTTCTCTTTCCTGCTTTGTCTCTT (SEQ ID NO: 154) |
| DG8S1772 | 128442434-128442652 | F: AAATCTGGCCATCCTCCTCT (SEQ ID NO: 155) |
| | | R: AATCCTGTCCCAGGCAGAC (SEQ ID NO: 156) |
| DG8S603 | 128447576-128447735 | F: CCCTGAACTCAGGAACAAGC (SEQ ID NO: 157) |
| | | R: CAAAGCCGTGTCTTTCCTTC (SEQ ID NO: 158) |
| DG8S916 | 128450374-128450524 | F: GGGATAGCCCATGGATAGGA (SEQ ID NO: 159) |
| | | R: TGAATTGTTGCACAAATAAAGG (SEQ ID NO: 160) |
| DG8S1761 | 128452659-128453051 | F: TTGAAATTGCAATCCCATCA (SEQ ID NO: 96) |
| | | R: CCTCCCTACTTATTCCCATGC (SEQ ID NO: 97) |
| DG8S1090 | 128466777-128467062 | F: TGGGAAGAATAAGAGGTCCAGA (SEQ ID NO: 161) |
| | | R: TCAGTTCAGCTGTCCAGCAA (SEQ ID NO: 162) |
| DG8S1776 | 128469902-128470203 | F: GGGCATAGTGCTTTCTGCTT (SEQ ID NO: 163) |
| | | R: TGATGCATTCCTTTATTCTCCA (SEQ ID NO: 164) |
| DG8S422 | 128475211-128475589 | F: AAATGCAAGCAAAGCCAAGT (SEQ ID NO: 98) |
| | | R: GCTCCACACACAGAGGTCAA (SEQ ID NO: 99) |
| DG8S1768 | 128482506-128482838 | F: CCAAGCTCTCTTCTGGCTTC (SEQ ID NO:165) |
| | | R: TTGCATCCCATCTTTCCTTC (SEQ ID NO: 166) |
| DG8S1777 | 128486146-128486367 | F: TGGTGAAGGGACTCTTCCTG (SEQ ID NO: 167) |
| | | R: CCCATGGTAGAACTGGCAAA (SEQ ID NO: 168) |
| DG8S1773 | 128488657-128488789 | F: TTCTCTCCAGATTGATACACAGC (SEQ ID NO: 169) |
| | | R: TGGCCATATAGTAAGCCTTGG (SEQ ID NO: 170) |
| DG8S1764 | 128489121-128489371 | F: TCCACCTATCCAAGCAACAA (SEQ ID NO: 171) |
| | | R: TGTAGTGATATGCCAATGTGGT (SEQ ID NO: 172) |
| DG8S817 | 128493580-128493825 | F: TTTCCAAACCAAGGTCAGATTT (SEQ ID NO: 173) |
| | | R: GCCCTGCTTCAGTGAATGTT (SEQ ID NO: 174) |
| DG8S738 | 128493793-128493883 | F: TCCATGCACAGAAACATTCA (SEQ ID NO: 175) |
| | | R: TCATTTATTACTTTGCATTTGGCTTA (SEQ ID NO: 176) |
| DG8S1503 | 128496744-128497027 | F: CAGTCACGTAGAGAGCAGCAG (SEQ ID NO: 177) |
| | | R: CTGGGCCACAGAGTGAGAC (SEQ ID NO: 178) |
| DG8S1502 | 128496756-128497097 | F: GAGCAGCAGTAATCCCGAAT (SEQ ID NO: 179) |
| | | R: GGCAGAAGAATCGCTTGAAC (SEQ ID NO: 180) |
| DG8S1504 | 128496803-128497049 | F: TGCACAGTATTTCTTTCCATTGTT (SEQ ID NO: 181) |
| | | R: GATCGCACCATTGCACTCTA (SEQ ID NO: 182) |
| DG8S1185 | 128500590-128501013 | F: GCTCTTGGTGAAAGAGAGAAGG (SEQ ID NO: 183) |
| | | R: CAGTTCATGTTTCGGGAGGT (SEQ ID NO: 184) |
| DG8S1769 | 128501385-128501647 | F: CCTCCCAAACACACAGAGTTG (SEQ ID NO: 100) |
| | | R: TGTTAAACCTAAGGGTTCCTTCC (SEQ ID NO: 101) |
| DG8S350 | 128502740-128503092 | F: CTGCTCTCCTCTCAGCTTGC (SEQ ID NO: 185) |
| | | R: AAAGGCTCTCTTGATCATGTCC (SEQ ID NO: 186) |
| DG8S1407 | 128503459-128503695 | F: CCAATAGCCTTCAATGTATCAAA (SEQ ID NO: 102) |
| | | R: TGAGGAAGAGCCACAACAGA (SEQ ID NO: 103) |

*Start and stop of amplimer is in bp and according to the UCSC browser NCBI Build34

TABLE 22
A protective haplotype consisting of markers/alleles:
rs12542685 allele T and rs7814251 allele C

| p-value | RR | Count Aff | Aff Freq. | Count Ctrl | Ctrl Freq |
|---|---|---|---|---|---|
| 0.00015 | 0.7504 | 1280 | 0.194 | 995 | 0.242 |

The teachings of all relevant publications cited herein are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of diagnosing a susceptibility to a cancer in a subject, comprising detecting a marker or haplotype associated with LD Block A, wherein the presence of the marker or haplotype is indicative of a susceptibility to cancer.

2. The method of claim 1 wherein the marker or haplotype is a marker selected from the group consisting of the markers in Table 13.

3. The method of claim 2 wherein the marker is the rs1447295 A allele or the DG8S737 −8 allele.

4. The method of claim 1 wherein the marker or at risk haplotype is an at risk haplotype comprising a haplotype selected from the group consisting of: haplotype 1 and haplotype 1a.

5. The method of claim 1 wherein the marker or haplotype is a haplotype that comprises one or more markers selected from the group consisting of the markers in Table 13.

6. The method of claim 5 wherein the haplotype comprises the rs1447295 A allele or the DG8S737 −8 allele.

7. The method of claim 1 wherein the cancer is selected from the group consisting of prostate cancer, breast cancer, lung cancer and melanoma.

8. The method of claim 7 wherein cancer is prostate cancer, and the marker or haplotype has a relative risk of at least 1.5.

9. The method of claim 8 wherein the prostate cancer is an aggressive prostate cancer as defined by a combined Gleason score of 7(4+3)-10.

10. The method of claim 8 wherein the prostate cancer is a less aggressive prostate cancer as defined by a combined Gleason score of 2-7(3+4).

11. The method of claim 8 wherein the presence of the marker or haplotype is indicative of a more aggressive prostate cancer and/or a worse prognosis.

12. The method of claim 7 wherein the cancer is breast cancer, and the marker or haplotype has a relative risk of at least 1.3.

13. The method of claim 7 wherein the cancer is lung cancer, and the marker or haplotype has a relative risk of at least 1.3.

14. The method of claim 7 wherein the cancer is melanoma, and the marker or haplotype has a relative risk of at least 1.5.

15. The method of claim 7 wherein the melanoma is malignant cutaneous melanoma.

16. The method of claim 1 wherein the presence of the marker or haplotype is indicative of a different response rate of the subject to a particular treatment modality.

17. The method of claim 1, wherein the presence of the marker or haplotype is indicative of a predisposition to a somatic rearrangement of Chr8q24.21 in a tumor or its precursor.

18. The method of claim 17 wherein the somatic rearrangement is selected from the group consisting of an amplification, a translocation, an insertion and a deletion.

19. The method of claim 1, wherein the marker or haplotype comprises one or more markers associated with Chr8q24.21 in strong linkage disequilibrium, as defined by (|D′|>0.8) and/or r²>0.2, with one or more markers selected from the group consisting of the markers in Table 13.

20. The method of claim 19, wherein the one or more marker comprises the rs1447295 A allele or the DG8S737 −8 allele.

21. A method of diagnosing a susceptibility to a cancer comprising detecting a marker or haplotype associated with Chr8q24.21, wherein the presence of the marker or haplotype is indicative of a susceptibility to cancer.

22. (canceled)

23. A method of predicting an increased risk for aggressive prostate cancer in a subject comprising detecting a marker or haplotype associated with LD Block A, wherein the presence of the marker or haplotype is indicative of an increased risk for aggressive prostate cancer.

24. (canceled)

25. A kit for assaying a sample from a subject to detect a susceptibility to a cancer, wherein the kit comprises one or more reagents for detecting a marker or haplotype associated with LD Block A.

26-28. (canceled)

29. A method for diagnosing an increased risk of cancer in a subject, comprising screening for a marker or haplotype associated with LD Block A, wherein the marker or haplotype is more frequently present in a subject having the cancer than in a subject not having the cancer, and wherein the presence of the marker or haplotype increases the risk of the subject having the cancer.

30. (canceled)

31. A method for diagnosing a susceptibility to cancer in a subject, comprising:
i) obtaining a nucleic acid sample from the subject; and
ii) analyzing the nucleic acid sample for the presence or absence of at least one marker or haplotype associated with LD Block A, wherein the presence of the marker or haplotype is indicative of a susceptibility to the cancer.

32-34. (canceled)

35. A method of diagnosing a Chr8q24.21-associated cancer in a subject, comprising detecting the presence of a marker or haplotype associated with Chr8q24.21, wherein the presence of the marker or haplotype is indicative of the Chr8q24.21-associated cancer.

36-38. (canceled)

39. A method of diagnosing a susceptibility to prostate cancer in an individual, comprising:
1) detecting marker DG8S737, wherein the presence of a −8 allele in DG8S737 is indicative of a susceptibility to prostate cancer; and/or
2) detecting marker rs1447295, wherein the presence of an A allele in rs1447295 is indicative of a susceptibility to prostate cancer.

40-43. (canceled)

44. A method of diagnosing an increased risk of prostate cancer in an individual, comprising:
1) detecting marker DG8S737, wherein the presence of a −8 allele in DG8S737 is indicative of an increased risk of prostate cancer; and/or
2) detecting marker rs1447295, wherein the presence of an A allele in rs1447295 is indicative of a susceptibility to prostate cancer.

45. A method of predicting an increased risk for prostate cancer in a subject comprising:
1) detecting marker DG8S737, wherein the presence of a −8 allele in DG8S737 is indicative of an increased risk for prostate cancer; and/or
2) detecting marker rs1447295, wherein the presence of an A allele in rs1447295 is indicative of a susceptibility to prostate cancer.

46. A method of predicting an increased risk for aggressive prostate cancer in a subject comprising:
1) detecting marker DG8S737, wherein the presence of a −8 allele in DG8S737 is indicative of an increased risk for aggressive prostate cancer; and/or
2) detecting marker rs1447295, wherein the presence of an A allele in rs1447295 is indicative of a susceptibility to prostate cancer.

47. A method of diagnosing a susceptibility to prostate cancer in a human having ancestry that includes African ancestry, comprising:
1) detecting marker DG8S737, wherein the presence of a −8 allele in DG8S737 is indicative of a susceptibility to prostate cancer; and/or
2) detecting marker rs1447295, wherein the presence of an A allele in rs1447295 is indicative of a susceptibility to prostate cancer.

48-55. (canceled)

56. A method of diagnosing a decreased susceptibility to prostate cancer in an individual, comprising detecting the haplotype shown in Table 22, wherein the presence of the haplotype is indicative of a decreased susceptibility to prostate cancer.

57. A method of diagnosing a decreased susceptibility to prostate cancer in an individual, comprising detecting a marker shown in Table 13 having a relative risk of less than one, wherein the presence of the marker is indicative of a decreased susceptibility to prostate cancer.

58. A method of diagnosing an increased susceptibility to prostate cancer in an individual, comprising detecting a marker shown in Table 13 having a relative risk of greater than one, wherein the presence of the marker is indicative of an increased susceptibility to prostate cancer.

59. A method for diagnosing a susceptibility to cancer in a subject, comprising analyzing a nucleic acid sample obtained from the subject for the presence of at least one marker or haplotype associated with LD Block A, wherein the presence of the marker or haplotype is indicative of increased susceptibility to the cancer.

60-84. (canceled)


SEQUENCE LISTING (180 sequences)

SEQ ID NO: 1 - DNA, 92001 bp, Homo sapiens
tctttgacaaataaattagcatacctagaaggaagccagatgctattccattaagacaatggaagaatgacaccaaagtcatttcaaagatcttggaagccgccactctcataacaggctcagagtgccagggccctgaaggcagaacaatttcataggtggtaccttgaggcttcatcacccaggactgcctcaggcttctgctccactcattctggcccaacactctttgtctgttgcagctgtagatcctcaagtgcacccaggtgtggttcaaagtgctagtccggagagcatgaggtaaaccctggcagcatccatgtggtgctaactgagcaaatgctcagagtgcgtgagctaagcaggcatggctacctcctcctcgatttaaaaggatgcttatgagagccttggggcgtaggcagagaactgtcacagtggcagggctaccacagatagcccctactagggcaatgcttagtgggggcagggccactcctgagaccccccagcctgtgcagccaccagcatgcaatgccagcctgcaagaataacaggtatgtgactccaacctgtgaaagctgcagcataagccgcgcctagcaaagccacggggatgaggctgcccagagtcttgtggggaccaacccatataccagtgtgtcaagtagtcaggaatggagtcaaataaaattattttcagtcctccatatttgagtgttatttgccttgttgggtttcgaatgggagtgagacctattactcctttcttctttcctatttttttcccctttggaatgggaatatccatcctatgcctgtcccaccactgcatttgaagcacataataaaatgtattcattctatcccaataattccaaaaatcttaattaatccaacatcaactttaacttataaatctagagtatcatctaagtatcatctaaatcagatatgggggaattcttctccagcagtctgcaaaatctgacaagatatagatatctatatgtccacatacgtaactataactatctgttgaattcattctctaaccacttctatcaagtcactgtgctaaaacttcttacttaatgctcattagtgttacacacacacacacaaagacacacacacacaaagacacacacacttgctgctagattttactctcttggtggattttactctcttgctgctagataaggggtacctaatgcaagatgtacctatcttagagtttgcacatcatctgcattattgaacgtaaagtaaaagccaaatgagggaaaagagacgttgactgtttcatgatcattgtcttacctgaaacaattaggaagtcaccgttcagctttgcaggctatgtaattgtcatatatcatgaatggctgataaggggttgaatctgtgaggttttcccatagaggaaagggaaatactgattctcactttagccaacagcagttgtctctaactcacccaaactgctgagaagaacttaagaactattttactgtgctctttttcttatttatttatttagagacagagtcacattctgtcacccaggctggagtgcagtggcgatctcgcctcactgcaacctctgcctcctgggttcaggtgattctcctgtctcagcctccctaatagctgggattacaagcatgcaccaccatgcccagctaatttttgtatttttagaagatatgaggttttgtcatgtcggccaggctgctctcaaactcctggtctcgtgatccaccttcctcagcctcccaaagtgctggaatcacaggcatgagccaccgcacccagcctttaagagctattttatttccaatgcagaatggaacctcaacaccatcatggatgagttccagattcataagactttaggcctacagcggacatcagacacaatctaactgagctccttcactgtagagatgtgggaaaggaggcccaagataggaaagagacttgcccaaggccacacatctggttaatggagaacaagagacaatactcatttctcccaaccaccatgccagtgggagttccaggtccaccctgcctgagagcttcatgaccacctccaagatggcccaacgctgcccttcaaaaggggtcagtacctgggcaatctgatggtgaattcattagaggtgaaataaatgcttataaaagggaaaatggagacttgtaattctacctcttgattctaagaaatcctttcattgggtctagagtgttcataaatctctataatttgaaaattgaacagcacagttttctatgaacaagtgcaaaaacggggtcagaagtcaggaaaattgtaaagatgggatgtgacattgctgcttccccaatctttacctacaaaacctctccttgctctcttccattctatcaaatcctagtctcagactattcatttacactaagatgtaaatgactgacagcaaagtggccactttcaggggcatttggagacaaagggatgacattgcgattattgacctatcacactggggatatcagagccagagagaagacgtggagtctgaaaagaaaagctgttcccacaacaaagaattatccagccctaaatatccatagtgcagaggttgagatatcctgctctgggttacagaaagttgcataaattcctagcatgaatcatcaggtgggcaccttgagagcaatctgccatctccacaactttgtacctttgcctttttttccaaaaagaaagggtctaaccaaaccatgcttatgaccagcctcagcctcccatcaactggcaccattggcatcctggcccttgtgtgtctgcacagtgcctgctacacactttcattaagtaactctcaaccttacaataattcagggaaaggagacagtattgttattcataccccacatacaaagaaacaaagaaggaggcaatttaccaggttttcacagggaggaagaggaagggtcagaattccaatccagactgatcctagctccagaaactaaactatcacactatcacacttcagacaggagtgcagattatcaaacactaactattttaagaaatgttcaggggcggagcaagagcacaggtgccatccagtgctgtttgtggagaggaggtattcggaggccacaacgtagggcaaggcatttcttcctaggctgggtatgataggtagaataatggctcccccaaagatgtccacatcttaacccctgtaacctgtgactattaccttacaccatgaaagtgactttgcagatgtgattgaattaagtgccctgagttggggagattttcctggaagaatcaggagggctgcatggaatcacaagggcccttataagagggaggcaggagagtcagagtcagaggaggtgtgaggatggaagcagaatttgtaggggtatgggcgagaagccaaggaatgcagatgacctctagaagccggagaagcaaaatggattctctcccagagtctcacaaggagtgcagtcctgtgaacaccttgatcttagcccagtgaagctgatttccagcttcagacctccaggactttaatgataataaatttgtgttgttttcagccaccaagtgtgcaataatttgttatggaaatcacaggaagctaatatatgaggattgctacataaagtatagaacacacagtccaacttgaacttcagataaacatcaaataattttctagtatgagcatgctccatgcaatattcatgtgtcctgtattttcatttgctaaaactggtagccctaaccggcttccccctcccactcagcccctgcatccccaaagccctttcccaggtctgctctgttacagtggtatcattatacctgctattctggggttagtctatgactcaaccgagtgaaactgttaaggcctcagataaatagctctgagggaaagttattattctatatcattgcctatcaattatttatagtttataatgcaatgccatttgtaatgggtaatatgtttttctattgtgttgtaatatcatccaactcatcagaacaaccattggggagatggattggcttgatgtaaatagcagtgcctagtaacaagctgtgtgcatttttaaacctggaaatcactgctctttcagataagaggaaactcttctttgaattcaaacagaagggtccaagccaccttttctgacaagttgatttcctgagaagcagggcaggaactagggcaaggcaaatgtgttcagggtacaaaatgtaaggaggctctccatttcaggtgccgaccctgcacttgcagaacgtgaaaatgagtgctgctttaaatcttgcaccctgggagactcacttgcttcactctagtatcgactctgctaagaaaactcacatgaagccatcaacaaacgccaaagctaacctagcctctttacatggcttatgggaaactgaggcccaaaaaaatgtggaccagccaagattcaatttgcttagtgactagtggacctaatggttgctactgcagcatcagctgcttctgccaacacctacctactgagtttcagccagaggcttgaggttcgcactgccctcccagccctgggacaaggccctgtctcaactggattaagcaaatcagtataacttcgtatccctgatcccagtgattgaacacttaactcagtcctaagccagtaaataggttgtatgtccctggccacagttgttagctccaggatatacatgtggggcagggtgaagctcatgactttgattcacggagagagaaaatgattcctctttcctttggagggtgtggaatacaaatagaaagtctggaactgctgcaaccatttattaccatgagaaaagccagaatgggaatgaaagcaatcatatatagaagggaaaaggtgagagaatcataaagggaaaccagattcttcaccgactcccatctaaaggacccactacctctggactattctgttatatgcaccaataaattcagattaatatttaatccagtttgagttaaaatattctgttacttgtacaaaagaggttcctaactggacaatgaccttagaaaagccaagttctacagctacattgccccatagaactttctgcattgatgcaactgttctatatctgtgctgttcgatacagtaagccactacctgcgtgtggctattgaacatttgaaatgtggctaatgtgactgagaaatggacttttaaattttgttaatactaattcctttcaatttaaacagccacacagaccagtggttaccatattggtccatgcagccctacaagcttcttttccttcatttcatttgttgcctcctccagaaaccagctccagatcctttctttcctccagagttggttaggctgatcttttctgtgatcctatagcaactgagcattcctctaaaattgtgattgcctctcttagaataatatcttttaccgtgtgtatctcccacctttggttgaggttttaaaaggcaaggcccattcctgtgtccaagcactgagcagaagcattgccatggtgacttttgaattggatgagataatctgtggcccaaatgtgtaccagatgttgaaaggctttgtagcttttaagaatgtaagacaatataaaaatgtttgctatcattattagttattgttactatttctgattgttaaccttgatccacttctcaattttctacaggagtctcagtcactgccctcgaatgactagaaatttgcccagccttgtgacatctgtgaccatcagtgtcaggctcactggcttcttacccctctgctgccttgactttctttttcttttctttcttttttttttttttccttgagacggagtctcgctctgtcaccaggctggagtgcagtggcgtgatcttggctcactgcaacttccgactcccctgttccctgctttaagcaattctcctgactcagcctcctgagtagctgggactacaggcacacaccaccacgcccagctaatttttgtatttttaataggcacagagtttcaccatgatggccaggatggtcttgatctcctgacctcgtgatccgcccaccgcggcctcccaaaactgccttgactttctagctcaaatgtcctatttccctaattatctggcttggtcctcttagtctgccacctacacacttgtgggtgagagaggaggagctggaagacaatgcagagctgcagttaaggccattgcaattgccagggtgtgctatgtggcttcgacatgcttagaagccagtatggtttggctgtgtccccacccaactctcatcttgaattgtagttctcataattcccacatgttgtgggagggacccagtaagagataattgaattttgaggccagtttcccctatactgttttcatggtaatgaataagtctcatgagatctgctatttttataaggagttccccctttcacttggctcttattctctcttgcccgctgccatgtaagacatgcctttcaccttccaccatgactgtgatgcctccccagctcccatgtggaactgtgagtccattaaacctctttttctttttataaattacccagtctcggctttgtctttattagcagcatgagaacaggctaatacagaatcctcctccttgagatgtgcttcctgatcccaggcctttacaccaggggaattcatcaaattcattcatctccttaacaaatgttactgagttcctaaaaattgtcaagcatgtgatgaggcactggggttctgtcagtgaaaaagacaaagtttctgtcatactgaaggtcacgttctaataggggagacagacagaaataatacaccatataaataaatggctacagtaaagaacaatcgagtgtgggaatagaggggaagcaggcaggtaactgcagtggaccagaaaaaagcaatggtgacaaagtccagagtggtggcaggtgagacaatggagtgttatcagatttgagaccaggaacttcttaagagtcctttttcttttgcctttggtctcactccttgatttattccagctctgccagatcagatcttgtctttacatgcccccacccattctcagataccctctgtgccttaacaactaataacctgttggttctttccagggaataacttgtactcagcttctggggttccctaggcttttccactggaataaaattttctcctagctgagcccactgtccaattaaggatcagacaaaatttagaagaagcaagtcacttctcctttctggatctgtttcttcctgtgcaaaaagagatgttgcacaagataatctctggtgccccttccaatttaatattttataattacagcctaggttttgaagaataaaaatataataagagaagagagaaagaaagagatgaggagtggggcgggggaagaaaggggaggaagaggaggaggaggaaaagcaggagaaaggggaagggagagggggaaggaagaaagggagggagggaagaaacctatttgtaaggccaagttaaaaattcattgccacaataaatacttcggtgtgaggctgtagaaattggcaaaatgaaaaaattctggcatgtaaacccacaggtgagtcctaggctaagagaagcatcttctacctccctcacttcctgatccccatcccattagcatttctgaaatgcctggaaagatgctccctagcagggtagagtcaggaacccactgtctgttcctttctcatctaatctgagcattggaaagatgctccctagcagggtggagtcaggagcctgctgtctgttcctttctcatttaatctgagcataatctggtgctttgggctgtggagcggatttctctctccagtctgtttctgcctgtcctgtcttgaggtgcactaggcagggatctcaagtgtgtccttggcctgcagtggggacttgtgcatctgaaggagcaatcaggcaggtggcctggggcagcctgctccataacgatgccctcattcccaggagcaagactgaatgccagttactcaaatcccattcctggctttctgcttcagtcattactaagagtttttcatatcttctggtgttcaggaaaatatttctattttcaggagaagcaactctgccttatactcgaaaattaaccagacattctattattgctgaaattctttcctcgtattgcattttatttaggcttttaaaaatcctttatttatgcagtttgaatcaggggtattttctggcttacaggttagtagagaattacaaaacattcaaaatttcaaagattccctaagggagaagtgatctattccaatatgggcttatattccagaaaaaaaaaaaaaaaaagcgtactcccagaaagatttgtctgggaaatgattgcaaatgagaaaactcactcagatgtgagaaatgataccgccctggactttcatgctgggccctaagaattatgacatggatgtaaaagaagattgccacaagtcgccagcttgcctgctccttaagatgcttgaagtccccagtgaaagctgagaaacatgaaattacttctgaatgtgcccacagacaggacagtgtaaattgtaatgacagagaaaaagacctgctgcaggcctccctcacgcgatcccaagtgagttttacagtcgtgtagtaggatagagtgagcctcattctaaggctgacgaagctgaggctcagtaaccccagtttgatcaacttgagtcagcaagctcaaaactggtgaaagccagattttcttattttttttttctacccaggccttctacaacaggtctctacaactagtgtcttttatgacccagtgctgctttctgattgggaacataccaagttcaaggtccccatgtggtcctgtatagtattcacaatactatatagtatgcaactgttctatatctgtgctgttcaatacagtactatatatagtatgcacaatttccatgcaaggaaaacatttaatatcttacaatgcactttcacacttagcatcccacttgacccttacaacaacttcatggggtaaatatttatttccctgtttatgaataaagaaattgaggctcacgttctgttaaagccattgttatggccttaacccattctattatgcctccaagaacacacaaaatctggtggctgttctgggagctaacaaactctgctttaagtcctaactctgccattctccaactttgtgacctcaggcaaaatgcaaattttttctgcatctcagtgttcacatgcgataactagagggaaagagtacttgcctcatacggcagattcttcattcattctacaagcatttattcagcatctactatgctctaagttctggagatacagtaatgaacaaaacaaaagcctcagcatttgtggagttatgttctacttatgggagacagaaaagaaaatatattgcatttgaaggtgataaatactaagaaatataaaacagggaaagagaatagggagtattggaaggaggagttcagttcaaatcagaatgaccaggaaagactgcaattgagaaagcaacatgcagtgaagacttcaaaaaggagtgaggggccaggcgcactggaggaggattactcttgtaatcctcgcactttgagaggccaaggcaggccaaacacttgagctaaggtgttggaggccagcctgggcaacacagcgaaaccccatctctacaaatacaaaaaaattagctaggcatggtggtgggctcctgtagttccagctactcagaaggtggggatgggagaaacacttgagtccaggaggtcaaggctgcagtgagctgagattgtgtcactgcactccagcttgggcaacagagtgagaccctgtctcaaaaaataaagggaggagtgggcggtgaaagagcaagctttgtaagtatctggaagaagagctttctaggcaagaggaaacagcaagtttagaggcccagaggcaagacactgggagggtggtgcctctgtgttccagaaacagggaggccatgtggctgcactggagtgagcgagggagaaagaagtaggaggaagatcagagatgtaactgatagtgcgaggatgggacactaattatgcaatgctttatgggaccattacaaggacttagattgttttctctaaaagtaattgggagccattggaggcttttgacgtaggagtgccatgatctgacttcagttttaccaggatcactcaagctgctatgttgtgttagaccggagggatcaggtgtgggagccagttagggagatgttaaatgttctctaggaaaagaggatggtggtttgggctgcaatggtagccattgagatggagaaatggtaggaattgggttttattttgaaatcgagccaacagggtttgctgatgagttatatgtgagtgggagagaaggaatgggactacgagtttcttggtctgagtaactgcttttcacaagaatgagagtatagaagaagcagctctgcaagagtgagaaagaactgaacaatgaatgtatattttctccagctatacagtgtccagcacagagtatgtgttccataacaggtagatattactattaataatgtatctgctcacctggttaaatatttctgcagacctttgccctacatccaagcactctgagaaagcacatggtcaggctgttttccaccagcaaatggaccacagaatcttggtaccatagtggtttttaaagcccattggctacccaaaacccctttgacccatcaagcgtgctctggactcaatacctttgcacaggctgatctccctctccacccacccttccttcctctcagcttctccatacaggaaatggacaaaggcacaaactggaagtcagtgcagattcaagctttggttttgttcagagagtattccttaatgctcacagaaaatctagcccagcttctcctattacaaaactggaagctgaggactagaaagatgaagtggcttgcagcttccccatcagtatgacaaggaaactgtattaaataattcccaaggattcttctagctctcacatgctttcaggttttcaataaacacaagatacaataaaaagcaatcaaaataatgaccagctgattttgctttccttcattaccctcatctaagccaccagcaaactctgctaactcttcctccaaaatagatcccactctgtcactccttttttttttttttttttttttttttttgagacggagtcttgctctgtcacctaggctggagtggagtggcgcgatctcggctcactgcaagctccgcctcccgggttcacgccattctcctgcctcggcctcccgagtagctaggactacaggcacccaccaccacgcccagctttttttgtatttttagtagagatggggtttcaccgtattagccacgatggttttgatctcctgacctcctgatccacccgccttggcctcccaaagtgctgggattacaggcttgagccactgcgcccggccactgtcactctctttttatggtcaccaccatgagacaaaccaccaccaccttgaaatgaacagccagagcagcctctaactggcttctttcccctccttaccctggccctcctacttgctatatgcatgtgacagtctgaacgatctgttgagaatttgaaccaaatgtatttacttcttttcccaaaacctaccagtaactttctatcatgtcttaccttggccaatcaggccttacatgatctggcccccatttttctctttgaccagatcttgcaccactatccccttatctacaagcctctctgaccttctgcctgtgcctttgatattccaggcttcccctctctttggcattcaggctcctaagagggccaccccacccacagtgattcccacctctccatattcacatcctttgtgtaggctggacctagtgctttgcctttaaagaacagaatatggcaaaggtgatgggatgtcacatccacgattaggttgcaaaagactgtaacttccatcttattggctctctctctctctctctctctctccttctcactttctcactcagatgaagcaagtttccatgttgtgagctgccctgtgagaaggtttacttgctaggaactaagagtggcctccagccaacagccagcaaggaagtgaagccctcagtctaacagcttgggagaaactgaattgtgccatcaatcatgtgagtgagcttggaagctcttctatccccagtccaaccataacaggactacatccctggccaacactttaatgacagcttgtgagagaccctgagacagaagactcagctaagccaggcccagattcctgacccatagaaactgtgagataaaaaatattgctttaaaccatgcaattttggagtaacatattatacaacaatagacaactaaaacaccctctcggaacattttctcttctttttctctcagccagaaattctatttctactcaggtcttccaatggttggctctatcatgcagcctctcagcccagatgtccccttctcaggccttattccctagctaactctacactcagcccattactctgtttcattatgtttatccacttttaactgctggaaagattttatttatttactgatttacctgtcttctctatcgtaataaaagctcctcaagaagaggaaccttctcagatatgttcatggcggaatctcagctcctaaaatagagcctggaacaaagcccccttcaataattacatgttgaataaataaatgaagaaattatgaatgaaaaggagttgggggtgggggaggaatggagatgctcttccacatgatttttttaaagctctaggacattggaccagcatttgctctcctgatttatcccatttgtctgcttgagtacatttaaattttgaaggaccccaggttgtttgtttctttcaaagattaccccctaacttcaattttcctgcttgagttttttgcagatctcagctgaatttcagggtggcaaaaacccacatcctcttccgctggctccacctttctcttctcctcttctgcaacccaccgactagtttcaacacatctttccttctaagtgaagagcatttaaaagattgtaaagcttattgaactcttacaacaccatatctttatttgttaagtaccaatgactcaaaaatagagtagtctctcctgaaattcatgtggttttacaaattacggaggaagttctaggctcagtgtggattgccaagtggtgaaaatttgttatgtatcttttgcaaggctccgttttcttcctttctactgtcattttgtctgtagcttgaaggaatagagtgacttatatccccatattgtcacagagaatagagaataaaagatcatcccatttttaagggcccctccaccgaaaaataccagaggattttgtgtttgcttcattcttaaatgcggcccataaatgagaacattcatccaaatgtccaaataatatttgaaaaggggtttcattgagaatttcattagtaattggggatgaaaaataaaaacactattacacattccaaaaattgacttagacgtgaaagattagaaattccaagtatgggccagaatgtggaacaatgaaaactgtcatacttctggtcaaagtgtaaactggcacaagtattttagaaaactatttggtattatctactgaaatttaatataatactatgacccagcagttctaggtatatatcctcagaaatgtgtgcacatgtggaccaaaagacatgtacagaaaagtttatagcagtattagttatgatagccccaatccagtaacaacaatagttctagttcaacaatagtagatggataaacaagttttggtatatttgtacaatggaatgctacataataatgaaaacaaactactactatatataacagtatggatgaatctcaaagatataatgtagaacaaaagaaactagacacttgaaaatgcctaagttatgatttgatttatatggaatccaaaaaaaaaaaaaaagacaagcaaacttggctacctttggggagaggggaggggctggatatggggtgatataggcgctcccagagtggtagcaatgttctgtttctttgacctgggttgtggtcagtttgtcataatttagtgacttgttcacttaagatttgttcacttttctgcgtatttgctatagttcaagtaaacagtttaaaaattcaaaaaaataaaaataaataaaaacaaataaatgaagacatacatcagttcaataccaaaacatcagtaacatgtctggccaaaggcaaggcctatcaaaatgggtaacaaagcaaaatcttccctctgccgttttcaagatacatgcctataacaaaatgcacaaaatatagtcaaagatattcatcatacaatattcatattagcacaaaattcatttagatctaaatgtccaataaaagaggatagtgaaagtactttacagtgtatttctacaacagagtatgatatagccagtaaaactcgtgttctcaaagacttaagtaaaatgctcctggtatctcggtaaatgagattcattcaaagtgaaacatataacctcagccctgtttgtatgtgcttacaccttctaaagtaatttttgtcttcagttgctctgttttcctttctcccttctgtgtagcttctgtcctcccatccttttcattgtcctccaaaagaaatctcattctcagcagccatgatttcacgagacaagattaggagaatgggtagagaaagactggaatgcaaaggtagtaaaactttagataaaattctttcttattgggtgtggtttcaggttctcctctattatggaaattctagataatgagcaaagaagaaagcaaatttaaaaggagaccagatacttatggacataaagatggcagcaatagaaactggggactactagatgggggatggagggagctagaggggaaagagttgaaaaactattgagtactatgctcagtacctggatggtggaattagctgcaccccaaatctcagcatcatgcaatatacccaagttacaaaccttcacatgtacctcctgaatctaaaataaaatttgaggaaaaaaagaaggccagagaaactcttccttactatatttaaaagaagcttaaaatagggtaagaaagggttatgggagacttataaagaggtgaggataatttggggtaggttggagaatccagaagctatcatatagattttgggtatggaaaggtaggagagtaatagaaacaaaggtttaggaagttttaccatgggttatgaattacattccacttaacattatttgaagaccaaaggtaaagcctgtatttgtcctaataattatctatggctaattgagtagcgattttctgagtatccatgaacacatgaagtcctagattatattaagagcataaagtaagacattgaggtcatcagaaaagcttgtcatgaaaaaatccagcactgtttatttctctacagtggaactagaattaatttatattttgttctttatatttttctcaattttttccaagctttcttatataagaaagtgcatgacttatatagccagaaaaatgcatatataatatatattattcagaaaaaagttccaaaaaacttactattgtctgtcccttcaattgctgctggaagaatgtttgatagcgaagcagaaaaagaaagcatggagaaagggctctctcagtatatggggtggggatgggagaggaacagccgtggaacccctcccaccatggccttggccttgttcacaggagagcagtttgccctaagtagttttgtagaaggcattaaaaaaattgcatcaggctgggcacagtggctcacgcctgtaatcctagcactttgggaggccgaggcaggtggatcacgaagtgaggagtttgagaccagcctggccaagatggtgaaaccttgtctctactaaaaatacaaaagttagccaggcacggtggtgggcgcctgtaatcccagctacttaggaggctgaggtgggagaatcgcttgaatctgagaggcagaggttgcagtgagctgagatcatgccactgcactctagcttgggcaacagagcaagactcagtctcaaaataataataattattataataataataattgcatcaataaaacagcaacttgccatagaaatagttatgacagtctgttttatatataatcaaaagatctgtacacagaatttgatgtgaatgatgtatccacagagagccagtaatttaagtgcacccagagatgactgcctgccctgattatactcctgcagatgctgccaggggaggagcagtgtggtctggaaaaagcatggacttgggttctctgaagttagacaagcctagattggaatatcagttttgccacttactggctgtgtgacttcagtcaagttattaattgtttttgtgcctccgcttctccatctgtaacatggatttaattaagtccatatcacctgggtgctatgaacaataatattgagaaatgggattatataatatacatttaagcacctagtggaactctgagaagtaagaggtgcccaattagctttatccttactgccattcccacttatagctcccaccccccaccacatctcttctgccctgccccaaagttctcaaagcaaggggggctgggtggggataggaggggttggaggcagggaaggagtcagggagggaaactgactggaaagattattttatcataaaataattttcctgccaagttccccacttactcctggatttatttttccttttgtgcatagataagggtatgtgttagcgtattcctgtctgaatttagaggcatttcttaaaaagtcatccagcatcatattacattagttcttaaactccacatacaaggaagcattccagagtactcatatgtcttgggatgtaccttttatcaaacaatcaagaaattataataagcaactctgatataatctttatgaagtgccaggaacttgtctaaattttttaccatacaaagtaggcgccatgacaatcttcattttatgcatgaggaaattgaggcacagagaggctgaataacctgaccaagattattcttcaggccaatgtcaagtctggatttaagctcagagcagaagtcaagaaagtgcagctggtgggccaaatacagctcatcagatattgtatagagaagacttctgtagccagccttctcctcctcaaggccacctcatcatcactccctttctctgttactaatcctagtgttctgtttttatagctctcagaacccgatcctatttatgcactgacttgtgtatcatcgtgtatacatacatacattgtatattgtatgactcaagttaaaaataatttctacctttttttctcttttcccacactacactgatgtaagtttttacttttttaaataaatatatattaaatacagtccctgactcacaatggtttgacttacaattctttgactttacaatggtacaaaagtgagatgtatttggaagaaacagtacttcaagtactcatacagctattctgtttttcactttcaatacagccttgaaaaaattgagatattcaaagtttattataaaataggctttatgttaaatgattttccctagctataagctaatagaagtattctgaatactcttaaggtaggctaggctaagctgtgatgttcaataggttaggtgtattcagtgtatttgtgacttaaaatgatattttgaacttcaaattaatttatcaagacatagccccatcataagttaaagaatgtctatagttatacaataaaattctcaatgtttccttttggccgtccaaacctaaaatatgtacaacccatttcttcacaaaagaagtttgctgctcccttatctagtgtatcttaatcactataaatattccctctcattacatgcagtttcaagcacaatctgcaacccacagcaataaaattatcagttatcaagcaggaaggcatcatcatcatcatcccttccactgtacagtcggtttagggtaacacaagcagcattgtatgcaggcatggcctgccaatcagtgacacaaatgctctgggctctcaggggctatcagtaactaggataggtgctgaggcaaatacagtggtagatgacactaccttcatcctgaagatttaccatcaaaatatcccaaaatagtgcattaagagctatccaagtctttaggtgaactaaaccttcagccaaaatattctctagtagattatcaagccacctagtgagagagagaaatcagacaaacataaatttctgtgtttggggggaaatttgtcactcactctgtgcccaatttcctcttccacacaatggggttggtgatgacatctacctcatggagtcattgtaggatttgactgggacaatagatgtcaagtagttagcactttaagtagttgagacataaactctcaataaatgtctagtattaccggtattgccccagaatttcttagtggtagaacaaagaaagccctctgtagaaaggcttcagcagggttatagtcaccctgaaattgcactaaaattttatatttaagatgcatttttctggggagagtttctgtaacgtacttcagattctctgagaaatctggaactcagaaatattaagtgccacaactcaaaatgatatgaactaggggagatacatgaaggcttttgggagaaagatgtgcgcatccccagacatgtcccctctgatgcaccacagaaacctgtcagttggtactgatctaccctcctcctcctccttctcctacacacacacacacacacacacacacacacacacacacacacacacttcatcctactctccagcattcagggaagaaaacagaggcaaatgttgtgagctgcatccttgcagtcaactgctcttgggacttctgtagccagtcttcctccctcagtgccccctcatcatcattcccattctctgtcaactgatcctagtgttcttttttatagctttcagaaattgatgctatttatgcagttacttgagtatcattgccatctactatttgaatataggctccatgaagataggaacttggtccgtcttgctcatgactgaattctccgtatttggctcaatagacatttgatcaacaagtaaaagagaccccaaaaatccccagaaaatttcagttcagcttgcacatgaacaatgaaaggcagcttgagaatcttgactctactgtgaatgccaaatagcctgcttaaccaaggttgtccaaattcagctaagggatgcatcaggatggacaccatgagtgttttgtgtggaaacagagtgagggtttgcctatcattaagcaacttgtgagtaacgagtgtataacatccaggatgttttgacatttgggcttcttacatctgactgctgccacctcgggccagtacataacttaggggtagggtagctacgcttatagacctgaaattctagtataataataagacaaaatatctgaattaagacagtcataaaaataatcttgggtttcttttatttattcatacattcaacaaatattcatggagccttgttatgcactaggcactgggaactgagctgtgaacaaaacagataaatccttatacttgggatatttatatccaagtggaggactaatgagaaatccttctcctcccccagtcctattctctagagcccttcaatatctgatttgatcaaacttaccaaaaatactttttgcacaaggcagcacacccacatgcccctcgtatatagacagtgcatcaaatgtgttatggttgacagtagttgttctcaaccaagggccatgttgccttctctctagaagacatttctcaatgtctggagacatttttagtatcacaagaagggggatgtgctaatggcaccagtggggaatgctgccaaccattctacaatgcacaggacagctcctcacaacaaagaattatctggtcccagattacctaagtgctgaggttgagatgccctggtctacagtcgacatctaatagccagatccaagcagcagttatactttccctgatttcctttaatataataatatccagcattaaagagccattgtagacattgggagttttcccactgctcagaagtttttaaacacccttccctcatctaagaaacctttcacatccccaaggtggaaacaagaaactcactttcccattctcctttgtaatgctccacctcagccataccaaccacaatgagtgagagtatcactgaaaagtgagcaagatgagagagccgatgctgggtagaaactattttttggctagagatggcagtgatggctggaagtggaatttctgacatcctgagcccagaatgtgagctggggtctctgtcctcatagcatcgatggagtctgtactggttcattcccattgtatggtatggatgtaatccttctccatagagcctcccagtgtgactctccagccatcttggagacgtgaagcactaatattcttcaaaaaattcttttttcccctaaacccacttaatgggttctgtttgcattttttcataagaacaagcaaaaaaactgttcacagtcgataggaaatctaagaaggatggactcacaattcacctagaactcactcactggcttacactaatcatcttattttggggtaaaaaccaccttggctactaacacagtaaaacaccctagtgtgcgctgcatgaaaaaggtatttcaaagataatgtcttcatcaacagcaaaagaagaaaccttgtcctctgtcttatcaaatagccaatgccttcatccagccagtttttccccagctgtaaatacaacctatgtgagtttgtctctattttgcaagcacaggaaataggaatcataagccacctgtgctcctttcacatcaaattaaaagggagataaaaagattgaaaggaagcgtgggaaaacaaaacctccataaaaattttaaagtcagactgcccttgagccagcttgaacgttggtttaagtggattatgatgccagctttcgaggacatgctctaccatgggaatggagagacggtgcatggaagatgcaccaccttcatcttgtatttgccactgtgggagaagactgacctgttagccttttctgctcaccagttcatcttttgctgggagagagaattctgagtgcagaattcttcacattatccatgcagaactaggaaaattgccaaaagttatgggtctgtacagagttagtgtcacagtaagaatctcattgcccaagcaatagggtctaaaatcacgatcttattcaaagtaacagcgaccacttacctcatgcctcatatgtgccagatacttttcttacattatttttaatctccatagcaattatctaaggtagataatatctagagatgaggaaactggggctctaggagtatgcaagatttgtccaaggtctcacagcaatatcttagtagagtctgtctagaatcaaagccaatttgtctttttgccctatcatggttcatctctacttcactctaactccatcctaaaaaccaccttccccatccactatataaatgaatgatagcaccaccctttcagtaaaaggatctagacattcaccatctctctaccatcctagcagcaactgcaatgcttggaaaatagtcgaggattagtaagagcttgtcaaatgagacacagtttgttgttctggccctgacatgaaacaggtaatcaagtaaacgtatattttatatatagtcacttcactttcctagtcactaatttccttatctataagacaagggtattgggccaaaagtctagtcttaaaggttcctttcaagtcatttattgaaagtttgtctgatactttattttttactaaactttatatattccttaaatacacactcaaagaaacatatacaggtaaatacagacaagctctatctaatggtgttaactgtcacttagtatataaagacatcttctctcagagaaattggtcacatgttctttctttagacaactgctcatcatgtcctttgactaatcataagccaacagtaagaagttaagagtgccaagaaaaggtaactgtgttaagttgcatttgtatttttccaagtatttactctcccattctttcatatctataagaggattatccatccccacccactggcatgtgcgtacagtgcctccatgaggggcgtttatctgtttttcttcacaatgaatttatcacattccttgctttggccaatagaatgtgagtgggcatacgatgtgtgcatgtctgaacagaagtcatgaaacaattgcctggttctgatttatctcctgctttttttttctttggcgttaaattggtatgtgcgagatagaggttgatctttcaactttgacctggtattgagaaggcacctgaggcaaaaccagagctgatctagagttgacatacacagtggacatataaaatgaataaaagataaaacttttagattgtaagccactgtaatttggaagatgtttgttactgcagcataacctatcaaaggctgacttataaaaaatatttcagataccgttagttctcactgttcacagtagttatgttttatgaagtttccatggatactgaatgagcgaacagtgaactaatgttcctaggtaaaatagaagattaggttcccgtgagctctgggcaaaacattttcatcatccaagcaatacataatcttgctttatgtgtgtttctatgtaaagacaccttattcaatatattttgttgattcattaaaattaaactcatggccagcagcattatagctcatgcctaaatgaggcttatctaacatgtatatattttctataagacatttcacagtcttcttgactcaagaacactacacagcacttcagcactatgctgaaatggggccattttaaacagaaaaatcaccaccaacaaaaattagctgggagtggtggtgcacacctgtagttcaagctacttgggagactgaggcagaagaatcgtttgaacctaggaggcagaggtcgcagtgagacaagattgcaccactgtacttcagcctgggcaacagagtgagtctccatctcaagaaagaaaacaaaacaaaaacaaaacaaaagaaacaaacaaaaaaacacttcatcaaaaagcataaaaatggggaaaatgtggagctaagtagattgaaagggacacttattacaatgtgagagctgaaatgagatagcagaggctcaccttgtttaccctcagctaggatcatgtgtgtcaactgacttagatgttttgccacccactctaagcatgtccaagaatcactgtaaaagcaccatgagtattgatttggggatttaagtatgttttagcaagtaggtgagttcacaaatatagaatccatgaataataaagatctactctatttacatgtgccacatcatttaaccctcataacaatcccaaaaggtgagtatttttatcaccattgtagagaaaagtaagattctgacagtgaaataatgtgtccaaaggtataactaattcgtgataaagcaagaatttaaaggtttttaaagtcttctaggtttgcatggctccaaaatcacagtatggctcttttctctgccccatattgtctcccagtagaaggaaagttgacatgtggcaccaagactcaacttatctgcctggatttcagggccctgcttaatctgattgctcatggtattttccatctccattcaatccatgctcaccaacaccctcctcttcagtgaagagattctttcctcccattgtgctcagcctccatttttagttctactttgacacggaatatgctcttctgtcacgcatctggtccacattctcaaggctggttggagaagcctggagattatttggtccaatcttgatgcttccagctcagacccagagagggtcagtgattttcccaatgttagtgagagtctgagtaaggacttgaacacagatttgctgaccttggggcaattatccctgggattgtttcacttttcctcccaccccaaaagcaattacctttaaccttagagcaaacaaatgggcagaggaggggcgggagtggcaaaagcaaatcattaccactcagaggtatttacaaatgatttcaaagggtttttaggaaggagacagctgatgactttgtcagtcaaacagcctcctgttctaaaagaagccaacaaaccattgaagaataatagcagagaccttctggtctttgaattcatacagcacttttcacctctgaagctcaaggctccttccggatgttatctcattcttcttcccagatcactagtagagagtagttgcaagtattgtacccattttgctgatagaaacattaagcctcaaacgcagtaagagatttgatcagaatcagaaaaatcaacaaccttattctgcagaaccagggttaaagctgagaactagaatcaaaacttagccctttcaaatcttatcctaagccatagaaggaattgtctagaaatgagactgaaacaagggggaaaagaggactggcatttgttgaagatgtgttgtgtcaaatgtctcatattaaagatatcacaggtccttggtatccatcctgtgaagtagaaattgttatcctcactttatagacagagaaaagatttgaccacagatattcataacatcaaagccagcatcattgataaaactgcagaaagtgcctggtatttgcagaagactatgctaggcccaatgagcgtacagtgaagcacaagatgaagatgtagcaagaacttattatgcaccaggcatcagagatcggaagataactaagacatgccctccaggagtcaacactctatttgaacagagaagaggagcatgtggagcatacagagaataatccagaacccactctcttgttgagtactttctacgtgccaggcactgtgctcttcatggtagagattcaacagggaacaaaaacacagttcttgccttcgtgtagctcactatgcttgattgtcaatgtattccaagggatgctgcatacccaaagattcggggcaggcacaggagaaaagggaggactttgactttgtgtgctttatcttaaaactcatgcaaatttaacatcctactctataaaatatggaggttcattttaacattactatatatttcctaataatcaagttaaattatttgccttttttttctttatcaacctttccaaaacagttgagacttcagggtaccattcacattgactctggagcataaatgaactcctaacaaactagagctataccatcaggatgcacaagcctgatttaaaagtgctggcaaataggcacagtcctattctgtcgacaccactttgattcaatatcgttagtgtctactatgtgctattatgcagtgttcagtgctgggggaaacaaaacaaaacaaaacaaaacaaaaacaaccaaaaccaaaaactgggataatggagaagaaaacttcaaattatttctattcaatgaagttggagctatggccaaaatagctagaggggccaagaggggccacccaaaccagattaataagttccaggttcctctcatatttgcacattaaaagcacacaataaatgtttttggctgatagagtcacatcagttttgtcttaatcttacaaaatatgtgttccttataaagcacataatcacacccagccttgcagctccacgtggggggcacaaagagggtagggctgctttctggacccaggagtctaattaactctctcatcaagccagtccagttgggcctccagcctcctaccccacccccaccttggatgtgccctttggcagcatttacaggagtggtctccctctcattccccaagcaaagaaagtttctcacatggtgatctatgaatgtgtctgctcatgacttctctggaagttaattctattaccttgtgggttggattgacaactgccaaaggatttcctcacgcctctgggacatggacctcctggcagtgactctttctacactgctgcaacctgacaaatttaaaaatgaatcattggccagacgtagtggctcatgcctgtaatcccagcactttgggaggccgaggtgggcaaatcacgaggtcaagagatcaagataatcctggctaacatggtgaaaccccatctctactaaaaatataaaaaattagccgggtgtggtggcaggcacctgtagtcccagctactagggaagctgaggcaggagaatggcgtgaacccgggaggtggggcttgcagtgagccaagatcatgccactgcactgcagcctgggcaacagagcaagactccgtctcctaaaaaaaaaatgcatcattacattctatctacatcaaaatcctttatttttccctccttgttatgaactagcccagaagcctcagacttactgcactttctattgtcagtatattcagattaaactaacatttactgagaacccattgtgtgtctctcatgtttataaatattatttaatcatatgtcctgattagccctgggaattaaatgagtgttaccattttcaagatgggttctctgaagcttaaaaaaataagttactaagccaagtctccaaattccagttctactacttttccctctcaacagtgttgcctaaacctttggatggatagatggatggatggatagagaaagagacaaagcaggaaagagaaaagagaaaggcatatatatatttttttcttcattctgggggcccaccctgaaactactgaatcacagtctctagaggttctcaggcaactagcccagctgtttttgccaactggaatttatgagccaccgcaagagaccacatgcagcttcatgtaaaacaaattatttttaagcacgcagactgagcagtgatatgaggagtgcacaggagtgcctacgcctactcctggtctccatgagtctcctttgcaaagtcaagtattacaagattctagaacacatattgcctgccactgataatttagttgttcagcaaacattcatttgttgagttgcacgccagacactatactagatgatgggacaactaaagggtaatgaacagttctgtctctatgtaaaaataataatgatgatgatgatgagatgggacttcaattgaggaagtgccattggggaggtatgtaaaaagtgctatggaaaaaaagcaacaggaaccccttgatagaaaaaaaaatgctggtgggggtagggatttctgcctgtgttcttcagaatggggtatgggaaaatctgggaggaaaagaaatttaagtaagagcagagactttgcaaaatttgttgtgttgacttttcctcatgctgcttcccctggcatgggaagtcattagctggataagagagacttcacaagaactgcaatgaatcaagatgtgctggttttgttttgacacatggaattcttagggatttgatgttttttttcccagtcttctccatcaaagttgttttcaaccagtcctgattggaccgattgactcatcctcagatatcatagttttcccactacaaaagcatggaactgatgccaataaacccactccttattcccagagggctagggtgagtccttgcagaggggaattgctagggatggcacctggcagaaatagaccatctgtctttcctccacaattatggtccctgccattgtgaaggaaacatttacctcctcctcaccctcaggcccccttttcctgcacttagggtctcattgcccctccccaccctccgacaagtagctggtgctttctccttgacctctgactcactgtggggagaactctgcctcaagaaacatcttttcatctccctctctagctccaactgtccctttgcctccatggggagctccttgctctctccttcggtatttctctgagggctcactcagaccactccactccacctgccaccagtggtctcatactgaactataaatctggccatcctcctctcctgtttggtatcttccatccccttcccatgtctggtagatgatgatggcctcaacccccaggtgactcttggaccacgtaccatccatgcctttcccattgagctactgtggaaagcccatgcttgcttctaagtgctccatggtttggtttggtttggttttctgtcatgtcattctgtctgcctgggacaggatttctccctatcaacactgagagatctcctgctgaccctgtgctcaatgattgtgtctgccttgtccctagcatccagatcaacacctagcaaataaacaagtgctcaagaatgtatgttaaatgagtgaataagctagttagcaagagagtgaaagagaatgaatgaatccttggagagcgcaggccttcactgtgaggcctctagaaccctaagtgaatgacatactccttcctctgggctaacagcatgtgaaatatccctctgctgtaacccttatcacttttacgatgtggaatctcaggctcccttcttgggcacatagtctgtatcacattttgtatcagcagcacataatagccatgcaatatataatcaacaatttagttttacgaatccatttgaccatccacattccccacatctcctcacctctttctacagcattttctgactcctcacttttcattctatctctcttaccttcagaaaattccagaccttgctcctttaccctagtattactgcctgaaatgccctctcctgcatcctttcctgtgtgtatttaaaacctaaactcctcacagctaacaaaagaccatcctcccaagcctcccaggctttcaagaacattcctcctgtggctcccaagtcacacacatgtctctttgacaataataatataaacaatcatttttcagtaccagaatgaatctcaagtgctttatgtattacataaattcagatatatctctttttattgtacttctctttactgtgctttgcagagatattgcattttttttttacaaactgaagatttgtggcaaccctgcatctaggaaatctatcagtgccaattttccaacagtgtaggcttatttcatttcactgtgtcccattttgttaattctttcataagttcaagtttattgttattattctatctgttatggtgatctgtgatcagtgatctttgatattactattgtaattgttttggggtgccacacactgcacccatatcagagagtaaactggtaaatgtgtgtgttctgactgctccactccaaccagccatgtcccccatctctccccatctcctctggcctttctattccttgagtcagaaaaaaatattgaaattacgtgaattaataatcttacaatgaactcttaagtattcacatgaaaggaagggtcacaagtctttcattgtaaataaaatactagaaatgattcagtgagccgagatcgcaccactgcactccagcctggatgacagagtaagactctgtctaaaaaaaaaaaaaaaaaaaaaaaaaaaaactagaaatgattaagcttagtaagtatggcatgtcaaaagctcagcctcttgtgccaaacagccaagttgtgaatgcaaaggcaaagttcttaaaggaaactaaaagtgctattcccgtgaacgtattaatgataagaagcaaaacaatctttttgtgatatggataaagttttagtagtctggatagatcaaactagccacaacattcccttaagtcaaaggctaattcagagcaagcccctaactctcttcaattctgtgaagtttgagaaagatgaggaagctgcagaaaatgagtttgaagctagcagaggctggttcatgaagtttaaggaaagaagccatcaccataacataaaagtgaaaggtgaagtagcaagtgctgatggagaggctgcagtaagttaaccagaagatctagttaagataagttatccagaaaatctagttaagataattgataaaagtagctacactaagcaacagaatttcattttagatcaagcagccttatactgcaagaagatgccatctaggactttcgtagctacagaggagaagtcaatgcctggctttaaagcttcaccaggcagggtaactctcttgttaggggccaatgcagatggagactttaggttaaagtcaatgctcatttaccattcttttttttttctttttattattatactttaagttttagggtacaagtgcacaacgtgcaggttagttacatatgtatccatgtgccatgttagtgtgctgcacccattaactcatcatttaacattaggtatatctcctaatgctatccctcccccctccccaccccacaacaggccccagtgtgtgatgttccccttcctgtgtccatgtgttctcattgttcgattcccacctatgagtgagaacatgtggtgtttggtttttttggccttgcaatagtttgctgagaatgatgatttccagcttcatccatgtccctacaaaggaaatgaactcatcattttttatggctgcatggtattccatggtgtatatgtgccatattttcttaatccagtctatcattgatggacatttgggttggttccaagtctttgctattgtgaatagtgccgcaataaacatacgtgtgcatgtgtctttatagcagcatgatttataatcctttgggtatatacccagtaatgggattgctgggtcaaatggtatttctagttctagatcatttactattcttaaattcttagggccctttagaatcatgctaaatctactctgtatgtgttctgtaaatagaacaacaaagcctaggtgacagcacatctgattacagaatggtttgctgaatattttaagcccgtgcttgagacctgctgctgaaaatcaaagattcctttcaataatatagctacccaagagtcttgataatgcttttagttatccaagagttttgatgaagatgtgcaaggagattaatgttattttgtgcctgtgaacacagcattcctgccatagcccatggattgagaaataatttgactttcaaacctcataatttaagaaatacatgttataaggctacagttgccatagatagtgagtcttctgatgaatctgagcaaattaaattgaaaactttctggaaagaattcagcattttagaggtcattaagactatttgtgatgcatgggaagaagtcaaaatatcagcattaataggagtttaaaagaagtggattctaaccctcatggatgactttgaaagctttagtggaggcaagaactgtggatgtggtagaaacagtaagatagccagaattagaagtggagcctgaagatgtgacagaattgttacaatctcgtgataaaacttcagtggatgagaagttgcttcttatgagcaaaaaaaaaaaaaaaaaaaaaaacaaaaaaatagtttcttgagatgggatccactcccagtgaagatgctgtgagcattgttgaaatgacaacaaaagattcactacaatcaacttagttgataaagcagcagtagggcttgagaagattgacttcaattatgaaagatctataatactatgggtaaagtgctgttaaacagcattgcaagctacagagaaatctttcataaaaagaagagtcaatcaatgcactaaatttcattcttgtcttattttaggaaattgtcatagctacctaaccttcagcaaccaccaccctgatcagtcagcagccatcaacattgagacaagaccctccaccagcaaaaacagattatgatttgctgaaggcccaggtttattatttagcaataaagtttttttgtttgtttgtttgtttttgagatggagtctcgctctgttgcccaggctggaatgcagtggcatgatctcggctcactgtaacctctgcttcctgtgttcaagtgattctcctgtctcagcctccctagtagctggaactacaggcgcccaccaccacacctggctaatttttgtatttttagtagagacaaagtttcaccatattggccaggctagaactcctgacttctggtgatcctcctgcctcagtcttccaaaatgctgggattacagggatgagccaccacgcccagctagcaataaaatattttttaattaggatgtgtacatttaagaatgggcatggtggctcacacctgtaatcccggcactttgggaggccgagacgggaggatcacttgaggtcaggagtttgagatcagcctgaccaacatggtaaaaccctgcctctactaaaaaaaaaaaaaaaagtttttaaaaattagctgagtgtggtggcatgcacctctgtaatctcagctacttgggaggccaaggcaggagaatcacttgaacttggaaggtggaggttgcagtgagccgagatcgcatcactgcactccagcctgcgtgacagactgagactcagtctcaaaaaaattaaaaaagtaaaataagatgtgtacatgctttagacataatgctatttcacacacagtagactacagcagtgtaaacataacttttatatgcactggaaaatcaaaaacttcacgtgacttgctttatggcaatatttgctttattgcagtggtctgtaaccaaacccacaatatctcaagttatgcttgtatatacatttttataatctcctacacataatatggaaggataagaaattgaggcacaaacaggttaaatatctgtcttaaagctgggcaactggtaaatgatagagccagtatttaaacttggccattattcattcatttattcaccgattcatttaatcattaaacaaatagttatggagtgcccatgaggcattatttccagacttcaacactttgtggtattattcacttggcaaactaaactattctgttgcttagacaaagtgaaggcagacaaaggaggaaaaccagaagggattacaggctctgaatcaaagaaaggcagaaaaatggaagagtgagagacaagtttcagtttcaggtgagatacctgaaaggggaaaaattcctagggaatagctttcctcctccagggctccaggttgaatgaattccctgaactcaggaacaagcgtcaagacttgttttattggtatttacaaaaataggttttaaaggatcagtgatttgcagattccagttggatgtgtcacacacacacatctctcattcctcacgtctaagagaggggagaaggaaagacacggctttgaaagcctagatattttgaaaggggtgttttgggatttcagcgatttctttttccagcagcaactgtatcttggactgtggtgtatctctctttcccatctcccaggcaggtcagttaacccttttgggctgtgttcaatcagggttagtgcgtaataaagatcctctgcatttagaattacctttttttaacctctaaaccctcttcaagaggctgcaaactcttcaaagacagaagctgctgtacggatttttcacctgcttcagtgcctagagaggaacaagggagagaaggggagagaaaaaggaggaaggaagaaacaggagcaagggatatctacttggtactttacaaggtagtgttaatgaaatacacttaaatcaagagttctgggttcaagttccagtcctgacggtgcctatacatccttgggtcatcactcagactctcagagcctcaatttcttactctgagaaatggaaataataatgcctacttcaccagatcattaggagaattaaataaggtaatgtattgggtaacattttgtaattgtgtgctactgtgtaagttaaaattgtatgaaggtttaataaaaggaattgttctttaagacttcaaggactttctgtcagaagttgaaactagaggatttaccaggatgtttctggctctataggggtcatgtccctttccaagatgcctcagttgaccagtaaacacattcttttcaaggcttaatattaaaatagtatcaaattctgatctaatctggatcagaagagaaaatactccaacctaccattgtcaaccgcaacactggttgtgtttaatggtgatggaattctgtcacttccaccaacactttttgagcattcgctgcatgcggggcattctggtgtatactgataagaaacaacaaccaaagataattaagattgacctcaccctccgagatgtttacagtttactttgcttccctaagcttcagtttttccctctgccaaaacagaagctcggaagagatgtctccaagacactttctaccatgaccttttgaatttcataactctctaattccagaattcaaagagtgactgttttacctcccactgaatgattcctctgagaaaaaagctctctgctagctaagctagccaagacaccatacctgaagataatacctaaacttctgtttgtgtcccaggaatcccaaattgatgtcacaccaaaacaatgtcatttatacaattcattatatgtcactcattaatacatttgcttagttgattttcattaaaatcctgcagggagcattattacactcatttttcaaaagagataactgagtcgtaaaaggattaaattatttgcccaggtcacacggaaagaaattaacgtggccacggcataaaaccagattttctgtttttaaacatatttttttcgctgacctccaccctgtaagagcttttattaccaagcgattgagaagcacaggctcagggacactgaatttgaccaaagaagccaatagaactattccaaaaacctatggttccccctaaagcattagaaagactcagaacgggttaagtgctccctggctcattcccaacagacactacattcacctgtgcttgctctgaaataaatcagtgtccctttctgctgctgctgttgtctggaaataatgcaaatgcaatgggcctttactgacattgtgcttccctggaaggatacacataataaattatcccttaatactgttaaagagacattttcctcttactcaggagcttttggggttggactgggctactcacccagcaaggaggaggacatgtgtcttgtcactggcccggttattcatgtggcctctcattgctccttggctcactgcattgcaagattcaaggatgcacttccaggcctccacatcaagtcataggacttgccggtaacctagattggttttctcatttgtaatttgaatttattttatgttatgcatttgtatgtttatttattcggatgctcagaagctgaagataactagtgctcctggtccatgccattcatcaattggaagaatgccaagctgtttccgctgaggacagaaggcattggtctcccctgcaggaagccactgctgctccttaattgtttgctagaggaagaatcaagggtaaaatttaaagtaaatggctggccgagttgcactaattcatcaaagcatgtttcaagtcagtagtcagagcatgcatcagcccccggcgccaccagcttctacgagagtggaaaagccagcagacctccgagcagatgaaatcattaggaggcattcagcagggcttgaaaagcaaagagagaggaggcggggatttctctgcatgctccctttgccacatgggaaacaccagctgtctgtgacctagttatccaagaaaggaaacacggaagagaacccacaaaactgtttgctacatgagaaccccattctccaaagacatgctggatgttgagaaaacaattagcatcttctagtttgactctatttttttttttttttgcttagagatttttggtagcaataaagacaagccctattacagtagcctaagaaaatggaatttttagggatagcccatggataggaagtaaaaatcttggttcatgaaagatgggaagtaggaactggaatgtttttggaaaatctatcagcatctcacctctttctcttgctctctctctctcattacatggccctttatttgtgcaacaattcattttcctacttctctatgaaggtctctcttcttgcttttaagcatgaagccactcattcattcagtaaatatgtatttaatgctaacaggtgctggcactgcccaagatgccagggctacagcaatcaacaaaacagacaaaatccctgccctcgtgaaatgtacactgtggtgaggcaggtgaggcaggcagaaactgaacaagataaataagtaaaagatacagtgtgcaagagggcagtgagtgcttggaagaaaagatcaagggaggagaagtgaaatactggcaaagtagttgaaatcttaaataaaatggtcatgggagaccccactgagaaagtcatttctgagtgaaacctgaaggaagtaagaagttagcattgcaattgtctggggcagagcatgctgaggagagggaatagcaggtgcaaagggcctggggtggttcaaggagctagcagagaggccattgaggaaggaggttaagcaagtgagggggattagttagaggtgaagtcatagacctgggggcacagatcacaaagcgttttctaggtcatagtgaggactttggcttttactctgagtgagatcagaagatcccaaaaggccaccacaagatgatattcacgtggtccacctaatccagtagtcagtccttattgctaacttgcacacagtcaagctcccttagtctccaaaagaggagatccaagcaacgatacttcatgagcagtcggcttcgagagtcatcctgagttttcaaggctgacacaaatatcagtctaactacgcagtccacctgtgtaaatatttggggaatagtggatggttaaggaagagacctagtatgagataaagtgtccaggccctgcacacatttgggtctcacagaattaactggcaaatgctagtaagagtatcaggaccttaggaaatagagattcctttagaaaactctaattcccagaaagattttcacataagaccttcacacaaagacaaaattagaatgtgtgttctctcaccatctccttatccagagagtccttagatgtggcagaaggacccacaagagttgtcagaggcagttgtgaggggttgccagtcatgttagtattaatagatatcatgagaagttagacacgtttttgaataaattagaatgaattaaatattaacccaaaatgtcattatgctcactccctaccctcccactatgctttcctgacagagaaagaaagggtggcactgtggctggaatacagagcccacaggacttcagcatgtcctacacataaacctcctcctttttgtgacccttggtggaaaaagtatgagagccacgtatcttagaaataactgcctttcctccctagatgcctgccacaaaaacagacatgttctagaactttcttctccctagttccaagaacactgaactcatggtaatggccaaacaatgatctttttctaaggacaatgtcagaatgctttactgtgccattttcataatcagacacaagaaagttaccatctgtagttcagccatagcacttcacatgaaagaggaaatgactaaatatatatttacatagctctcatgaatatgcattttgtaaatgcacaaatatgtacaattacttgatagactgttttggttcctatctttcgaatatttaatacaactgtaaatgtttaattcatatttactgagttgattgaacagtttctaaattatcccaagccttgctggaaacaagattgaatgtttccagcatatttgcacactaggatttggccaggagaatccacactaactggttaaatgatgattttcttagaaagcatattgaataagctgctggtgaaatcatcttgctgaccacaacatagagggacagcaccagatctaattccaaccatgacctgcagagcctctcatgactaggcccccctgatgcctctttgcacctctgtctgcactgcccctcattgctaagccacaatcaacccatgttccactttcagtcctctgcaccagctgctccatgtgtccagaatgctcttcacatagacatcaagcctttgctctaatgtcaccttttcaatgaggtctatcttaacctctgcatttgaaattgcaatcccatcatcccccagaactcctgatatcccctacactcccttatacttttttgtctatagcaaccacccctcaccactttataacatttatgctttgtagtctgtctgtgtccactcactagaattcaaatatcacaaaagcaggagtccactttttttttcattgaaaaactccaaatcctagaaggaagctggcatttaatatgtgctcaatagacattagaggaagaaaagaaggaaggaaggaaagaagggagggagggagggagggagggaggaaggaaggaaggaatgaaggaaggaaggaaggaagaaaggaaggaaagaaagaaagtcaagagacctgggctcaaatccagcatgggaataagtagggaggaaagaagggaagtcaagagacctgggatcaaatccagcttgggcatgaggcaagaccagagcagaggagattgtccttgagctcaggtgacgcccatctggccaactctccacaaagctgggtcacactgtttagaggacagttcttggcagtagtagtagtgctggtggggagggctttgtcattatattgtgtgcgtgtggtgtgggactcttccttagttcagctaaagacaagctccctgtcacaaggccatgaaagattaggctcgcagacaatttgaagggtgagaataatgggatttattgagcaaaaggaaaaatgggggcagaacagggacgcagcagagccagagtcttcctagtatgtgcttcctgcttcacaattgaatcccagctaccacccaggaataggaagggccaggctcttccccactgcaaatggtgagaacttctgtggctccaccccagtgtgcactcctcccagtatgcaggctggttggagtttctctgtggaccctttcccatctggctgtctcacatgcaaaatgggagtatttgaatctccttctgagagtaatctgaaagttaaatgaggtaaagcaagtgaaaacatgctcatgtattaggtctagggaggaagcaaaaaggaagtagaaggattctcctgagtaggggataattcttttagggagatgcttaccccagaattattaatattcaaggaaaagccaggagcgactataaaacacagctcatcattgcagaccaaagacaaagcacctcaaaatatgtctactacagtaggcatattttgcagaaaaaaattagagaaaactacatctccttggagtaaagtgccacaggtatccaataacagaaaataggaaaagacatcattgcaaacataggaaaatagtagatgaagatgcaagatcctaaaatgtgtattttgggcagtttgtgacagatcaagtcacatttctgacagagtgagaattaacccaaagcaaaatcaagatatcatttaaatgtaaaatggaatgatagacacaattgaataggacaaggagataatgtgaaaataacagagaggcaaaggacaaagaataaaaattaaaagaaggggactcactccagaaccgaaacaagataccccactaattctcctgtctgcagacatcaccaatattctggtaaataaaattaatgataattaccagaatgacactatagttatcaatgaagagtgtttgtagcttgtcatacgtgtgtgtgaatgttcaaggggatgtttagtaaacggtaaatagtgtgggttcagttctgctctgctaatcctatatgccataattacaaggtctcaaataagtgcaaacccttcaattatcaggaagagatatctgagccaagtggaaccaactgacagtagcaatcaaaattctatcgcccacaagggtgtgggggaaagaagtactttcaaaactttagtggcattataaactggctaaaaatatatatacttctgggaggtaattttgcatacttgcatatgcaaatattcctgtaaaaatgtgcctaaaatttattcctctactcttctccatccacctctgtaatacaacttctgggagtgcagtctacagaagtaatacaaagatcctgggaaaacaatgttattttgctcataggtgtttatgaccacataatgttttatggtaaaaaataaaaaacaacatacataattattgtgggtatagtttaggatttcaagatgctttttttcatttttattaaacttggtgtgtagacatataatttacacagagttttatacattttaaaaacgtaaatttttagaaatgtatacagtcatgtaaccattgtcatcaaggtatgtaacacttgcctggagtcccagctactgggaagagtgaggcaggaggagcacgtgagcccgggagttccaggctgcagtgcactgtaatcatgcctgtgaatagccactgcactccagcctggacaacataatgagactttatttctaaattattttttaatttaagaaaagatatataaaacttgcatcatctcgaaatttccttcaaatagtcaatactcacttcctgccccagggaatccgctggtatagttatttccctatggttttgcattttctaggtcactatataaatgaaatcatacagtacacaaacttttgtgtatgccttttttcacctagcatgatgcatatgagatttatccatgttgttattgggtaatattctactgtatggatgcaacaaaatttgtttatctattaacgattcattttattttgatataattattgattgtcagtgcataattattagttatctaaattatagatttgaaagatagtactgagaggtcttgtatacccttcaccaaggttccttctatggttccaccttatacaactatcatacgatatcaaaatcaggaattgacagaggtacagtgtgtgggggtggggggacaggcatgggcatgcacaaatttgcatgggtgtgcgtgtgtagttctatgtcattttatcacttgtgtagatttctgtaagtgcttctgcaagatacagaagtattacctcccacaaagattattatactactcctttatagttacacacccccatgcttatttccgtttgctttctcccaacaataatgtaattattttagacatttcctgctttgatatacttttttaaccccattagacagtgttatattttttttcttcaatcatcaaacatgatctagaaatatcaagagaagaaagtcttcagtatttgctggtatttttactctttctgttgttcttccttccttcctgatgacccaagacttcttttgtcatttactttctttttagaggaattctttctgagcacctacctcccttgctagcaacatattcccttagttttacttcatcggagaatatctttattacaccttcatcctagaaggatattgtctctggatatagactaattttggaaagacatagttcttgtctttcagcacttaaaaaatatgccattttcttttggcctccatggtttctgatgagaagtgtgttgtcatttgtatagttttttctctgtaaataaaatgttgtttctaattcactgatttctttttttttttttttttttttttttttttgagacggagtctcgctgtcgcccaggctggagtgcagtggcgcaatctcggctcactgcaggctccgccccctggggttcacgccattctcctgcctcagcctccggagtagctgggactacagacgcccgccacctcgcccggctaattttttttttttgtatttttagtagagacggggtttcaccgtgttagccaggatggtctcgatctcctgacctcgtgatccgcccgccttggcctcccaaagtgctgggattacaggcgtgagccaccgcgcccggccctaattcactgatttctagaatttttgtctttagtttttaaatgttttgctatggtgtgtcttggctatctcttatatcttgtactattgtactttggatttctttgggcttatcgtgttcagatttcttgggtttatcatgtttgaggttcactcagtatcttgaatctgtaggtttatgtgttttgtcaaattcaagaaatgcagcaattatttctttaaagacatttcagctatgccttctttctcttcttctgggaattcaacaatgttaatattcagtcttttgtattagtcccaaagctctctgaggctctgttcaattcttttccaattttttttctgttattcagatttgaaaattctccattattttgtcttctgtttcactgatattttttactgtgtccttccactttgttattgagactatctaatgagttgtttatttcagtttattgtatttttcagctctaaaatttttattcagttctttatatcttctttgccaataatttctacttttttctctttttttccaaacatgttcataatttctcactgaggcatttttgtaatggctgctttaaaatcattgccagacaattctagaatctctgtcatcaaggtaatggtgttctttaattatattttcttattcaaaatttctgatttcttatataataagtgatttttttaatggaatcctagacatttggattatgaaactctagatttcctttaaagtccttgttttagagggcttcctctgataccaagccagagggaaaagaatggcactatgttgacactatcaggcgaaggtggatgtccagatactccactcagcctccgctgagactcagaaagggggaggattcctcatgactgctgggcagaagtgggaattcaggcccctgccaggcctctgctggtaccactctggctggtagtggggtggatggtggtagggaggtgaggggtgtgggggagttggactccctgctgaccatcgaggatgaaattctcaaggtcctactttaccttttctgatgccacccaggcaagggggttgggcacctccttacatcttggtgagggtgaaagtcaaagcttcctgcacagccctggctgataagatggaatgggctactgtttttacaatggtgtttggtagttgatgtctgaaaatttttttttcttgctaggctgccctttcctggtcctctggctgtaagcaatcttgtcttgggactcttttcacttgcattcattgacacttccaggttgccaactcctcaagaggccagtcctgggatagatgagaccaaaagaaaacccaggaatcccactgtcatgtcatactctgagtcctgaggcctccaacaagttgccttcttttctccacctttcaatgtcttctcctgcttatattatacataatatccaaagtttttagctttacttatccaaaagaatgggaaaaaatatccatccattacatttcaaaagatgtttataagacgatgttggagcacagaaaaagaaagttataatattaagggaagaaaaatgatataaaatgttctgcatactacatgcaaaatcgcatctctactaaaaatacaaataattagctgggcatggtggtgcacgtctgtaatcctagctactcgggaggctaagagatgagaattgcttgaacctgggaggtagaggttgcagtgagccaagataatgccactgcactccagcctgggcgacagacacgtggtctaaaaaaaaaaaaaaaaaaaaaaaaaatctgcaaactgtggttactactatgcatgtattttttaaatcacagatatgcaagaaaaaacactgaaagaaaatatgacagatgttaatatgcactgagttagtgaagcagaaagagggataaagcaattctcttctatttcagtgttctatatgatagcaagattacattaaacatataaattagattaacaaaaagtattcagccactcagacaagaaaagctcttaaaaatgagacctgcaaagagatccatttcaggatggctatgctgctcattcataaaactacatcgtaaacttcacagtttatgaagtatataaaggacaggcattggggttgctttgttgaacaaatctagcagatatttgaatgagaagagtaatatagtcagtagaaaaaaagtgcaagaaataagtagagaaagaagggatattttctgctgaagcatgtattctctggcacaagcccacaataaattgaaattgacaccaacagttggctcaaaaataatcaactacaaatatgctcaacacataagcattctcttggacagaaccacaaagcatggtctgcattgttcctaacaactctttagaagtcaccagatgcagtttaagctacaataacatagtgaggtacaagttaattacatagttaccagaaagtcacagacttttttttcagtaataatgtagtaaataaatacatgctcactccatgggaaatggtggcaattattaagagcacacattcacaccatcatattgcttactgataactgtgcagttaaccaatggcagtgtgctaaaatggatatctgtgtttccctgagttttgcatgctacatgcgatgcatgtgaaaaccaagcatagggaatttcaagtatgaacttcagcgtgtgagtgttgtttgtggtccaatctccgtccccaaacatccccagaataaggcttctgctttttaacaatgtatatctattttaaccaattgtctagcgtataattaatgctctataaactctttgttaaatgcattcacagaaggtaacaaaagatttttgtgacacgagtaaaccaaaaggaacaaataaacttgaattactttatgtttgtgttggtgtttcagaaaagagctttggctttgaattcagaagttcctaatctgaataccaggtctaccaattattaattaaggaatatcaaatgaattacttgcagtatttgaatttcagatttctcaattataacaaggatgtaaagaggtttattatgtggctcaaataagaaaatgcatgtaaaaacacttgtaaaccaaacaagtactatacacacaggaaacaatgttattatggggctactatttaaacagtagcaaattcaggcctatcttcaaagaattatacgttatctaaggacctctcactgatagtaagaaagaggtggtaagagagtcagcacaattattattcataagggcaagggaatggcctaagaggaatccataatgagtgatctgcagtgtaaataatccgcaaattttggacacaaaggctcaaatctagaaccaaatctatggcaggaagtttctaaaaatataaacgctgtgggatttttgtgaagttacgtacccctatgacatataaatgagctcaaataatttatttttctcatttcaattgactcatatggcatgaataacgcactctccagtaataaggatagaggaaggataaacaactgtggaaatttattgcctccaagccaaaactgtgcatctctccaaagctctgctttgtactatcagaaataactctggttcttaggttaatggcgttcaggaaaaacacatcacatgtagaacagcttttacaagctagaaaagaagtcaacaactagatttttcttatttattacaattcatcaatagaataaaacctttctattcaatatgaatggattcaaagttggttgtttaattggaaaatttgttgcaatgggttaacatctatttagctgaaatatatgtaaatgtttatgtcatcacaaatcttttcttttaaggctcagatcttctctttgatgataggtttggtcattgacattcaaatcatgttacttctgtaagccataactcctggtttttcactggaggggagtgataacttaaaacacttggaaagaaatctgactaagaccctagtagattcctatgaggttttcctcgaacttttacagttgtcttcctcacacctaattcaacattaattttgttagtaagttgtctttatcaagactgtgcggagcatttatcttatacttcatcgtctctccttttgcactcatatttcatcagtttgtataataattaagtgctataactacaagatatagtacagctgagacaaacaaatgtatgtacaaataaaaccaaaatacagaataagtcctgggtccctacttttccttttctgacaaacacagacattcagaaaaagaagagagcatttcttaaaccaaggtaaactgaagcgaatttttaaagttctttccatttccactaccctctgcaaaaatgtttgctatatgcttattgtcaccatcaaagatgaagtcactctctctctcccctgaaaaatacagaattttattttgtttacctgaaagctgtgtgatatgacttttaattgaaagtatttattgtctataataagatatgtgttacattggtagctggtctttttgtaagtaaacttttcttattttaaaacagcttcatatttgcagaaaaatctcaacaacagtacagaatactcatataccccccatcaggtttcccttatagttaacagttgctggctttttgctgtaactgtaaagcatttaatctggaacaaaataatctcccattttatagaagtcatcaaaatgtacattctttcttttgtaaagttattatgccatttctgttcctcactgctaatccaaaactacagctaccaaaggcatgtacacttttattgtgttgaccacccaggaacccttccttcaaatggcacacaagcacctaatttccagtttggggaacaactccaccccaactcacagtttttggcattcaggtggggtgattccaggggtagacatggggctcaggcctggccaatcagagcacccctcactccagccacagtaatcaacgagtggctgggggagattgttgccaccttgtgggagcagccttggattatctcagtttacacctattgtcctgagaggatcattaatggattttcctttcaccttcaaaagccaaaaatgatacagtccctccagtgaaagtcctgagctattctatgcatgactttagctgcctccataattatatgaaccaataaataccatagaagaaaggggagaatattggcttaaagtcatttgacaagcaaagagtgtcacttgccctgttcctcatcacaagttcatggttgcaatcaccaaatctttctatttggccacttaaagttcatacacgcataatctgcactggcaatactggaacatttccttagaagaagcacatgcaaagacctggaagcaagagaggggagaatatgtttccaaaatgaaagggagatcaagaaggagagagatcaagtgcagagggaggtgagtgtgggagggaagatatcaggcagggagggtaagcagggggccatgttgcgcaggctgcgtagaagcttgtgtgttattctaaggacaataaggaaccaggaaaggattcaagacagaaggagggtcaagaccatatgctcattttggaacagtctctctgaatgcttagagagagtgaaaggcagatctgaagacaggaagctgcctcaggaagcaatgatcttgagttggggctatgatagtggcaagggtaagaaatgtatgtattaaagatatagccaggcaattctctcgggtcaaatttggagtctcattaacggaaattcctgtctaaccctggtctcctttccctttgcagaccaaaccattagggcccagaatacagccctgcacaaataaatacttgttcaattaatgagtctcaatatatttgttcaattaatgagtctcaatagtttgatagttgaatacaattagagatgcaagccacctccaaattctctagcaaagaaattttgtcttggttcctaggttttacgcattcatgggggaagtaatcatgcattatctacctcaatatggcagcctcaattgcttgagcaaaagctgaagtcaaacctctaagattttcaaatactttgttaagtgtaactattcatattcttttgccctttaaacagtagagaatcagtaaagtattttcttggttccgttgatattagatagaactattgactttcctcctcttttatggcaccataaaacatgttgtactgaaataaaggtttactttgcaactgagaattcataggcaaggcttcctgctgactccacagaacccaggacaagggtgtcactgtttttgttgatggaggtattcttagaaccaattaactactctttccagaggaacctttggatcataattagctttatgtgtgtgtgcacgcacacatgttctggagaaaagagagaaagtgtactacatataaatgagtaattaggtttccgctaaattaaaaattttcccaagtcatggtctgcacttagatgatctgtttctattctgctaatttctgctcagagcaatcactgcccatgaaaccatcacgttttcttattccacagaggtcatagaagttcttttcttctaatggagagagaaacccttgcctattttcccctgtgcctgcaaaggtcagagagatgatgaagtaatgctccaaaaagatgtttagccaaaggaactttagctcaaggctttgacttttaccccttagactaaaaggccacaattttgagtttcagtgtcatgagatcatgaaagaggaagggacctctgtgttcagctaggcagagaggtaacaagaaaaagagatgtgctctagaattaggcttgagttctgtcgggggcacctcctggctgtgaggccttgggaaagatgctctgtctcatttgagcctcattttccacatctgtaaattgggggcagcaaatactgagggtgacatcatcttcactgcattgtagtgagggtcacatgagatagtggcaagaaaatgcacctgtgaactgtaaataactaaaggaatgcatattgttttttgctatctagtttgcccctttttccaaaatggaaacagaggtgcaggacttacccagagcagcagcctggtgaccatctagagtcccagctcccaattcagagcaccttcacctctgcacatgaccttccagttattgacactcctccagcagggctatgtttacctaatgtgtcaaactcaatcctgaggacagccactctgaccagtagtacagcagttaacatttagatgggaaatcttagtgtgtttggacttatcatatcttagtttaaaccaaataattcaaccaaagcataagagtattcctttttctgagccatctcattctcagaatcacaattattgatgtcacccaaagtgaacacctagctctaatttatctgaggccttccctttttgaacacggaaagttcctcatgccaggaacttcctcagtcccaagaaaactgaggcagtcatccatcccaaatgtccatatttctctgaatgtctacaatatgccaggcactgttctgagctctttatttacaatgttctacttattcatttggaaatatagatgaggtagggttatttccagtttatgaggtgaggaaactcagttccgagtgatccaataacttgcccaagtcacacagccaatgtgcattggagccaggacccatggcattgtgaatccagaactgtgtcatgtaacactgactccccgaatgccctgaacattctcaggaattgtgggtgggtggtgtaaactgcttcacaccagcttattttatagtctacctgccagttgtaatggttctgcattgattggaaagtggttctagtattcaaacttgggagtgtgatagtgaaaagaccactaagaaaatgcatataaagccctcaattcaagttcctaccacattgtttcttgactttacatcttgaagtctttttttccttatcatatgaaatgaaaataataaaacctacatcaaagtaataccttgagactcaaagtgtttagaaaacattttgtaagctgagaaacccaatatattagcaaagtggggcttaatttatttacattaggttaattatgttattgaggatttatatggtgtcactgtacaagcttaatggctctgttagcctctaatttttttggtttatttcccaaaatttactttcacaaccagtatcttccagacaccgtatagcagacatctgtggttgcctgcacagcaggaattcttccatttctcttaatgagatcctgatttccacgtgggaacatcatcctgtccccatccttattctcaaacatgagctttagccaatcagcacatgaaacaatcagtgtcagtgagacatttgtgagaacttctgcgaaacacaagttctcgtcatcttccatgggcaaatttatcttccccactggatgtgaacaataatagttaaagccactaaagctatcaacagtagacttaaaaagagttttgagtgagggagagaagacgaatcctcacttcatgaaagcagaattaaaaccagaaagagaaatggattttttttatatcaatttagccttaaatcaagtcacatacaaattccagctctgcactttcaagactgagtgagcttgggtgtggccattaacttctctgagcttttgtttcctcatctgtaaaatagggatcaatcacttactccttatagtgccgtggtgagtgttgaactggataacaattgtcaagttgcctaccacaaggcctggcaaataagtgctcagtgagtgagggctttccctcgctgccctgcccctagtggggaagactgggcagtgactatataaaaaatgatttgaaaataagattcttgtttggagtttaataagactccaagaactttgctcatgttactgtgtgtgatacaataaagggaaaggaggctaaattatttcttattgtctcattttcatatctgtttacataattcaatttgcattttaatttgcacattaattcactttccaagatgaaccctggaccaaatcctgatgcagtattttttgtgtgtgtgcctgtgtatgggatataaggtatggctttgggaagaggacaaccatgaccctgataaacaactggctagaagacacaattcctccagttccagccatccttaacctcctcattcccagtctctttgaccatatactaaagctgtgctcactcagtctaggattgtcccatattaacataaatctgaaagagagaacaatctttattattagaggagagtaggtcttctctggctgttcagctgtatcttggaaacccaaagatgcagctggccactcttcaacaacagcagcagataaggaagtaacatgtctgctccatctgaacaccatccaaaaggaaatcctctccaagcctacaggaaactccaagactacacctggcaagagaacacagggaataagaagcggctaggtttaaaggcaggagcatagaaatcaagaattttcagccacctctgcttcaactatgtatttcccactctgggaaccagggttcccacctgtaaaatgggcagtcagaaataatgaagagtaagaaacaatctcaacatcaggtcagctacatccagcacaatcctggagtgtgagcacatcctagggtatgtgagcacataccctagacctataggataatggatggagtgaaccctgcaggagtacaataagtacgccagtgctccttcaaaaacttccttccatttcacaggattcttttcttcccagcaaaatccttatcatctctgaggcccagctcaaatgctgtctcttctgtgatgtttttgcaacactcattgcccaagtaatcccagttattaaagctcttaaccccacaatgccctgtccacagactctgaaagatgctgatgcattgttgtgtcccatgtctgtttccccagcaggttgtgagttctcagttgaattcagtttcttgttgcagagtctttatcaaaccacagaagaatcaaagttgaacaacatggagtatctacaccggagcagcccacagttcagggatggacacagaacaagagagattcattacagacataaagcacagagatgttggggttttctctgttgggaagaataagaggtccagaaaagcttcccaaagtgatggcacctcaagggtcaggacctcaccttattaatctccatgacccagcatctactacagcatctgtcacaactgggctctgagaatgttggctaaataaatgaatgaatgatatcaatacacagggtttttccccattttctgaatattctggactaggggatatctcagaacagtacttagcacctagtgtgtgctcaataaattcttgttaaaccactaaaaattgctggacagctgaactgaaaattactcacagccccattcaactgcatcagccatgaaaatcaactcagaatttgcaaatctatgctggcatttagcacttaagatgtaaatacagagtgtcagccatgtggctaagatcagctttaattcagtgttcatctctgaaattcattaatgattaaatacttttttcctttgctctctatgggagttgaaacaagtatcatgtatccaaagaccagggttcagtttggcccaacattaattcacttaatgtttcaacaaaaatttattgaccatctactaagtgctgagtgctagaatccattgactacctactaatgaagtgctagattttaacacagggacatctgtggtaaaacagtaaattctctaacctcatctagaggggttgaaggttctgcctttgcctaccttctatagtcagagactactggtatttcaatccataagtattaactgaaagtcactctagttctctgcacatgtgaagcagagcatattattattttgtccttgttgctctatctaaatgtcattccctcttctccatcccatcacttattttgtccttggaattccttctggatttcctcaagattatatagaagctccatgaaggcaggacactgtctgactcattggctcctgagtcccttgaacgtagtgctgagcttggcatatagcaagggctcaataaatgtttattgaatgaatatatgacatgatgaatgattagatgaaaacgtctccttttccaggaagttttccctgtttctaaaaaggagttaagggcctctgctgagggctccccaagtctctcctctgggtccagcagagcacattctccttctccattttctcctgtctccactgctcaatggagagttcctctaaggcacaggccatgagtgtttcatctctaatttcctgactctagcagagggatgttcacaaaggatgtgctcagataataaatattgattaaactcaacagacctaaacgaagggaatctctaaactcctgggtaactcactgcagattggcccttgatcggtttctagggatccactctatcccctgaaaactccggtggtactgaagagcctcttaaagttaaaaagcatcagtgggatggaggtgggatttatgttgatcatcagaggccaaaagtccagtcacagctgtgggaatgaggaaaagagtgaatactattcgtatttcttaccattaaaactaaaaatcatattctgatactgtacacggaagaatcaagtgcattctccagcaggcaattgacactggcctgcttattggaattgtgaaagaataagcaaccacaaaggcaagaatacacaagtactgttctgcttatcccagagaggtgattcatgggctcttcctgccatctggtttcctggcaggaatggtagaggcaaatctgaggtgggaccacttgctgcttaggatagcaatgtggtaagaagcatcaccaacctgccatcatcaccaatcagacaacttggggctctcccctcttgatagcgctgccctcaggctgctctgtcctgacggtaattttggtcatggggaaaaggcgccaattttacccttactttctcccccataatggtacctgatggccctcataattgtaaaagcacacaatcatagcagctattaacttgctgacacatccttgagttactcttctaaaatatatccctaagtggattgcagaagtctctgggtgaccgtaggatcagaccatccaatgcaagacatcaccagtccattgctgtcactgaaacctggctttgttgggatggcattggtctgcagcaaccttcccggcactggttcctcagcccactggagccaaggatttctggtgggaacatctggtaaaccataggatctttctgttctccacattacccagacatgcatgcattaggggagaaaacataaacatttatactctcaatctgctccacccctcaaaaacggtataatgcctaaaacaggtctggtttctatcggggatagttgggccatctctctgtattccacttccctcatatattaaagtaagaatacatacttccatttaaagttaaaaagaagaaggatcagcttcttttgagtatgtaaagtctcagtggcatcaaccaataagtggagactgttaaactctccagtggtggaataatgcggacaaccttgtgagtttacatgtttgtgcatgcttatcatgcgctagagagtgtgctaagcactttacaagtcttattgcttctaattttcataacaaacctaggagacacatattattaccatctccattttagaggtgaggacacaggctcagagataaagtgatatggcccaaatccctatggctagttaaatagtggtgcctgggtttgaatccaagtggcctaagttcaaagtccaggctcttaaccaatactctgggttagggtaagaagaaaattggataatgtatgaatcagagatattccagattaaaggaagcttgaggagctcctccccaatgttttgatgttgcaggtggggaagtttttgaaattggaagctgcttaaggcttaaattgttatgagccaacacttggcaagaacgagttgcccaagttctttgcaattctgatggctaggcccatatactctagcagaacattttgtacatgattgttgctcaagtaatattatttgaagaaagaatgacagtttgactcaaaatgagaagatcttgactcatacttcaccatcaaaactgattccttttctgtggccatttctttatctgtaaaaagggcatagtgctttctgcttaactttaagacaccaagataaaaggttacataaacccactgaggcatattcctacaatgcacatttatacatccttagtggcattctgatggctgataaaagtaagacttactgaaaataaaaacaagaaagaaagaataaatcaaaataaattttcatatttttgccagactgaaaactcccttagaataaggatggtgtttggattggtcgccactgtatctgtaatgccaagtagttatcactgtttgtatacatttggagaataaaggaatgcatcaccaagaatgtttaatacaatgcaaaaggaattttgagaaaaaggtaggtgggaaaatgtaggatacaaatttgcacatatggtataaacccagctccataaaaaagcaagaaatacgtaggagagaaaaagaatttaagcccagaaggaaatacacaaaacacagagtggtttgcctctggataatagagtaatgaggaaatcatttttctgccccattcagtttcttctgaattctttaaattttccataatgatcagttgttaatggataatcaggaaaaaatgaaaaataaaactattcacagagtccagcttagaaacccaagtcctagcataaggaggtcactgagtctgagcagcttgggtgtggggatctcggctgtgagctaccccactggacaccattagagcagacgtttagccctgagtcatattccaaagccatattatacacttgaacttgccttccccaaccctacagctgcacagcagacatgctcaaagtcccatatgcttctttagtgcagattattcaagaaacacaggctgggggaaatggcattgtcaggaatattgcaagacaggcaagacttgcaggtgcacagcagcagaaacccacagccatggttattcaagtgaagctggaatctaaagggatgaacagacctagaagctccttgtgcggtcactttgtctgtgtctccatagccagccttctcaattagttgcgacttataaataagcatgtttctgaaatctccaacacatttcctatgggaccaaagaagataaagagatggggccgagtgtggatgagtggagaatacagatatgctcaggccgggcatttcatatggcttgcagagattttactgagtgatggaaggttgcaggccccatcagtccatcaggggatgttcattttgaagtgaagatctggacccttctgctccacgtggcccgctctggtttatggctctgtctctggttgtctattaacgtgagttagtgccagtactttatagtctaaacctttgcaacagaaaacagccattctttgagctcagcgagcccagcaagagcaacaatgtatggaacgaggagcgtcatttctctggacacttactttctgccccttgccctaatgtacaactacagtcttctaagcacaatacttctttttagcaaggctttaaaatacagttctgcctctctctatttctattctcactactcctgttcaagggcctagtctccctcacctctctcctgcactgctggcttctacccttgccccctctcttccattctccactggtagccagagtgagcttacagaaacagaattgagatcacacatcactcccctgcttaaaataacttagaactctccacattggtatgcaaagggctgttggacttgagcttccttcctgacacccccttgagctacttgtctccttttctatgttctagccatgctgtttgcaaacctacctagcttgttcctaccttgggaccctggcactgacaactctgtctaccaaaaaggctccccacctgagtgtaaattagttcaaccattgtggaagacagtgtggcaactcctcaaggatctagaaccagaaataccatttgacccagcaatcccattacggggtatatacccaaacgattataaatcattctactataaagacacatgcacacgtatgaaacttgcagcactgttcactataacaaagacttggaaccagcccaagtgcccatccatgatagactggataaagaaaatgtgacacatatacatcatggaatactatgcagccataaaaaaggatgagttcatgtccttttcagggacatggatgaagctggaaaccatcattctcagcaaaccaacacaggaacagaaaatcaaacaccgcatgttctcattcataagtgggagctgagcaatgagaacactggacacagggaggggaatatcacacaccagggcctgttgggggtggggggttagggaagggatagcattaggagaaatacctaatgtagatgacgggttgatgggtgcagcaaaccaccatgtcacacgtatacctatgtaacaaacctacacactctgcacatgtataccagaatttaaagtataataaattttttttaaaaaaggctccccattctcacttacaatcttcttttagtttgttcttgataagctttcagctcttgaccacaaataggctctccagggtccatctagttcaattctctgtttaagatttattcctaagaaggaggaagggagtggggaaagggctgaaaaactatctactggttactatggttagtaacttggtgacagggtcaatcatacccaaaacctcagcatcacacaatataccaatataacaaacttgcacatatacgcccaaactcaaaataaaaatttaaatttaaattttaaaaaatatttcttaagatatttattcctaactttgaagtatattgttctttacagtctgtttccctctataagaaagcaaaacccataagagactaaggatcttgacaatttaaagctgtatccccagtacctaggacgttggcaatatcaattcgtatgtttttaatgcattcctgaatgaacgaataaaacaaattcaagcctaaatggaacccaaaatcacagaaatgctgttcttcagtacattactaagtgcagtgcctccttgacccgtctaccctaaatcatcaaaggaaaaagaaaaccttcaagaagtgattccgaatcctagtttcaccatttactccatgattctagacaagtcttaaattctctgcacctcagttttctcttctaaaatagaagatgtaagagaaataaaaacagctgtacctagagttgtgatgcctattcaatgagtgagtatatgttgggcactttgaactgtatctggcacatgcattaccatgaggatattctccccatgttgacagcattgtgtaataaaaaagctcttagctctgccgctggaaataggcaggagggggcccagcagggagcaagtctgaaagcagggagaccctgtaaggagattgatccccccacagctgactggtaatgaggctgggccaggggagaggcagtgcaagcagagatgagctgataaattaacagagcaatgcagaagtagaattaataggatttgtctattgttggagccattggagggaggagtcaagagcaggaagctgaggatgcagacctggccacccgcgatagagatgagcagctgtctactcacatccactcataagccaacccaggagatgctcagggcacaaatcacatacaacctggaacctgccttgaggacgtgggtgcaaagaggggaaccagcagagacaaaaaaaataagtgatgagggtcaccctgggggtacgaacagaggacaatgtggtcacagaatgggatacgatttcttcaagtataggagatttggataggcatcacagaaaaagtgacagcccgcaacttttttattgcactccttacagcatacccgaaagcattggtgaggacacaaaaactacagataagaatcagattctaaaaagacaattctcttttccattcctgtcctctcccctgcaacttcccaatccctcacctctaattaacccgcccaccccttcactagcttctgatttcaggcaacgtccagtacttgttccacctttctctctgaccagccatcaagaagatcttgtatgtttctcctacacacccctgcccctggacccaggaattcttccatttttccatatttgggctatattaagtaataagcccacatgctttctgttgagaaaatacaaaaagatgtttccctctgtcataaagaaaaagaggtaacccagggaacattttgtccctctagttatcttcccacaggcccatcaagaatcaggcagtaggtgaaaaagaaacacagagaacctaggaacacaataggaagaccaccatgggcccttagggagtcagcgaaggcttatgatgcaaaaagaaggtcccaggtaccttaaaaactccacttccctctctaggatccccaagagagcttgacagcgtccctctatgcagatgttcataaatcaggcatatgtaactctgcggtttcctgcacataattgatcacagttgagctgctcagacattaaatccaaaggacatcagagaaggacgagttcagtaaagaacactgagaaagaagtggaccctgagcatagatcttggcatacatgcgtgggaaatggcctctcaaggggtcattatccattcaattacacacacgttaatttggaaagagaaagttcctgccttgagtaaattgctgtctgttagggaaagtgaaaatccactaggggtaacaaataacaaatttaaatgccttctgggtccagcagataccatcaatacctatcatgaccaaggaggtgggtggcttgtgaaaaaccagagagtccagggccacctgaaacaccctcaatttcagaaacattttacatttcatgactagcagataaatacccctggggtagtgaattttcaaaatctcacacaggtctccttagagcagagtttctcatctccagcaatattgacatttggagtcagataattatttttgggttggggggtgggcactgatatgttcattgtaggatgtttagcaagatctctggactctgcacactagataccagtagcacccccatagtggtgacaattaactgtgtccccagacattgccaaatgtatcctggggagcaaaatcatctcctattctcacctcctgagaaagaagtgcaggatatcacaatagcagagggcaatggaagatgacagtcccatgctagaagctgctttaccaacacagtcagctgctatctccacaacaggcgggtgaggaaggattcatgaccctcaatgaaatgaacaaatgcaagcaaagccaagttgccattgaatgtggcagttattgtttatttattttattatttattttatttatttatattttaatttctctctctcttttttcttttttcttttttttttttttttttagagagagattgggtctcactgtgttgcccaggctggtctcaaatgtctggcttcaagcaatcctctcaccttagactcccaaagtgcactccgccctgccagagttactatttgaatccagacattctgactctgaggctgcgttttaaccagcctgacatcacgcctcaagcaggggatttttcaaaggacaggatgatggagctgaggctcaagagacagtcagccttgacctctgtgtgtggagcatccctccagcgattaccctgtccatggtgtagaagatgggctggcgagaggccacagatgtccggaggctgctgcagttgtattgatgataaatgacaagggtctgcactttaagcagtgggagtagggatctagaagatactaaaactatttaggctgggggcaatgaatcacgcctgtaatcccagccctttgagaggccaaggcaggcagataatttgaggccaggagttcgaaaccagcctggcccatatggtgaaaccccatctctactaaaaatacaaaaattagccgggcgtggtggcaggtacctgtaatcccagatactcaaaagactgaggcgggagaatctctggaacctgggaggcggaggttgcagtgagccaagaccaagatcatgccaccatgctccagcttaggcaacagagtgagacactgtctttaaaagaaaaaaaacaaaaattaaagacaaaattgacaggattcatgattgattgaactagggagtttttaaaaattctagaatattccacaattagtcataatacttaatatcaactggtttcacaaaccaactaactatcacagcagcttaacaataagttttatttctcacccatgttggtccaattgcgtgtttggccagcagtcttcaaagaagtgactcagggattcaaaatccctgcatcttgtggcatcggcctactctaggtcaggggtcatcaaactactgcctgtggaccaaatccagcactgcttgtgggccaaatccagccactgcctgtctttgtaaacacattttttatagggacacagtcatgctcatttgtttacgtattatctatgggggtttcacattacaacagcagagttgaataattgcaacagatgcagagactgtatcatccataaggtctacaacatttactatctggccgtttgctggaacagtttgctgacccttgccctcagctctccttggagtctctcactggatttcatgtttctattggccaatgagaaaaaagagaaagtaagagggctcacacgagaggtttatcaccagctggacctgacgcatgcccttcctgtggccacacactgttcactggtcaagactgagactcacggccccactcactgcaggaaagctgggaagcatgcaggcaatagtggataaggctgagaacccacagtctacgccacacatccatccatggctctggaggaacttccttttgctgattatcatcctttggtttccttagaatggaaaactcacatattcttgcaaagtgctataaacaaccatatctcactgagatgcattaatgaccaacataaactactgatccctgagtttaaacaaatgtattatgttagttgataatcttagaataaacatgacctcaattaataaattttctcagagctatatactctctcttagttcaagtaagcgtacaaatggaaattcagtttaactggataatacagagtgtctactttgtgctcagcactgtgccaagcagaaagaaaaataaagacctaccggatacctgccaggacctcatcctatgcgtgaaatacataaatatagaaataacaaaagaagtattatgtctaagttcaaaaatatgaagttttatcttcttatcagctcagagaaagaatatatcataaggactgggatagttgagagtcacttcctggagtgggggaaaaagccattataatttacaacagtgttcaataaaaattaaaaagagtgaaagttgatcaaatgtaaaaaacactttaaaacttagaagtcattctcactatgtgatcttctttaatattcatcaaaattctgtaatttccagtgtcatcaggactagctcagtttcagtgaattccagcaagggatgtcaagtcacctgagacctggaacacacagcctgcgaggagagcagcaggcctgaaacgagggcgggatttttctttttcttttttattttctctccttaccccctctttctgatttttactccagggctctttctactaccacattgtcattaaaaataaaataatgtcatttgacagacttaaactgagataaccacagaaaactccagatctgttccaaagcaaacagcagccaggaaatcctttccatgcacagacagtaacatttctccttagctcaaggatggtgcagccaagggtaaaggaaatgaggacacaggttgaatgctgtccccgaagagtgctccttccaggagctcaggtcaaaggcagctttgagattaaaacacagctatcctgataggggaaggaagtagcactgtgagggttgtcaattttatcaagttcagtaaatgaactagacaaagaacaattgatcaagaatatgcagccctttgttggcagttctgaagaattctggcttctctctgggtcatgctcccaggtccccgtggcccatgctgtttgtcatctacactcacaggccgctgggacggtaagctcctgaatgacaaaggatatctgggtgtgccgcacatgtatgtacacagtgttctgcctacatatgcaggaggtctccatgttgtggtagagtagacacctgctgttatggcccacttgagccaagcatgtccatagggtgaactgtagcagtcaccatgacaagtctggcaagaggacttccctttgatgaggaagtcaccatggaaatgggagacaaaatgaagtcatttgccttggcccttagaaataaagttcacatccttagtgtgacatatctgtgccctgttccccattcattggggcaatctttctgtgtcaactcctgcttttcagtgtcccaccaatatgagacgctcagtacattactcaccatatacctgggcttgcttataattccacttcctgcttcctggtgtgccactcccactttctccacctggaaaacccctatccatgtgccatacttagctattaatgtcagttcatatgtgacaatttctccaaggtgtttttttagtgtcaggaagccactctttttgctttgcaattgtaactctttgtatgcattattaatatagcacatgttacattctgttgacatactctatgttcttctctgtttctccaaccagtctggatagttgaatggacaggcatgatgtctaactcttttctgttttctccagtcacctatcagagtgtctgacatgtagtagacattcaacaagtgtttgctggagatggtaaatctcaggggaaaatctggttggaaggaaactgtgaggaagtggaaggaggcaagaattgaagcttgaagcattccggccccaccactctgtgcccttgaaatcatggacacttcagcaagtatctatttcttcatttagaaagtgggagtattttgtctaccttacatattaataacaaagtcaagattaggcaataaaaagctaggaggaaatagttgattaattgtgtcaacagtgaattaataaggacatgaaaggaatttgcaacatgtacttttagcaacactgattcattgctacagagtgataagtcaagaaggaaaaaacagaagatggtatcaactggtaggctaagaggagggtctaaccttttggccaatttgctgagcaatcaattcgaatttacatccaaatggtcatttggtctcagaataatgaagaacagagaaataagaaggtagaaactcactaaatgaaatcatgactactctaactctccttatccttccagatgctgtttaatcacatcatttgttggtgtttgcatttgtttattagtatttgtattcactgagaggcccacacagctcactctcagagtctggcagctccaaggagatcacttccactgtaggtagatttgctcttaccagcatcattgccttagaaggggagagtgttccatctgccttagtcaacagtagtaatagtagtagcaatggtggtgacagaaataggtagcaatggttgtgttatggaaattcacatttattgaaatctcatcagtgaccatgtactggccaatcatttaactacatctcagttaatgttcaaaactcacctgcaagtgacttacaatcttttattcaactaatatttaatcacaccttctaggcactggggatatagcactgaacacaccagatgagatcttcagcctcatgtcaattattgcttgctggtgagagaagaaagacaacagacaaaaaaaaaaaaaaaaaagtaaccgcactagataacgcattttctttttctttattttttttttaaattatactttaagttctagggtacatgtgcaaaatgtgcaagtttgttatactttaagttctagggaacatgtgcaaaatgtgcaggtttgtttcatatgtatacatgtgccacgttggtgtgctgcactcattaacttgtcatttacattaggcatatctcctaatgctatccctccccctccccccacctcatgacaggccccggtgtgtgatattccccttcctgtgtccaagtgttctcattgttcaattcccacctatgagtgagaacatgataacgcattttctaacatgtgacagaaagtgactgaaaggtgaactctgtgttaggtaggagaaaacttattagtctcattttaccaatgaggcattagaggctcagagaaattaagtaacttctccaagatcacatagctacccacggacagagcaagtctgtctaaataggaagcaataaaatagcatacacagcagaatatagtcaagaattagatgtattatgctttaagcctttgtttcgtagcttcttaagaataggaagaaaagtcagtgttggctacattggctagaaatcttcacgaaggaagaaaaagttaagcttgtcttggagcgtggttaggatttagacaggtagaaagaaagaaaagagttgtgagcaggaagaaatgggtatttatcgaggacttattaagtggcaagaacaagacaaaaagttttagatatggtgttttagtaaatatttaaagttttttttttgtttttgtttttgtgtttttttgagacggagtctcactctgtcgcccaggctggagtgcagtggcgcagtctcggctcactgcaagctctgcctcccgggttcacaccattctcctgcctcagcctcccgagtagccgggactaccaggcggccgccaccacgtccggctaattttttgtattttttagtagagacggggtttcactgtgttagccaggatggtctcgatctcctgaccttgtcatccgcctgcctcggcctcccaaagtgctgggattacaggcgtgagccactgtgcccagccaaatatttaaagttttttaaattcaggctggttacatgacctttcgaggtgacataagatgctgggtgttgggcatgtaggtagcaactggcacgctgtatgcagggaacactaaaatggctcaggtacctgaggaagaaatttacattggggactattgagaagtataattcaatgggctagcaaaccaatgagaggcaactgaagacattgaaggagaggtggtggaagctggaagtggcataggagacaacaggaactgtagcaattttttcaacaaggaagtaaaactcagatgttggaactgaaaccatcttttaccatactctttcatcttggagtcatcactctcaaatatggctccccagagatgctcccttctcgtatgatattagaattagttgtatcacttatttgctagactgggctacagaataagaacacagactatgtcaacccctctatcctcttagaatctggcatgggatcaggcacacattgtttgatttcaactggcaaggccatggcaatcaggcagacaggttagctagaaaatggtgaaaaaatggcaccaaattagggcatcacagcaccagataaaactaagcagagattcacctgaggtggcccgaggcagcagagccggaggggcctgactctgatcctacaaaggggaactcagaatcatcactgtgaaggtgagaagagccatcaattctaaaggatgaaggaggataaactgatagaagaaaaatgagccaggacatcagaaagaaaattaaaaacaaagtggaatacagtgtgaagattgatttggggcaaaagatttgaaactaagaccatgaacaatgagattcgttaatggagtttccctttgtatgatgcctagacccagcaacagggcagttgcagtgatttaaggatgactcacagggatggatacctgttgaacacaccttaaaaaggtgaaagaaaggtaggaaggaaacgagatagatgtgagaaacatagataaggacagggaactgaggaaagggaaaaaggaaataatccatgatattttcagaaatgatttataacagggattggtaaactatgaccctgagacaaattcagtatactgtttctataaataaagctttgttaaatcacagccacacctattcatttaagtaatatctatgcctgctttcaaaccatagttgcagggttgttgcaagaaagattatctagctcacaaaacccaaaatattaactatctagcccttaacagaaaaggtttgccaccctagtctatagtaaaggagtattgcaatatggatgctaagtggctaaagataaagctgacattctatatcagggccagaattttgaatgccttgctaaggtgttagaactttctctgcaggcaataaggatccatgcaaatagcctaaaatttttctctacataagcataattcaaaaccaatcaaagaactcgacagtggcaatcaatgtggcacatgaacaataagaacactactggcatgtttaatcaaccaagctctcttctggcttcaactgcctaagacttaagaactgtatgctggtcaccagtctgccaaattagagagtcacaatgccactgtggtctttaaatttctaatgtccaaaatgggggaaataatcccacctgtgggcttattatgaacataaaatttagataatagagaaaaaaatatattaaataaataaaatattttacaaaggtctgacatcactcattcacttactcaatgaaaaaaaatgtatttagtgcctgttgtgcgcagaggaatccagagatgagggagattaaggcccatagactagaaggaaagatgggatgcaaacggcagtgccaagttcacacataaagcaaagtagggaggaaaccactcagcttgagaagaagtaggctgtcaggaaaagggtccataggaagggtttccagcaaggaagaaaagatcatgatgctgaggaaggagatgaagctgctttctatgtacaacaagtgcaaaggcctaatgtagttcaaagtgagtggcacctagaagccaactgtggatgcaaacagaaatgaagcaaaagatggagggaggatatgaaaggttttctatgctatgccaaggaacttggactttatccattggaagtagtaagcctttgaaggatttggcttccaggaaagacatggtcagaactgggtcacaaatgtctccattgatcagactgttagttgagattccagcttttcctcccctgcatgaatgaggggggcagagcttaagactggtcatgacagacctctcaatgacctttccatttcaggcaagtcaaggttaccactcatagtcatcgtcctagcagccattggggaaaagcagctggacaagagaaaagaatgacggcacatgaaacacccttgaataatccccctaaattgacccccggccctgggacatctgcctacaaaagaaaccgtgcctgcatctgtgctatgtttcacatttcccaggcccgtatcttctccaaacgcaatctggacagccaatatttatcaagcaagagaggaaacaatcaacatattcaggagaggatggaaagagcaagataatttcttgatttgggtttacacagtggatcagggtttgtgttgttctctcacctccaaaaacctcattgatatcttctccaccttagaggaaaagaggactaggaagttattgatataaaaagaattgccatgggaaggatgcccacgtgggacagtgccaaagctctctctcctgatgggaccctctgctaatggaaccacttgaccaagaaggacaattttttatttttgttcctaaaacttcagagtgtcatggaagagaaaacaggacccggttgcagtttgacatgcacagaagcccaaaaaggactcattgctgctgagaggtagtgtcatcaaaaaggaaagtatgggtcttgtaggcagataaatgtgggtctaaaactgggtcccaagccatacctcctgtgtgaccttagggcacatgctaaactttcctaagcctcattttctcatctataaaatgggtaatatattctaatgaataggtaatatattccaaatgctaagcacagtatttaatgtgtactcaatataccccattattaaaattatgcatagcttaatgctcaagatgagttcccaggccatttatttagtaaagtatgatgtgctttaaaaacaaaaataataatcctacaacatgaaagtcataatacactaaaagaaatgtttgaagaaatggcatccataaaataaaataattactatttaagattttctacagatttcttaattagcatagagtagatattcaggaaattttgttgaatgtttagataaattattgagctaattaatttaatacatgatataggcagattttaagtctataaacctgcataagacaaagcagtattagtataaagaaagcttacatagaccgacaaaactgaaaaagtacaattgccaaaggattgaaagaacaacgcagagaagaggaatttgaaattaccaataagcctttgagcaaaccaatgttcaaacaatacatagttgtataaacacaaatgcagactaaagcattattcactatcgagttgtaaaagatttttaaactaacagcatcctatgattgtttgggtgtcatgagatgaagcaattcaggcacagttagtcagaatggacatttactagcctttctggaaagccacttttcagcactactacaagccttgaatgatctgcgtagtttgactcaataattctacccctaaaagcctccaatatagaaataggagagccatagacaaggatttatctatagaaatgtttgtcacactattatttatatagtatcaaaagaagaataaaaggagaaggaggaggaaagaggagaggaagaaggaaaacgaagaagaagaggaaagaagactaaatgtttagcaagagaagaatatttaataacataaggcttgctgtttatatacttttaaataaaagtagtgaataaataatattaaatatcagtatgatcataatttagtgaataaatattagatagatgtagatagatacgaggacaaatccaaaaatagattaaagacagaacataaattcaacaaaatattatgtagttattttgcatagttatttgtgtaaataaaaaaaggctgcttaagagagaataaaaattgcattcttatctaacttctaaatagcaaaaaatgggaacccaggacattcagatgcttcttagcttataatgagagtatgttccaataactctatcttaagttaaaaaacatttaatacacctcacttcctgaagaccacagcttagcctagcctactttaaacatgctcagagcgcttatatttgtctacagttgaggaaaatctaccacaaagcttattgtataataaagtgttgaatgtcttatgtaatttattgaatgctgaaagtacactttctactgaatgaatatcacttatacaccatcatgaagtccaaacatctaagtcaaaccattgtgaattaggatagtgaagtaatatcatcaaccagctaagagaaaaccatgctcaatctgcagttgtatgcacaaagaggggtaacttgtgggaactagaggttgacacaatagaattaaaatatagggcagcaatagcaagggagtttggatgaaaaataatcagttaaaatattatgtatttttatattgttcaagtgtatagaaaagataatacatttaaactttgttaacatggtatgtgtgttaaaataggtaggtaactactaaaatgatagtcacacagcatataattgccaaataagtagagggagaaaaaatggaaaaaaactaaatcaatcacaatgaaggcaagaaagaaaaaaaagaaaagaaaagaaacataaaagacagacgaagtggaaagcacataaccaaatttttcaaataaatccaaatatatcagtgcctagtctgctccagctgccatagcagaatatcatagactaggtgacttaaacaacagaagtttacttctcgcaattctggaggctggaaatttgacatcagggttccagtatgaccgagctttggtgaagggactcttcctggtttgcagagagccaccttctcactgtgtgctcacatggccttccctctgtgtgtgcatggggtgagagagagagagagaaacagcaaaataaatggtactgaacaacaaaataggatggcaaacaaatcagtgcctggattttgtgacagtttggtttattgttcagaaatattcacccttttgccagttctaccatgggtgtgataactgccccacccactgaagtgacttgttttaaccaacagtatatgatatgatttggatgtttgtccctgccaagtctcatgtggaaatgtgatctccagtgatggaggtagagcctggtgggaggtgtttgggtcattcagatgaatcccttaggaatggcttgatgccttcatattctcactctgtgttgcacgcaagatccagttgtttaaaaaagtgtggcaccttccccctttctctcttgcttccattcttgctctatgataccctgcctcctcctttgccttctgccatgtgtcgaagcttcctgagtccctcaccagaagcatagctggccctgtttcatgtacagccagcagaactgtgagccaaaataaaacctcttttctttatacattaccccgtctcaggtattcctttacagcaatacaaaaacagactaacacagtataaggatagatataattggagcaaaggcttaaatgtgtttatatatataaaatggggtttggccttttatacttctgtcataatgatgagaaaaacatgttctagtctatggtctaaaaagggtgagagacacatggaacagacctgagcttaacctgtaatttggaaaaaaaacaaacagcagaatcccacctagatcagccaaacccagccaattcacacatgtgaatcagtgtgaatcagaatgagacactgagttttgtagtagttccttacacagcatattttgtggcaacagctaactgatacagagctatatttataaacctgtatcagccagccccagagagatctataaatatatgactgacagtaagtcaccaagtgtattgggtaacagctaagtgatataaagtaatatttataaacttcaatctttatgttgaaagaataacaaccaacagttaacggattagaaaaatgacaaagaatgtacacatgaaaagagaacaatgacaaaatcagaaagcaattacattctaaatcaacatccatatctctgcaaaggacttgatatacgcttatagatccaaaaagctcagcaaaccacaagctggacaaacccaaagaaatctatggcaaggttcattatagacaaacttctgaaaattaaggacaaataaaaaaattgaaaacagtgggaaaaaaatgactttttatagagaaaaaaatatgaatgactgatttctcatcagaaaccatgaaagccagaaagaagtgacgtaacacatttaaagtgctgaaaaaaaatgtcaactcaaaattctgtaaccaaaaaaaaatatttccctgcaatgagggggatatcaagacattctcagatgaaaaataacaagaatattttgtcaacagaactaccctaaaaaaatgactaaaggaatttctccaaacagcaaggacatctggaagaaagaacaacagaaagaggaaaattacaggtaaatatattttgcttcccttgagttttctaatttacatttgatagtcgaagcaacatttttaatattgtcttacgtggttctgaatgtatgtacagaaaatacttgagacaatacattataaatgaagataaaaaggaaggaatgtttttacacttcacttgaactagtaaaatttcaataccagtagactatgataagctatgtatatacaatttaacatatatagtagcaactatttttaaaagctatacaaaaagataccatcaaaaatattataggtaagtcaacatgaaattataaaccatgtttaactaacccacaagaaaaacagaaaaagaaaacagatacatgaaaatctgagaggaaaaaaaaaaaacagagaacacaatgggaagcttcattcaatgtaagggtactagaagttctagccagtgcaattaagaggaaaaaaataaataaaaaggcatatgtgttgaaaggaagaaattaaactgtctttatttgcaaatgacatgattatcagcacagataatcaagataaatatataaaaagatttctgaaactaataagttagttcagtaaggtcgtaagctataagacaaacaaaggaaaatcaattgtatttgaatgtatcgacagtaaacatatggacattaaaattaacaatacaatataatttatatttattaaaaatataaaatgcttaggcataaatctaacaaaacccccacagtacttgtaggtgaaaactataaaatactgattaaaaatgatctaaataaatggaataacatagcatgtccatggattgaaatactcaacatagtcagttctctccagattgatacacagctttaatgcaattcttataaaaatctctgcaagatttttttgtaaatatagctaaaacaatattggaaaaaaaatagtgaagtggtattccaaggcttactatatggccagagtagtccagactgtggtattggcagaggcattgtattagtccgttttcacactgctgtaaagatactacctgagaccggataatttataaataaaagaagattaattgactcacagttctgcatggctggtgaagcctcaggaaacttacaatcatggtagaaagcagggagaagcaaacaccttcttcacaaggcagcaggagagagagaggggcagggaaaggaaacactcataaaaatggatcttattaaaattaagaattttgctctctgaaagaccctgttaaggggttaaaaagataagctacagttgtaggaaatatttgcaatccacctatccaagcaacaaccaatatctagaatatataaagaactctcaaaactcaatattaaatgcaaataatacaattagaaaatggacaaagtacatgaaaagatgtttcaccaaagagggtgtgtgtttgtgtgtgtgtttgtgtgtgtgtatgtactgtatatggcaaataaacacataaaaatattcaatataattagctgtcaaagaaatccaaattaaaaccacattggcatatcactacacatccatcagaatagctaaaataaaaaatacaacactaaagtcattatcacaaagaaatacaaatttatgttctacaggaatttgtacataaatgttcatagcagctttattcacaataaccaataggtaaatggttaaatcaactgtggtacatccacaccatggaatactactcagcaatagaaaggaataaaatatcgacatacacaacaacttggatgaacttccagagaattatgctgagcaaaaacagtgactcctaaaaggctacacacggcataattctctttacataacattcttgaaatgataaaattatagaaatggagaaaaaaatcagtggttgctaggggttatggaaagggtgggggtcaggagaagggagtgtggctataaaatagcaacataagggaaacttccgtggtgaaaatgttcttatcttgattgttatcagtatcaatatcctggctgtaataatgtactatcattttgagagatgttaggattagcagaaagttagtagagaatacatgggatctctctgtattatttctcaaccccaagtaaatctacaattatttcaaaagaaaagtttaatttaaaaaagcaatacttgtacaaatattttaatgaggtatcctaatatttcacaagatacctcttgcagttttccgagaaagaatttttaatgaatctttaagaatcagactcaattgattcttatgactttaaaatggaaatcttggcttatcaaaaaaacttatacatcctcaaaaaatgtaattggtgtttacatgtgatatactaaaattaaattttttttattgaggaaattttaaagtttgggtcagaatttataaacaaaaagagttagaaagacagaaaatataaatcaatttttcccagtaaaaatcagatagaaaatataattctaaagagatatatttatagtggtattattaaatataatgcacacaataagaaaaagtgtaagccatttatggataatattgtaaaacttaatggaaagacattaaaggaaatctaaataaatggtaagctatataatattgtaagtaagacagtatcttaaagatgtgaattttccccaaatagatctataaatttaatgatattttaccaaaaatcctaacagcatgttttcatataccttaactgattctaaaatttgaaaaaaagttcaagaatggccaagatgctcctgaggatgtaaatcaaagttatgggacatccccagtaggatatcaataactgttacagagatttagggattaagccagcgtggtgctggtgcagaaattaacaaaatgacaaatggaaaaaaatagagtgcctatgaacaaacatatacccaaaatggaaactgttatttacaaaatggtcattgcagatcacaggaaaaataactttttaataaattgcaccggaataatgattatttataagtattacatcgatatttgatcccagccaaagatctatttcaagtggaatgatttgaaggttaaggagcaaactataaagctattaaaagaacataagagaatatatttgtggttttggggtgtagaagtatgtttttaaaaaaacacaaaccttaaaagaaaaaatagacaaatttcactacattgaagttaagaattttcttcatcaaaagacacaataaagaagcaaaaagcctcaagctgagtgaagacttttttaaacataatcaataaaggctttgtatctagcatctataaatatttcctacaaaccagatgtttttaaaagacagccaaatagattgggaaaaagttataaacaagcattgcacagagaaaaggacacacaaatggtccaaaaacatgtaatgcttagcctctttggtaaccagggaaatatatattgaacccacaaacagacaattaccatttcatgcatgacagactggcaaaaattttaaaggtgcacaacaccaagtgtcagttaaattgtggatcaaagaaaactataacacatggctggtgcgaaagtacattggcacagccatttgtggaatgttttggcactgagcaataaacgtgaacatgtacagtggatataccacaaaattataatatctgagttcttgaggcaaacaggcaaggtctggagctgccacagacttttctaccccaacacccctacaaacaatgtcctccatgtcctgcaggtaaaccctacagacaagcacaagaacagataacaacttctggtgaccactgcttcactcgtcaaaaggaatgaatacagctatgcacattaatatggatcaatttcaaaacacaatggcaggagaaagaagcaagtcacagaaaaacatatgtggtatgattctgttcacataaagcccaaacagacagaactaagccttatatcatttaagaaattgtacataggtgacaaacttataaagaaataataaaataaacatagagagttttacatttgcttacaatttggtcaaagaaaaatgggggccacgctgggcacggtggctcacacctgtaatcccagcactttgggaggctgaggcgggcggatcacgaggtcaggagatcgagaccatcctggctaacacggtgaaaccccgactccactaaaaataaaaattaagaaaaaaaattagccaggcgtggtggcgggcgcctgtagtcccagctactcgggaggctgaggcaggagaatggcgtgaacccgggaggcggagcttgcagtgagcctagatcgcaccactgcactccagcctgggcgacagagactccctctcaaataaatgaatattaaaaaaaaaaaatgggggccaggcacggtggctcacgcctgtaatctcagcactttgggaggccgaggcgggtggatctcctgaggtcaggagttcaagaccagcctggccaacatagtgaaaccccatctctactaaaaatacaaaaaattagccgggcgtggtggcaggcacctgtaatcccagctactcgggaggctgaggcaggagaatcgtttgaacccgggaggcggaggttgcagtgagtcgagattgcgccatttcactacagcctgggcaacaagagcaaaactctgtctcaggaaaaaaaaaaaaaaggtggggggcaaagggtagaggaaagtcggggaggggtggctgatgaggaacaaggagagggagagagtagaatagataattatgcacactggagtcaattatttgagaattaataccttaatgtcttcattactgagttctacataagctctaatcacctttgccctaggctctgggaatttcaatgtatatgttgataatgattcctcaaagcacagtgttctaaaaggaaaatgctattttggagaaataagcctgttaccaaaatccattctgcaccaagccatccaaacatcctgccccttaaaaggtggtcttaaaatgaaataacgctgaagaggtgaatcagatgaggaaatgaacttacaagcagatagggaaagggcaattctaatgtctaattcaccccatataaaccagtgtatttatccgcctcttcccatcgctttgcatttccatgatgtctatcatcatgccaccagggccgttagatgcaagatgttaaattggaagaaagctgttaaataaatggggctttagagagcctaatgaaatgtactttccatacacaacacgtcctcagagggaagaattgccagtttacaggatgatttatgtgcaccctgaagttaggtctatgccagaatttttagtactgggtaaactcatttaatcctatatttattaagcataggctaataatttctgacagttttacatgatggccatagcacagtgcatgggaaatagaagaaatgggtgctacactaagtactataattcagcaacggtgctactttgggcaaaccattgaccttagctctttccaagtataatcatgtaaaatgcctacctacagacccatgggtctgtagaagcatcaaatggcttattatatgaaaaaaatgaacaggtagcataaaagctcaggccaatttaaatgggcaatcacatatacttaaatctggagccaccaaacaaaattccaggttgcggtcctttttcctatggaaaaccaaccaacatccaaggagcaaaaactaagatttttgtaatctacccttttccaaaccaaggtcagatttgtacttaggctgaccaaaatgtttaattctgatgaggctgtatccttatcttacaaaatgggagaggataatggagggagggtacaatgatgcagatgccagtctatatttctagtgagtccaattctacaactgtcttaagaataaaacccaatacacaatacaataaaatctacaatacaagagggaaatccatgcacagaaacattcactgaagcagggcttgtaatgcaaaaagcgggtggggtgggtaagtaagccaaatgcaaagtaataaatgaactgtgaacacctattatgtgccagccaccaccttggacacttcacgtttattttctcatagggacttcacacctatcctatgagatatttatttctatccccattttgcagaggaggaaacaggttctgagatagtaactatcaagtaatttgtctaaggatacatgcatagtctaaatctgtctaattcactccactatgaattcttgtcatcattccttagagtgtagacattggggagggcttataggtgggaccacaggaaattaggtaatgtactgggtaccatctgtgtgctcatccacctccattcttgccccttctccacctgttctcagctgaagagactgacaagtgtgggctacatcagtggtctctccacatctccctcactgccaggtggccacagattggaagcaggaagacagaaaagtcagggtttttggtttagagctggctgtgtccctcaacccaaggtctgagcttccatctcgaggaggctctttctccagtgtcctgaaatcattctggcagtacttcactcattcaggcaagggggtaatacggccccactgtcctaggtcagaaaactgcattatcccctgtggcttctctacatcccaaactctcctcaattacagaatcatcatgtgccactgtactttaaaacccaagtgtgatccaaaggcaaaatgaattgaggctcctcaatgaccagcagagtgaacttcgctgggggatgaatgtggccactgattatggggctgagggtttcttgagctggtgcaatttcattcccaagatccctactccagaagctcagtcaaaacactgagactgccaacctagttcccagaaatagaggtgtctttcctgcagacctcagaataatacagatgctatggtttgctggatttaatcccttaatgttttacccagggaatggtaatagtctaatgaacatggcaggtatttgggactaagagcagaattatggtgcaagagcttacacgtagcatgggagtccaacagatgcagagtcagttccctgactcaccagctgaccttgggcaagccattcctcagtttctccagccataaaatagggataatcatagttcccaccagagatactcttcatcaggacctttgatgtcagtttccacagacaaagggctggtataattatcccaggtgcagtaatgagaaaggcagaaagccagactgttttttataggacttctcaggtagagtttccggcttctgtccatgagtcatatggacaatgggcccagcttgctcatacagtggatctgccagggagaagagtggagggctgggggatgtttttccaaactttgtaggtttgaggggtgccatggttgaataaagacaaacaggacttcagaacctgacaggtgtgaattcttatctctttcctgttgcttatttcatctatgatcttggaaaagttgcttaagctttctgagcctgagttctctaatctgctgctcgttgttgttgttgtagttgttgttttgaggtggggtctcactctgtcactccccaggttggagtgcaggggtgaaatctcggctcactgcaaccaccacatcccagactcaagtgatcctcctacgtcagtctcccaggtagttggaaccacaggtgtgcgctaccacgccccactaactttgtattttgagtagaaatggggttttgccatattgcccaggctgatctcaaactcctgagctcaagtaatccatctgcctgaagttcccaaagtgctgggattacaggcttaagccaccacgcccagcccacagcttttgaatggatagatgtgatcgtttaatcaaaatgtctaccagaatgcctggcacattgtaggtgcaaaaatgtccattctttctctttttaaaaataaaccttattttttagggaaaatttatcttcacaggaaaattgagcagaaagtacaaagagctcctgtatatcccctacccccacacattcacagcctccctcattaccaacatttcccactagagtggtgcattttgtacaattgggtctatgttgacacgtcattttcatagtttccatcagggttcattcttggcattgcacattatatgggtttgaacaaatgtataatggcacatatccaccattatagtatcaaatagagtcattttattacctcaaaaactctctgtgccctatctatttatccatccctctaccctaattcctggaaaccactgatctttttactgtctctatattttgcctatcccagaatgtaatatagttgaaattatacatcatgtagccttttcagactggtttctttcacctagtaatatgcatttaagattcttctgcgtgtttgcatggcatgatagctcatcgctttttagagtggaataatagtccactgtctggatataccacagttgacttatctgttcaccagttgaaaaacatcttggttattttcaagatttggcacttttaataaagccgctatacacatacatgtgcaagtttttgcgtagacataaattttcaactcattgggtaaatatcaaggagggcaatggctagattgtatggtaagaatcagtttagttttgtaagaaactgccaaattgtcttttaaagtggctgtaccgttttgcatccccaccagcaatgcatgagagtttgtattgctccacatctccatcagcatttgctgttgttggtgctttggattttgccattctaagagaaggtgagtaccttctctttttaggaatcccaaggatttgaagataaacctggaaaatctcagctatgacttggtgttaagcagtcacgtagagagcagcagtaatcccgaatagtaataagaccctaaccactacattttgcacagtatttctttccattgttatatatatgtgtgtgtatatatatgtgtgtatacatatatatgtgtatatgtatttgtgcatatatatgtatatatgtgtatatatgtatatatatttctgcatatattatgtgtgtgtgtgtgtgtgtgtgtgtgtgtgtgtgtatatatatatatatatttttttttttttgagatggagtctcactctgtggcccaggctagagtgcaatggtgcgatctcagctcactgcaacctctgcctccctggttcaagcgattcttctgcctcagcctcccgagtagctgggaccacaggtgcgtgccaccaagcccggctaatttttgtatttttagtagagatggggtttcaccatattggccaggatggtcttgaactcctgacctcatgatccacccgcctcagcctcccaaagtgctgggattacaggcgtgagccaccatgcccggcccattgtttaatatatcatcagctggtatttatcacactttctactcagtttgtttcaatggcaatataaaccaacacagaatctctgcccaataaaatagacacattttggccatatctagagccaagaaagtgaacatgagcttagaataacacagacacctactttccatttgtttcatcagtaaatattaatccagtaccttctggattctcaaaaggttttgacaaaaagggcaaatatttgtgcagagatgagactagtgacccttagaacaagaggaagattgggatcaggagaggctggaagcttttacatttggagaaaaccacacaagccaagctcctgagaaaagcttgttttgtgggacaggaagataaagaagaggatagcaaagactccagcttatctagttatgatccagaattggatcaaaactggcaaaaactaattggtgatattagggtccatattgctgagcaaagatagttgaagagatgaaacatctgtactatgatcaacataatttgcataaggacacctgccgacatcttaaggaacagcctttaatctcattcgttatagtgtattgcttttattagcctgtgacagatgcattttaaagcttgtttctataaagtggaaaacggagttatgtctatgcagtttaaccaaaatataggtcaatttgggttgtcaaatagccagttatttggcactcattttggttttctttcttcttatgttttgcttgtttcattttgcattttccaaaatgatgatattggagataacaaactgttaggtccttgttattctgtgcatatatgattttgtcctaagacaagatgaaataatcatatctcattttactatccagttatttggggtgtcatcttaactagcagttaggattagcatgttactcaagctcacaaagacatagctgggatgacaacatgttctttgttcagagtatttgccacattgaggactcctggcaaaaataaataacttataagaaaggtaacttattttgactttaaaataatcgatgactaaaactcatttttcctcagaccatgagagcaatttaccaagctttattaatgggcatcttcatatccttagcaagcttaattgctaattaattaaaagatgattggataaacaatggattgtactacaaaatgaagatagcaaaatttactgtcatggtgtctaatgagcattctttacctattgccctaccaatctttcagctccataatttctgaagtaaagatccccaagagccatttcctgaaaattagagttaaatcagatcaacgttaaaggacttctgggtcaaactatgttgagggccagccacaggcaatcataatttaattaaagcaagagagagaaaaaaaatcatgccaagtgaaacagcctggaagagtgacaaaagcctttgtcttaaaatcagaatacctatgctctaaacatttactactgtggaaactagtgaaagataatctaatttttctgagcttcatttttctcatctataaaatggatatgatcagttcagctgcaagtaaaagaagcccaaaagtaacagaggactaagcaagacaggagtttatttttctaacttgcaaaagatccaaaggtagacagtcaagaactcacagcagctctgctccacggaaatttcagagcctaggttccttctatgttgtttttcctccatgctatagtctaaaaagacttctcaaatcctagccctcatgcccaagttcaaaccagcaggaacaaatgtataaagaaacaggggcaaagcatctacaccagatctctgttaaggaaagtatctggaagtttccacacaacacttcatcttacatcccactggagaagctagtcatatggccacatctagctgcaagggaggtgggaaaatgtagtgttattctggactgccatgtgtccagcagaagggattttatcactaataagaagtggtgagtggatgcctggcacggtggctcatgcctgtaatccagcattttgggaggccgaggagggtggatcacgaggtcaggagatagagaccatccgggctaacacagtgaaaccccgtctctactaaaaaaattacaaaaaaattagccaggcctggtggcaggtgcctgtagtcccagctactcgggaggctgaggcaggagaatggcatgaacccgggaggcagagcttgcaatgagcagagattgcgccactgcactccagcctcggtgacagagcgagactctgtctcaaagaaaaagaaaaaaaaaagaagtgggaagtggaaatcagaaaacgcctagctgtctctaatccaagatatagctcaaagctttgttaggagagtacacggagagcatggatatgaaatagctagcagagtgtctggctgattaaaaaaaaaaagccagaaatgtttaataacttctgtctgaatcagatagacaaaaaaatagaataaggttttcctgagaaccttgacccattagagaagaacgggagtaggctctcttagtacctgcatctacagcaggattaaattccccagggcagagatgagacagggaatggcttttctctgaaccaagcttctgttctagtgtaaggagccaagacaagcatctcattcctccatgtctttgattatacacttttctctctccaaatcttcttcttgcccatttttcaccttgccaagactcagttcaaatattacttcataaaagaatccttcctgaccccccaggctgggttagatgcccttttgctgaattatcgtaagagttggtgcatactgcttccacagaaattcttgctgtgttgaaattagtctgtttgcacgtctctaccactgaagtgtgaactccttgaggaaaaatataaagccttagatatcatcatcttccccaaatttttcaaaatattagatctcaatcccttatttctatgcagggaactagaatgtttgatgaacattacaagacatagttggcaaaatgataatataacattttgtgcatgacttgggaatagaatagatatatggttctctttgttgattcactcaatatctatgggcagcatatggcacatattaattgggtccttggtcaatgcttgttcaacacaataatacaacttgttcaacacaataatacaacttgttcaacacaataattgaaggttaatatttattgagaagccaataatccagagtgtgtagtgacaagtttagaaaagataaagcactccgtatttatgtgctcttggtgaaagagagaaggatgaggctaggtgcagtggctcatgcctaatgtaatcctaacattttgggaggccaaggcagaaagattgcttgagtccagaaacttgagaccagcctgggcaacacagcgagactctgtctccacgaatatatattatatacattagccaggcatggtggtaggcacctgtggtcccaactactcaggaggctaaagtgggaggattgcttgagcctgggagtttgagcctgcagtgagctatgatcacaccactgcactccagacgggatgacagagtgagacaaaacaaacaaacaaaacaaacaaataaacaacaacaacaacaaaaaacagaaagaaggatgaaaaaacaaaatcaaaagatatgtgttctttttaacctcccgaaacatgaactgagaataattcccatgatacaacattattagaaaaacaacaacaattagaaactgaaagactgagagctctgttccaccactgacaagcgtgtcattttaagtatttgttttatttctcctggtcaatgtgttggggtaatggtgtgggttttagctcttaaatcggattcttagccttggcagcattgacagtttggaccagataattgtttgttgtgcaggctgtcttgtgcactgtaggatatttagcagcattcccggtctctgcccactaaatgccagtagcacccactcgtaaacatagactgtgacaaccaaaacagtctccagacatttccaaatgtcccctaggtgggtaacagtgctctgccccctcccaaacacacagagttgaaaaccacagtgtagacttaaataaaattactaaagaccggtctatggaaaataatatacttccaaaattaacatatactttctttctcagtctcagttcttttccctaaaaataaaataaaataaaataaataggctgttgcactctagaaactactctaaaacaactacagatcaattatgcaaaaaaaagtctgaaagttacagtacatgaggggggaaggaacccttaggtttaacatagaattatctcagttaaggtgactgcataatgaatctgacataaacatcaatttgactgcatgttgctttcattaaagcaaagaaaccagaaaggtggaagaatccttataccttatgctgcatgcatcacaacacaccaagtatactagacctagttctgggaacctcatttcaagagcaatggtgcaaaggagagcagccagaatgaggagaggccaacagaccaggtccactctattccacagtgattcaagaaacgttactgaacatgttgactcctatgttccaggagctgtagagacggagttggatgccacattgacgcttccctctagaaacttacattctagtagagggagccagtgtgcaatagaatatcatggcaataaacacagggctatactgaatagtgggactgttgcatagctaagagttatgcaagcaccaagtataaagaagcagcttctgagttgatagtgctgttttgtgccttttcagaggtatgttttagaaaaaataactctaatggcagaataaataatggaaataagacagtgaaactaaaagtaaaagaaagccactgggaacccttgcagtaattcccgtgaaaaatgataacctcacaaactaaagtagtggtgatgaaaatcgagaagaaaagatgttctgagagctagtttagaaggtagaatcatgagaactcggtgactggataagtatgatggggaatgtagaggaaaagacatccaagatgactctagcttcaaataagagaaaggattgaggaacaagggaagtttggcattaaacaaacaaacaaaaaaaagactacagggaggcaaggctgttgttcccatgtatcaaggacattatcctgtgaaaaaaagtactaggtgtgttctatatggtcccaaagctttaaactggagcaaagagtagaagttcagaaggattttgcctgaatggcagaaataattttctgagactcattgttatccaaaaattaacattccgcaggaggtagaagctcatcaagacagcgcctagggagataatggacagctaccatgaaggacatctagagattttcactgctctcctctcagcttgcttcttctagtaatgtcctgattgttaccccatcctgattgttcttcagggaaccaaagcctccttctgtcaattacttgattcagatggaatcaaggctcttctctcctgcaccaagggtggtcctgtggcttcagcctgcacaggaaaaagtctcagagaatggccccaagatgagcatgtgatctaaattatggaaagaggctcctcatcagaatttttgcagaaacaattaaggaaggcttgctctctctctgtgtggtgtagctaagagggtagaaagtaagagtgagagagagagaaagactcaaggacatgatcaagagagcctttagatatagctgtccctcagagtagttacactccaagatttcattatcacctgtgatcttttgatctattatttttttttagccagtcagtttgagataggtctattgttatctgctccccaacccccaaagaattcctcttgtggctacttgtacaggaagaaaattcaggcatagaatgagaagcgactcccagacaataggtcactatcagcaaagcttttagacaaatgtattttgaaaacaactgaaaatctttagattcagaagaaatcaaaaaagatatctcacttactgtaaggtgttaaaataaacatacaaggtaataataaagatgtctttcattataatgttacttagagaatttaccaatagccttcaatgtatcaaaagctggcacattactggttctgctcttgtttttttttaaattatagtactttctttcagaaatatactaacaaagaaaaaaagacaattgaaatttccaaatctggaacaactggattggagaaaaatatacaaaataaaccccacgaggttttaattctaagtactttagaccttacaagcaccataaacattctgttgtggctcttcctcacttagaatgcatgttaatgccgttagcacttacctctaagaccggtagcatactaagtagaactgaaatgttttttattacactactggatcattcttttaataggggatacaatctcattacaagctctagtagtcatccagattaaaatcttaattgtcaggattggtaaaagcgtaataatatatacttatcttttttttggaaatggcataattaaagaagagcaagaatgtttttctgtaagcaaggcttctcatcctcagactacgcagattttccccctttcaagtggtgtattcatgcagtacccattcttgagaaactatacgatattttaaagattctcttacattttagggaatacatgaagtggttcaccctcctgccttccaaaatatctcttttcttacttctcttcaaatgtgtccatgtaatcaatgtgaggagaatcaactttggaaacagaatatctgtgctcatgtgcaaaggaatcttcacttcctactactgttgactttgagtaaatcagttaaggtatctgagactcagtctttctcatccatgacatggaactgcaaatactacatatgtggatcgattttttttaaaaaatgtaaatattctaaaactgtaagttcttttattatttttagaatgaatgttcactgagtgcttgctgtgggtgatgcactgaggcaatgcatgttacctgttgtttgctattctaatatagaacctcagaattggagtggatcttaaggactcccaaggaccaggcacatccccttgagctaaataaccatcctttcctagtttcctctttaaagcaggtttcaagctgccatggtggtatggatttgaacatgtagcctggagaattatggccaagagtgaagctcaaccttagaaccttggacagaatggtattagcaggtacagatgagggtcagaagtggaaactacccaggaatcaatcatggaagttaaatgaagatggatgaatgaacatgtattcaatatagactacaaagatgaggaccaggagctggaaatgagtcagtaacaggaattaaatgaaaagacaaaggaagaatccaaaagttttatcagaataattaagcaaattgttaagtctggtaaaagtcacaggtgcaagttacgtgaaatagaactctgtagtcagaaataagagtaatttggagagttgtaaataatcacacatacagttctggtccatgtgataacgtgcctgctgaggttgtttatctagccatactctccattcgtccttgcctcgcttcttttactcatcaagtcctcctgtaagaaggaacatgtttgcttattcagttagctaattttttagagcccaacttttcccatgacagatctgaagatgctaacagaaaaaaaaaatacaaaataaaattgttaaatataaattctaaaacctggactagagaaaaatataaattagtataggtggagattacaaaagatagataaaagaattaatagatttgggagagaaagagactgcaaacaacaatcataaagtttttaccattagctgcagttgagatgagatttgtgttctgagctccctgtgggccagaaattaatagaaaatacagacagaaggtatatcttttattatccatagggcaaaaggagataggtgttttccttgaacatacctctaaataaaaattttcatgagaaacttccaataaaaggttttatgtgatggaagaacaatgttctacagcatctccagaacaaaggagaagcagatatttccttcaaggaaaagattgtttccattgaggactcactattggaaatacagatgtcaatgtgaagaaaagagtggggcaacagcctctaatcccacaggactcttttcacccagtgtgtggagcccctccccaccccttcttaaagctctccatggttctgacccctgtctctcatcaaccctaactttatgatccaggttcagcctacacatccatgccttcatctctcttgccacatcccacaaagatcaaccccacttttttatcaacttgaatgtgaaattatctaaggactagcattcttttaggctccaattaaaacttttgagaatactggaaatgctcaaagtgtgtcaactctcactcatataaccacttaattctgttctcaagtcttagtccacacttgtgctctgagacatttaacactctatacttagtacctaacaaataaaatgacaattactaattaataactcttataaagtacttatgatgtttca

SEQ ID NO: 2 - DNA, 23 bp, Homo sapiens
ccaatagccttcaatgtatcaaa

SEQ ID NO: 3 - DNA, 20 bp, Homo sapiens
tgaggaagagccacaacaga

SEQ ID NO: 4 - DNA, 237 bp, Homo sapiens
  misc_feature (52)..(52): n is a, c, g, or t
ccaatagccttcaatgtatcaaaagctggcacattactggttctgctcttgntttttttttaaattatagtactttctttcagaaatatactaacaaagaaaaaaagacaattgaaatttccaaatctggaacaactggattggagaaaaatatacaaaataaaccccacgaggttttaattctaagtactttagaccttacaagcaccataaacattctgttgtggctcttcctca

SEQ ID NO: 5 - DNA, 21 bp, Homo sapiens
cctcccaaacacacagagttg

SEQ ID NO: 6 - DNA, 23 bp, Homo sapiens
tgttaaacctaagggttccttcc

SEQ ID NO: 7 - DNA, 263 bp, Homo sapiens
  misc_feature (206)..(206): n is a, c, g, or t
cctcccaaacacacagagttgaaaaccacagtgtagacttaaataaaattactaaagaccggtctatggaaaataatatacttccaaaattaacatatactttctttctcagtctcagttcttttccctaaaaataaaataaaataaaataaataggctgttgcactctagaaactactctaaaacaactacagatcaattatgcnaaaaaaaagtctgaaagttacagtacatgaggggggaaggaacccttaggtttaaca

SEQ ID NO: 8 - DNA, 20 bp, Homo sapiens
ttgaaattgcaatcccatca

SEQ ID NO: 9 - DNA, 21 bp, Homo sapiens
cctccctacttattcccatgc

SEQ ID NO: 10 - DNA, 392 bp, Homo sapiens
ttgaaattgcaatcccatcatcccccagaactcctgatatcccctacactcccttatacttttttgtctatagcaaccacccctcaccactttataacatttatgctttgtagtctgtctgtgtccactcactagaattcaaatatcacaaaagcaggagtccactttttttttcattgaaaaactccaaatcctagaaggaagctggcatttaatatgtgctcaatagacattagaggaagaaaagaaggaaggaaggaaagaagggagggagggagggagggagggaggaaggaaggaaggaatgaaggaaggaaggaaggaagaaaggaaggaaagaaagaaagtcaagagacctgggctcaaatccagcatgggaataagtagggagg

SEQ ID NO: 11 - DNA, 20 bp, Homo sapiens
tgatgcaccacagaaacctg

SEQ ID NO: 12 - DNA, 20 bp, Homo sapiens
caaggatgcagctcacaaca

SEQ ID NO: 13 - DNA, 169 bp, Homo sapiens
tgatgcaccacagaaacctgtcagttggtactgatctaccctcctcctcctccttctcctacacacacacacacacacacacacacacacacacacacacacacacttcatcctactctccagcattcagggaagaaaacagaggcaaatgttgtgagctgcatccttg

SEQ ID NO: 14 - DNA, 1002 bp, Homo sapiens
gtttttaaacatatttttttcgctgacctccaccctgtaagagcttttattaccaagcgattgagaagcacaggctcagggacactgaatttgaccaaagaagccaatagaactattccaaaaacctatggttccccctaaagcattagaaagactcagaacgggttaagtgctccctggctcattcccaacagacactacattcacctgtgcttgctctgaaataaatcagtgtccctttctgctgctgctgttgtctggaaataatgcaaatgcaatgggcctttactgacattgtgcttccctggaaggatacacataataaattatcccttaatactgttaaagagacattttcctcttactcaggagcttttggggttggactgggctactcacccagcaaggaggaggacatgtgtcttgtcactggcccggttattcatgtggcctctcattgctccttggctcactgcattgcaagattcaaggatgcacttcgcaggcctccacatcaagtcataggacttgccggtaacctagattggttttctcatttgtaatttgaatttattttatgttatgcatttgtatgtttatttattcggatgctcagaagctgaagataactagtgctcctggtccatgccattcatcaattggaagaatgccaagctgtttccgctgaggacagaaggcattggtctcccctgcaggaagccactgctgctccttaattgtttgctagaggaagaatcaagggtaaaatttaaagtaaatggctggccgagttgcactaattcatcaaagcatgtttcaagtcagtagtcagagcatgcatcagcccccggcgccaccagcttctacgagagtggaaaagccagcagacctccgagcagatgaaatcattaggaggcattcagcagggcttgaaaagcaaagagagaggaggcggggatttctctgcatgctccctttgccacatgggaaacaccagctgtc

SEQ ID NO: 15 - DNA, 1002 bp, Homo sapiens
cccaaattatcctcacctctttataagtctcccataaccctttcttaccctattttaagcttcttttaaatatagtaaggaagagtttctctggccttctttttttcctcaaattttattttagattcaggaggtacatgtgaaggtttgtaacttgggtatattgcatgatgctgagatttggggtgcagctaattccaccatccaggtactgagcatagtactcaatagtttttcaactctttcccctctagctccctccatcccccatctagtagtccccagtttctattgctgccatctttatgtccataagtatctggtctccttttaaatttgctttcttctttgctcattatctagaatttccataatagaggagaacctgaaaccacacccaataagaaagaattttatctaaagttttactacctttgcattccagtctttctctacccattctcctaatcttgtctcgtgaaatcatggctgctgagaatagagatttcttttggaggacaatgaaaaggatgggaggacagaagctacacagaagggagaaaggaaaacagagcaactgaagacaaaaattactttagaaggtgtaagcacatacaaacagggctgaggttatatgtttcactttgaatgaatctcatttaccgagataccaggagcattttacttaagtctttgagaacacgagttttactggctatatcatactctgttgtagaaatacactgtaaagtactttcactatcctcttttattggacatttagatctaaatgaattttgtgctaatatgaatattgtatgatgaatatctttgactatattttgtgcattttgttataggcatgtatcttgaaaacggcagagggaagattttgctttgttacccattttgataggccttgcctttggccagacatgttactgatgttttggtattgaactgatgtatgtcttcatttatttgtttttatttatttttatt

SEQ ID NO: 16 - DNA, 2002 bp, Homo sapiens
cagaactaggaaaattgccaaaagttatgggtctgtacagagttagtgtcacagtaagaatctcattgcccaagcaatagggtctaaaatcacgatcttattcaaagtaacagcgaccacttacctcatgcctcatatgtgccagatacttttcttacattatttttaatctccatagcaattatctaaggtagataatatctagagatgaggaaactggggctctaggagtatgcaagatttgtccaaggtctcacagcaatatcttagtagagtctgtctagaatcaaagccaatttgtctttttgccctatcatggttcatctctacttcactctaactccatcctaaaaaccaccttccccatccactatataaatgaatgatagcaccaccctttcagtaaaaggatctagacattcaccatctctctaccatcctagcagcaactgcaatgcttggaaaatagtcgaggattagtaagagcttgtcaaatgagacacagtttgttgttctggccctgacatgaaacaggtaatcaagtaaacgtatattttatatatagtcacttcactttcctagtcactaatttccttatctataagacaagggtattgggccaaaagtctagtcttaaaggttcctttcaagtcatttattgaaagtttgtctgatactttattttttactaaactttatatattccttaaatacacactcaaagaaacatatacaggtaaatacagacaagctctatctaatggtgttaactgtcacttagtatataaagacatcttctctcagagaaattggtcacatgttctttctttagacaactgctcatcatgtcctttgactaatcataagccaacagtaagaagttaagagtgccaagaaaaggtaactgtgttaagttgcatttgtatttttccaagtatttactctcccattctttcatatctataagaggattatccatccccacccactggcatgtgcgctacagtgcctccatgaggggcgtttatctgtttttcttcacaatgaatttatcacattccttgctttggccaatagaatgtgagtgggcatacgatgtgtgcatgtctgaacagaagtcatgaaacaattgcctggttctgatttatctcctgctttttttttctttggcgttaaattggtatgtgcgagatagaggttgatctttcaactttgacctggtattgagaaggcacctgaggcaaaaccagagctgatctagagttgacatacacagtggacatataaaatgaataaaagataaaacttttagattgtaagccactgtaatttggaagatgtttgttactgcagcataacctatcaaaggctgacttataaaaaatatttcagataccgttagttctcactgttcacagtagttatgttttatgaagtttccatggatactgaatgagcgaacagtgaactaatgttcctaggtaaaatagaagattaggttcccgtgagctctgggcaaaacattttcatcatccaagcaatacataatcttgctttatgtgtgtttctatgtaaagacaccttattcaatatattttgttgattcattaaaattaaactcatggccagcagcattatagctcatgcctaaatgaggcttatctaacatgtatatattttctataagacatttcacagtcttcttgactcaagaacactacacagcacttcagcactatgctgaaatggggccattttaaacagaaaaatcaccaccaacaaaaattagctgggagtggtggtgcacacctgtagttcaagctacttgggagactgaggcagaagaatcgtttgaacctaggaggcagaggtcgcagtgagacaagattgcaccactgtacttcagcctgggcaacagagtgagtctccatctcaagaaagaaaacaaaacaaaaacaaaacaaaagaaacaaacaaaaaaacacttcatcaaaaagcataa

SEQ ID NO: 17 - DNA, 1002 bp, Homo sapiens
taaagctcttaaccccacaatgccctgtccacagactctgaaagatgctgatgcattgttgtgtcccatgtctgtttccccagcaggttgtgagttctcagttgaattcagtttcttgttgcagagtctttatcaaaccacagaagaatcaaagttgaacaacatggagtatctacaccggagcagcccacagttcagggatggacacagaacaagagagattcattacagacataaagcacagagatgttggggttttctctgttgggaagaataagaggtccagaaaagcttcccaaagtgatggcacctcaagggtcaggacctcaccttattaatctccatgacccagcatctactacagcatctgtcacaactgggctctgagaatgttggctaaataaatgaatgaatgatatcaatacacagggtttttccccattttctgaatattctggactaggggatatctcagaacagtacttagcacctagtgtgtgcgtcaataaattcttgttaaaccactaaaaattgctggacagctgaactgaaaattactcacagccccattcaactgcatcagccatgaaaatcaactcagaatttgcaaatctatgctggcatttagcacttaagatgtaaatacagagtgtcagccatgtggctaagatcagctttaattcagtgttcatctctgaaattcattaatgattaaatacttttttcctttgctctctatgggagttgaaacaagtatcatgtatccaaagaccagggttcagtttggcccaacattaattcacttaatgtttcaacaaaaatttattgaccatctactaagtgctgagtgctagaatccattgactacctactaatgaagtgctagattttaacacagggacatctgtggtaaaacagtaaattctctaacctcatctagaggggttgaaggttctgcctttgcctaccttctatagtcagagactactggtatttcaatcc

SEQ ID NO: 18 - DNA, 1002 bp, Homo sapiens
gaccaaaattaccgtcaggacagagcagcctgagggcagcgctatcaagaggggagagccccaagttgtctgattggtgatgatggcaggttggtgatgcttcttaccacattgctatcctaagcagcaagtggtcccacctcagatttgcctctaccattcctgccaggaaaccagatggcaggaagagcccatgaatcacctctctgggataagcagaacagtacttgtgtattcttgcctttgtggttgcttattctttcacaattccaataagcaggccagtgtcaattgcctgctggagaatgcacttgattcttccgtgtacagtatcagaatatgatttttagttttaatggtaagaaatacgaatagtattcactcttttcctcattcccacagctgtgactggacttttggcctctgatgatcaacataaatcccacctccatcccactgatgctttttaactttaagaggctcttcagtaccaccggagtctttcaggggatagagtggatccctagaaaccgatcaagggccaatctgcagtgagttacccaggagtttagagattcccttcgtttaggtctgttgagtttaatcaatatttattatctgagcacatcctttgtgaacatccctctgctagagtcaggaaattagagatgaaacactcatggcctgtgccttagaggaactctccattgagcagtggagacaggagaaaatggagaaggagaatgtgctctgctggacccagaggagagacttggggagccctcagcagaggcccttaactcctttttagaaacagggaaaacttcctggaaaaggagacgttttcatctaatcattcatcatgtcatatattcattcaataaacatttattgagcccttgctatatgccaagctcagcactacgttcaagggactcaggagccaatgagtcagacagtgtcctgccttcatggagcttctatataatcttgaggaaatcc

SEQ ID NO: 19 - DNA, 1002 bp, Homo sapiens
aaattaacatatactttctttctcagtctcagttcttttccctaaaaataaaataaaataaaataaataggctgttgcactctagaaactactctaaaacaactacagatcaattatgcaaaaaaaagtctgaaagttacagtacatgaggggggaaggaacccttaggtttaacatagaattatctcagttaaggtgactgcataatgaatctgacataaacatcaatttgactgcatgttgctttcattaaagcaaagaaaccagaaaggtggaagaatccttataccttatgctgcatgcatcacaacacaccaagtatactagacctagttctgggaacctcatttcaagagcaatggtgcaaaggagagcagccagaatgaggagaggccaacagaccaggtccactctattccacagtgattcaagaaacgttactgaacatgttgactcctatgttccaggagctgtagagacggagttggatgccacattgactgcttccctctagaaacttacattctagtagagggagccagtgtgcaatagaatatcatggcaataaacacagggctatactgaatagtgggactgttgcatagctaagagttatgcaagcaccaagtataaagaagcagcttctgagttgatagtgctgttttgtgccttttcagaggtatgttttagaaaaaataactctaatggcagaataaataatggaaataagacagtgaaactaaaagtaaaagaaagccactgggaacccttgcagtaattcccgtgaaaaatgataacctcacaaactaaagtagtggtgatgaaaatcgagaagaaaagatgttctgagagctagtttagaaggtagaatcatgagaactcggtgactggataagtatgatggggaatgtagaggaaaagacatccaagatgactctagcttcaaataagagaaaggattgaggaacaagggaagtttggcattaaacaaacaaacaaaaa

SEQ ID NO: 20 - DNA, 1002 bp, Homo sapiens
tagagaaagagacaaagcaggaaagagaaaagagaaaggcatatatatatttttttcttcattctgggggcccaccctgaaactactgaatcacagtctctagaggttctcaggcaactagcccagctgtttttgccaactggaatttatgagccaccgcaagagaccacatgcagcttcatgtaaaacaaattatttttaagcacgcagactgagcagtgatatgaggagtgcacaggagtgcctacgcctactcctggtctccatgagtctcctttgcaaagtcaagtattacaagattctagaacacatattgcctgccactgataatttagttgttcagcaaacattcatttgttgagttgcacgccagacactatactagatgatgggacaactaaagggtaatgaacagttctgtctctatgtaaaaataataatgatgatgatgatgagatgggacttcaattgaggaagtgccattggggaggtatgtaaaaacgtgctatggaaaaaaagcaacaggaaccccttgatagaaaaaaaaatgctggtgggggtagggatttctgcctgtgttcttcagaatggggtatgggaaaatctgggaggaaaagaaatttaagtaagagcagagactttgcaaaatttgttgtgttgacttttcctcatgctgcttcccctggcatgggaagtcattagctggataagagagacttcacaagaactgcaatgaatcaagatgtgctggttttgttttgacacatggaattcttagggatttgatgttttttttcccagtcttctccatcaaagttgttttcaaccagtcctgattggaccgattgactcatcctcagatatcatagttttcccactacaaaagcatggaactgatgccaataaacccactccttattcccagagggctagggtgagtccttgcagaggggaattgctagggatggcacctggcagaaatagaccatctgtctttcctcc

SEQ ID NO: 21 - DNA, 1002 bp, Homo sapiens
tggttttctttcttcttatgttttgcttgtttcattttgcattttccaaaatgatgatattggagataacaaactgttaggtccttgttattctgtgcatatatgattttgtcctaagacaagatgaaataatcatatctcattttactatccagttatttggggtgtcatcttaactagcagttaggattagcatgttactcaagctcacaaagacatagctgggatgacaacatgttctttgttcagagtatttgccacattgaggactcctggcaaaaataaataacttataagaaaggtaacttattttgactttaaaataatcgatgactaaaactcatttttcctcagaccatgagagcaatttaccaagctttattaatgggcatcttcatatccttagcaagcttaattgctaattaattaaaagatgattggataaacaatggattgtactacaaaatgaagatagcaaaatttactgtcatggtgtctaactgagcattctttacctattgccctaccaatctttcagctccataatttctgaagtaaagatccccaagagccatttcctgaaaattagagttaaatcagatcaacgttaaaggacttctgggtcaaactatgttgagggccagccacaggcaatcataatttaattaaagcaagagagagaaaaaaaatcatgccaagtgaaacagcctggaagagtgacaaaagcctttgtcttaaaatcagaatacctatgctctaaacatttactactgtggaaactagtgaaagataatctaatttttctgagcttcatttttctcatctataaaatggatatgatcagttcagctgcaagtaaaagaagcccaaaagtaacagaggactaagcaagacaggagtttatttttctaacttgcaaaagatccaaaggtagacagtcaagaactcacagcagctctgctccacggaaatttcagagcctaggttccttctatgttgttt

SEQ ID NO: 22 - DNA, 1002 bp, Homo sapiens
taaaggacaggcattggggttgctttgttgaacaaatctagcagatatttgaatgagaagagtaatatagtcagtagaaaaaaagtgcaagaaataagtagagaaagaagggatattttctgctgaagcatgtattctctggcacaagcccacaataaattgaaattgacaccaacagttggctcaaaaataatcaactacaaatatgctcaacacataagcattctcttggacagaaccacaaagcatggtctgcattgttcctaacaactctttagaagtcaccagatgcagtttaagctacaataacatagtgaggtacaagttaattacatagttaccagaaagtcacagacttttttttcagtaataatgtagtaaataaatacatgctcactccatgggaaatggtggcaattattaagagcacacattcacaccatcatattgcttactgataactgtgcagttaaccaatggcagtgtgctaaaatggatatcttgtgtttccctgagttttgcatgctacatgcgatgcatgtgaaaaccaagcatagggaatttcaagtatgaacttcagcgtgtgagtgttgtttgtggtccaatctccgtccccaaacatccccagaataaggcttctgctttttaacaatgtatatctattttaaccaattgtctagcgtataattaatgctctataaactctttgttaaatgcattcacagaaggtaacaaaagatttttgtgacacgagtaaaccaaaaggaacaaataaacttgaattactttatgtttgtgttggtgtttcagaaaagagctttggctttgaattcagaagttcctaatctgaataccaggtctaccaattattaattaaggaatatcaaatgaattacttgcagtatttgaatttcagatttctcaattataacaaggatgtaaagaggtttattatgtggctcaaataagaaaatgcatgtaaaaacacttgtaaaccaaaca

SEQ ID NO: 23 - DNA, 26 bp, Homo sapiens
caagtttagctgtgatgtacaggttt

SEQ ID NO: 24 - DNA, 20 bp, Homo sapiens
ttccagaaccaaagccaaat

SEQ ID NO: 25 - DNA, 23 bp, Homo sapiens
aactgcctctgacaactcttgtg

SEQ ID NO: 26 - DNA, 23 bp, Homo sapiens
ttaagatgcttgaagtccccagt

SEQ ID NO: 27 - DNA, 23 bp, Homo sapiens
aactgcctctgacaactcttgtg

SEQ ID NO: 28 - DNA, 23 bp, Homo sapiens
aagctgctgtacggatttttcac

SEQ ID NO: 29 - DNA, 23 bp, Homo sapiens
ggagagcctatttgtggtcaaga

SEQ ID NO: 30 - DNA, 23 bp, Homo sapiens
aagtggattgcagaagtctctgg

SEQ ID NO: 31 - DNA, 23 bp, Homo sapiens
ctaattgagaaggctggctatgg

SEQ ID NO: 32 - DNA, 22 bp, Homo sapiens
gtaggatcagaccatccaatgc

SEQ ID NO: 33 - DNA, 23 bp, Homo sapiens
cagggattttgtctgttttgttg

SEQ ID NO: 34 - DNA, 23 bp, Homo sapiens
tttattcggatgctcagaagctg

SEQ ID NO: 35 - DNA, 24 bp, Homo sapiens
gcaggaagccactgctgctcctta

SEQ ID NO: 36 - DNA, 27 bp, Homo sapiens
gcagtgccagcacctgttagcattaaa

SEQ ID NO: 37 - DNA, 23 bp, Homo sapiens
tgcacaagcctgatttaaaagtg

SEQ ID NO: 38 - DNA, 23 bp, Homo sapiens
ccagtttttggttttggttgttt

SEQ ID NO: 39 - DNA, 24 bp, Homo sapiens
ccagacatgttactgatgttttgg

SEQ ID NO: 40 - DNA, 23 bp, Homo sapiens
ccagagtggtagcaatgttctgt

SEQ ID NO: 41 - DNA, 23 bp, Homo sapiens
ggaatgcttccttgtatgtggag

SEQ ID NO: 42 - DNA, 23 bp, Homo sapiens
gagggaaactgactggaaagatt

SEQ ID NO: 43 - DNA, 23 bp, Homo sapiens
gcacaagcctgatttaaaagtgc

SEQ ID NO: 44 - DNA, 23 bp, Homo sapiens
cagggattttgtctgttttgttg

SEQ ID NO: 45 - DNA, 23 bp, Homo sapiens
gcacaagcctgatttaaaagtgc

SEQ ID NO: 46 - DNA, 25 bp, Homo sapiens
cttctgtcctcagcggaaacagctt

SEQ ID NO: 47 - DNA, 23 bp, Homo sapiens
tctgtttctttgacctgggttgt

SEQ ID NO: 48 - DNA, 23 bp, Homo sapiens
cagggattttgtctgttttgttg

SEQ ID NO: 49 - DNA, 23 bp, Homo sapiens
tctgtttctttgacctgggttgt

SEQ ID NO: 50 - DNA, 25 bp, Homo sapiens
cttctgtcctcagcggaaacagctt

SEQ ID NO: 51 - DNA, 23 bp, Homo sapiens
ggagggaaactgactggaaagat

SEQ ID NO: 52 - DNA, 23 bp, Homo sapiens
cagggattttgtctgttttgttg

SEQ ID NO: 53 - DNA, 23 bp, Homo sapiens
ggagggaaactgactggaaagat

SEQ ID NO: 54 - DNA, 25 bp, Homo sapiens
cttctgtcctcagcggaaacagctt

SEQ ID NO: 55 - DNA, 23 bp, Homo sapiens
ccagagtggtagcaatgttctgt

SEQ ID NO: 56 - DNA, 23 bp, Homo sapiens
ccagtttttggttttggttgttt

SEQ ID NO: 57 - DNA, 23 bp, Homo sapiens
ccagagtggtagcaatgttctgt

SEQ ID NO: 58 - DNA, 23 bp, Homo sapiens
ggaatgcttccttgtatgtggag

SEQ ID NO: 59 - DNA, 23 bp, Homo sapiens
gagggaaactgactggaaagatt

SEQ ID NO: 60 - DNA, 23 bp, Homo sapiens
ccagtttttggttttggttgttt

SEQ ID NO: 61 - DNA, 24 bp, Homo sapiens
gcaggaagccactgctgctcctta

SEQ ID NO: 62 - DNA, 27 bp, Homo sapiens
gcagtgccagcacctgttagcattaaa

SEQ ID NO: 63 - DNA, 25 bp, Homo sapiens
aagctgtttccgctgaggacagaag

SEQ ID NO: 64 - DNA, 25 bp, Homo sapiens
cttctgtcctcagcggaaacagctt

SEQ ID NO: 65 - DNA, 23 bp, Homo sapiens
tatacaccagaatgccccgcatc

SEQ ID NO: 66 - DNA, 25 bp, Homo sapiens
gatagggccgctaccatttggaaag

SEQ ID NO: 67 - DNA, 24 bp, Homo sapiens
tgtcaaccgcaacactggttgtgt

SEQ ID NO: 68 - DNA, 25 bp, Homo sapiens
ctggagtgcctctcttcctttttgc

SEQ ID NO: 69 - DNA, 24 bp, Homo sapiens
aagatgccagggctacagcaatca

SEQ ID NO: 70 - DNA, 24 bp, Homo sapiens
tgattgctgtagccctggcatctt

SEQ ID NO: 71 - DNA, 25 bp, Homo sapiens
ttgcttttaagcatgaagccactca

SEQ ID NO: 72 - DNA, 24 bp, Homo sapiens
ggcatggaccaggagcactagtta

SEQ ID NO: 73 - DNA, 24 bp, Homo sapiens
aacacaaccagtgttgcggttgac

SEQ ID NO: 74 - DNA, 26 bp, Homo sapiens
tgaaacaacagtaagcactggctctc

SEQ ID NO: 75 - DNA, 21 bp, Homo sapiens
gatgcggggcattctggtgta

SEQ ID NO: 76 - DNA, 25 bp, Homo sapiens
actcaattgttgccatgggcttgat

SEQ ID NO: 77 - DNA, 657 bp, Homo sapiens
ttgctcctcaggaaccctattttggactgacgtttaatacaacatggaagccaccaaggcttacagaatgtgctttccagagctgtgacctgaactgtacctggggccttttgagtgaggctggaactggagtggcctggatgcagagagcagtgtcctaaggctgtgcaggttgcaagaaagctcaagtagcctatggagaggatgcaaggcttccagctgatgccctcagccaggctcagtagcagccagaactagcctaccaacgaacctgctgatcatgtgcataagccaccttgaacgtcgatcctcctgcctggtggagccatcccagctgatgccacatgaagcagacacaagctgtccctactaagctctgctcaagttggatattcatgagtgaaataaatgactgttactaagtaattaatttttgggtggctgttatgtagcagtagataattggaacaaagcttattgacataatacatctatatcmcatcctccaatccatttttttaagtaataaagttgatgtttgttttgaaaaaaaaaaaaaaaaaaaaaaagacctgcccgggcggccgctcgagccctatagtgagtaagggcgaatccagcacactggcgccgtactagtgatccgagctcgtagca

SEQ ID NO: 78 - DNA, 20 bp, Homo sapiens
ccacttgggtggtatcaggt

SEQ ID NO: 79 - DNA, 19 bp, Homo sapiens
actcaaggaaagggccaaa

SEQ ID NO: 80 - DNA, 21 bp, Homo sapiens
tcagaagggcacataagagga

SEQ ID NO: 81 - DNA, 20 bp, Homo sapiens
gctgctttcaggatcaggag

SEQ ID NO: 82 - DNA, 24 bp, Homo sapiens
gggataccaacaacatctatcaca

SEQ ID NO: 83 - DNA, 22 bp, Homo sapiens
gctctttctatttgcacaccaa

SEQ ID NO: 84 - DNA, 20 bp, Homo sapiens
tgcagactgtgcagcagata

SEQ ID NO: 85 - DNA, 21 bp, Homo sapiens
ctgctagagatgtgtgcccta

SEQ ID NO: 86 - DNA, 20 bp, Homo sapiens
atgggtcttgatggacatgc

SEQ ID NO: 87 - DNA, 20 bp, Homo sapiens
gtggatggatccagagagga

SEQ ID NO: 88 - DNA, 20 bp, Homo sapiens
cagagcatcacctcaaacga

SEQ ID NO: 89 - DNA, 20 bp, Homo sapiens
atcctgccaaccttaagtcc

SEQ ID NO: 90 - DNA, 20 bp, Homo sapiens
ggcaagaaacacaaggcaat

SEQ ID NO: 91 - DNA, 20 bp, Homo sapiens
aggttgaatgagccagatgc

SEQ ID NO: 92 - DNA, 20 bp, Homo sapiens
ccacagtgattcccacctct

SEQ ID NO: 93 - DNA, 20 bp, Homo sapiens
agtgttggccagggatgtag

SEQ ID NO: 94 - DNA, 20 bp, Homo sapiens
tgatgcaccacagaaacctg

SEQ ID NO: 95 - DNA, 20 bp, Homo sapiens
caaggatgcagctcacaaca

SEQ ID NO: 96 - DNA, 20 bp, Homo sapiens
ttgaaattgcaatcccatca

SEQ ID NO: 97 - DNA, 21 bp, Homo sapiens
cctccctacttattcccatgc

SEQ ID NO: 98 - DNA, 20 bp, Homo sapiens
aaatgcaagcaaagccaagt

SEQ ID NO: 99 - DNA, 20 bp, Homo sapiens
gctccacacacagaggtcaa

SEQ ID NO: 100 - DNA, 21 bp, Homo sapiens
cctcccaaacacacagagttg

SEQ ID NO: 101 - DNA, 23 bp, Homo sapiens
tgttaaacctaagggttccttcc

SEQ ID NO: 102 - DNA, 23 bp, Homo sapiens
ccaatagccttcaatgtatcaaa

SEQ ID NO: 103 - DNA, 20 bp, Homo sapiens
tgaggaagagccacaacaga

SEQ ID NO: 104 - DNA, 22 bp, Homo sapiens
cagagagacagaaatggtctca

SEQ ID NO: 105 - DNA, 20 bp, Homo sapiens
ttcttaacacgcagcacatt

SEQ ID NO: 106 - DNA, 21 bp, Homo sapiens
gccctatttcctaacacatgc

SEQ ID NO: 107 - DNA, 22 bp, Homo sapiens
gctaacatgctaatgtgcttcc

SEQ ID NO: 108 - DNA, 19 bp, Homo sapiens
aaacaatcaaaggcccagg

SEQ ID NO: 109 - DNA, 20 bp, Homo sapiens
cccattggaaacagagttga

SEQ ID NO: 110 - DNA, 20 bp, Homo sapiens
caaggagggtggatcacttg

SEQ ID NO: 111 - DNA, 20 bp, Homo sapiens
agaggctccaaagggagatt

SEQ ID NO: 112 - DNA, 22 bp, Homo sapiens
ccctaaatgcagatggttatga

SEQ ID NO: 113 - DNA, 21 bp, Homo sapiens
gcttgtgctatctgtcccttg

SEQ ID NO: 114 - DNA, 21 bp, Homo sapiens
tgcacaaagctgttctacaca

SEQ ID NO: 115 - DNA, 20 bp, Homo sapiens
actgcttccagccagacatt

SEQ ID NO: 116 - DNA, 20 bp, Homo sapiens
ctgcactcccaagacagaca

SEQ ID NO: 117 - DNA, 20 bp, Homo sapiens
gttgaagcaggctttctgga

SEQ ID NO: 118 - DNA, 20 bp, Homo sapiens
cagcaaccgtttcctttcat

SEQ ID NO: 119 - DNA, 20 bp, Homo sapiens
tttgaggttggtgtcactgg

SEQ ID NO: 120 - DNA, 20 bp, Homo sapiens
acatttcccgtatcgtccaa

SEQ ID NO: 121 - DNA, 18 bp, Homo sapiens
aatgggctggcacagaaa

SEQ ID NO: 122 - DNA, 20 bp, Homo sapiens
gctgggatcttctcagccta

SEQ ID NO: 123 - DNA, 20 bp, Homo sapiens
gctgcaaattgcttggtatg

SEQ ID NO: 124 - DNA, 20 bp, Homo sapiens
tcagtcctatgctgcctcct

SEQ ID NO: 125 - DNA, 22 bp, Homo sapiens
atgggctattgtgtaagcctct

SEQ ID NO: 126 - DNA, 21 bp, Homo sapiens
tccctaccacacctacatcca

SEQ ID NO: 127 - DNA, 18 bp, Homo sapiens
ctgcgtcggccagattac

SEQ ID NO: 128 - DNA, 20 bp, Homo sapiens
attcaagcccggtaacacag

SEQ ID NO: 129 - DNA, 20 bp, Homo sapiens
ctgacagttgatgcccagtc

SEQ ID NO: 130 - DNA, 24 bp, Homo sapiens
aaacacacattggatttcagagac

SEQ ID NO: 131 - DNA, 19 bp, Homo sapiens
gctgggcaacaggtgagac

SEQ ID NO: 132 - DNA, 19 bp, Homo sapiens
atgcttcctgccctcagac

SEQ ID NO: 133 - DNA, 20 bp, Homo sapiens
tcctgcctcagcctctgtat

SEQ ID NO: 134 - DNA, 20 bp, Homo sapiens
gcctctggagtggctaggat

SEQ ID NO: 135 - DNA, 20 bp, Homo sapiens
atgagatggccaggtcaaag

SEQ ID NO: 136 - DNA, 20 bp, Homo sapiens
cggtccaacatggtgaaata

SEQ ID NO: 137 - DNA, 20 bp, Homo sapiens
ccaaaccgaaacctcaagac

SEQ ID NO: 138 - DNA, 20 bp, Homo sapiens
ctcgctctgcagtcttggtt

SEQ ID NO: 139 - DNA, 19 bp, Homo sapiens
catggtgaaagggcaactg

SEQ ID NO: 140 - DNA, 20 bp, Homo sapiens
agcaagaagggagaggtgtg

SEQ ID NO: 141 - DNA, 20 bp, Homo sapiens
tggccacatccctttaaatc

SEQ ID NO: 142 - DNA, 26 bp, Homo sapiens
tgttgaattcattctctaaccacttc

SEQ ID NO: 143 - DNA, 23 bp, Homo sapiens
tgatcatgaaacagtcaacgtct

SEQ ID NO: 144 - DNA, 20 bp, Homo sapiens
gcccactgtccaattaagga

SEQ ID NO: 145 - DNA, 20 bp, Homo sapiens
tctacagcctcacaccgaag

SEQ ID NO: 146 - DNA, 20 bp, Homo sapiens
tgtgggtttacatgccagaa

SEQ ID NO: 147 - DNA, 22 bp, Homo sapiens
gatcccactctgtcactccttt

SEQ ID NO: 148 - DNA, 20 bp, Homo sapiens
tgggtgcctgtagtcctagc

SEQ ID NO: 149 - DNA, 20 bp, Homo sapiens
cttggccttgttcacaggag

SEQ ID NO: 150 - DNA, 22 bp, Homo sapiens
tttctatggcaagttgctgttt

SEQ ID NO: 151 - DNA, 20 bp, Homo sapiens
aggatgcacaagcctgattt

SEQ ID NO: 152 - DNA, 20 bp, Homo sapiens
ttggccatagctccaacttc

SEQ ID NO: 153 - DNA, 26 bp, Homo sapiens
tctccaaattccagttctactacttt

SEQ ID NO: 154 - DNA, 24 bp, Homo sapiens
tttctctttcctgctttgtctctt

SEQ ID NO: 155 - DNA, 20 bp, Homo sapiens
aaatctggccatcctcctct

SEQ ID NO: 156 - DNA, 19 bp, Homo sapiens
aatcctgtcccaggcagac

SEQ ID NO: 157 - DNA, 20 bp, Homo sapiens
ccctgaactcaggaacaagc

SEQ ID NO: 158 - DNA, 20 bp, Homo sapiens
caaagccgtgtctttccttc

SEQ ID NO: 159 - DNA, 20 bp, Homo sapiens
gggatagcccatggatagga

SEQ ID NO: 160 - DNA, 22 bp, Homo sapiens
tgaattgttgcacaaataaagg

SEQ ID NO: 161 - DNA, 22 bp, Homo sapiens
tgggaagaataagaggtccaga

SEQ ID NO: 162 - DNA, 20 bp, Homo sapiens
tcagttcagctgtccagcaa

SEQ ID NO: 163 - DNA, 20 bp, Homo sapiens
gggcatagtgctttctgctt

SEQ ID NO: 164 - DNA, 22 bp, Homo sapiens
tgatgcattcctttattctcca

SEQ ID NO: 165 - DNA, 20 bp, Homo sapiens
ccaagctctcttctggcttc

SEQ ID NO: 166 - DNA, 20 bp, Homo sapiens
ttgcatcccatctttccttc

SEQ ID NO: 167 - DNA, 20 bp, Homo sapiens
tggtgaagggactcttcctg

SEQ ID NO: 168 - DNA, 20 bp, Homo sapiens
cccatggtagaactggcaaa

SEQ ID NO: 169 - DNA, 23 bp, Homo sapiens
ttctctccagattgatacacagc

SEQ ID NO: 170 - DNA, 21 bp, Homo sapiens
tggccatatagtaagccttgg

SEQ ID NO: 171 - DNA, 20 bp, Homo sapiens
tccacctatccaagcaacaa

SEQ ID NO: 172 - DNA, 22 bp, Homo sapiens
tgtagtgatatgccaatgtggt

SEQ ID NO: 173 - DNA, 22 bp, Homo sapiens
tttccaaaccaaggtcagattt

SEQ ID NO: 174 - DNA, 20 bp, Homo sapiens
gccctgcttcagtgaatgtt

SEQ ID NO: 175 - DNA, 20 bp, Homo sapiens
tccatgcacagaaacattca

SEQ ID NO: 176 - DNA, 26 bp, Homo sapiens
tcatttattactttgcatttggctta

SEQ ID NO: 177 - DNA, 21 bp, Homo sapiens
cagtcacgtagagagcagcag

SEQ ID NO: 178 - DNA, 19 bp, Homo sapiens
ctgggccacagagtgagac

SEQ ID NO: 179 - DNA, 20 bp, Homo sapiens
gagcagcagtaatcccgaat

SEQ ID NO: 180 - DNA, 20 bp, Homo sapiens
ggcagaagaatcgcttgaac